(19) United States
(12) Patent Application Publication
(10) Pub. No.: US 2021/0355058 A1
(43) Pub. Date: Nov. 18, 2021

(54) POLYCYCLIC COMPOUNDS

(71) Applicant: MITSUBISHI GAS CHEMICAL COMPANY, INC., Tokyo (JP)

(72) Inventors: Munenori Shiratake, Ibaraki (JP); Kentaro Ishihara, Tokyo (JP); Koji Hirose, Tokyo (JP); Shinya Ikeda, Niigata (JP); Noriyuki Kato, Tokyo (JP); Mitsuteru Kondo, Tokyo (JP); Shoko Murata, Tokyo (JP); Kensuke Oshima, Ibaraki (JP); Florian Stolz, Freiburg (DE)

(21) Appl. No.: 17/286,353

(22) PCT Filed: Oct. 18, 2019
(86) PCT No.: PCT/JP2019/042232

§ 371 Date: Apr. 16, 2021

(30) Foreign Application Priority Data
Oct. 19, 2018 (JP) 2018-197436
Dec. 28, 2018 (JP) 2018-247874

(51) Int. Cl. C07C 43/295; C08G 64/06

(52) U.S. Cl. CPC C07C 43/295; C07C 2603/18; C07C 2602/10; C08G 64/06

(57) ABSTRACT

The present invention relates to compounds of the formula (I), which are suitable as monomers for preparing thermoplastic resins having beneficial optical properties and which can be used for producing optical devices: where A¹, A² are selected from mono- or bicyclic aromatic radicals and mono- or bicyclic heteroaromatic radicals, X represents e.g. a single bond, O, NH, CR⁶R⁷, Y is e.g. absent or represents a single bond, O, NH, CR⁸R⁹; R¹, R² are hydrogen, a radical Ar′ or a radical R^{a}; R³ is Alk, O-Alk′-, O-Alk′-[O-Alk′]₀, O—CH₂—Ar—C(O)—, O—C(O)—Ar—C(O)— or O-Alk-C(O)—, where in the last five moieties the left O is bound to A¹ and A², respectively, m, n are 0, 1 or 2; o is an integer from 1 to 10; R⁴, R⁵ are e.g. selected from CN and a radical R^{a}; R⁶, R⁸ are e.g. selected from hydrogen, a radical Ar′ and a radical R^{a}; R⁷, R⁹ are e.g. selected from hydrogen, C₁-C₄-alkyl and a radical Ar′; R^{a} is selected from the group consisting of C≡C—R¹¹ and Ar—C≡C—R¹¹; R¹¹ is e.g. selected from hydrogen, methyl, mono- or polycyclic aryl having from 6 to 26 carbon atoms and mono- or polycyclic hetaryl having a total of 5 to 26 atoms, which are ring members, where 1, 2, 3 or 4 of the ring atoms of hetaryl are selected from nitrogen, sulphur and oxygen, while the remainder of these atoms are carbon atoms, where mono- or polycyclic aryl are unsubstituted or substituted; and Ar is e.g. phenylene or naphthylene. The invention also relates to thermoplastic resins comprising a polymerized unit of the compound of formula (I) and to optical devices made of such resins.

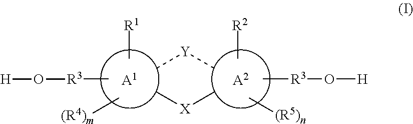

The present invention relates to polycyclic compounds that are suitable as monomers for preparing thermoplastic resins, such as polycarbonate resins, which have beneficial optical properties and can be used for producing optical devices.

### BACKGROUND OF INVENTION

Optical glass or optical resins are frequently used as a material for an optical lens in optical systems of any of various types of cameras such as a camera, a camera having a film integrated therewith, a video camera and the like. While optical glass is beneficial in heat resistance, transparency, size stability, chemical resistance and the like, its material costs are high. Moreover the moldability is low and thus mass production is difficult.

Optical devices, such as optical lenses, made of optical resin instead of optical glass are advantageous in that they can be produced in large numbers by injection molding. Nowadays, optical resins, in particular, transparent polycarbonate resins, are frequently used for producing camera lenses. In this regard, resins with a higher refractive index are highly desirable, as they allow for reducing the size and weight of final products. In general, when using an optical material with a higher refractive index, a lens element of the same refractive power can be achieved with a surface having less curvature, so that the amount of aberration generated on this surface can be reduced. As a result, it is possible to reduce the number of lenses, to reduce the eccentric sensitivity of lenses and/or to reduce the lens thickness to thereby achieve weight reduction.

In an optical system of a camera, the aberration correction is usually performed by a combination of a plurality of concave and convex lenses. More specifically, a convex lens having a color aberration is combined with a concave lens having a color aberration of an opposite sign to that of the convex lens, so that the color aberration of the convex lens is synthetically cancelled. In this case it is required that the concave lens is highly dispersive, i.e. it must have a low Abbe number.

EP2034337 describes a copolycarbonate resin which comprises 99 to 51 mol % of a repeating unit derived from 9,9-bis(4-(2-hydroxyethoxy)phenyl)fluorene and 1 to 49 mol % of a repeating unit derived from bisphenol A. The resin is suitable for preparing an optical lens having a low Abbe number of 23 to 26 and a refractive index from 1.62 to 1.64.

JP H06-25398 discloses a copolycarbonate resin including a repeating unit derived from 9,9-bis(4-hydroxyphenyl)fluorene and a repeating unit derived from bisphenol A.

In an example of this document, it is described that the refractive index reaches 1.616 to 1.636.

U.S. Pat. No. 9,360,593 describes polycarbonate resins having repeating units derived from binaphthyl monomers of the formula (1):

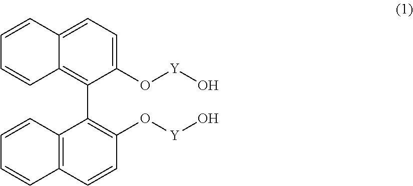

where Y is C₁-C₄-alkandiyl, in particular 1,2-ethandiyl. It is said that the polycarbonate resins have beneficial optical properties in terms of a high refractive index, a low Abbe's number, a high degree of transparency, low birefringence, and a glass transition temperature suitable for injection molding.

Co-Polycarbonates of monomers of the formula (1) with 10,10-bis(4-hydroxyphenyl)-anthrone monomers and their use for preparing optical lenses are described in US 2016/0319069. The copolycarbonates are reported to have a good moisture resistance. Refractive indices of about 1.662 to 1.667 have been reported.

So far, thermoplastic resin, such as a polycarbonate resins having a high refractive index and a low Abbe number have not been provided yet. Moreover, various electronic devices should have high moisture resistance and heat resistance. A “PCT test” (pressure cooker test” has been established to evaluate the moisture resistance and the heat resistance of such electronic devices. In this test, penetration of moisture into a sample is increased for a certain time period to evaluate the moisture resistance and the heat resistance. Therefore, an optical lens formed of an optical resin useable for an electronic device needs to have a high refractive index and a low Abbe number, and is also required to maintain high optical properties even after the PCT test.

Despite the advances made in the field of optical resins, there is still an ongoing need for monomers for preparing optical resins, in particular polycarbonate resins, which monomers result in a high refractive index, in particular which provide for a higher refractive index than the monomers of formula (1). Apart from that, the monomers should not impair the other optical properties of the optical resins, such as low Abbe's number, a high degree of transparency and low birefringence. Moreover, the monomers should be easy to prepare. The resins obtained from these monomers should have also a good moisture and heat resistance and they should have a glass transition temperature suitable for injection molding.

### SUMMARY OF INVENTION

It was surprisingly found that compounds of the formula (I) as described herein are suitable for preparing optical resins of high transparency and high refractive index. In particular, when used as monomers in the preparation of optical resins, compounds of the formula (I) result in higher refractive indices than the monomers of formula (1).

Therefore, the present invention relates to compounds of the formula (I)

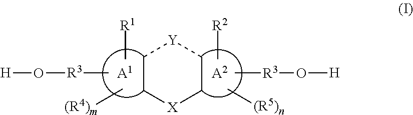

where
A¹, A² are selected from mono- or bicyclic aromatic radicals and mono- or bicyclic heteroaromatic radicals,
X represents a single bond, O, NH, CR⁶R⁷ or a moiety of the formula A,
Y is absent or represents a single bond, O, NH, CR⁸R⁹ or a moiety of the formula A,

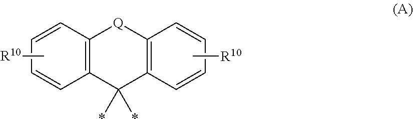

where
* indicate the points of attachment to the ring carbon atoms of A¹ and A², respectively, and
Q is absent or represents a single bond, O, NH, C═O, CH₂ or CH═CH;
R¹, R² are hydrogen, a radical Ar′ or a radical R^{a};
R³ is Alk, O-Alk′-, O-Alk′-[O-Alk′]₀, O—CH₂—Ar—C(O)—, O—C(O)—Ar—C(O)— or O-Alk-C(O)—, where in the last four moieties the left O is bound to A¹ and A², respectively,
m, n are 0, 1 or 2;
is an integer from 1 to 10;
R⁴, R⁵ are selected from the group consisting of fluorine, CN, R, OR, CH_{w}R_{3-w}, NR₂, C(O)R, C(O)NH_{2I} a radical Ar′ and a radical R^{a};
R⁶ is selected from the group consisting of hydrogen, a radical Ar′ and a radical R^{a};
R⁷ is selected from the group consisting of hydrogen, C₁-C₄-alkyl and a radical Ar′;
R⁸ is selected from the group consisting of hydrogen, a radical Ar′ and a radical R^{a},
R⁹ is selected from the group consisting of hydrogen, C₁-C₄-alkyl and a radical Ar′;
R¹⁰ is selected from the group consisting of hydrogen, fluorine, CN, R, OR, CHₖR₃₋ₖ, NR₂, C(O)R, C(O)NH₂ and a radical R^{a};
R^{a} is selected from the group consisting of C≡C—R¹¹ and Ar—C≡C—R¹¹;
R¹¹ is selected from hydrogen, methyl, mono- or polycyclic aryl having from 6 to 26 carbon atoms and mono- or polycyclic hetaryl having a total of 5 to 26 atoms, which are ring members, where 1, 2, 3 or 4 of the ring atoms of hetaryl are selected from nitrogen, sulphur and oxygen, while the remainder of these atoms are carbon atoms, where mono- or polycyclic aryl are unsubstituted or substituted by 1, 2, 3 or 4 identical or different radicals R¹²; R¹² is selected from fluorine, phenyl, CN, OCH₃, CH₃, N(CH₃)₂, C(O)CH₃, C≡CH, C≡C—CH₃, CH₂—C≡CH and CH₂—C≡C—CH₃;
Alk is C₁-C₄-alkandiyl, where 1 or 2 of the hydrogen atoms of C₁-C₄-alkandiyl may be replaced by Ar′;
Alk′ is selected from the group consisting of C₂-C₄-alkandiyl, where 1 or 2 of the hydrogen atoms of C₁-C₄-alkandiyl may be replaced by Ar′, and CH₂—Ar—CH₂;
Ar is a bivalent radical selected from phenylene and naphthylene, which are unsubstituted or carry 1, 2, 3 or 4 radicals R^{Ar};
Ar′ is selected from the group consisting of mono- or polycyclic aryl having from 6 to 26 carbon atoms and mono- or polycyclic hetaryl having a total of 5 to 26 atoms, which are ring members, where 1, 2, 3 or 4 of these atoms are selected from nitrogen, sulphur and oxygen, while the remainder of these atoms are carbon atoms, where mono- or polycyclic aryl and mono- or polycyclic hetaryl are unsubstituted or carry 1, 2, 3 or 4 radicals R^{Ar};
R^{Ar} is selected from the group consisting of fluorine, bromine, chlorine, CN, R, OR, CHₖR₃₋ₖ, NR₂, C(O)R, C(O)NH₂ and a radical R^{a}, it being possible that R^{Ar} is identical or different, if more than 1 is present on each ring;
R is selected from methyl, mono- or polycyclic aryl having from 6 to 26 carbon atoms and mono- or polycyclic hetaryl having a total of 5 to 26 atoms, which are ring members, where 1, 2, 3 or 4 of the ring atoms of hetaryl are selected from nitrogen, sulphur and oxygen, while the remainder of these atoms are carbon atoms, where mono- or polycyclic aryl are unsubstituted or substituted by 1, 2, 3 or 4 identical or different radicals R¹²;
w on each occurrence is 0, 1, 2 or 3;
k on each occurrence is 0, 1, 2 or 3;

and, if R³ is O—CH₂—Ar—C(O)—, O—C(O)—Ar—C(O)— or O-Alk-C(O)—, the esters thereof, in particular the C₁-C₄-alkyl esters thereof;

provided that the compound of formula (I) bears at least 1 radical R^{a} and in particular 2 to 4 radicals R^{a}.

The above compounds are particularly useful in the preparation of thermoplastic resins, in particular for optical resins as defined herein, especially for polycarbonate resins.

When used as monomers for the preparation of optical resins, in particular polycarbonate resins, the compounds of the formula (I) provide for higher refractive indices of the resins than the monomers of the formula (1). Moreover, Compounds of formula (I) provide for high transparency of the resins and they do not significantly impair other optical properties and the mechanical properties of the resins. In particular, these resins fulfil the other requirements of optical resins, such as low Abbe's number, a high degree of transparency and low birefringence. Apart from that, the monomers of formula (I) can be easily prepared and obtained in high yields and high purity. In particular, the compounds of formula (I) can be obtained in crystalline form, which allows for an efficient purification to the degree required in the preparation of optical resins. In particular, the compounds of formula (I) can be obtained in a purity which provides for low haze, which is in particular important for the use in the preparation of optical resins. Compounds of formula (I), which do not bear color-imparting radicals, such as some of the radicals R¹¹, Ar′ and R, can also be obtained in a purity, which provides for a low yellowness index Y.I., as determined in accordance with ASTM E313, which may also be important for the use in the preparation of optical resins.

The invention also relates to a thermoplastic resin comprising a polymerized unit of the compounds of formula (I), i.e. a thermoplastic resin comprising a structural unit represented by formula (II) below.

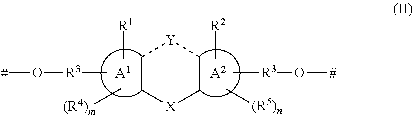

where

#represents a connection point to a neighboring structural unit;

and where A¹, A², n, m, R¹, R², R³, R⁴, R⁵, X and Y are as defined herein.

The invention further relates to a thermoplastic resin selected from copolycarbonate resins and copolyester resins, where the thermoplastic resin in addition to the structural units of formula (II) also comprises structural units of the formula (V),

#—O—R^{z}-A³-R^{z}—O—#- (V)

where
#represents a connection point to a neighboring structural unit;
A³ is a polycyclic radical bearing at least 2 benzene rings, wherein the benzene rings may be connected by A′ and/or directly fused to each other and/or fused by a non-benzene carbocycle, where A³ is unsubstituted or substituted by 1, 2 or 3 radicals R^{aa}, which are selected from the group consisting of halogen, C₁-C₆-alkyl, C₅-C₆-cycloalkyl and phenyl;
A′ is selected from the group consisting of a single bond, O, C═O, S, SO₂, CH₂, CH—Ar″, CHAr″₂, CH(CH₃), C(CH₃)₂ and a radical A″

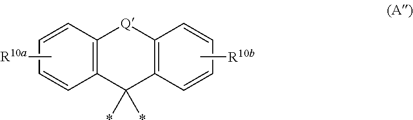

where
Q′ represents a single bond, O, NH, C═O, CH₂ or CH═CH; and
R^{10a}, R^{10b}, independently of each other are selected from the group consisting of hydrogen, fluorine, CN, R, OR, CHₖR₃₋ₖ, NR₂, C(O)R and C(O)NH₂;
Ar″ is selected from the group consisting of mono- or polycyclic aryl having from 6 to 26 carbon atoms and mono- or polycyclic hetaryl having a total of 5 to 26 atoms, which are ring members, where 1, 2, 3 or 4 of these atoms are selected from nitrogen, sulphur and oxygen, while the remainder of these atoms are carbon atoms, where Ar″ is unsubstituted or substituted by 1, 2 or 3 radicals R^{ab}, which are selected from the group consisting of halogen, phenyl and C₁-C₄-alkyl;
R^{z} is a single bond, Alk¹, O-Alk²-, O-Alk²-[O-Alk²-]ₚ- or O-Alk³-C(O)— where O is bound to A³, and where
p is an integer from 1 to 10;
Alk¹ is C₁-C₄-alkandiyl;
Alk² is C₂-C₄-alkandiyl
Alk³ is C₁-C₄-alkandiyl.

The invention further relates to an optical device made of a thermoplastic resin as defined above.

### DETAILED DESCRIPTION OF INVENTION

If X is a single bond and Y is absent the compounds of formula (I) may have axial chirality, due to the limited rotation along the bond between the moieties A¹ and A². In that case the compounds of the formula (I) may therefore exist in the form of their (S)-enantiomer and their (R)-enantiomer. Consequently, the compounds of formula (I) may exist as a racemic mixture or as non-racemic mixtures or in the form of their pure (S)- and (R)-enantiomers, respectively. The present invention relates to both the racemic and the non-racemic mixtures of the enantiomers of the compounds of formula (I), where X is a single bond and Y is absent, and also to their pure (S)- and (R)-enantiomers.

In terms of the present invention, the term “C₁-C₄-alkandiyl group” is alternatively also designated “alkylene group having 1, 2, 3 or 4 carbon atoms” and refers to a bivalent, saturated, aliphatic hydrocarbon radical having 1, 2, 3 or 4 carbon atoms. Examples of C₁-C₄-alkandiyl are in particular linear alkandiyl such as methandiyl (CH₂), 1,2-ethandiyl (CH₂CH₂), 1,3-propandiyl (CH₂CH₂CH₂) and 1,4-butandiyl (CH₂CH₂CH₂CH₂), but also branched alkandiyl such as 1-methyl-1,2-ethandiyl, 1-methyl-1,2-propandiyl, 2-methyl-1,2-propandiyl, 2-methyl-1,3-propandiyl and 1,3-butandiyl.

In terms of the present invention, the terms “monocyclic aromatic radical” and “monocyclic aryl” refer to phenyl and, in case of a bivalent radical, to phenylene, such as 1,2-, 1,3- or 1,4-phenylene.

In terms of the present invention, the term “bicyclic aromatic radical” refers to naphthyl, and, in case of a bivalent radical, to naphthylene, such as 1,2-, 1,3-, 1,4-, 1,5-, 1,6-, 1,7-, 1,8-, 2,3-, 2,6- and 2,7-naphthylene.

In terms of the present invention, the terms “monocyclic heteroaromatic radical” and “monocyclic hetaryl” refer to a mono- or bivalent heteroaromatic monocyclic radical, where the ring member atoms are part of a conjugate π-electron system, where the heteroaromatic monocycle has 5 or 6 ring atoms, which comprise as heterocyclic ring members 1, 2, 3 or 4 nitrogen atoms or 1 oxygen atom and 0, 1, 2 or 3 nitrogen atoms, or 1 sulphur atom and 0, 1, 2 or 3 nitrogen atoms, where the remainder of the ring atoms are carbon atoms. Examples include furyl (=furanyl), pyrrolyl (=1H-pyrrolyl), thienyl (=thiophenyl), imidazolyl (=1H-imidazolyl), pyrazolyl (=1H-pyrazolyl), 1,2,3-triazolyl, 1,2,4-triazolyl, tetrazolyl, oxazolyl, thiazolyl, isoxazolyl, isothiazolyl, 1,3,4-oxadiazolyl, 1,3,4-thiadiazolyl, pyridyl (=pyridinyl), pyrazinyl, pyridazinyl, pyrimidinyl and triazinyl.

In terms of the present invention, the term “bicyclic heteroaromatic radical” refers to mono- or bivalent bicyclic hetaryl radicals, which bear a monocyclic hetaryl ring as defined above and one further aromatic ring selected from phenyl and heteroaromatic monocycles as defined above, where the aromatic rings of bicyclic hetaryl are fused to each other. Examples include benzofuryl, isobenzofuryl, benzothienyl, isobenzothienyl, furo[3,2-b]furanyl, thieno[3,2-b]thienyl, furo[2,3-b]furanyl, thieno[2,3-b]thienyl, furo[3,4-b]furanyl, thieno[3,4-b]thienyl, indolyl (=1H-indolyl), isoindolyl (=2H-isoindolyl), indolizinyl, benzopyrazolyl, benzimidazolyl, benzoxazolyl, benzisoxazolyl, benzothiazolyl, benzo[cd]indolyl, 1H-benzo[g]indolyl, quinolinyl, isoquinolinyl, quinazolinyl, quinoxalinyl, cinnolinyl, 1,5-naphthyridinyl, 1,8-naphthyridinyl, pyrrolo[3,2-b]pyridinyl, pteridinyl and puryl.

In terms of the present invention, the term “polycyclic aryl” refers to

(i) a mono- or bivalent aromatic polycyclic hydrocarbon radical, i.e. a completely unsaturated polycyclic hydrocarbon radical, where each of the carbon atoms is part of a conjugate π-electron system,

(ii) a mono- or bivalent polycyclic hydrocarbon radical which bears 1 phenyl ring which is fused to a saturated or unsaturated 4 to 10-membered mono- or bicyclic hydrocarbon ring,

(iii) a mono- or bivalent polycyclic hydrocarbon radical which bears at least 2 phenyl rings which are linked to each other by a covalent bond or which are fused to each other directly and/or which are fused to a saturated or unsaturated 4 to 10-membered mono- or bicyclic hydrocarbon ring.

Usually polycyclic aryl has from 9 to 26, e.g. 9, 10, 12, 13, 14, 16, 17, 18, 19, 20, 22, 24, 25 or 26 carbon atoms, in particular from 10 to 20 carbon atoms, especially 10, 12, 13, 14 or 16 carbon atoms.

In this context, polycyclic aryl bearing 2, 3 or 4 phenyl rings which are linked to each other via a single bond include e.g. biphenylyl and terphenylyl. Polycyclic aryl bearing 2, 3 or 4 phenyl rings which are directly fused to each other include e.g. naphthyl, anthracenyl, phenanthrenyl, pyrenyl and triphenylenyl. Polycyclic aryl bearing 2, 3 or 4 phenyl rings which are fused to a saturated or unsaturated 4- to 10-membered mono- or bicyclic hydrocarbon ring include e.g. 9H-fluorenyl, biphenylenyl, tetraphenylenyl, acenaphthenyl (1,2-dihydroacenaphthylenyl), acenaphthylenyl, 9,10-dihydroanthracen-1-yl, 1,2,3,4-tetrahydrophenanthrenyl, 5,6,7,8-tetrahydrophenanthrenyl, cyclopent[fg]acenaphthylenyl, phenalenyl, fluoranthenyl, benzo[k]fluoranthenyl, perylenyl, 9,10-dihydro-9,10[1′,2′]-benzenoanthracenyl, dibenzo[a,e][8]annulenyl, 9,9′-spirobi[9H-fluoren]yl and spiro[1H-cyclobuta[de]naphthalene-1,9′-[9H]fluoren]yl.

Polycyclic aryl includes, by way of example naphthyl, 9H-fluorenyl, phenanthryl, anthracenyl, pyrenyl, acenaphthenyl, acenaphthylenyl, 2,3-dihydro-1H-indenyl, 5,6,7,8-tetrahydro-naphthalenyl, cyclopent[fg]acenaphthylenyl, 2,3-dihydrophenalenyl, 9,10-dihydroanthracen-1-yl, 1,2,3,4-tetrahydrophenanthrenyl, 5,6,7,8-tetrahydrophenanthrenyl, fluoranthenyl, benzo[k]fluoranthenyl, biphenylenyl, triphenylenyl, tetraphenylenyl, 1,2-dihydroacenaphthylenyl, dibenzo[a,e][8]annulenyl, perylenyl, biphenylyl, terphenylyl, naphthylenphenyl, phenanthrylphenyl, anthracenylphenyl, pyrenylphenyl, 9H-fluorenylphenyl, di(naphthylen)phenyl, naphthylenbiphenyl, tri(phenyl)phenyl, tetra(phenyl)phenyl, pentaphenyl(phenyl), phenylnaphthyl, binaphthyl, phenanthrylnaphthyl, pyrenylnaphthyl, phenylanthracenyl, biphenylanthracenyl, naphthalenylanthracenyl, phenanthrylanthracenyl, dibenzo[a,e][8]annulenyl, 9,10-dihydro-9,10[1′,2′]benzoanthracenyl, 9,9′-spirobi-9H-fluorenyl and spiro[1H-cyclobuta[de]naphthalene-1,9′-[9H]fluoren]yl.

In terms of the present invention, the term “polycyclic hetaryl” refers to mono- or bivalent heteroaromatic polycyclic radicals, which bear a monocyclic hetaryl ring as defined above and at least one, e.g. 1, 2, 3, 4 or 5, further aromatic rings selected from phenyl and heteroaromatic monocycles as defined above, where the aromatic rings of polycyclic hetaryl are linked to each other by a covalent bond and/or fused to each other directly and/or fused to a saturated or unsaturated 4 to 10-membered mono- or bicyclic hydrocarbon ring. The term “polycyclic hetaryl” also refers to heteroaromatic polycyclic radicals, which bear at least one saturated or partially unsaturated 5- or 6-membered heterocyclic ring bearing 1 or 2 heteroatoms selected from oxygen, sulphur and nitrogen as ring atoms, such as 2H-pyran, 4H-pyran, thiopyran, 1,4-dihydropyridin, 4H-1,4-oxazin 4H-1,4-thiazin or 1,4-dioxin, and at least one, e.g. 1, 2, 3, 4 or 5, further aromatic rings selected from phenyl and heteroaromatic monocycles, where at least one of the further aromatic rings is directly fused to the saturated or partially unsaturated 5- or 6-membered heterocyclic radical and where the remainder of further aromatic rings of polycyclic hetaryl are linked to each other by a covalent bond or fused to each other directly and/or fused to a saturated or unsaturated 4 to 10-membered mono- or bicyclic hydrocarbon ring. Usually polycyclic hetaryl has 9 to 26 ring atoms in particular 9 to 20 ring atoms, which comprise 1, 2, 3 or 4 atoms selected from nitrogen atoms, sulphur atoms and oxygen atoms, where the remainder of the ring atoms are carbon atoms.

Examples of polycyclic hetaryl include, but are not limited to, benzofuryl, benzothienyl, dibenzofuranyl (=dibenzo[b,d]furanyl), dibenzothienyl (=dibenzo[b,d]thienyl), naphthofuryl, naphthothienyl, furo[3,2-b]furanyl, furo[2,3-b]furanyl, furo[3,4-b]furanyl, thieno[3,2-b]thienyl, thieno[2,3-b]thienyl, thieno[3,4-b]thienyl, oxanthrenyl, thianthrenyl, indolyl (=1H-indolyl), isoindolyl (=2H-isoindolyl), carbazolyl, indolizinyl, benzopyrazolyl, benzimidazolyl, benzoxazolyl, benzothiazolyl, benzo[cd]indolyl, 1H-benzo[g]indolyl, quinolinyl, isoquinolinyl, acridinyl, phenazinyl, quinazolinyl, quinoxalinyl, phenoxazinyl, phenothiazinyl, benzo[b][1,5]naphthyridinyl, cinnolinyl, 1,5-naphthyridinyl, 1,8-naphthyridinyl, phenylpyrrolyl, naphthylpyrrolyl, dipyridyl, phenylpyridyl, naphthylpyridyl, pyrido[4,3-b]indolyl, pyrido[3,2-b]indolyl, pyrido[3,2-g]quinolinyl, pyrido[2,3-b][1,8]naphthyridinyl, pyrrolo[3,2-b]pyridinyl, pteridinyl, puryl, 9H-xanthenyl, 9H-thioxanthenyl, 2H-chromenyl, 2H-thiochromenyl, phenanthridinyl, phenanthrolinyl, furo[3,2-f][1]benzofuranyl, furo[2,3-f][1]benzofuranyl, furo[3,2-g]quinolinyl, furo[2,3-g]quinolinyl, furo[2,3-g]quinoxalinyl, benzo[g]chromenyl, thieno[3,2-f][1]benzothienyl, thieno[2,3-f][1]benzothienyl, thieno[3,2-p]quinolinyl, thieno[2,3-p]quinolinyl, thieno[2,3-g]quinoxalinyl, benzo[g]thiochromenyl, pyrrolo[3,2,1-hi]indolyl, benzo[g]quinoxalinyl, benzo[f]quinoxalinyl, and benzo[h]isoquinolinyl.

In terms of the present invention, the term “optical device” refers to a device that is transparent for visible light and manipulates light beams, in particular by refraction.

Optical devices include but are not limited to prisms, lenses and combinations thereof, especially lenses for cameras and lenses for glasses.

In terms of the present invention, the phrase “if R³ is O—CH₂—Ar—C(O)—, O—C(O)—Ar—C(O)— or O-Alk-C(O)—, the esters thereof, in particular the C₁-C₄-alkyl esters thereof” is understood that the hydroxyl group of R³—OH together with the group C(O)— forms a carboxyl group which may be esterified with an alcohol, in particular with an aliphatic alcohol, more particularly with a C₁-C₄-alkanol such as methanol, ethanol, n-propanol, isopropanol, n-butanol, sec-butanol, isobutanol or tert.-butanol.

The remarks made below as to preferred embodiments of the variables (substituents) of the compounds of formula (I) and of the structural units of formula (II) are valid on their own as well as preferably in combination with each other, as well as in combination with the stereoisomers thereof.

The remarks made below concerning preferred embodiments of the variables further are valid on their own as well as preferably in combination with each other concerning the compounds of formula (I) and the structural units of formula (II), where applicable, as well as concerning the uses and methods according to the invention and the composition according to the invention.

In formula (I) and likewise in formula (II), the variables A¹, A², X, Y, R^{a}, R¹, R², R³, R⁴, R⁵, m and n on their own or preferably in any combination preferably have the following meanings:

Preferably the variables A¹ and A² in formulae (I) and (II) are independently of one another selected from phenylene, naphthylene, pyridindiyl, pyrazindiyl, pyridazindiyl, pyrimidindiyl, quinolindiyl, isoquinolindiyl, quinazolindiyl, quinoxalindiyl, cinnolindiyl, benzofurandiyl, isobenzofurandiyl, benzothiophendiyl, isobenzothiophendiyl, indoldiyl and isoindoldiyl, and in particular from phenylene and naphthylene. A¹ and A² may be identical or different. Frequently, A¹ and A² are identical to each other.

In a preferred embodiment of the invention A¹ and A² have identical meanings and are bound in the same position to the moiety X, i.e. in case A¹ and A² are naphthylene, they are both bound either in position 1 or in position 2 to X. According to this embodiment A¹ and A² are particularly selected from 1,2-phenylene, 1,4-phenylene, 1,2-naphthylene, 1,3-naphthylene, 1,4-naphthylene, 2,3-naphthylene, 2,6-naphthylene and 2,7-naphthylene, where the positions of substitution refer to the attachment points of A¹ and A² to X and R³, respectively.

In a group (1) of embodiments of the invention A¹ and A² have identical meanings and are selected from phenylene.

In a group (2) of embodiments of the invention A¹ and A² have identical meanings and are selected from naphthylene.

In a group (3) of embodiments the variables X in formulae (I) and (II) represent a single bond, O, NH, or a moiety of the formula A. In this context the moiety Q in formula A preferably represents a single bond, O, NH, C═O or CH₂, more preferably a single bond, O or C═O, and in particular a single bond, and the two substituents R¹⁰ are preferably both hydrogen or CN or, alternatively, are identical radicals R^{a}. Here, R^{a} is especially C≡C—R¹¹, where R¹¹ is as defined herein. In this group (3) of embodiments the two identical substituents R¹⁰ are particularly selected from hydrogen, CN, 2-phenylethynyl and 2-naphthylethynyl, specifically 2-(1-naphthyl)-ethynyl, and are preferably attached to carbon atoms that are either located in the positions 2 and 7 or in the positions 3 and 6 of the moiety of formula A.

In a group (4) of embodiments the variables Y in formulae (I) and (II) are absent. In this group (4) of embodiments, a subgroup (4′) relates to those compounds where X represents a single bond, and a subgroup (4″) relates to those compounds where X represents a radical of the formula A or a radical CR⁶R⁷ with both R⁶ and R⁷ being Ar′.

In a particular group (4a) of embodiments the variables Y in formulae (I) and (II) are absent, and the variables X represent a radical CR⁶R⁷, provided that R⁶ is different from H if R⁷ is Ar′ further provided that R⁷ is different form H, if R⁶ is Ar′.

In a group (5) of embodiments the variables Y in formulae (I) and (II) represent a single bond, a group CR⁸R⁹ or a moiety of the formula A. In this context the substituent R⁸ is preferably a radical Ar′ or a radical R^{a}, and the substituent R⁹ is preferably hydrogen or C₁-C₄-alkyl, where Ar′ and R^{a} have one of the meanings defined herein, in particular one of the preferred meanings. In particular, R⁹ is hydrogen and R⁸ is a radical Ar′, preferably is phenyl or naphthyl which both may optionally carry one or two substituents R^{Ar} and, in particular, are unsubstituted. Further, in this context, the moiety Q in formula A preferably represents a single bond, O, NH or CH₂, more preferably a single bond or O, and in particular a single bond, and the two substituents R¹⁰ are preferably both hydrogen or, alternatively, are identical radicals R^{a}. Here, R^{a} is especially C≡C—R¹¹, where R¹¹ is as defined herein. In this group (5) of embodiments the two identical substituents R¹⁰ are particularly selected from hydrogen, 2-phenylethynyl and 2-naphthylethynyl, specifically 2-(1-naphthyl)-ethynyl, and are preferably attached to carbon atoms that are either located in the positions 2 and 7 or in the positions 3 and 6 of the moiety of formula A.

In a group (6) of embodiments the radicals R¹ and R² in formulae (I) and (II) are selected independently of one another from hydrogen, mono- and polycyclic aryl and a radical R^{a}. Preferably, R¹ and R² have identical meanings selected from hydrogen, optionally substituted phenyl, optionally substituted naphthyl, phenanthryl, and R^{a}, i.e. C≡C—R¹¹ or Ar—C≡C—R¹¹. In particular, R¹ and R² are selected from hydrogen, phenyl, naphthyl, ethynyl, cyanophenyl, dicyanophenyl, cyanonaphthyl, dicyanonaphthyl, methylethynyl, phenylethynyl, naphthylethynyl, biphenylylethynyl, phenanthrylethynyl, dibenzofuranylethynyl, dibenzothiophenylethynyl, thianthrenylethynyl, triphenylenylethynyl, pyridinylethynyl, quinolinylethynyl, methylethynylphenyl, phenylethynylphenyl, methylethynylnaphthyl, phenylethynylnaphthyl, naphthylethynylphenyl, naphthylethynylnaphthyl, phenanthrylethynylphenyl, biphenylylethynylphenyl, triphenylenylethynyl)phenyl, pyridinylethynylphenyl, quinolinylethynylphenyl, dibenzofuranylethynylphenyl, dibenzothiophenylethynylphenyl and thianthrenylethynylphenyl, and especially from hydrogen, ethynyl, phenyl, 3-cyanophenyl, 4-cyanophenyl, 3,5-dicyanophenyl, 4-cyano-1-naphthyl, 6-cyano-1-naphthyl, 6-cyano-2-naphthyl, 2-phenylethynyl, 2-(1-naphthyl)ethynyl, 2-(2-naphthyl)ethynyl, 2-(2-phenylphenyl)ethynyl, 2-(4-phenylphenyl)ethynyl, 2-(triphenylen-2-yl)ethynyl, 2-(pyridin-2-yl)ethynyl, 2-(pyridin-3-yl)ethynyl, 2-(pyridin-4-yl)ethynyl, 2-(quinoline-2-yl)ethynyl, 2-(quinoline-3-yl)ethynyl, 2-(quinoline-4-yl)ethynyl, 2-(quinoline-8-yl)ethynyl, 2-(9-phenanthryl)ethynyl, 2-(2-dibenzofuranyl)ethynyl, 2-(4-dibenzofuranyl)ethynyl, 2-(2-dibenzothiophenyl)ethynyl, 2-(4-dibenzothiophenyl)ethynyl, 2-(1-thianthrenyl)ethynyl, 2-(2-thianthrenyl)ethynyl, 4-(2-phenylethynyl)phenyl, 4-(2-(1-naphthyl)ethynyl)phenyl, 4-(2-(2-naphthyl)ethynyl)phenyl, 4-(2-(2-phenylphenyl)ethynyl)phenyl, 4-(2-(4-phenylphenyl)ethynyl)phenyl, 4-(2-(9-phenanthrenyl)ethynyl)phenyl, 4-(2-(2-dibenzofuranyl)ethynyl)phenyl, 4-(2-(4-dibenzofuranyl)ethynyl)phenyl, 4-(2-(2-dibenzothiophenyl)ethynyl)phenyl, 4-(2-(4-dibenzothiophenyl)ethynyl)phenyl, 4-(2-(1-thianthrenyl)ethynyl)phenyl, 4-(2-(2-thianthrenyl)ethynyl)phenyl, 4-(2-(2-triphenylenyl)ethynyl)phenyl, 4-(2-(2-pyridinyl)ethynyl)phenyl, 4-(2-(3-pyridinyl)ethynyl)phenyl, 4-(2-(4-pyridinyl)ethynyl)phenyl, 4-(2-(2-quinolinyl)ethynyl)phenyl, 4-(2-(3-quinolinyl)ethylenyl)phenyl, 4-(2-(4-quinolinyl)ethynyl)phenyl, 4-(2-(8-quinolinyl)ethynyl)phenyl, 4-(2-phenylethynyl)-1-naphthyl and 6-(2-phenylethynyl)-2-naphthyl. Especially, R¹ and R² are selected from hydrogen, phenyl, naphthyl and Rₐ, where R^{a} is in particular 2-phenylethynyl, 2-(1-naphthyl)ethynyl or 2-(2-naphthyl)ethynyl.

In a preferred group (7′) of embodiments the moieties R³—OH or R³—O—#, respectively, in formulae (I) and (II) are C₁-C₄-alkandiyl-OH or C₁-C₄-alkandiyl-O—#, respectively, where C₁-C₄-alkandiyl is preferably methylene or a linear C2-C₄-alkandiyl, such as e.g. 1,2-ethandiyl (CH₂—CH₂), 1,3-propandiyl or 1,4-butandiyl, and in particular is methylene. Thus, according to the group (7′) of embodiments the variables R³—OH or R³—O—#, respectively, are in particular CH₂—OH or CH₂—O—#, respectively.

In a group (7″) of embodiments the moieties R³—OH or R³—O—#, respectively, in formulae (I) and (II) are O—C₂-C₄-alkandiyl-OH or O—C₂-C₄-alkandiyl-O—#, respectively, where C₂-C₄-alkandiyl is preferably a linear moiety, such as e.g. 1,2-ethandiyl, 1,3-propandiyl or 1,4-butandiyl, and in particular is 1,2-ethandiyl. Thus, according to the group (7″) of embodiments the variables R³—OH or R³—O—#, respectively, are in particular O—CH₂—CH₂—OH or O—CH₂—CH₂—O—#, respectively.

In a preferred group (7′″) of embodiments the variables R³—OH or R³—O—#, respectively, in formulae (I) and (II) are O—C₁-C₄-alkandiyl-C(O)—OH or O—C₁-C₄-alkandiyl-C(O)—O—#, respectively, where C₁-C₄-alkandiyl is preferably methylene or a linear C2-C₄-alkandiyl, such as e.g. 1,2-ethandiyl (CH₂—CH₂), 1,3-propandiyl or 1,4-butandiyl, and in particular is methylene. Thus, according to the group (7′″) of embodiments the variables R³—OH or R³—O—#, respectively, are in particular O—CH₂—C(O)—OH or O—CH₂—C(O)—O—#, respectively.

In a group (8) of embodiments, the radicals R⁴ and R⁵ in formulae (I) and (II) are selected independently of one another from fluorine, CN, phenoxy, benzyl, methyl, mono- and polycyclic aryl, and a radical R^{a}. Preferably, R⁴ and R⁵ have identical meanings selected from fluorine, methyl, optionally substituted phenyl, optionally substituted naphthyl, CN, and R^{a}, i.e. C≡C—R¹¹ or Ar—C≡C—R¹¹. In particular, R⁴ and R⁵ are selected from phenyl, naphthyl, CN, ethynyl, cyanophenyl, dicyanophenyl, cyanonaphthyl, dicyanonaphthyl, methylethynyl, phenylethynyl, naphthylethynyl, biphenylylethynyl, phenanthrylethynyl, dibenzofuranylethynyl, dibenzothiophenylethynyl, thianthrenylethynyl, triphenylenylethynyl, pyridinylethynyl, quinolinylethynyl, methylethynylphenyl, phenylethynylphenyl, methylethynylnaphthyl, phenylethynylnaphthyl, naphthylethynylphenyl, naphthylethynylnaphthyl, phenanthrylethynylphenyl, biphenylylethynylphenyl, triphenylenylethynyl)phenyl, pyridinylethynylphenyl, quinolinylethynylphenyl, dibenzofuranylethynylphenyl and dibenzothiophenylethynylphenyl, thianthrenylethynylphenyl and especially from CN, ethynyl, phenyl, 3-cyanophenyl, 4-cyanophenyl, 3,5-dicyanophenyl, 4-cyano-1-naphthyl, 6-cyano-1-naphthyl, 6-cyano-2-naphthyl, 2-phenylethynyl, 2-(1-naphthyl)ethynyl, 2-(2-naphthyl)ethynyl, 2-(2-phenylphenyl)ethynyl, 2-(4-phenylphenyl)ethynyl, 2-(triphenylen-2-yl)ethynyl, 2-(pyridin-2-yl)ethynyl, 2-(pyridin-3-yl)ethynyl, 2-(pyridin-4-yl)ethynyl, 2-(quinoline-2-yl)ethynyl, 2-(quinoline-3-yl)ethynyl, 2-(quinoline-4-yl)ethynyl, 2-(quinoline-8-yl)ethynyl, 2-(9-phenanthryl)ethynyl, 2-(2-dibenzofuranyl)ethynyl, 2-(4-dibenzofuranyl)ethynyl, 2-(2-dibenzothiophenyl)ethynyl, 2-(4-dibenzothiophenyl)ethynyl, 2-(1-thianthrenyl)ethynyl, 2-(2-thianthrenyl)ethynyl, 4-(2-phenylethynyl)phenyl, 4-(2-(1-naphthyl)ethynyl)phenyl, 4-(2-(2-naphthyl)ethynyl)phenyl, 4-(2-(2-phenylphenyl)ethynyl)phenyl, 4-(2-(4-phenylphenyl)ethynyl)phenyl, 4-(2-(9-phenanthrenyl)ethynyl)phenyl, 4-(2-(2-dibenzofuranyl)ethynyl)phenyl, 4(2-(4-dibenzofuranyl)ethynyl)phenyl, 4-(2-(2-dibenzothiophenyl)ethynyl)phenyl, 4-(2-(4-dibenzothiophenyl)ethynyl)phenyl, 4-(2-(1-thianthrenyl)ethynyl)phenyl, 4-(2-(2-thianthrenyl)ethynyl)phenyl, 4-(2-(2-triphenylenyl)ethynyl)phenyl, 4-(2-(2-pyridinyl)ethynyl)phenyl, 4-(2-(3-pyridinyl)ethynyl)phenyl, 4-(2-(4-pyridinyl)ethynyl)phenyl, 4-(2-(2-quinolinyl)ethynyl)phenyl, 4-(2-(3-quinolinyl)ethylenyl)phenyl, 4-(2-(4-quinolinyl)ethynyl)phenyl, 4-(2-(8-quinolinyl)ethynyl)phenyl, 4-(2-phenylethynyl)-1-naphthyl and 6-(2-phenylethynyl)-2-naphthyl. Especially, R⁴ and R⁵ if present, are selected from halogen, phenyl, naphthyl, and Rₐ, where R^{a} is in particular 2-phenylethynyl, 2-(1-naphthyl)ethynyl and 2-(2-naphthyl)ethynyl.

The variables n and m in formulae (I) and (II) are preferably 0 or 1. It is also preferred that the values of the variables n and m are identical. Accordingly, particular preference is given to variables n and m which are both either 0 or 1.

A skilled person will readily appreciate that the meanings of A¹ and A² of group (1) of embodiments may be combined with the meanings of Y of group (4) or group (5) of embodiments, with the meanings of R³ of groups (7′), (7″) or (7′″) of embodiments and also with the meanings of X, R¹, R², R⁴ and R⁵ of groups (3), (4′), (6) and (8), respectively. A skilled person will also appreciate that the meanings of A¹ and A² of group (2) of embodiments may be combined with the meanings of Y of group (4) or group (5) of embodiments, with the meanings of R³ of group (7′), (7″) or (7′″) of embodiments and also with the meanings of X, R¹, R², R⁴ and R⁵ of groups (3), (4′), (6) and (8), respectively.

According to the invention, the compound of formula (I) bears at least one radical R^{a}, in particular at least 2 radicals R^{a}, more particularly 2 to 4 radicals R^{a} and especially 2 or 3 radicals R^{a}. These radicals R^{a} may be bound directly to A¹ or A², respectively, e.g. as radicals R¹, R², R⁴ or R⁵, bound to the moiety X, e.g. as a radical R⁶, bound to the moiety Y, e.g. as a radical R⁸, or bound to the moiety of the formula A, i.e. as radicals R¹⁰.

Preferably, the radical R^{a} is selected from ethynyl, methylethynyl, phenylethynyl, naphthylethynyl, phenanthrylethynyl, biphenylylethynyl, dibenzofuranylethynyl, dibenzothiophenylethynyl, thianthrenylethynyl, triphenylenylethynyl, pyridinylethynyl, quinolinylethynyl, methylethynylphenyl, phenylethynylphenyl, methylethynylnaphthyl, phenylethynylnaphthyl, naphthylethynylphenyl, naphthylethynylnaphthyl, phenanthrylethynylphenyl, phenanthrylethynylnaphthyl, biphenylylethynylphenyl, triphenylenylethynyl)phenyl, pyridinylethynylphenyl, quinolinylethynylphenyl, dibenzofuranylethynylphenyl, dibenzothiophenylethynylphenyl and thianthrenylethynylphenyl.

More preferably, R^{a} is selected from ethynyl, 2-methylethynyl, 2-phenylethynyl, 2-(1-naphthyl)ethynyl, 2-(2-naphthyl)ethynyl, 2-(2-phenylphenyl)ethynyl, 2-(4-phenylphenyl)ethynyl, 2-(triphenylen-2-yl)ethynyl, 2-(pyridin-2-yl)ethynyl, 2-(pyridin-3-yl)ethynyl, 2-(pyridin-4-yl)ethynyl, 2-(quinoline-2-yl)ethynyl, 2-(quinoline-3-yl)ethynyl, 2-(quinoline-4-yl)ethynyl, 2-(quinoline-8-yl)ethynyl, 2-(9-phenanthryl)ethynyl, 2-(2-dibenzofuranyl)ethynyl, 2-(4-dibenzofuranyl)ethynyl, 2-(2-dibenzothiophenyl)ethynyl, 2-(4-dibenzothiophenyl)ethynyl, 2-(1-thianthrenyl)ethynyl, 2-(2-thianthrenyl)ethynyl, 2-(2-phenylethynyl)phenyl, 3-(2-phenylethynyl)phenyl, 4-(2-phenylethynyl)phenyl, 2-(2-(2-naphthyl)ethynyl)phenyl, 3-(2-(2-naphthyl)ethynyl)phenyl, 4-(2-(2-naphthyl)ethynyl)phenyl, 2-(2-(1-naphthyl)ethynyl)phenyl, 3-(2-(1-naphthyl)ethynyl)phenyl, 4-(2-(1-naphthyl)ethynyl)phenyl, 4-(2-(2-phenylphenyl)ethynyl)phenyl, 4-(2-(4-phenylphenyl)ethynyl)phenyl, 4-(2-(9-phenanthrenyl)ethynyl)phenyl, 4-(2-(2-dibenzofuranyl)ethynyl)phenyl, 4-(2-(4-dibenzofuranyl)ethynyl)phenyl, 4-(2-(2-dibenzothiophenyl)ethynyl)phenyl, 4-(2-(4-dibenzothiophenyl)ethynyl)phenyl, 4-(2-(1-thianthrenyl)ethynyl)phenyl, 4-(2-(2-thianthrenyl)ethynyl)phenyl, 4-(2-(2-triphenylenyl)ethynyl)phenyl, 4-(2-(2-pyridinyl)ethynyl)phenyl, 4-(2-(3-pyridinyl)ethynyl)phenyl, 4-(2-(4-pyridinyl)ethynyl)phenyl, 4-(2-(2-quinolinyl)ethynyl)phenyl, 4-(2-(3-quinolinyl)ethylenyl)phenyl, 4-(2-(4-quinolinyl)ethynyl)phenyl, 4-(2-(8-quinolinyl)ethynyl)phenyl, 2-(2-phenylethynyl)-1-naphthyl, 3-(2-phenylethynyl)-1-naphthyl, 4-(2-phenylethynyl)-1-naphthyl, 5-(2-phenylethynyl)-1-naphthyl, 6-(2-phenylethynyl)-1-naphthyl, 7-(2-phenylethynyl)-1-naphthyl, 8-(2-phenylethynyl)-1-naphthyl, 1-(2-phenylethynyl)-2-naphthyl, 3-(2-phenylethynyl)-2-naphthyl, 4-(2-phenylethynyl)-2-naphthyl, 5-(2-phenylethynyl)-2-naphthyl, 6-(2-phenylethynyl)-2-naphthyl, 7-(2-phenylethynyl)-2-naphthyl, 8-(2-phenylethynyl)-2-naphthyl 2-(2-(1-naphthyl)ethynyl)-1-naphthyl, 3-(2-(1-naphthyl)ethynyl)-1-naphthyl, 4-(2-(1-naphthyl)ethynyl)-1-naphthyl, 5-(2-(1-naphthyl)ethynyl)-1-naphthyl, 6-(2-(1-naphthyl)ethynyl)-1-naphthyl, 7-(2-(1-naphthyl)ethynyl)-1-naphthyl, 8-(2-(1-naphthyl)ethynyl)-1-naphthyl, 1-(2-(1-naphthyl)ethynyl)-2-naphthyl, 3-(2-(1-naphthyl)ethynyl)-2-naphthyl, 4-(2-(1-naphthyl)ethynyl)-2-naphthyl, 5-(2-(1-naphthyl)ethynyl)-2-naphthyl, 6-(2-(1-naphthyl)ethynyl)-2-naphthyl, 7-(2-(1-naphthyl)ethynyl)-2-naphthyl 8-(2-(1-naphthyl)ethynyl)-2-naphthyl 2-(2-(2-naphthyl)ethynyl)-1-naphthyl, 3-(2-(2-naphthyl)ethynyl)-1-naphthyl, 4-(2-(2-naphthyl)ethynyl)-1-naphthyl, 5-(2-(2-naphthyl)ethynyl)-1-naphthyl, 6-(2-(2-naphthyl)ethynyl)-1-naphthyl, 7-(2-(2-naphthyl)ethynyl)-1-naphthyl, 8-(2-(2-naphthyl)ethynyl)-1-naphthyl, 1-(2-(2-naphthyl)ethynyl)-2-naphthyl, 3-(2-(2-naphthyl)ethynyl)-2-naphthyl, 4-(2-(2-naphthyl)ethynyl)-2-naphthyl, 5-(2-(2-naphthyl)ethynyl)-2-naphthyl, 6-(2-(2-naphthyl)ethynyl)-2-naphthyl, 7-(2-(2-naphthyl)ethynyl)-2-naphthyl and 8-(2-(2-naphthyl)ethynyl)-2-naphthyl.

In particular, R^{a} is selected from ethynyl, 2-phenylethynyl, 2-(1-naphthyl)ethynyl, 2-(2-naphthyl)ethynyl, 2-(2-phenylphenyl)ethynyl, 2-(4-phenylphenyl)ethynyl, 2-(triphenylen-2-yl)ethynyl, 2-(pyridin-2-yl)ethynyl, 2-(pyridin-3-yl)ethynyl, 2-(pyridin-4-yl)ethynyl, 2-(quinoline-2-yl)ethynyl, 2-(quinoline-3-yl)ethynyl, 2-(quinoline-4-yl)ethynyl, 2-(quinoline-8-yl)ethynyl, 2-(9-phenanthryl)ethynyl, 2-(2-dibenzofuranyl)ethynyl, 2-(4-dibenzofuranyl)ethynyl, 2-(2-dibenzothiophenyl)ethynyl, 2-(4-dibenzothiophenyl)ethynyl, 2-(1-thianthrenyl)ethynyl, 2-(2-thianthrenyl)ethynyl, 4-(2-phenylethynyl)phenyl, 4-(2-(1-naphthyl)ethynyl)phenyl, 4-(2-(2-naphthyl)ethynyl)phenyl, 4-(2-(2-phenylphenyl)ethynyl)phenyl, 4-(2-(4-phenylphenyl)ethynyl)phenyl, 4-(2-(9-phenanthrenyl)ethynyl)phenyl, 4-(2-(2-dibenzofuranyl)ethynyl)phenyl, 4-(2-(4-dibenzofuranyl)ethynyl)phenyl, 4-(2-(2-dibenzothiophenyl)ethynyl)phenyl, 4-(2-(4-dibenzothiophenyl)ethynyl)phenyl, 4-(2-(1-thianthrenyl)ethynyl)phenyl, 4-(2-(2-thianthrenyl)ethynyl)phenyl, 4-(2-(2-triphenylenyl)ethynyl)phenyl, 4-(2-(2-pyridinyl)ethynyl)phenyl, 4-(2-(3-pyridinyl)ethynyl)phenyl, 4-(2-(4-pyridinyl)ethynyl)phenyl, 4-(2-(2-quinolinyl)ethynyl)phenyl, 4-(2-(3-quinolinyl)ethylenyl)phenyl, 4-(2-(4-quinolinyl)ethynyl)phenyl, 4-(2-(8-quinolinyl)ethynyl)phenyl, 4-(2-phenylethynyl)-1-naphthyl and 6-(2-phenylethynyl)-2-naphthyl.

Especially, the radical R^{a} is selected from the group consisting of 2-phenylethynyl, 2-(1-naphthyl)ethynyl, which is also termed naphthalene-1-ylethynyl, and 2-(2-naphthyl)ethynyl, which is also termed naphthalene-2-ylethynyl.

Apart from that and if not stated otherwise, the variables R⁶, R⁷, R⁸, R⁹, R¹⁰, R¹¹, R¹², Alk, Alk′, Ar′, R^{Ar}, R and k either alone or preferably in combination have the following meanings.

Preferably, the radicals R⁶ and R⁸ are independently of one another selected from hydrogen, methylethynyl, phenylethynyl, naphthylethynyl, phenanthrylethynyl, methylethynylphenyl, phenylethynylphenyl, methylethynylnaphthyl, phenylethynylnaphthyl, naphthylethynylphenyl, naphthylethynylnaphthyl, phenanthrylethynylphenyl and phenanthrylethynylnaphthyl.

Preference is also given to R⁶ and R⁸ being independently of one another selected from hydrogen, phenyl, naphthyl, phenanthryl, 1,2-dihydroacenaphthylenyl, 9H-fluorenyl, biphenylenyl, biphenylyl, dibenzo[b,d]furanyl, pyrrolyl, indolyl, pyridyl, quinolinyl, isoquinolinyl and pyrimidinyl, which may be unsubstituted or substituted by 1 radical R^{Ar}, with R^{Ar} having one of the meanings defined herein, in particular one of the preferred meanings.

Particularly, the radicals R⁶ and R⁸ are independently of one another selected from hydrogen, phenylethynyl, naphthalin-1-ylethynyl, naphthin-2-ylethynyl, phenanthren-9-ylethynyl, 4-(phenylethynyl)-phenyl, 4-(naphthin-1-ylethynyl)-phenyl, 4-(phenylethynyl)-1-naphthyl, 6-(phenylethynyl)-2-naphthyl, phenyl, 3-cyanophenyl, 4-cyanophenyl, 3,5-dicyanophenyl, naphthyl, specifically 1- or 2-naphthyl, 4-cyano-1-naphthyl, 6-cyano-1-naphthyl, 6-cyano-2-naphthyl and phenanthryl, specifically 9-phenanthryl. The radicals R⁶ and R⁸ independently of one another are particularly preferred selected from hydrogen, phenyl, cyanophenyl, specifically 3-cyanophenyl or 4-cyanophenyl, dicyanophenyl, specifically 3,5-dicyanophenyl, naphthyl, specifically 1- or 2-naphthyl, cyanonaphthyl, specifically 4-cyano-1-naphthyl, 6-cyano-1-naphthyl or 6-cyano-2-naphthyl, and phenanthryl, specifically 9-phenanthryl.

Preferably, the radicals R⁷ and R⁹ are independently of one another selected from hydrogen, methyl, ethyl, n-propyl, isopropyl, n-butyl, sec-butyl, tert-butyl, phenyl, naphthyl, phenanthryl, 1,2-dihydroacenaphthylenyl, 9H-fluorenyl, biphenylenyl, biphenylyl, dibenzo[b,d]furanyl, pyrrolyl, indolyl, pyridyl, quinolinyl, isoquinolinyl and pyrimidinyl, where the (het)aryl groups mentioned above are unsubstituted or substituted by 1 or 2 radicals R^{Ar}, with R^{Ar} having one of the meanings defined herein, in particular one of the preferred meanings.

Particularly, the radicals R⁷ and R⁹ are independently of one another selected from hydrogen, methyl, ethyl, isopropyl, phenyl, naphthyl, specifically 1- or 2-naphthyl, and phenanthryl, specifically 9-phenanthryl. Especially, R⁷ and R⁹ are independently of one another selected from hydrogen and methyl.

Preferably, the radicals R¹⁰ are selected from hydrogen, fluorine, CN, methyl, phenyl, naphthyl, phenanthryl, pyridyl, phenoxy, benzyl, cyanophenyl, dicyanophenyl, cyanonaphthyl, dicyanonaphthyl, methylethynyl, phenylethynyl, naphthylethynyl, biphenylylethynyl, phenanthrylethynyl, dibenzofuranylethynyl, dibenzothiophenylethynyl, thianthrenylethynyl, triphenylenylethynyl, pyridinylethynyl, quinolinylethynyl, methylethynylphenyl, phenylethynylphenyl, methylethynylnaphthyl, phenylethynylnaphthyl, naphthylethynylphenyl, naphthylethynylnaphthyl, phenanthrylethynylphenyl, phenanthrylethynylnaphthyl, biphenylylethynylphenyl, triphenylenylethynyl)phenyl, pyridinylethynylphenyl, quinolinylethynylphenyl, dibenzofuranylethynylphenyl, dibenzothiophenylethynylphenyl and thianthrenylethynylphenyl.

The radicals R¹⁰ are particularly selected from hydrogen, fluorine, CN, methyl, phenyl, 3-cyanophenyl, 4-cyanophenyl, 3,5-dicyanophenyl, 4-cyano-1-naphthyl, 6-cyano-1-naphthyl, 6-cyano-2-naphthyl, 2-phenylethynyl, 2-(naphthalin-1-yl)ethynyl, 2-(naphthalin-2-yl)ethynyl, 2-(2-phenylphenyl)ethynyl, 2-(4-phenylphenyl)ethynyl, 2-(triphenylen-2-yl)ethynyl, 2-(pyridin-2-yl)ethynyl, 2-(pyridin-3-yl)ethynyl, 2-(pyridin-4-yl)ethynyl, 2-(quinoline-2-yl)ethynyl, 2-(quinoline-3-yl)ethynyl, 2-(quinoline-4-yl)ethynyl, 2-(quinoline-8-yl)ethynyl, 2-(9-phenanthryl)ethynyl, 2-(2-dibenzofuranyl)ethynyl, 2-(4-dibenzofuranyl)ethynyl, 2-(2-dibenzothiophenyl)ethynyl, 2-(4-dibenzothiophenyl)ethynyl, 2-(1-thianthrenyl)ethynyl, 2-(2-thianthrenyl)ethynyl, 4-(2-phenylethynyl)-phenyl, 4-(2-(1-naphthylethynyl)-phenyl, 4-(2-(2-naphthylethynyl)-phenyl, 4-(2-(2-phenylphenyl)ethynyl)phenyl, 4-(2-(4-phenylphenyl)ethynyl)phenyl, 4-(2-(9-phenanthrenyl)ethynyl)phenyl, 4-(2-(2-dibenzofuranyl)ethynyl)phenyl, 4-(2-(4-dibenzofuranyl)ethynyl)phenyl, 4-(2-(2-dibenzothiophenyl)ethynyl)phenyl, 4-(2-(4-dibenzothiophenyl)ethynyl)phenyl, 4-(2-(1-thianthrenyl)ethynyl)phenyl, 4-(2-(2-thianthrenyl)ethynyl)phenyl, 4-(2-(2-triphenylenyl)ethynyl)phenyl, 4-(2-(2-pyridinyl)ethynyl)phenyl, 4-(2-(3-pyridinyl)ethynyl)phenyl, 4-(2-(4-pyridinyl)ethynyl)phenyl, 4-(2-(2-quinolinyl)ethynyl)phenyl, 4-(2-(3-quinolinyl)ethylenyl)phenyl, 4-(2-(4-quinolinyl)ethynyl)phenyl, 4-(2-(8-quinolinyl)ethynyl)phenyl, 4-(phenylethynyl)-1-naphthyl and 6-(phenylethynyl)-2-naphthyl. In particular the radical R¹⁰ is selected from the group consisting of hydrogen, fluorine, CN, methyl, phenyl, naphthyl, 2-phenylethynyl, 2-(1-naphthyl)ethynyl and 2-(2-naphthyl)ethynyl.

Preferably, the radical R¹¹ is selected from hydrogen, methyl, phenyl, naphthyl, phenanthryl, biphenylyl, triphenylenyl, dibenzo[b,d]furanyl, dibenzo[b,d]thiophenyl, thianthrenyl, pyrrolyl, indolyl, pyridyl, quinolinyl, isoquinolinyl and pyrimidinyl, and in particular from hydrogen, methyl, phenyl, naphthyl, specifically 1- or 2-naphthyl, phenanthryl, specifically 9-phenanthryl, biphenylyl, specifically 2-phenylphenyl or 4-phenylphenyl, triphenylenyl, specifically 2-triphenylenyl, dibenzo[b,d]furanyl, specifically 2-dibenzofuranyl or 4-dibenzofuranyl, dibenzo[b,d]thiophenyl, specifically 2-dibenzothiophenyl or 4-dibenzothiophenyl, thianthrenyl, specifically 1-thianthrenyl or 2-thianthrenyl, pyridyl, specifically 2-pyridyl, 3-pyridyl or 4-pyridyl, and quinolinyl, specifically 2-quinolinyl, 3-quinolinyl, 4-quinolinyl or 8-quinolinyl, where the (het)aryl groups mentioned above are unsubstituted or substituted by 1 or 2 radicals R¹², with R¹² having one of the meanings defined herein, in particular one of the preferred meanings. In particular, R¹¹ is phenyl or naphthyl.

Preferably, the one or more radicals R¹², if present, are independently selected from fluorine, phenyl, CN, OCH₃, CH₃, C≡CH and C≡C—CH₃, in particular from fluorine, phenyl, CN and C≡CH.

Preferably, the variable Alk is selected from methylene and linear C2-C₄-alkandiyl, such as e.g. 1,2-ethandiyl (CH₂—CH₂), 1,3-propandiyl or 1,4-butandiyl, and in particular is methylene.

Preferably, the variable Alk′ is selected from linear C₂-C₄-alkandiyl moieties, such as e.g. 1,2-ethandiyl (CH₂—CH₂), 1,3-propandiyl or 1,4-butandiyl, and in particular is 1,2-ethandiyl.

The mono- or polycyclic aryl moieties suitable as radical Ar′ are preferably selected from phenyl, naphthyl, phenanthryl, biphenylyl, 2,3-dihydro-1H-indenyl, 1H-indenyl, 5,6,7,8-tetrahydronaphthalenyl, 1,2-dihydroacenaphthylenyl, acenaphthylenyl, 9,10-dihydroanthracen-1-yl, 1,2,3,4-tetrahydrophenanthrenyl, 5,6,7,8-tetrahydrophenanthrenyl, fluorenyl, anthracenyl, pyrenyl, biphenylenyl, triphenylenyl, tetraphenylenyl, 5H-dibenzo[a,d][7]annulenyl, perylenyl, 9,9′-spirobi[9H-fluoren]yl, 10,11-dihydro-5H-dibenzo[a,d][7]annulenyl and dibenzo[a,e][8]annulenyl, more preferably selected from phenyl, naphthyl, specifically 1- or 2-naphthyl, phenanthryl, specifically 9-phenanthryl, 1,2-dihydroacenaphthylenyl, specifically 1,2-dihydroacenaphthylen-5-yl, anthracenyl, specifically 9-anthracenyl, 9H-fluorenyl, specifically 9H-fluoren-2-yl, pyrenyl specifically 3-pyrenyl, and biphenylyl, specifically 3- or 4-biphenylyl, and in particular selected from phenyl, naphthyl, specifically 1- or 2-naphthyl, phenanthryl, specifically 9-phenanthryl, 1,2-dihydroacenaphthylenyl, specifically 1,2-dihydroacenaphthylen-5-yl, 9H-fluorenyl, specifically 9H-fluoren-2-yl, biphenylenyl and biphenylyl, specifically 3- or 4-biphenylyl, where the mono- or polycyclic aryl moieties mentioned before may be unsubstituted or substituted by 1 radical R^{Ar}, with R^{Ar} having one of the meanings defined herein, in particular one of the preferred meanings.

The mono- or polycyclic hetaryl moieties suitable as radical Ar′ are preferably selected from furyl, benzofuryl, naphthofuryl, dibenzofuranyl, thianthrenyl, 9H-xanthenyl, 2H-chromenyl, 4H-chromenyl, 2H-benzo[g]chromenyl, 4H-benzo[g]chromenyl, 3H-benzo[f]chromenyl, 1H-benzo[f]chromenyl, furo[3,2-b]furanyl, furo[2,3-b]furanyl, furo[3,4-b]furanyl, 2,3-dihydro-1,4-benzodioxinyl, oxanthrenyl, furo[3,2-f][1]benzofuranyl, furo[2,3-f][1]benzofuranyl, pyrrolyl, indolyl, isoindolyl, carbazolyl, indolizinyl, benzo[cd]indolyl, 1H-benzo[g]indolyl, 3H-benzo[e]indolyl, 1H-benzo[f]indolyl, pyridyl, quinolinyl, isoquinolinyl, acridinyl, phenanthridinyl, benzo[f]isoquinolinyl, benzo[h]isoquinolinyl, imidazolyl, pyrazolyl, pyrazinyl, pyridazinyl, pyrimidinyl, benzopyrazolyl, benzimidazolyl, quinazolinyl, quinoxalinyl, cinnolinyl, 1,5-naphthyridinyl, 1,8-naphthyridinyl, dipyridyl, pyrido[4,3-b]indolyl, pyrido[3,2-b]indolyl, pyrrolo[3,2-b]pyridinyl, phenazinyl, benzo[b][1,5]naphthyridinyl, phenanthrolinyl, benzo[b][1,8]naphthyridin-3-yl, pyrido[2,3-g]quinolinyl, pyrido[3,2-p]quinolinyl, benzo[g]quinoxalinyl, benzo[f]quinoxalinyl, 1,2,3-triazolyl, 1,2,4-triazolyl, triazinyl, pyrido[2,3-b][1,8]naphthyridinyl, tetrazolyl, oxazolyl, isoxazolyl, 1,3,4-oxadiazolyl, 1,2,4-oxadiazolyl, benzoxazolyl, phenoxazinyl, furo[3,2-g]quinolinyl, furo[2,3-g]quinolinyl and furo[2,3-g]quinoxalinyl, and in particular selected from dibenzo[b,d]furanyl, specifically 2- or 3-dibenzo[b,d]furanyl, pyrrolyl, specifically 2- or 3-pyrrolyl, indolyl, specifically 3-indolyl, pyridyl, specifically 2-, 3- or 4-pyridyl, quinolinyl, specifically 2-, 3- or 4-quinolinyl, isoquinolinyl, specifically 1- or 4-isoquinolinyl, and pyrimidinyl, specifically 5-pyrimidinyl, where the mono- or polycyclic hetaryl moieties mentioned before may be unsubstituted or substituted by 1 radical R^{Ar}, with R^{Ar} having one of the meanings defined herein, in particular one of the preferred meanings.

The one or more radicals Ar′, if present, are preferably unsubstituted or bear 1 or 2 radicals R^{Ar}, and in particular are unsubstituted or bear one radical R^{Ar}.

The one or more radicals R^{Ar}, if present, are preferably independently selected from the group consisting of fluorine, chlorine CN, R, OR, CHₖR₃₋ₖ, NR₂, C(O)R, C(O)NH₂, C≡C—R¹¹ and Ar—C≡C—R¹¹, where the variables k, R, R¹¹ and Ar have the meanings defined herein, in particular the preferred meanings.

Preferably, the one or more radicals R^{Ar}, if present, are independently selected from the group consisting of fluorine, chlorine, CN, CH₃, OCH₃, phenyl, naphthyl, anthracenyl, phenanthryl, 9H-fluorenyl, biphenylyl, where the last six moieties may optionally carry one or two radicals R¹² selected from fluorine and CN, dibenzofuranyl, pyrrolyl, indolyl, pyridyl, quinolinyl, isoquinolinyl, pyrimidinyl, phenoxy, naphthyloxy, benzyl, N(CH₃)₂, C(O)CH₃, C≡C—R¹¹ and Ar—C≡C—R¹¹, where Ar is as defined herein and R¹¹ is preferably selected from hydrogen, methyl, phenyl, naphthyl, phenanthryl, biphenylyl, triphenylenyl, dibenzofuranyl, dibenzothiophenyl, pyrrolyl, indolyl, pyridyl, quinolinyl, isoquinolinyl and pyrimidinyl.

More preferably, the one or more radicals R^{Ar}, if present, are selected from the group consisting of fluorine, chlorine, CN, CH₃, phenyl, naphthyl, phenanthryl, ethynyl, cyanophenyl, dicyanophenyl, cyanonaphthyl, dicyanonaphthyl, methylethynyl, phenylethynyl, naphthylethynyl, biphenylylethynyl, phenanthrylethynyl, dibenzofuranylethynyl, dibenzothiophenylethynyl, triphenylenylethynyl, pyridinylethynyl, quinolinylethynyl, methylethynylphenyl, phenylethynylphenyl, methylethynylnaphthyl, phenylethynylnaphthyl, naphthylethynylphenyl, naphthylethynylnaphthyl, phenanthrylethynylphenyl, phenanthrylethynylnaphthyl, biphenylylethynylphenyl, triphenylenylethynyl)phenyl, pyridinylethynylphenyl, quinolinylethynylphenyl, dibenzofuranylethynylphenyl and dibenzothiophenylethynylphenyl.

In particular, the one or more radicals R^{Ar}, if present, are selected from the group consisting of CN, CH₃, phenyl, naphthyl, specifically 1-naphthyl or 2-naphthyl, phenanthryl, specifically 9-phenanthryl, ethynyl, cyanophenyl, specifically 3-cyanophenyl or 4-cyanophenyl, dicyanophenyl, specifically 3,5-dicyanophenyl, cyano-naphthyl, specifically 4-cyano-1-naphthyl, 6-cyano-1-naphthyl or 6-cyano-2-naphthyl, 2-phenylethynyl, 2-naphthylethynyl, specifically 2-(1-naphthyl)ethynyl or 2-(2-naphthyl)ethynyl, and especially selected from CN, CH₃, phenyl, naphthyl, specifically 1-naphthyl or 2-naphthyl, ethynyl, cyanophenyl, specifically 3-cyanophenyl or 4-cyanophenyl, dicyanophenyl, specifically 3,5-dicyanophenyl, cyano-naphthyl, specifically 4-cyano-1-naphthyl, 6-cyano-1-naphthyl or 6-cyano-2-naphthyl, and 2-phenylethynyl.

Preferably, the mono- or polycyclic aryl moieties suitable as radical R are selected from the group consisting of phenyl, naphthyl, anthracenyl, phenanthryl, 9H-fluorenyl, biphenylyl, dibenzofuranyl, pyrrolyl, indolyl, pyridyl, quinolinyl, isoquinolinyl and pyrimidinyl. In particular, the radical R are selected from the group consisting of phenyl, naphthyl, specifically 1- or 2-naphthyl and phenanthryl, specifically 9-phenanthryl.

Preferably, the variables p and k are independently of one another selected from 1, 2 and 3, and in particular from 2 and 3.

In a particular group (9) of preferred embodiments of the present invention the compound of the formula (I) and likewise the structural unit of formula (II) bear at least one, preferably 2 or 4, and in particular 2 of the radicals R¹, R², R⁴, R⁵, R⁶, R⁸ or R¹⁰ which are selected from R^{a}, i.e. from C≡C—R¹¹ and Ar—C≡C—R¹¹, where the radicals Ar and R¹¹ have one of the meanings defined herein, in particular one of the preferred meanings. In particular Ar is 1,4-phenylene. R¹¹ is in particular phenyl or naphthyl.

A skilled person will readily appreciate that the particular group (9) of embodiments may be combined with the meanings of A¹ and A² of one of group (1) or group (2) of embodiments, with the meanings of Y of group (4) or group (5) of embodiments, with the meanings of R³ of group (7′), group (7)″ or group (7′″) and also with the meanings of X, R¹, R², R⁴ and R⁵ of groups (3), (4′), (6) and (8), respectively.

In this group (9) of embodiments, the radical R¹¹ has one of the meanings defined herein, and preferably is selected from phenyl, naphthyl, specifically naphth-1-yl or naphth-2-yl, phenanthryl, specifically phenanthren-9-yl, biphenylyl, specifically 2-phenylphenyl or 4-phenylphenyl, dibenzofuranyl, specifically 2-dibenzofuranyl or 4-dibenzofuranyl, dibenzothiophenyl, specifically 2-dibenzothiophenyl or 4-dibenzothiophenyl, thianthrenyl, specifically 1-thianthrenyl or 2-thianthrenyl, triphenylenyl, specifically 2-triphenylenyl, pyridinyl, specifically 2-pyridinyl, 3-pyridinyl or 4-pyridinyl, quinolinyl, specifically 2-quinolinyl, 3-quinolinyl, 4-quinolinyl or 8-quinolinyl, and in particular is selected from phenyl, naphth-1-yl, naphth-2-yl, phenanthren-9-yl, 2-dibenzofuranyl, 4-dibenzofuranyl, 2-dibenzothiophenyl, 4-dibenzothiophenyl, 1-thianthrenyl and 2-thianthrenyl.

In the group (9) of embodiments of the present invention a particular subgroup (9′) of embodiments relates to the compounds of the formula (I) and the structural units of formula (II), which bear at least one, preferably 4 or 2 and in particular 2 of radicals R¹, R², R⁴, R⁵ or R¹⁰ which are selected from C≡C—R¹¹ and Ar—C≡C—R¹¹, where the radicals Ar and R¹¹ have one of the meanings defined herein, in particular one of the preferred meanings. In the particular subgroup (9′) of embodiments R¹¹ has one of the meanings defined herein, and preferably is selected from the group consisting of phenyl, naphthyl, specifically naphth-1-yl or naphth-2-yl, phenanthryl, specifically phenanthren-9-yl, biphenylyl, specifically 2-phenylphenyl or 4-phenylphenyl, dibenzofuranyl, specifically 2-dibenzofuranyl or 4-dibenzofuranyl, dibenzothiophenyl, specifically 2-dibenzothiophenyl or 4-dibenzothiophenyl, thianthrenyl, specifically 1-thianthrenyl or 2-thianthrenyl, triphenylenyl, specifically 2-triphenylenyl, pyridinyl, specifically 2-pyridinyl, 3-pyridinyl or 4-pyridinyl, quinolinyl, specifically 2-quinolinyl, 3-quinolinyl, 4-quinolinyl or 8-quinolinyl, and in particular is selected from phenyl, naphth-1-yl, naphth-2-yl, phenanthren-9-yl, 2-dibenzofuranyl, 4-dibenzofuranyl, 2-dibenzothiophenyl, 4-dibenzothiophenyl, 1-thianthrenyl and 2-thianthrenyl.

In a particular subgroup (2a) of groups (2) and (4′) of embodiments, where X represents a single bond, the compound of the formula (I) is a compound of the formula (Ia):

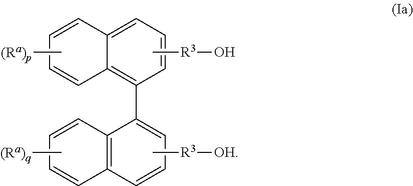

where the radicals R^{a} and R³ have one of the meanings defined herein, in particular one of the preferred meanings, and the variables p and q are independently of one another 1 or 2. Preferably, the variables p and q have the same meaning and are both either 1 or 2, in particular are both 1. It is also preferred that the radicals R^{a} and R³—OH are located in positions 2, 2′,3, 3′,6, 6′, 7 or 7′ of the naphthyl rings, and that the positions of radicals R^{a} and R³—OH on the first naphthyl ring correspond to the positions of the radicals R^{a} and R³—OH on the second naphthyl ring, i.e. if for example the radical R³—OH is located in position 2 on the first naphthyl ring, the other radical R³—OH is preferably located in position 2′ on the second naphthyl ring.

In this subgroup (2a) of groups (2) and (4′) of embodiments the structural unit of the formula (II) is a structural unit of the formula (IIa):

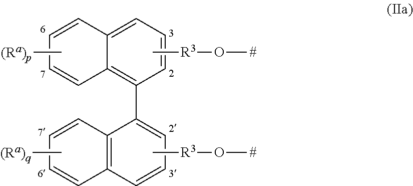

where #represents a connection point to a neighboring structural unit and where the radicals R^{a} and R³ have one of the meanings defined herein, in particular one of the preferred meanings, and the variables p and q are independently of one another 1 or 2, in particular are both 1. The preferred meanings of the variables p and q as well as the preferred positions of the radicals R^{a} and R³—OH described above in the context of formula (Ia) also apply for formula (IIa), where the positions of R³—OH apparently correspond to those of R³—O—#.

In the context of formulae (Ia) and (IIa) R^{a} is in particular C≡C—R¹¹ where R¹¹ is as defined herein and especially phenyl or naphthyl.

In the context of formulae (Ia) and (IIa) particular preference is given to those compounds and structural units, where p=1, q=1, where the two radicals R^{a} are C≡C—R¹¹ where R¹¹ is phenyl, 1-naphthyl or 2-naphthyl, where the two radicals R^{a} are located in 6 and 6′ positions and where the two radicals R³—O—H and R³—O—#, respectively, are located in the 2 and 2′ positions. These compounds and structural units have the following formulae (Ia′) and (IIa′), respectively:

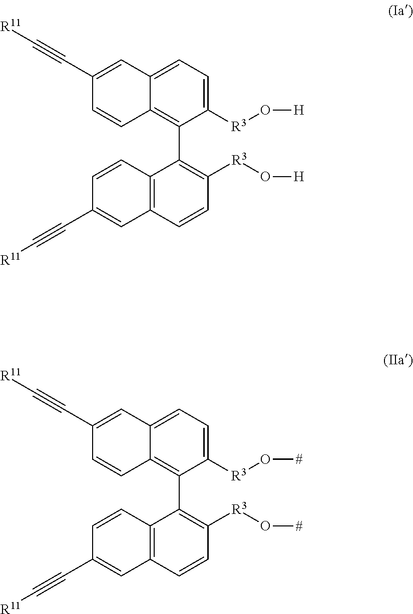

where R¹¹ is phenyl, 1-naphthyl or 2-naphthyl and where R³ is as defined herein and in particular O—C₂-C₄-alkandiyl, especially O—CH₂CH₂, where O is bound to the naphthyl moieties of formulae (Ia′) and (IIa′), respectively.

In a particular subgroup (2a. 1) of groups (2a) and (7″) of embodiments, the compound of the formula (I) is a compound of the formula (Ia-1):

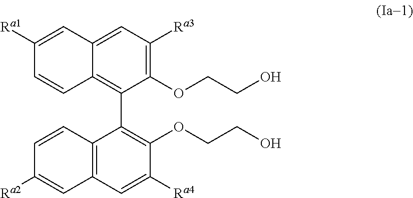

wherein the radicals R^{a1}, R^{a2}, R^{a3} and R^{a4} are independently of one another selected from hydrogen and R^{a}, provided that at least two of R^{a1}, R^{a2}, R^{a3} and R^{a4} are R^{a}, with each of the radicals R^{a} having one of the meanings defined herein, in particular one of the preferred meanings.

In this subgroup (2a. 1) of groups (2a) and (7″) of embodiments the structural unit of the formula (II) is a structural unit of the formula (IIa-1):

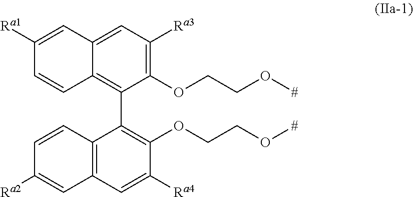

where #represents a connection point to a neighboring structural unit and where the radicals R^{a1}, R^{a2}, R^{a3} and R^{a4} are independently of one another selected from hydrogen and R^{a}, provided that at least two of R^{a1}, R^{a2}, R^{a3} and R^{a4} are R^{a}, with each of the radicals R^{a} having one of the meanings defined herein, in particular one of the preferred meanings.

Preferably, the radicals R^{a1}, R^{a2}, R^{a3} and R^{a4} in formulae (Ia-1) and (IIa-1) are independently of one another selected from hydrogen and R^{a}, where the radical R^{a} is C≡C—R¹¹ or Ar—C≡C—R¹¹ with Ar being preferably phenylene or naphthylene, more preferably phenylene and in particular 1,4-phenylene, and with R¹¹ being preferably selected from phenyl, naphthyl, specifically naphth-1-yl or naphth-2-yl, phenanthryl, specifically phenanthren-9-yl, biphenylyl, specifically 2-phenylphenyl or 4-phenylphenyl, dibenzofuranyl, specifically 2-dibenzofuranyl or 4-dibenzofuranyl, dibenzothiophenyl, specifically 2-dibenzothiophenyl or 4-dibenzothiophenyl, thianthrenyl, specifically 1-thianthrenyl or 2-thianthrenyl, triphenylenyl, specifically 2-triphenylenyl, pyridinyl, specifically 2-pyridinyl, 3-pyridinyl or 4-pyridinyl, quinolinyl, specifically 2-quinolinyl, 3-quinolinyl, 4-quinolinyl or 8-quinolinyl. In particular, R¹¹ is phenyl or naphthyl. In the context of formulae (Ia-1) and (IIa-1) R^{a1}, R^{a2}, R^{a3} and R^{a4} are in particular, independently of one another, selected from hydrogen and R^{a}, where R^{a} is C≡C—R¹¹ where R¹¹ is as defined herein and especially phenyl or naphthyl.

In a particular embodiment of the invention the radicals R^{a1} and R^{a2} in formulae (Ia-1) and (IIa-1) are identical radicals R^{a}, which preferably have one of the meanings mentioned above as preferred, and the radicals R^{a3} and R^{a4} are both hydrogen.

In a further particular embodiment the radicals R^{a1} and R^{a2} in formulae (Ia-1) and (IIa-1) are both hydrogen and the radicals R^{a3} and R^{a4} are identical radicals R^{a}, which preferably have one of the meanings mentioned above as preferred.

In yet another particular embodiment the radicals R^{a1}, R^{a2}, R^{a3} and R^{a4} in formulae (Ia-1) and (IIa-1) are identical radicals R^{a}, which preferably have one of the meanings mentioned above as preferred.

Examples of the particular subgroup (2a. 1) are the compounds of the formula (Ia-1) and the structural units of formula (IIa-1), where the combination of the radicals R^{a1}, R^{a2}, R^{a3} and R^{a4} is as defined in any one of the rows in table A below.

TABLE A

| | R^{a1} | R^{a2} | R^{a3} | R^{a4} |
|---|---|---|---|---|
| 1 | R^{a1}-1 | R^{a1}-1 | H | H |
| 2 | H | H | R^{a1}-1 | R^{a1}-1 |
| 3 | R^{a1}-1 | R^{a1}-1 | R^{a1}-1 | R^{a1}-1 |
| 4 | R^{a1}-2 | R^{a1}-2 | H | H |
| 5 | H | H | R^{a1}-2 | R^{a1}-2 |
| 6 | R^{a1}-2 | R^{a1}-2 | R^{a1}-2 | R^{a1}-2 |
| 7 | R^{a1}-3 | R^{a1}-3 | H | H |
| 8 | H | H | R^{a1}-3 | R^{a1}-3 |
| 9 | R^{a1}-3 | R^{a1}-3 | R^{a1}-3 | R^{a1}-3 |
| 10 | R^{a1}-4 | R^{a1}-4 | H | H |
| 11 | H | H | R^{a1}-4 | R^{a1}-4 |
| 12 | R^{a1}-4 | R^{a1}-4 | R^{a1}-4 | R^{a1}-4 |
| 13 | R^{a1}-5 | Rai-5 | H | H |
| 14 | H | H | R^{a1}-5 | R^{a1}-5 |
| 15 | R^{a1}-5 | R^{a1}-5 | R^{a1}-5 | R^{a1}-5 |
| 16 | R^{a1}-6 | R^{a1}-6 | H | H |
| 17 | H | H | R^{a1}-6 | R^{a1}-6 |
| 18 | R^{a1}-6 | R^{a1}-6 | R^{a1}-6 | R^{a1}-6 |
| 19 | R^{a1}-7 | R^{a1}-7 | H | H |
| 20 | H | H | R^{a1}-7 | R^{a1}-7 |
| 21 | R^{a1}-7 | R^{a1}-7 | R^{a1}-7 | R^{a1}-7 |
| 22 | R^{a1}-8 | R^{a1}-8 | H | H |
| 23 | H | H | R^{a1}-8 | R^{a1}-8 |
| 24 | R^{a1}-8 | R^{a1}-8 | R^{a1}-8 | R^{a1}-8 |
| 25 | R^{a1}-9 | R^{a1}-9 | H | H |
| 26 | H | H | R^{a1}-9 | R^{a1}-9 |
| 27 | R^{a1}-9 | R^{a1}-9 | R^{a1}-9 | R^{a1}-9 |
| 28 | R^{a1}-10 | R^{a1}-10 | H | H |
| 29 | H | H | R^{a1}-10 | R^{a1}-10 |
| 30 | R^{a1}-10 | R^{a1}-10 | R^{a1}-10 | R^{a1}-10 |
| 31 | R^{a1}-11 | R^{a1}-11 | H | H |
| 32 | H | H | R^{a1}-11 | R^{a1}-11 |
| 33 | R^{a1}-11 | R^{a1}-11 | R^{a1}-11 | R^{a1}-11 |
| 34 | R^{a1}-12 | R^{a1}-12 | H | H |
| 35 | R^{a1}-12 | R^{a1}-12 | R^{a1}-12 | R^{a1}-12 |
| 36 | R^{a1}-13 | R^{a1}-13 | H | H |
| 37 | R^{a1}-13 | R^{a1}-13 | R^{a1}-13 | R^{a1}-13 |
| 38 | R^{a1}-14 | R^{a1}-14 | H | H |
| 39 | R^{a1}-14 | R^{a1}-14 | R^{a1}-14 | R^{a1}-14 |
| 40 | R^{a1}-15 | R^{a1}-15 | H | H |
| 41 | H | H | R^{a1}-15 | R^{a1}-15 |
| 42 | R^{a1}-15 | R^{a1}-15 | R^{a1}-15 | R^{a1}-15 |
| 43 | R^{a1}-16 | R^{a1}-16 | H | H |
| 44 | R^{a1}-16 | R^{a1}-16 | R^{a1}-16 | R^{a1}-16 |
| 45 | R^{a1}-17 | R^{a1}-17 | H | H |
| 46 | R^{a1}-17 | R^{a1}-17 | R^{a1}-17 | R^{a1}-17 |
| 47 | R^{a1}-18 | R^{a1}-18 | H | H |
| 48 | R^{a1}-18 | R^{a1}-18 | R^{a1}-18 | R^{a1}-18 |
| 49 | R^{a1}-19 | R^{a1}-19 | H | H |
| 50 | H | H | R^{a1}-19 | R^{a1}-19 |
| 51 | R^{a1}-19 | R^{a1}-19 | R^{a1}-19 | R^{a1}-19 |
| 52 | R^{a1}-20 | R^{a1}-20 | H | H |
| 53 | H | H | R^{a1}-20 | R^{a1}-20 |
| 54 | R^{a1}-20 | R^{a1}-20 | R^{a1}-20 | R^{a1}-20 |
| 55 | R^{a1}-21 | R^{a1}-21 | H | H |
| 56 | H | H | R^{a1}-21 | R^{a1}-21 |
| 57 | R^{a1}-21 | R^{a1}-21 | R^{a1}-21 | R^{a1}-21 |
| 58 | R^{a1}-24 | R^{a1}-24 | H | H |
| 59 | H | H | R^{a1}-24 | R^{a1}-24 |
| 60 | R^{a1}-24 | R^{a1}-24 | R^{a1}-24 | R^{a1}-24 |
| 61 | R^{a1}-25 | Rai-25 | H | H |
| 62 | H | H | R^{a1}-25 | R^{a1}-25 |
| 63 | R^{a1}-25 | R^{a1}-25 | R^{a1}-25 | R^{a1}-25 |
| 64 | R^{a1}-26 | R^{a1}-26 | H | H |
| 65 | H | H | R^{a1}-26 | R^{a1}-26 |
| 66 | R^{a1}-26 | R^{a1}-26 | R^{a1}-26 | R^{a1}-26 |
| 67 | R^{a1}-27 | R^{a1}-27 | H | H |
| 68 | H | H | R^{a1}-27 | R^{a1}-27 |
| 69 | R^{a1}-27 | R^{a1}-27 | R^{a1}-27 | R^{a1}-27 |
| 70 | R^{a1}-28 | R^{a1}-28 | H | H |
| 71 | H | H | R^{a1}-28 | R^{a1}-28 |
| 72 | R^{a1}-28 | R^{a1}-28 | R^{a1}-28 | R^{a1}-28 |
| 73 | R^{a1}-29 | R^{a1}-29 | H | H |
| 74 | H | H | R^{a1}-29 | R^{a1}-29 |
| 75 | R^{a1}-29 | R^{a1}-29 | R^{a1}-29 | R^{a1}-29 |
| 76 | R^{a1}-30 | R^{a1}-30 | H | H |
| 77 | H | H | R^{a1}-30 | R^{a1}-30 |
| 78 | R^{a1}-30 | R^{a1}-30 | R^{a1}-30 | R^{a1}-30 |
| 79 | R^{a1}-31 | R^{a1}-31 | H | H |
| 80 | H | H | R^{a1}-31 | R^{a1}-31 |
| 81 | R^{a1}-31 | R^{a1}-31 | R^{a1}-31 | R^{a1}-31 |
| 82 | R^{a1}-32 | R^{a1}-32 | H | H |
| 83 | H | H | R^{a1}-32 | R^{a1}-32 |
| 84 | R^{a1}-32 | R^{a1}-32 | R^{a1}-32 | R^{a1}-32 |
| 85 | R^{a1}-33 | R^{a1}-33 | H | H |
| 86 | H | H | R^{a1}-33 | R^{a1}-33 |
| 87 | R^{a1}-33 | R^{a1}-33 | R^{a1}-33 | R^{a1}-33 |
| 88 | R^{a1}-34 | R^{a1}-34 | H | H |
| 89 | H | H | R^{a1}-34 | R^{a1}-34 |
| 90 | R^{a1}-34 | R^{a1}-34 | R^{a1}-34 | R^{a1}-34 |
| 91 | R^{a1}-35 | R^{a1}-35 | H | H |
| 92 | H | H | R^{a1}-35 | R^{a1}-35 |
| 93 | R^{a1}-35 | R^{a1}-35 | R^{a1}-35 | R^{a1}-35 |
| 94 | R^{a1}-36 | R^{a1}-36 | H | H |
| 95 | H | H | R^{a1}-36 | R^{a1}-36 |
| 96 | R^{a1}-36 | R^{a1}-36 | R^{a1}-36 | R^{a1}-36 |

where:
R^{a1}-1=2-phenylethynyl,
R^{a1}-2=2-(1-naphthyl)ethynyl,
R^{a1}-3=2-(2-naphthyl)ethynyl,
R^{a1}-4=2-(2-phenylphenyl)ethynyl,
R^{a1}-5=2-(4-phenylphenyl)ethynyl,
R^{a1}-6=2-(phenanthren-9-yl)ethynyl,
R^{a1}-7=2-(dibenzofuran-2-yl)ethynyl,
R^{a1}-8=2-(dibenzofuran-4-yl)ethynyl,
R^{a1}-9=2-(dibenzothiophen-2-yl)ethynyl,
R^{a1}-10=2-(dibenzothiophen-4-yl)ethynyl,
R^{a1}-11=2-(triphenylen-2-yl)ethynyl,
R^{a1}-12=2-(pyridin-2-yl)ethynyl,
R^{a1}-13=2-(pyridin-3-yl)ethynyl,
R^{a1}-14=2-(pyridin-4-yl)ethynyl,
R^{a1}-15=2-(quinoline-2-yl)ethynyl,
R^{a1}-16=2-(quinoline-3-yl)ethynyl,
R^{a1}-17=2-(quinoline-4-yl)ethynyl,
R^{a1}-18=2-(quinoline-8-yl)ethynyl,
R^{a1}-19=4-(2-phenylethynyl)phenyl,
R^{a1}-20=4-(2-(2-naphthyl)ethynyl)phenyl,
R^{a1}-21=4-(2-(1-naphthyl)ethynyl)phenyl,
R^{a1}-22=4-(2-(2-phenylphenyl)ethynyl)phenyl,
R^{a1}-23=4-(2-(4-phenylphenyl)ethynyl)phenyl,
R^{a1}-24=4-(2-(phenanthren-9-yl)ethynyl)phenyl,
R^{a1}-25=4-(2-(dibenzofuran-2-yl)ethynyl)phenyl,
R^{a1}-26=4(2-(dibenzofuran-4-yl)ethynyl)phenyl,
R^{a1}-27=4-(2-(dibenzothiophen-2-yl)ethynyl)phenyl,
R^{a1}-28=4-(2-(dibenzothiophen-4-yl)ethynyl)phenyl,
R^{a1}-29=4-(2-(triphenylen-2-yl)ethynyl)phenyl,
R^{a1}-30=4-(2-(pyridin-2-yl)ethynyl)phenyl,
R^{a1}-31=4-(2-(pyridin-3-yl)ethynyl)phenyl,
R^{a1}-32=4-(2-(pyridin-4-yl)ethynyl)phenyl,
R^{a1}-33=4-(2-(quinoline-2-yl)ethynyl)phenyl,
R^{a1}-34=4-(2-(quinoline-3-yl)ethylenyl)phenyl,
R^{a1}-35=4-(2-(quinoline-4-yl)ethynyl)phenyl, and
R^{a1}-36=4-(2-(quinoline-8-yl)ethynyl)phenyl.

Amongst the compounds of formula (Ia-1) and the structural units of formula (IIa-1) recited in table A, particular preference is given to those compounds and structural units of formulae (Ia-1) and (IIa-1), where R^{a1} and R^{a2} are identical and selected from the group consisting of phenylethynyl, naphthalene-1-ylethynyl and 2-naphthalene-2-ylethynyl and where R^{a3} and R^{a4} are hydrogen. In other words, particular preference is given to the following compounds of the formula (Ia-1):
2,2′-bis(2-hydroxyethoxy)-6,6′-di(naphthalene-2-yl-ethynyl)-1,1′-binaphthalene (R^{a1} and R^{a2} are naphthalene-1-ylethynyl, R^{a3} and R^{a4} are hydrogen: D2NACBHBNA),
2,2′-bis(2-hydroxyethoxy)-6,6′-di(naphthalene-1-yl-ethynyl)-1,1′-binaphthalene (R^{a1} and R^{a2} are naphthalene-1-ylethynyl, R^{a3} and R^{a4} are hydrogen: D1NACBHBNA), and
2,2′-bis(2-hydroxyethoxy)-6,6′-di(phenylethynyl)-1,1′-binaphthalene (R^{a1} and R^{a2} are phenylethynyl, R^{a3} and R^{a4} are hydrogen: DPACBHBNA),

and to the structural units derived therefrom.

In a particular subgroup (4″a) of group (4″) of embodiments the compound of the formula (I) is a compound of the formula (Ib):

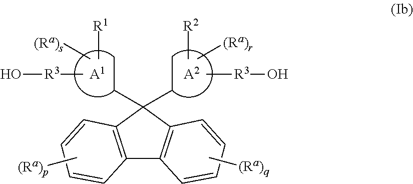

wherein the variables p, q, r and s are identical or different and are 0 or 1, and wherein the radicals A¹, A², R¹, R², R³ and R^{a} have the meanings defined herein, in particular one of the preferred meanings, provided that at least one of R¹ and R² is a radical R^{a}, if p, q, r and s are all 0. The radicals R¹ and R² are preferably identical. The variables r and s in formula (Ib) have preferably the same value. In case r and s are both 1, the two respective substituents R^{a} are preferably identical. Likewise, in case the variables p and q are both 1, the two respective substituents R^{a} are preferably identical and are located either in positions 2 and 7 or 3 and 6, in particular in positions 2 and 7, of the fluorenyl moiety of the compounds of formula (Ib).

In this subgroup (4″a) of group (4″) of embodiments the structural unit of the formula (II) is a structural unit of the formula (IIb):

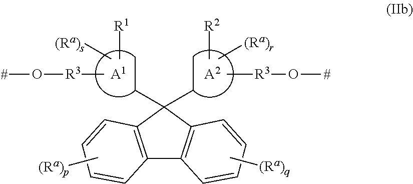

where #represents a connection point to a neighboring structural unit and where the variables A¹, A², R¹, R², R³, R^{a}, p, q, r and s have one of the meanings defined herein, in particular one of the preferred meanings. The statements on preferred meanings of the variables p, q, r and s as well as on preferred meanings and positions of the radicals R¹, R² and R^{a} provided above in the context of formula (Ib) also apply for formula (IIb).

In a particular subgroup (4″a.1) of groups (4″), (1) and (7″) of embodiments, the compound of the formula (I) is a compound of the formula (Ib-1):

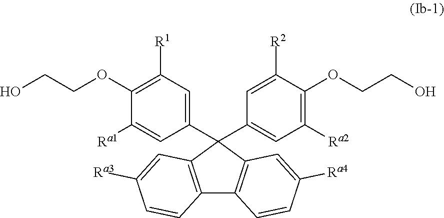

wherein the variables R¹ and R² are hydrogen, phenyl or a radical R^{a} and the variables R^{a1}, R^{a2}, R^{a3} and R^{a4} independently of one another are hydrogen or a radical R^{a}, provided that at least one of R¹, R², R^{a1}, R^{a2}, R^{a3} and R^{a4} in formula (Ib-1) is a radical R^{a}.

The radicals R¹ and R² in formula (Ib-1) preferably have the same meaning and are preferably selected from hydrogen, C₁-C₄-alkyl, phenyl, ethynyl, methylethynyl, phenylethynyl, naphthylethynyl, biphenylylethynyl, phenanthrylethynyl, dibenzofuranylethynyl, dibenzothiophenylethynyl, thianthrenylethynyl, triphenylenylethynyl, pyridinylethynyl, quinolinylethynyl, methylethynylphenyl, phenylethynylphenyl, methylethynylnaphthyl, phenylethynylnaphthyl, naphthylethynylphenyl, naphthylethynylnaphthyl, phenanthrylethynylphenyl, biphenylylethynylphenyl, triphenylenylethynylphenyl, pyridinylethynylphenyl, quinolinylethynylphenyl, dibenzofuranylethynylphenyl, dibenzothiophenylethynylphenyl and thianthrenylethynylphenyl, in particular from hydrogen, phenyl, 2-phenylethynyl, 2-(1-naphthyl)ethynyl, 2-(2-naphthyl)ethynyl, 2-(9-phenanthryl)ethynyl, 2-(2-dibenzofuranyl)ethynyl, 2-(4-dibenzofuranyl)ethynyl, 2-(2-dibenzothiophenyl)ethynyl, 2-(4-dibenzothiophenyl)ethynyl, 2-(1-thianthrenyl)ethynyl, 2-(2-thianthrenyl)ethynyl, 4-(2-phenylethynyl)phenyl, 4-(2-(1-naphthyl)ethynyl)phenyl, 4-(2-(2-naphthyl)ethynyl)phenyl, 4-(2-(9-phenanthrenyl)ethynyl)phenyl, 4-(2-(2-dibenzofuranyl)ethynyl)phenyl, 4-(2-(4-dibenzofuranyl)ethynyl)phenyl, 4-(2-(2-dibenzothiophenyl)ethynyl)phenyl, 4-(2-(4-dibenzothiophenyl)ethynyl)phenyl, 4-(2-(1-thianthrenyl)ethynyl)phenyl and 4-(2-(2-thianthrenyl)ethynyl)phenyl.

In particular, the radicals R¹ and R² in formula (Ib-1) are both either hydrogen or phenyl, or R¹ and R² together with the radicals R^{a1}, R^{a2}, R^{a3} and R^{a4} that are different from hydrogen are all identical radicals R^{a} which are preferably selected from phenylethynyl, naphthylethynyl, phenanthrylethynyl, dibenzofuranylethynyl, dibenzothiophenylethynyl, thianthrenylethynyl, phenylethynylphenyl, naphthylethynylphenyl, phenanthrenylethynylphenyl, dibenzofuranylethynyl)phenyl, dibenzothiophenylethynylphenyl and thianthrenylethynylphenyl. Especially, R¹ and R² are selected from hydrogen, phenyl, and Rₐ, where R^{a} is in particular 2-phenylethynyl, 2-(1-naphthyl)ethynyl or 2-(2-naphthyl)ethynyl.

In this subgroup (4″a.1) of groups (4″), (1) and (7″) of embodiments the structural unit of the formula (II) is a structural unit of the formula (IIb-1):

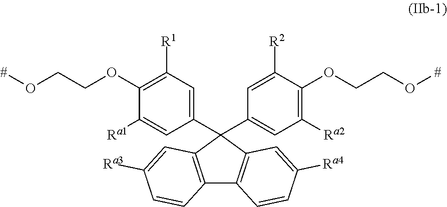

where #represents a connection point to a neighboring structural unit and where R¹, R², R^{a1}, R^{a2}, R^{a3} and R^{a4} have the same meanings defined above in the context of formula Ib-1, in particular the meanings mentioned as preferred.

In a further particular subgroup (4″a.2) of groups (4″), (2) and (7″) of embodiments, the compound of the formula (I) is a compound of the formula (Ib-2):

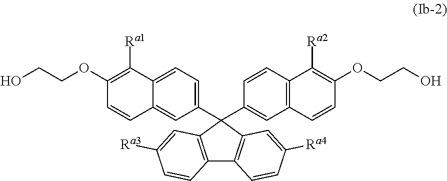

wherein the variables R^{a1}, R^{a2}, R^{a3} and R^{a4} independently of one another are hydrogen or a radical R^{a}, provided that at least one of R^{a1}, R^{a2}, R^{a3} and R^{a4} in formula (Ib-2) is a radical R^{a}.

In this subgroup (4″a.2) of groups (4″), (2) and (7″) of embodiments the structural unit of the formula (II) is a structural unit of the formula (IIb-2):

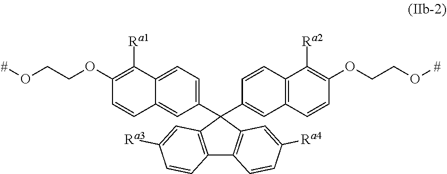

where #represents a connection point to a neighboring structural unit and where R^{a1}, R^{a2}, R^{a3} and R^{a4} have the same meanings defined above in the context of formula Ib-2.

The radicals R^{a1} and R^{a2} in formulae (Ib-1), (IIb-1), (Ib-2) or (IIb-2) have preferably identical meanings, while R^{a3} and R^{a4} may have different or identical meanings. If the meanings of R^{a3} and R^{a4} are different, it is preferred that one of R^{a3} and R^{a4} is hydrogen. The radicals R^{a1}, R^{a2}, R^{a3} and R^{a4} are preferably selected from hydrogen, C≡C—R¹¹ and Ar—C≡C—R¹¹, where the radical Ar is preferably phenylene or naphthylene, more preferably phenylene and in particular 1,4-phenylene, and where the radical R¹¹ is preferably selected from phenyl, naphthyl, specifically naphth-1-yl or naphth-2-yl, phenanthryl, specifically phenanthren-9-yl, biphenylyl, specifically 2-phenylphenyl or 4-phenylphenyl, triphenylenyl, specifically 2-triphenylenyl, dibenzo[b,d]furanyl, specifically 2-dibenzofuranyl or 4-dibenzofuranyl, dibenzo[b,d]thiophenyl, specifically 2-dibenzothiophenyl or 4-dibenzothiophenyl, thianthrenyl, specifically 1-thianthrenyl or 2-thianthrenyl, pyridyl, specifically 2-pyridyl, 3-pyridyl or 4-pyridyl, and quinolinyl, specifically 2-quinolinyl, 3-quinolinyl, 4-quinolinyl or 8-quinolinyl, and in particular is selected from phenyl, naphth-1-yl, naphth-2-yl, phenanthren-9-yl, 2-dibenzofuranyl, 4-dibenzofuranyl, 2-dibenzothiophenyl, 4-dibenzothiophenyl, 1-thianthrenyl and 2-thianthrenyl.

In particular, all variables R^{a1}, R^{a2}, R^{a3} and R^{a4} in formulae (Ib-1), (IIb-1), (Ib-2) or (IIb-2) different from hydrogen have identical meanings.

Examples of the particular subgroups (4″a.1) and (4″a.2) are the compounds and structural units of the formulae (Ib-1) and (IIb-1) or (Ib-2) and (IIb-2), where the combination of the radicals R¹, R², R^{a1}, R^{a2}, R^{a3} and R^{a4} or R^{a1}, R^{a2}, R^{a3} and R^{a4} is as defined in any one of rows 1 to 75 and 76 to 99, respectively, in table B below.

In a particular subgroup (4″b) of group (4″) of embodiments the compound of the formula (I) is a compound of the formula (Ic):

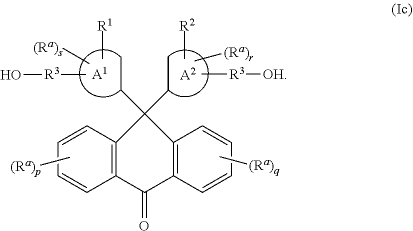

wherein the variables p, q, r and s are identical or different and are 0 or 1, and wherein the radicals A¹, A², R¹, R², R³ and R^{a} have the meanings defined herein, in particular one of the preferred meanings, provided that at least one of R¹ and R² is a radical R^{a}, if p, q, r and s are all 0. The radicals R¹ and R² are preferably identical. The variables r and s in formula (Ic) have preferably the same value. In case r and s are both 1, the two respective substituents R^{a} are preferably identical. Likewise, in case the variables p and q are both 1, the two respective substituents R^{a} are preferably identical and are located either in positions 2 and 7 or 3 and 6 of the anthronyl moiety of the compounds of formula (Ic).

In this subgroup (4″b) of group (4″) of embodiments the structural unit of the formula (II) is a structural unit of the formula (IIc):

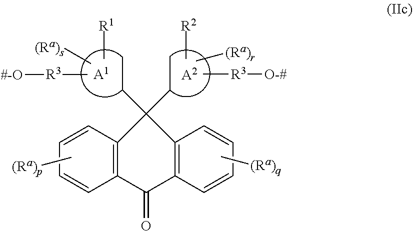

where #represents a connection point to a neighboring structural unit and where the variables A¹, A², R¹, R², R³, R^{a}, p, q, r and s have one of the meanings defined herein, in particular one of the preferred meanings. The statements on preferred meanings of the variables p, q, r and s as well as on preferred meanings and positions of the radicals R¹, R² and R^{a} provided above in the context of formula (Ic) also apply for formula (IIc).

In a particular subgroup (4″b.1) of groups (4″), (1) and (7″) of embodiments, the compound of the formula (I) is a compound of the formula (Ic-1):

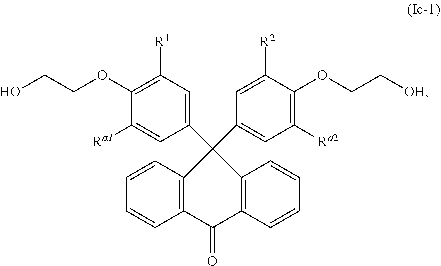

wherein the variables R¹ and R² are hydrogen, phenyl or a radical R^{a} and the variables R^{a1} and R^{a2} independently of one another are hydrogen or a radical R^{a}, provided that at least one of R¹, R², R^{a1} and R^{a2} in formula (Ic-1) is a radical R^{a}.

The radicals R¹ and R² in formula (Ic-1) preferably have the same meaning and are preferably selected from hydrogen, phenyl, ethynyl, methylethynyl, phenylethynyl, naphthylethynyl, biphenylylethynyl, phenanthrylethynyl, dibenzofuranylethynyl, dibenzothiophenylethynyl, thianthrenylethynyl, triphenylenylethynyl, pyridinylethynyl, quinolinylethynyl, methylethynylphenyl, phenylethynylphenyl, methylethynylnaphthyl, phenylethynylnaphthyl, naphthylethynylphenyl, naphthylethynylnaphthyl, phenanthrylethynylphenyl, biphenylylethynylphenyl, triphenylenylethynylphenyl, pyridinylethynylphenyl, quinolinylethynylphenyl, dibenzofuranylethynylphenyl, dibenzothiophenylethynylphenyl and thianthrenylethynylphenyl, in particular from hydrogen, phenyl, 2-phenylethynyl, 2-(1-naphthyl)ethynyl, 2-(2-naphthyl)ethynyl, 2-(9-phenanthryl)ethynyl, 2-(2-dibenzofuranyl)ethynyl, 2-(4-dibenzofuranyl)ethynyl, 2-(2-dibenzothiophenyl)ethynyl, 2-(4-dibenzothiophenyl)ethynyl, 2-(1-thianthrenyl)ethynyl, 2-(2-thianthrenyl)ethynyl, 4-(2-phenylethynyl)phenyl, 4-(2-(1-naphthyl)ethynyl)phenyl, 4-(2-(2-naphthyl)ethynyl)phenyl, 4-(2-(9-phenanthrenyl)ethynyl)phenyl, 4-(2-(2-dibenzofuranyl)ethynyl)phenyl, 4-(2-(4-dibenzofuranyl)ethynyl)phenyl, 4-(2-(2-dibenzothiophenyl)ethynyl)phenyl, 4-(2-(4-dibenzothiophenyl)ethynyl)phenyl, 4-(2-(1-thianthrenyl)ethynyl)phenyl and 4-(2-(2-thianthrenyl)ethynyl)phenyl.

In particular, the radicals R¹ and R² in formula (Ic-1) are both either hydrogen or phenyl, or R¹ and R² together with the radicals R^{a1} and R^{a2} that are different from hydrogen are all identical radicals R^{a} which are preferably selected from phenylethynyl, naphthylethynyl, phenanthrylethynyl, biphenylylethynyl, dibenzofuranylethynyl, dibenzothiophenylethynyl, thianthrenylethynyl, phenylethynylphenyl, naphthylethynylphenyl, phenanthrenylethynylphenyl, dibenzofuranylethynyl)phenyl, dibenzothiophenylethynylphenyl and thianthrenylethynylphenyl.

In this subgroup (4″b.1) of groups (4″), (1) and (7″) of embodiments the structural unit of the formula (II) is a structural unit of the formula (IIc-1):

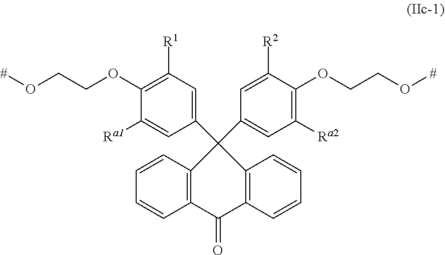

where #represents a connection point to a neighboring structural unit and where R¹, R², R^{a1} and R^{a2} have the same meanings defined above in the context of formula Ic-1, in particular the meanings mentioned as preferred.

In a further particular subgroup (4″b.2) of groups (2) and (7″) of embodiments, the compound of the formula (I) is a compound of the formula (Ic-2):

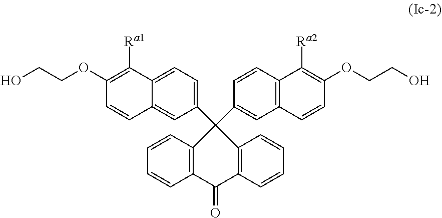

wherein the variables R^{a1} and R^{a2} independently of one another are hydrogen or a radical R^{a}, provided that at least one of R^{a1} and R^{a2} in formula (Ic-2) is a radical R^{a}.

In this subgroup (4″b.2) of groups (2) and (7″) of embodiments the structural unit of the formula (II) is a structural unit of the formula (IIc-2):

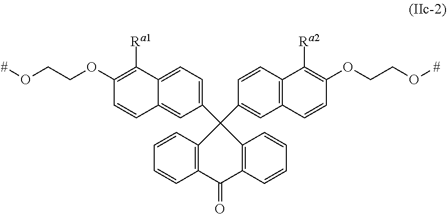

where #represents a connection point to a neighboring structural unit and where R^{a1} and R^{a2} have the same meanings defined above in the context of formula Ic-2.

Preferably, the radicals R^{a1} and R^{a2} in formulae (Ic-1), (IIc-1), (Ic-2) or (IIc-2) have the same meaning which is preferably selected from C≡C—R¹¹ and Ar—C≡C—R¹¹, where the radical Ar is preferably phenylene or naphthylene, more preferably phenylene and in particular 1,4-phenylene, and where the radical R¹¹ is preferably selected from phenyl, naphthyl, specifically naphth-1-yl or naphth-2-yl, phenanthryl, specifically phenanthren-9-yl, biphenylyl, specifically 2-phenylphenyl or 4-phenylphenyl, triphenylenyl, specifically 2-triphenylenyl, dibenzo[b,d]furanyl, specifically 2-dibenzofuranyl or 4-dibenzofuranyl, dibenzo[b,d]thiophenyl, specifically 2-dibenzothiophenyl or 4-dibenzothiophenyl, thianthrenyl, specifically 1-thianthrenyl or 2-thianthrenyl, pyridyl, specifically 2-pyridyl, 3-pyridyl or 4-pyridyl, and quinolinyl, specifically 2-quinolinyl, 3-quinolinyl, 4-quinolinyl or 8-quinolinyl, and in particular is selected from phenyl, naphth-1-yl, naphth-2-yl, phenanthren-9-yl, 2-dibenzofuranyl, 4-dibenzofuranyl, 2-dibenzothiophenyl, 4-dibenzothiophenyl, 1-thianthrenyl and 2-thianthrenyl.

Examples of the particular subgroups (4″b.1) and (4″b.2) are the compounds and structural units of the formulae (Ic-1) and (IIc-1) or (Ic-2) and (IIc-2), where the combination of the radicals R¹, R², R^{a1} and R^{a2} or R^{a1} and R^{a2} is as defined in any one of rows 100 to 145 and 146 to 158, respectively in table B below.

In a particular subgroup (4″c) of group (4″) of embodiments the compound of the formula (I) is a compound of the formula (Id):

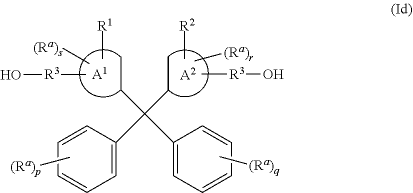

wherein the variables p, q, r and s are identical or different and are 0 or 1, and wherein the radicals A¹, A², R¹, R², R³ and R^{a} have the meanings defined herein, in particular one of the preferred meanings, provided that at least one of R¹ and R² is a radical R^{a}, if p, q, r and s are all 0. The radicals R¹ and R² are preferably identical. The variables r and s in formula (Id) have preferably the same value. In case r and s are both 1, the two respective substituents R^{a} are preferably identical. Likewise, in case the variables p and q are both 1, the two respective substituents R^{a} are preferably identical and are located either in positions 2 and 2′, 3 and 3′ or 4 and 4′ of the diphenylmethane moiety of the compounds of formula (Id).

In this subgroup (4″c) of group (4″) of embodiments the structural unit of the formula (II) is a structural unit of the formula (IId):

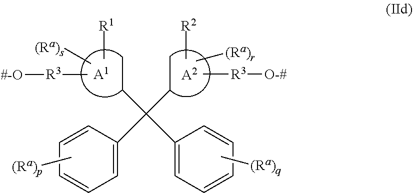

where #represents a connection point to a neighboring structural unit and where the variables A¹, A², R¹, R², R³, R^{a}, p, q, r and s have one of the meanings defined herein, in particular one of the preferred meanings. The statements on preferred meanings of the variables p, q, r and s as well as on preferred meanings and positions of the radicals R¹, R² and R^{a} provided above in the context of formula (Id) also apply for formula (IId).

In a particular subgroup (4″c.1) of groups (4″), (1) and (7″) of embodiments, the compound of the formula (I) is a compound of the formula (Id-1):

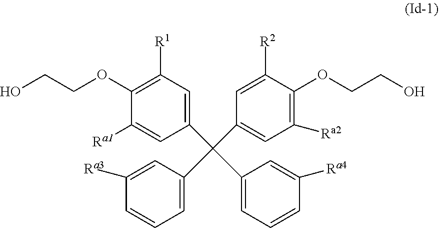

wherein the variables R¹ and R² are hydrogen, phenyl or a radical R^{a} and the variables R^{a1}, R^{a2}, R^{a3} and R^{a4} independently of one another are hydrogen or a radical R^{a}, provided that at least one of R¹, R², R^{a1}, R^{a2}, R^{a3} and R^{a4} in formula (Id-1) is a radical R^{a}.

The radicals R¹ and R² in formula (Id-1) preferably have the same meaning and are preferably selected from hydrogen, phenyl, ethynyl, methylethynyl, phenylethynyl, naphthylethynyl, biphenylylethynyl, phenanthrylethynyl, dibenzofuranylethynyl, dibenzothiophenylethynyl, thianthrenylethynyl, triphenylenylethynyl, pyridinylethynyl, quinolinylethynyl, methylethynylphenyl, phenylethynylphenyl, methylethynylnaphthyl, phenylethynylnaphthyl, naphthylethynylphenyl, naphthylethynylnaphthyl, phenanthrylethynylphenyl, biphenylylethynylphenyl, triphenylenylethynylphenyl, pyridinylethynylphenyl, quinolinylethynylphenyl, dibenzofuranylethynylphenyl and dibenzothiophenylethynylphenyl and thianthrenylethynylphenyl, in particular from hydrogen, phenyl, 2-phenylethynyl, 2-(1-naphthyl)ethynyl, 2-(2-naphthyl)ethynyl, 2-(9-phenanthryl)ethynyl, 2-(2-dibenzofuranyl)ethynyl, 2-(4-dibenzofuranyl)ethynyl, 2-(2-dibenzothiophenyl)ethynyl, 2-(4-dibenzothiophenyl)ethynyl, 2-(1-thianthrenyl)ethynyl, 2-(2-thianthrenyl)ethynyl, 4-(2-phenylethynyl)phenyl, 4-(2-(1-naphthyl)ethynyl)phenyl, 4-(2-(2-naphthyl)ethynyl)phenyl, 4-(2-(9-phenanthrenyl)ethynyl)phenyl, 4-(2-(2-dibenzofuranyl)ethynyl)phenyl, 4-(2-(4-dibenzofuranyl)ethynyl)phenyl, 4-(2-(2-dibenzothiophenyl)ethynyl)phenyl, 4-(2-(4-dibenzothiophenyl)ethynyl)phenyl, 4-(2-(1-thianthrenyl)ethynyl)phenyl and 4-(2-(2-thianthrenyl)ethynyl)phenyl.

In particular, the radicals R¹ and R² in formula (Id-1) are both either hydrogen or phenyl, or R¹ and R² together with the radicals R^{a1}, R^{a2}, R^{a3} and R^{a4} that are different from hydrogen are all identical radicals R^{a} which are preferably selected from phenylethynyl, naphthylethynyl, phenanthrylethynyl, biphenylylethynyl, dibenzofuranylethynyl, dibenzothiophenylethynyl, thianthrenylethynyl, phenylethynylphenyl, naphthylethynylphenyl, phenanthrenylethynylphenyl, dibenzofuranylethynylphenyl, dibenzothiophenylethynylphenyl and thianthrenylethynylphenyl.

In this subgroup (4″c.1) of groups (4″), (1) and (7″) of embodiments the structural unit of the formula (II) is a structural unit of the formula (IId-1):

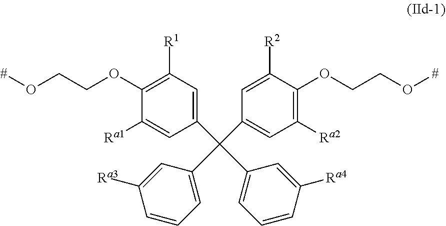

where #represents a connection point to a neighboring structural unit and where R¹, R², R^{a1}, R^{a2}, R^{a3} and R^{a4} have the same meanings defined above in the context of formula Id-1, in particular the meanings mentioned as preferred.

In a further particular subgroup (4″c.2) of groups (4″), (2) and (7″) of embodiments, the compound of the formula (I) is a compound of the formula (Id-2):

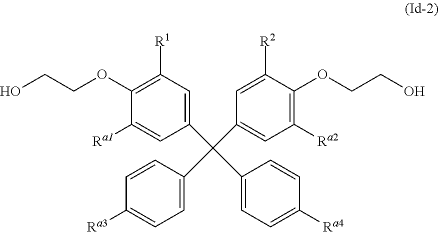

wherein the variables R¹ and R² are hydrogen, phenyl or a radical R^{a} and the variables R^{a1}, R^{a2}, R^{a3} and R^{a4} independently of one another are hydrogen or a radical R^{a}, provided that at least one of R¹, R², R^{a1}, R^{a2}, R^{a3} and R^{a4} in formula (Id-2) is a radical R^{a}.

The radicals R¹ and R² in formula (Id-2) preferably have the same meaning and are preferably selected from hydrogen, phenyl, ethynyl, methylethynyl, phenylethynyl, naphthylethynyl, biphenylylethynyl, phenanthrylethynyl, dibenzofuranylethynyl, dibenzothiophenylethynyl, thianthrenylethynyl, triphenylenylethynyl, pyridinylethynyl, quinolinylethynyl, methylethynylphenyl, phenylethynylphenyl, methylethynylnaphthyl, phenylethynylnaphthyl, naphthylethynylphenyl, naphthylethynylnaphthyl, phenanthrylethynylphenyl, biphenylylethynylphenyl, triphenylenylethynylphenyl, pyridinylethynylphenyl, quinolinylethynylphenyl, dibenzofuranylethynylphenyl, dibenzothiophenylethynylphenyl and thianthrenylethynylphenyl, in particular from hydrogen, phenyl, 2-phenylethynyl, 2-(1-naphthyl)ethynyl, 2-(2-naphthyl)ethynyl, 2-(9-phenanthryl)ethynyl, 2-(2-dibenzofuranyl)ethynyl, 2-(4-dibenzofuranyl)ethynyl, 2-(2-dibenzothiophenyl)ethynyl, 2-(4-dibenzothiophenyl)ethynyl, 2-(1-thianthrenyl)ethynyl, 2-(2-thianthrenyl)ethynyl, 4-(2-phenylethynyl)phenyl, 4-(2-(1-naphthyl)ethynyl)phenyl, 4-(2-(2-naphthyl)ethynyl)phenyl, 4-(2-(9-phenanthrenyl)ethynyl)phenyl, 4-(2-(2-dibenzofuranyl)ethynyl)phenyl, 4-(2-(4-dibenzofuranyl)ethynyl)phenyl, 4-(2-(2-dibenzothiophenyl)ethynyl)phenyl, 4-(2-(4-dibenzothiophenyl)ethynyl)phenyl, 4-(2-(1-thianthrenyl)ethynyl)phenyl and 4-(2-(2-thianthrenyl)ethynyl)phenyl.

In particular, the radicals R¹ and R² in formula (Id-2) are both either hydrogen or phenyl, or R¹ and R² together with the radicals R^{a1}, R^{a2}, R^{a3} and R^{a4} that are different from hydrogen are all identical radicals R^{a} which are preferably selected from phenylethynyl, naphthylethynyl, phenanthrylethynyl, biphenylylethynyl, dibenzofuranylethynyl, dibenzothiophenylethynyl, thianthrenylethynyl, phenylethynylphenyl, naphthylethynylphenyl, phenanthrenylethynylphenyl, dibenzofuranylethynyl)phenyl, dibenzothiophenylethynylphenyl and thianthrenylethynylphenyl.

In this subgroup (4″c.2) of groups (4″), (2) and (7″) of embodiments the structural unit of the formula (II) is a structural unit of the formula (IIb-2):

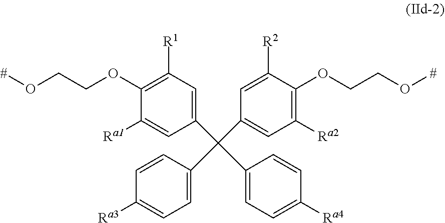

where #represents a connection point to a neighboring structural unit and where R¹, R², R^{a1}, R^{a2}, R^{a3} and R^{a4} have the same meanings defined above in the context of formula Id-1, in particular the meanings mentioned as preferred.

The radicals R^{a1} and R^{a2} in formulae (Id-1), (IId-1), (Id-2) or (IId-2) have preferably identical meanings, while the radicals R^{a3} and R^{a4} may have different or identical meanings. If the meanings of R^{a3} and R^{a4} are different, it is preferred that one of R^{a3} and R^{a4} is hydrogen. The radicals R^{a1}, R^{a2}, R^{a3} and R^{a4} are preferably selected from hydrogen, C≡C—R¹¹ and Ar—C≡C—R¹¹, where the radical Ar is preferably phenylene or naphthylene, more preferably phenylene and in particular 1,4-phenylene, and where the radical R¹¹ is preferably selected from phenyl, naphthyl, specifically naphth-1-yl or naphth-2-yl, phenanthryl, specifically phenanthren-9-yl, biphenylyl, specifically 2-phenylphenyl or 4-phenylphenyl, triphenylenyl, specifically 2-triphenylenyl, dibenzo[b,d]furanyl, specifically 2-dibenzofuranyl or 4-dibenzofuranyl, dibenzo[b,d]thiophenyl, specifically 2-dibenzothiophenyl or 4-dibenzothiophenyl, thianthrenyl, specifically 1-thianthrenyl or 2-thianthrenyl, pyridyl, specifically 2-pyridyl, 3-pyridyl or 4-pyridyl, and quinolinyl, specifically 2-quinolinyl, 3-quinolinyl, 4-quinolinyl or 8-quinolinyl, and in particular is selected from phenyl, naphth-1-yl, naphth-2-yl, phenanthren-9-yl, 2-dibenzofuranyl, 4-dibenzofuranyl, 2-dibenzothiophenyl, 4-dibenzothiophenyl, 1-thianthrenyl and 2-thianthrenyl.

In particular, all variables R^{a1}, R^{a2}, R^{a3} and R^{a4} in formulae (Id-1), (IId-1), (Id-2) or (IId-2) different from hydrogen have identical meanings.

Examples of the particular subgroups (4″c.1) and (4″c.2) are the compounds and structural units of the formulae (Id-1) and (IId-1) or (Id-2) and (IId-2), where the combination of the radicals R¹, R², R^{a1}, R^{a2}, R^{a3} and R^{a4} is as defined in any one of rows 159 to 242 and 243 to 271, respectively, in table B below.

In a further particular subgroup (4″c.3) of groups (4″), (2) and (7″) of embodiments, the compound of the formula (I) is a compound of the formula (Id-3):

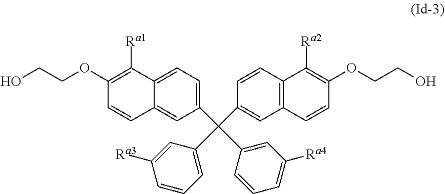

wherein the variables R^{a1}, R^{a2}, R^{a3} and R^{a4} independently of one another are hydrogen or a radical R^{a}, provided that at least one of R^{a1}, R^{a2}, R^{a3} and R^{a4} in formula (Id-3) is a radical R^{a}.

In this subgroup (4″c.3) of groups (4″), (2) and (7″) of embodiments the structural unit of the formula (II) is a structural unit of the formula (IId-3):

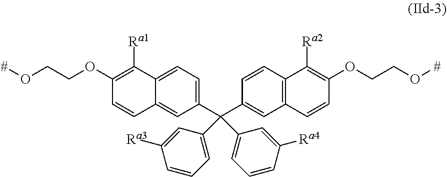

where #represents a connection point to a neighboring structural unit and where R^{a1}, R^{a2}, R^{a3} and R^{a4} have the same meanings defined above in the context of formula Id-3.

In a further particular subgroup (4″c.4) of groups (4″), (2) and (7″) of embodiments, the compound of the formula (I) is a compound of the formula (Id-4):

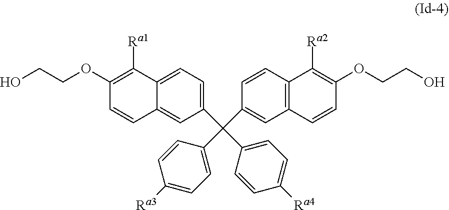

wherein the variables R^{a1}, R^{a2}, R^{a3} and R^{a4} independently of one another are hydrogen or a radical R^{a}, provided that at least one of R^{a1}, R^{a2}, R^{a3} and R^{a4} in formula (Id-4) is a radical R^{a}.

In this subgroup (4″c.4) of groups (4″), (2) and (7″) of embodiments the structural unit of the formula (II) is a structural unit of the formula (IId-4):

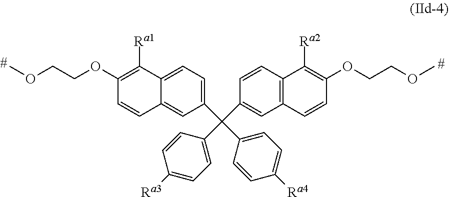

where #represents a connection point to a neighboring structural unit and where R^{a1}, R^{a2}, R^{a3} and R^{a4} have the same meanings defined above in the context of formula Id-3.

The radicals R^{a1} and R^{a2} in formulae (Id-3), (IId-3), (Id-4) or (IId-4) have preferably identical meanings, while the radicals R^{a3} and R^{a4} may have different or identical meanings. If the meanings of R^{a3} and R^{a4} are different, it is preferred that one of R^{a3} and R^{a4} is hydrogen. The radicals R^{a1}, R^{a2}, R^{a3} and R^{a4} are preferably selected from hydrogen, C≡C—R¹¹ and Ar—C≡C—R¹¹, where the radical Ar is preferably phenylene or naphthylene, more preferably phenylene and in particular 1,4-phenylene, and where the radical R¹¹ is preferably selected from phenyl, naphthyl, specifically naphth-1-yl or naphth-2-yl, phenanthryl, specifically phenanthren-9-yl, biphenylyl, specifically 2-phenylphenyl or 4-phenylphenyl, triphenylenyl, specifically 2-triphenylenyl, dibenzo[b,d]furanyl, specifically 2-dibenzofuranyl or 4-dibenzofuranyl, dibenzo[b,d]thiophenyl, specifically 2-dibenzothiophenyl or 4-dibenzothiophenyl, thianthrenyl, specifically 1-thianthrenyl or 2-thianthrenyl, pyridyl, specifically 2-pyridyl, 3-pyridyl or 4-pyridyl, and quinolinyl, specifically 2-quinolinyl, 3-quinolinyl, 4-quinolinyl or 8-quinolinyl, and in particular is selected from phenyl, naphth-1-yl, naphth-2-yl, phenanthren-9-yl, 2-dibenzofuranyl, 4-dibenzofuranyl, 2-dibenzothiophenyl, 4-dibenzothiophenyl, 1-thianthrenyl and 2-thianthrenyl.

In particular, all variables R^{a1}, R^{a2}, R^{a3} and R^{a4} in formulae (Id-3), (IId-3), (Id-4) or (IId-4) different from hydrogen have identical meanings.

Examples of the particular subgroups (4″c.3) and (4″c.4) are the compounds and structural units of the formulae (Id-3) and (IId-3) or (Id-4) and (IId-4), where the combination of the radicals R^{a1}, R^{a2}, R^{a3} and R^{a4} is as defined in any one of rows 272 to 328 and 329 to 373, respectively, in table B below.

TABLE B

| | Formula | R¹ | R² | R^{a1} | R^{a2} | Ra³ | Ra⁴ |
|---|---|---|---|---|---|---|---|
| 1 | Ib-1 | H | H | H | H | R^{a1}-1 | H |
| 2 | Ib-1 | H | H | H | H | R^{a1}-3 | H |
| 3 | Ib-1 | H | H | H | H | R^{a1}-7 | H |
| 4 | Ib-1 | H | H | H | H | R^{a1}-8 | H |
| 5 | Ib-1 | H | H | H | H | R^{a1}-9 | H |
| 6 | Ib-1 | H | H | H | H | R^{a1}-10 | H |
| 7 | Ib-1 | H | H | H | H | R^{a1}-19 | H |
| 8 | Ib-1 | H | H | H | H | R^{a1}-20 | H |
| 9 | Ib-1 | H | H | H | H | R^{a1}-24 | H |
| 10 | Ib-1 | H | H | H | H | R^{a1}-25 | H |
| 11 | Ib-1 | H | H | H | H | R^{a1}-26 | H |
| 12 | Ib-1 | H | H | H | H | R^{a1}-27 | H |
| 13 | Ib-1 | H | H | H | H | R^{a1}-28 | H |
| 14 | Ib-1 | H | H | H | H | R^{a1}-1 | R^{a1}-1 |
| 15 | Ib-1 | H | H | R^{a1}-1 | R^{a1}-1 | H | H |
| 16 | Ib-1 | H | H | R^{a1}-2 | R^{a1}-2 | H | H |
| 17 | Ib-1 | H | H | R^{a1}-3 | R^{a1}-3 | H | H |
| 18 | Ib-1 | H | H | R^{a1}-6 | R^{a1}-6 | H | H |
| 19 | Ib-1 | H | H | R^{a1}-7 | R^{a1}-7 | H | H |
| 20 | Ib-1 | H | H | R^{a1}-8 | R^{a1}-8 | H | H |
| 21 | Ib-1 | H | H | R^{a1}-9 | R^{a1}-9 | H | H |
| 22 | Ib-1 | H | H | R^{a1}-10 | R^{a1}-10 | H | H |
| 23 | Ib-1 | H | H | R^{a1}-19 | R^{a1}-19 | H | H |
| 24 | Ib-1 | H | H | R^{a1}-20 | R^{a1}-20 | H | H |
| 25 | Ib-1 | H | H | R^{a1}-21 | R^{a1}-21 | H | H |
| 26 | Ib-1 | H | H | R^{a1}-25 | R^{a1}-25 | H | H |
| 27 | Ib-1 | H | H | R^{a1}-26 | R^{a1}-26 | H | H |
| 28 | Ib-1 | H | H | R^{a1}-27 | R^{a1}-27 | H | H |
| 29 | Ib-1 | H | H | R^{a1}-28 | R^{a1}-28 | H | H |
| 30 | Ib-1 | R^{a1}-1 | R^{a1}-1 | R^{a1}-1 | R^{a1}-1 | H | H |
| 31 | Ib-1 | R^{a1}-2 | R^{a1}-2 | R^{a1}-2 | R^{a1}-2 | H | H |
| 32 | Ib-1 | R^{a1}-3 | R^{a1}-3 | R^{a1}-3 | R^{a1}-3 | H | H |
| 33 | Ib-1 | R^{a1}-6 | R^{a1}-6 | R^{a1}-6 | R^{a1}-6 | H | H |
| 34 | Ib-1 | R^{a1}-7 | R^{a1}-7 | R^{a1}-7 | R^{a1}-7 | H | H |
| 35 | Ib-1 | R^{a1}-8 | R^{a1}-8 | R^{a1}-8 | R^{a1}-8 | H | H |
| 36 | Ib-1 | R^{a1}-9 | R^{a1}-9 | R^{a1}-9 | R^{a1}-9 | H | H |
| 37 | Ib-1 | R^{a1}-10 | R^{a1}-10 | R^{a1}-10 | R^{a1}-10 | H | H |
| 38 | Ib-1 | R^{a1}-19 | R^{a1}-19 | R^{a1}-19 | R^{a1}-19 | H | H |
| 39 | Ib-1 | R^{a1}-20 | R^{a1}-20 | R^{a1}-20 | R^{a1}-20 | H | H |
| 40 | Ib-1 | R^{a1}-21 | R^{a1}-21 | R^{a1}-21 | R^{a1}-21 | H | H |
| 41 | Ib-1 | R^{a1}-24 | R^{a1}-24 | R^{a1}-24 | R^{a1}-24 | H | H |
| 42 | Ib-1 | R^{a1}-25 | R^{a1}-25 | R^{a1}-25 | R^{a1}-25 | H | H |
| 43 | Ib-1 | R^{a1}-26 | R^{a1}-26 | R^{a1}-26 | R^{a1}-26 | H | H |
| 44 | Ib-1 | R^{a1}-27 | R^{a1}-27 | R^{a1}-27 | R^{a1}-27 | H | H |
| 45 | Ib-1 | R^{a1}-28 | R^{a1}-28 | R^{a1}-28 | R^{a1}-28 | H | H |
| 46 | Ib-1 | phenyl | phenyl | H | H | R^{a1}-1 | H |
| 47 | Ib-1 | phenyl | phenyl | H | H | R^{a1}-3 | H |
| 48 | Ib-1 | phenyl | phenyl | H | H | R^{a1}-7 | H |
| 49 | Ib-1 | phenyl | phenyl | H | H | R^{a1}-8 | H |
| 50 | Ib-1 | phenyl | phenyl | H | H | R^{a1}-9 | H |
| 51 | Ib-1 | phenyl | phenyl | H | H | R^{a1}-10 | H |
| 52 | Ib-1 | phenyl | phenyl | H | H | R^{a1}-19 | H |
| 53 | Ib-1 | phenyl | phenyl | H | H | R^{a1}-20 | H |
| 54 | Ib-1 | phenyl | phenyl | H | H | R^{a1}-24 | H |
| 55 | Ib-1 | phenyl | phenyl | H | H | R^{a1}-25 | H |
| 56 | Ib-1 | phenyl | phenyl | H | H | R^{a1}-26 | H |
| 57 | Ib-1 | phenyl | phenyl | H | H | R^{a1}-27 | H |
| 58 | Ib-1 | phenyl | phenyl | H | H | R^{a1}-28 | H |
| 59 | Ib-1 | phenyl | phenyl | H | H | R^{a1}-1 | R^{a1}-1 |
| 60 | Ib-1 | phenyl | phenyl | R^{a1}-1 | R^{a1}-1 | H | H |
| 61 | Ib-1 | phenyl | phenyl | R^{a1}-2 | R^{a1}-2 | H | H |
| 62 | Ib-1 | phenyl | phenyl | R^{a1}-3 | R^{a1}-3 | H | H |
| 63 | Ib-1 | phenyl | phenyl | R^{a1}-6 | R^{a1}-6 | H | H |
| 64 | Ib-1 | phenyl | phenyl | R^{a1}-7 | R^{a1}-7 | H | H |
| 65 | Ib-1 | phenyl | phenyl | R^{a1}-8 | R^{a1}-8 | H | H |
| 66 | Ib-1 | phenyl | phenyl | R^{a1}-9 | R^{a1}-9 | H | H |
| 67 | Ib-1 | phenyl | phenyl | R^{a1}-10 | R^{a1}-10 | H | H |
| 68 | Ib-1 | phenyl | phenyl | R^{a1}-19 | R^{a1}-19 | H | H |
| 69 | Ib-1 | phenyl | phenyl | R^{a1}-20 | R^{a1}-20 | H | H |
| 70 | Ib-1 | phenyl | phenyl | R^{a1}-21 | R^{a1}-21 | H | H |
| 71 | Ib-1 | phenyl | phenyl | R^{a1}-24 | R^{a1}-24 | H | H |
| 72 | Ib-1 | phenyl | phenyl | R^{a1}-25 | R^{a1}-25 | H | H |
| 73 | Ib-1 | phenyl | phenyl | R^{a1}-26 | R^{a1}-26 | H | H |
| 74 | Ib-1 | phenyl | phenyl | R^{a1}-27 | R^{a1}-27 | H | H |
| 75 | Ib-1 | phenyl | phenyl | R^{a1}-28 | R^{a1}-28 | H | H |
| 76 | Ib-2 | — | — | H | H | R^{a1}-1 | H |
| 77 | Ib-2 | — | — | H | H | R^{a1}-3 | H |
| 78 | Ib-2 | — | — | H | H | R^{a1}-7 | H |
| 79 | Ib-2 | — | — | H | H | R^{a1}-8 | H |
| 80 | Ib-2 | — | — | H | H | R^{a1}-9 | H |
| 81 | Ib-2 | — | — | H | H | R^{a1}-10 | H |
| 82 | Ib-2 | — | — | H | H | R^{a1}-19 | H |
| 83 | Ib-2 | — | — | H | H | R^{a1}-25 | H |
| 84 | Ib-2 | — | — | H | H | R^{a1}-26 | H |
| 85 | Ib-2 | — | — | H | H | R^{a1}-27 | H |
| 86 | Ib-2 | — | — | H | H | R^{a1}-28 | H |
| 87 | Ib-2 | — | — | H | H | R^{a1}-1 | R^{a1}-1 |
| 88 | Ib-2 | — | — | R^{a1}-1 | R^{a1}-1 | H | H |
| 89 | Ib-2 | — | — | R^{a1}-8 | R^{a1}-8 | H | H |
| 90 | Ib-2 | — | — | R^{a1}-9 | R^{a1}-9 | H | H |
| 91 | Ib-2 | — | — | R^{a1}-19 | R^{a1}-19 | H | H |
| 92 | Ib-2 | — | — | R^{a1}-20 | R^{a1}-20 | H | H |
| 93 | Ib-2 | — | — | R^{a1}-21 | R^{a1}-21 | H | H |
| 94 | Ib-2 | — | — | R^{a1}-25 | R^{a1}-25 | H | H |
| 95 | Ib-2 | — | — | R^{a1}-26 | R^{a1}-26 | H | H |
| 96 | Ib-2 | — | — | R^{a1}-27 | R^{a1}-27 | H | H |
| 97 | Ib-2 | — | — | R^{a1}-28 | R^{a1}-28 | H | H |
| 98 | Ib-2 | — | — | R^{a1}-1 | R^{a1}-1 | R^{a1}-1 | H |
| 99 | Ib-2 | — | — | R^{a1}-1 | R^{a1}-1 | R^{a1}-1 | R^{a1}-1 |
| 100 | Ic-1 | H | H | R^{a1}-1 | R^{a1}-1 | — | — |
| 101 | Ic-1 | H | H | R^{a1}-2 | R^{a1}-2 | — | — |
| 102 | Ic-1 | H | H | R^{a1}-3 | R^{a1}-3 | — | — |
| 103 | Ic-1 | H | H | R^{a1}-6 | R^{a1}-6 | — | — |
| 104 | Ic-1 | H | H | R^{a1}-7 | R^{a1}-7 | — | — |
| 105 | Ic-1 | H | H | R^{a1}-8 | R^{a1}-8 | — | — |
| 106 | Ic-1 | H | H | R^{a1}-9 | R^{a1}-9 | — | — |
| 107 | Ic-1 | H | H | R^{a1}-10 | R^{a1}-10 | — | — |
| 108 | Ic-1 | H | H | R^{a1}-19 | R^{a1}-19 | — | — |
| 109 | Ic-1 | H | H | R^{a1}-20 | R^{a1}-20 | — | — |
| 110 | Ic-1 | H | H | R^{a1}-21 | R^{a1}-21 | — | — |
| 111 | Ic-1 | H | H | R^{a1}-24 | R^{a1}-24 | — | — |
| 112 | Ic-1 | H | H | R^{a1}-25 | R^{a1}-25 | — | — |
| 113 | Ic-1 | H | H | R^{a1}-26 | R^{a1}-26 | — | — |
| 114 | Ic-1 | H | H | R^{a1}-27 | R^{a1}-27 | — | — |
| 115 | Ic-1 | H | H | R^{a1}-28 | R^{a1}-28 | — | — |
| 116 | Ic-1 | R^{a1}-1 | R^{a1}-1 | R^{a1}-1 | R^{a1}-1 | — | — |
| 117 | Ic-1 | R^{a1}-2 | R^{a1}-2 | R^{a1}-2 | R^{a1}-2 | — | — |
| 118 | Ic-1 | R^{a1}-3 | R^{a1}-3 | R^{a1}-3 | R^{a1}-3 | — | — |
| 119 | Ic-1 | R^{a1}-7 | R^{a1}-7 | R^{a1}-7 | R^{a1}-7 | — | — |
| 120 | Ic-1 | R^{a1}-8 | R^{a1}-8 | R^{a1}-8 | R^{a1}-8 | — | — |
| 121 | Ic-1 | R^{a1}-9 | R^{a1}-9 | R^{a1}-9 | R^{a1}-9 | — | — |
| 122 | Ic-1 | R^{a1}-10 | R^{a1}-10 | R^{a1}-10 | R^{a1}-10 | — | — |
| 123 | Ic-1 | R^{a1}-19 | R^{a1}-19 | R^{a1}-19 | R^{a1}-19 | — | — |
| 124 | Ic-1 | R^{a1}-20 | R^{a1}-20 | R^{a1}-20 | R^{a1}-20 | — | — |
| 125 | Ic-1 | R^{a1}-21 | R^{a1}-21 | R^{a1}-21 | R^{a1}-21 | — | — |
| 126 | Ic-1 | R^{a1}-25 | R^{a1}-25 | R^{a1}-25 | R^{a1}-25 | — | — |
| 127 | Ic-1 | R^{a1}-26 | R^{a1}-26 | R^{a1}-26 | R^{a1}-26 | — | — |
| 128 | Ic-1 | R^{a1}-27 | R^{a1}-27 | R^{a1}-27 | R^{a1}-27 | — | — |
| 129 | Ic-1 | R^{a1}-28 | R^{a1}-28 | R^{a1}-28 | R^{a1}-28 | — | — |
| 130 | Ic-1 | phenyl | phenyl | R^{a1}-1 | R^{a1}-1 | — | — |
| 131 | Ic-1 | phenyl | phenyl | R^{a1}-2 | R^{a1}-2 | — | — |
| 132 | Ic-1 | phenyl | phenyl | R^{a1}-3 | R^{a1}-3 | — | — |
| 133 | Ic-1 | phenyl | phenyl | R^{a1}-6 | R^{a1}-6 | — | — |
| 134 | Ic-1 | phenyl | phenyl | R^{a1}-7 | R^{a1}-7 | — | — |
| 135 | Ic-1 | phenyl | phenyl | R^{a1}-8 | R^{a1}-8 | — | — |
| 136 | Ic-1 | phenyl | phenyl | R^{a1}-9 | R^{a1}-9 | — | — |
| 137 | Ic-1 | phenyl | phenyl | R^{a1}-10 | R^{a1}-10 | — | — |
| 138 | Ic-1 | phenyl | phenyl | R^{a1}-19 | R^{a1}-19 | — | — |
| 139 | Ic-1 | phenyl | phenyl | R^{a1}-20 | R^{a1}-20 | — | — |
| 140 | Ic-1 | phenyl | phenyl | R^{a1}-21 | R^{a1}-21 | — | — |
| 141 | Ic-1 | phenyl | phenyl | R^{a1}-24 | R^{a1}-24 | — | — |
| 142 | Ic-1 | phenyl | phenyl | R^{a1}-25 | R^{a1}-25 | — | — |
| 143 | Ic-1 | phenyl | phenyl | R^{a1}-26 | R^{a1}-26 | — | — |
| 144 | Ic-1 | phenyl | phenyl | R^{a1}-27 | R^{a1}-27 | — | — |
| 145 | Ic-1 | phenyl | phenyl | R^{a1}-28 | R^{a1}-28 | — | — |
| 146 | Ic-2 | — | — | R^{a1}-1 | R^{a1}-1 | — | — |
| 147 | Ic-2 | — | — | R^{a1}-2 | R^{a1}-2 | — | — |
| 148 | Ic-2 | — | — | R^{a1}-3 | R^{a1}-3 | — | — |
| 149 | Ic-2 | — | — | R^{a1}-7 | R^{a1}-7 | — | — |
| 150 | Ic-2 | — | — | R^{a1}-8 | R^{a1}-8 | — | — |
| 151 | Ic-2 | — | — | R^{a1}-9 | R^{a1}-9 | — | — |
| 152 | Ic-2 | — | — | R^{a1}-10 | R^{a1}-10 | — | — |
| 153 | Ic-2 | — | — | R^{a1}-19 | R^{a1}-19 | — | — |
| 154 | Ic-2 | — | — | R^{a1}-20 | R^{a1}-20 | — | — |
| 155 | Ic-2 | — | — | R^{a1}-25 | R^{a1}-25 | — | — |
| 156 | Ic-2 | — | — | R^{a1}-26 | R^{a1}-26 | — | — |
| 157 | Ic-2 | — | — | R^{a1}-27 | R^{a1}-27 | — | — |
| 158 | Ic-2 | — | — | R^{a1}-28 | R^{a1}-28 | — | — |
| 159 | Id-1 | H | H | H | H | R^{a1}-1 | H |
| 160 | Id-1 | H | H | H | H | R^{a1}-2 | H |
| 161 | Id-1 | H | H | H | H | R^{a1}-3 | H |
| 162 | Id-1 | H | H | H | H | R^{a1}-6 | H |
| 163 | Id-1 | H | H | H | H | R^{a1}-7 | H |
| 164 | Id-1 | H | H | H | H | R^{a1}-8 | H |
| 165 | Id-1 | H | H | H | H | R^{a1}-9 | H |
| 166 | Id-1 | H | H | H | H | R^{a1}-10 | H |
| 167 | Id-1 | H | H | H | H | R^{a1}-19 | H |
| 168 | Id-1 | H | H | H | H | R^{a1}-20 | H |
| 169 | Id-1 | H | H | H | H | R^{a1}-21 | H |
| 170 | Id-1 | H | H | H | H | R^{a1}-25 | H |
| 171 | Id-1 | H | H | H | H | R^{a1}-26 | H |
| 172 | Id-1 | H | H | H | H | R^{a1}-27 | H |
| 173 | Id-1 | H | H | H | H | R^{a1}-28 | H |
| 174 | Id-1 | H | H | H | H | R^{a1}-1 | R^{a1}-1 |
| 175 | Id-1 | H | H | H | H | R^{a1}-2 | R^{a1}-2 |
| 176 | Id-1 | H | H | H | H | R^{a1}-3 | R^{a1}-3 |
| 177 | Id-1 | H | H | H | H | R^{a1}-6 | R^{a1}-6 |
| 178 | Id-1 | H | H | H | H | R^{a1}-7 | R^{a1}-7 |
| 179 | Id-1 | H | H | H | H | R^{a1}-8 | R^{a1}-8 |
| 180 | Id-1 | H | H | H | H | R^{a1}-9 | R^{a1}-9 |
| 181 | Id-1 | H | H | H | H | R^{a1}-10 | R^{a1}-10 |
| 182 | Id-1 | H | H | H | H | R^{a1}-19 | R^{a1}-19 |
| 183 | Id-1 | H | H | H | H | R^{a1}-20 | R^{a1}-20 |
| 184 | Id-1 | H | H | H | H | R^{a1}-21 | R^{a1}-21 |
| 185 | Id-1 | H | H | H | H | R^{a1}-24 | R^{a1}-24 |
| 186 | Id-1 | H | H | H | H | R^{a1}-25 | R^{a1}-25 |
| 187 | Id-1 | H | H | H | H | R^{a1}-26 | R^{a1}-26 |
| 188 | Id-1 | H | H | H | H | R^{a1}-27 | R^{a1}-27 |
| 189 | Id-1 | H | H | H | H | R^{a1}-28 | R^{a1}-28 |
| 190 | Id-1 | H | H | R^{a1}-1 | R^{a1}-1 | H | H |
| 191 | Id-1 | H | H | R^{a1}-2 | R^{a1}-2 | H | H |
| 192 | Id-1 | H | H | R^{a1}-3 | R^{a1}-3 | H | H |
| 193 | Id-1 | H | H | R^{a1}-6 | R^{a1}-6 | H | H |
| 194 | Id-1 | H | H | R^{a1}-7 | R^{a1}-7 | H | H |
| 195 | Id-1 | H | H | R^{a1}-8 | R^{a1}-8 | H | H |
| 196 | Id-1 | H | H | R^{a1}-9 | R^{a1}-9 | H | H |
| 197 | Id-1 | H | H | R^{a1}-10 | R^{a1}-10 | H | H |
| 198 | Id-1 | H | H | R^{a1}-19 | R^{a1}-19 | H | H |
| 199 | Id-1 | H | H | R^{a1}-20 | R^{a1}-20 | H | H |
| 200 | Id-1 | H | H | R^{a1}-21 | R^{a1}-21 | H | H |
| 201 | Id-1 | H | H | R^{a1}-25 | R^{a1}-25 | H | H |
| 202 | Id-1 | H | H | R^{a1}-26 | R^{a1}-26 | H | H |
| 203 | Id-1 | H | H | R^{a1}-27 | R^{a1}-27 | H | H |
| 204 | Id-1 | H | H | R^{a1}-28 | R^{a1}-28 | H | H |
| 205 | Id-1 | R^{a1}-1 | R^{a1}-1 | R^{a1}-1 | R^{a1}-1 | H | H |
| 206 | Id-1 | R^{a1}-7 | R^{a1}-7 | R^{a1}-7 | R^{a1}-7 | H | H |
| 207 | Id-1 | R^{a1}-8 | R^{a1}-8 | R^{a1}-8 | R^{a1}-8 | H | H |
| 208 | Id-1 | R^{a1}-9 | R^{a1}-9 | R^{a1}-9 | R^{a1}-9 | H | H |
| 209 | Id-1 | R^{a1}-10 | R^{a1}-10 | R^{a1}-10 | R^{a1}-10 | H | H |
| 210 | Id-1 | R^{a1}-19 | R^{a1}-19 | R^{a1}-19 | R^{a1}-19 | H | H |
| 211 | Id-1 | R^{a1}-25 | R^{a1}-25 | R^{a1}-25 | R^{a1}-25 | H | H |
| 212 | Id-1 | R^{a1}-26 | R^{a1}-26 | R^{a1}-26 | R^{a1}-26 | H | H |
| 213 | Id-1 | R^{a1}-27 | R^{a1}-27 | R^{a1}-27 | R^{a1}-27 | H | H |
| 214 | Id-1 | R^{a1}-28 | R^{a1}-28 | R^{a1}-28 | R^{a1}-28 | H | H |
| 215 | Id-1 | phenyl | phenyl | R^{a1}-1 | R^{a1}-1 | H | H |
| 216 | Id-1 | phenyl | phenyl | R^{a1}-2 | R^{a1}-2 | H | H |
| 217 | Id-1 | phenyl | phenyl | R^{a1}-3 | R^{a1}-3 | H | H |
| 218 | Id-1 | phenyl | phenyl | R^{a1}-6 | R^{a1}-6 | H | H |
| 219 | Id-1 | phenyl | phenyl | R^{a1}-7 | R^{a1}-7 | H | H |
| 220 | Id-1 | phenyl | phenyl | R^{a1}-8 | R^{a1}-8 | H | H |
| 221 | Id-1 | phenyl | phenyl | R^{a1}-9 | R^{a1}-9 | H | H |
| 222 | Id-1 | phenyl | phenyl | R^{a1}-10 | R^{a1}-10 | H | H |
| 223 | Id-1 | phenyl | phenyl | R^{a1}-19 | R^{a1}-19 | H | H |
| 224 | Id-1 | phenyl | phenyl | R^{a1}-20 | R^{a1}-20 | H | H |
| 225 | Id-1 | phenyl | phenyl | R^{a1}-25 | R^{a1}-25 | H | H |
| 226 | Id-1 | phenyl | phenyl | R^{a1}-26 | R^{a1}-26 | H | H |
| 227 | Id-1 | phenyl | phenyl | R^{a1}-27 | R^{a1}-27 | H | H |
| 228 | Id-1 | phenyl | phenyl | R^{a1}-28 | R^{a1}-28 | H | H |
| 229 | Id-1 | phenyl | phenyl | H | H | R^{a1}-1 | R^{a1}-1 |
| 230 | Id-1 | phenyl | phenyl | H | H | R^{a1}-2 | R^{a1}-2 |
| 231 | Id-1 | phenyl | phenyl | H | H | R^{a1}-3 | R^{a1}-3 |
| 232 | Id-1 | phenyl | phenyl | H | H | R^{a1}-6 | R^{a1}-6 |
| 233 | Id-1 | phenyl | phenyl | H | H | R^{a1}-7 | R^{a1}-7 |
| 234 | Id-1 | phenyl | phenyl | H | H | R^{a1}-8 | R^{a1}-8 |
| 235 | Id-1 | phenyl | phenyl | H | H | R^{a1}-9 | R^{a1}-9 |
| 236 | Id-1 | phenyl | phenyl | H | H | R^{a1}-10 | R^{a1}-10 |
| 237 | Id-1 | phenyl | phenyl | H | H | R^{a1}-19 | R^{a1}-19 |
| 238 | Id-1 | phenyl | phenyl | H | H | R^{a1}-20 | R^{a1}-20 |
| 239 | Id-1 | phenyl | phenyl | H | H | R^{a1}-25 | R^{a1}-25 |
| 240 | Id-1 | phenyl | phenyl | H | H | R^{a1}-26 | R^{a1}-26 |
| 241 | Id-1 | phenyl | phenyl | H | H | R^{a1}-27 | R^{a1}-27 |
| 242 | Id-1 | phenyl | phenyl | H | H | R^{a1}-28 | R^{a1}-28 |
| 243 | Id-2 | H | H | H | H | R^{a1}-1 | H |
| 244 | Id-2 | H | H | H | H | R^{a1}-2 | H |
| 245 | Id-2 | H | H | H | H | R^{a1}-3 | H |
| 246 | Id-2 | H | H | H | H | R^{a1}-6 | H |
| 247 | Id-2 | H | H | H | H | R^{a1}-7 | H |
| 248 | Id-2 | H | H | H | H | R^{a1}-8 | H |
| 249 | Id-2 | H | H | H | H | R^{a1}-9 | H |
| 250 | Id-2 | H | H | H | H | R^{a1}-10 | H |
| 251 | Id-2 | H | H | H | H | R^{a1}-19 | H |
| 252 | Id-2 | H | H | H | H | R^{a1}-20 | H |
| 253 | Id-2 | H | H | H | H | R^{a1}-21 | H |
| 254 | Id-2 | H | H | H | H | R^{a1}-24 | H |
| 255 | Id-2 | H | H | H | H | R^{a1}-25 | H |
| 256 | Id-2 | H | H | H | H | R^{a1}-26 | H |
| 257 | Id-2 | H | H | H | H | R^{a1}-27 | H |
| 258 | Id-2 | H | H | H | H | R^{a1}-28 | H |
| 259 | Id-2 | H | H | H | H | R^{a1}-1 | R^{a1}-1 |
| 260 | Id-2 | H | H | H | H | R^{a1}-2 | R^{a1}-2 |
| 261 | Id-2 | H | H | H | H | R^{a1}-3 | R^{a1}-3 |
| 262 | Id-2 | H | H | H | H | R^{a1}-7 | R^{a1}-7 |
| 263 | Id-2 | H | H | H | H | R^{a1}-8 | R^{a1}-8 |
| 264 | Id-2 | H | H | H | H | R^{a1}-9 | R^{a1}-9 |
| 265 | Id-2 | H | H | H | H | R^{a1}-10 | R^{a1}-10 |
| 266 | Id-2 | H | H | H | H | R^{a1}-19 | R^{a1}-19 |
| 267 | Id-2 | H | H | H | H | R^{a1}-20 | R^{a1}-20 |
| 268 | Id-2 | H | H | H | H | R^{a1}-25 | R^{a1}-25 |
| 269 | Id-2 | H | H | H | H | R^{a1}-26 | R^{a1}-26 |
| 270 | Id-2 | H | H | H | H | R^{a1}-27 | R^{a1}-27 |
| 271 | Id-2 | H | H | H | H | R^{a1}-28 | R^{a1}-28 |
| 272 | Id-3 | — | — | H | H | R^{a1}-1 | H |
| 273 | Id-3 | — | — | H | H | R^{a1}-2 | H |
| 274 | Id-3 | — | — | H | H | R^{a1}-3 | H |
| 275 | Id-3 | — | — | H | H | R^{a1}-6 | H |
| 276 | Id-3 | — | — | H | H | R^{a1}-7 | H |
| 277 | Id-3 | — | — | H | H | R^{a1}-8 | H |
| 278 | Id-3 | — | — | H | H | R^{a1}-9 | H |
| 279 | Id-3 | — | — | H | H | R^{a1}-10 | H |
| 280 | Id-3 | — | — | H | H | R^{a1}-19 | H |
| 281 | Id-3 | — | — | H | H | R^{a1}-20 | H |
| 282 | Id-3 | — | — | H | H | R^{a1}-25 | H |
| 283 | Id-3 | — | — | H | H | R^{a1}-26 | H |
| 284 | Id-3 | — | — | H | H | R^{a1}-27 | H |
| 285 | Id-3 | — | — | H | H | R^{a1}-28 | H |
| 286 | Id-3 | — | — | H | H | R^{a1}-1 | R^{a1}-1 |
| 287 | Id-3 | — | — | H | H | R^{a1}-2 | R^{a1}-2 |
| 288 | Id-3 | — | — | H | H | R^{a1}-3 | R^{a1}-3 |
| 289 | Id-3 | — | — | H | H | R^{a1}-6 | R^{a1}-6 |
| 290 | Id-3 | — | — | H | H | R^{a1}-7 | R^{a1}-7 |
| 291 | Id-3 | — | — | H | H | R^{a1}-8 | R^{a1}-8 |
| 292 | Id-3 | — | — | H | H | R^{a1}-9 | R^{a1}-9 |
| 293 | Id-3 | — | — | H | H | R^{a1}-10 | R^{a1}-10 |
| 294 | Id-3 | — | — | H | H | R^{a1}-19 | R^{a1}-19 |
| 295 | Id-3 | — | — | H | H | R^{a1}-20 | R^{a1}-20 |
| 296 | Id-3 | — | — | H | H | R^{a1}-21 | R^{a1}-21 |
| 297 | Id-3 | — | — | H | H | R^{a1}-24 | R^{a1}-24 |
| 298 | Id-3 | — | — | H | H | R^{a1}-25 | R^{a1}-25 |
| 299 | Id-3 | — | — | H | H | R^{a1}-26 | R^{a1}-26 |
| 300 | Id-3 | — | — | H | H | R^{a1}-27 | R^{a1}-27 |
| 301 | Id-3 | — | — | H | H | R^{a1}-28 | R^{a1}-28 |
| 302 | Id-3 | — | — | R^{a1}-1 | R^{a1}-1 | H | H |
| 303 | Id-3 | — | — | R^{a1}-2 | R^{a1}-2 | H | H |
| 304 | Id-3 | — | — | R^{a1}-3 | R^{a1}-3 | H | H |
| 305 | Id-3 | — | — | R^{a1}-6 | R^{a1}-6 | H | H |
| 306 | Id-3 | — | — | R^{a1}-7 | R^{a1}-7 | H | H |
| 307 | Id-3 | — | — | R^{a1}-8 | R^{a1}-8 | H | H |
| 308 | Id-3 | — | — | R^{a1}-9 | R^{a1}-9 | H | H |
| 309 | Id-3 | — | — | R^{a1}-10 | R^{a1}-10 | H | H |
| 310 | Id-3 | — | — | R^{a1}-19 | R^{a1}-19 | H | H |
| 311 | Id-3 | — | — | R^{a1}-20 | R^{a1}-20 | H | H |
| 312 | Id-3 | — | — | R^{a1}-21 | R^{a1}-21 | H | H |
| 313 | Id-3 | — | — | R^{a1}-24 | R^{a1}-24 | H | H |
| 314 | Id-3 | — | — | R^{a1}-25 | R^{a1}-25 | H | H |
| 315 | Id-3 | — | — | R^{a1}-26 | R^{a1}-26 | H | H |
| 316 | Id-3 | — | — | R^{a1}-27 | R^{a1}-27 | H | H |
| 317 | Id-3 | — | — | R^{a1}-28 | R^{a1}-28 | H | H |
| 318 | Id-3 | — | — | R^{a1}-1 | R^{a1}-1 | R^{a1}-1 | R^{a1}-1 |
| 319 | Id-3 | — | — | R^{a1}-3 | R^{a1}-3 | R^{a1}-3 | R^{a1}-3 |
| 320 | Id-3 | — | — | R^{a1}-7 | R^{a1}-7 | R^{a1}-7 | R^{a1}-7 |
| 321 | Id-3 | — | — | R^{a1}-8 | R^{a1}-8 | R^{a1}-8 | R^{a1}-8 |
| 322 | Id-3 | — | — | R^{a1}-9 | R^{a1}-9 | R^{a1}-9 | R^{a1}-9 |
| 323 | Id-3 | — | — | R^{a1}-10 | R^{a1}-10 | R^{a1}-10 | R^{a1}-10 |
| 324 | Id-3 | — | — | R^{a1}-19 | R^{a1}-19 | R^{a1}-19 | R^{a1}-19 |
| 325 | Id-3 | — | — | R^{a1}-25 | R^{a1}-25 | R^{a1}-25 | R^{a1}-25 |
| 326 | Id-3 | — | — | R^{a1}-26 | R^{a1}-26 | R^{a1}-26 | R^{a1}-26 |
| 327 | Id-3 | — | — | R^{a1}-27 | R^{a1}-27 | R^{a1}-27 | R^{a1}-27 |
| 328 | Id-3 | — | — | R^{a1}-28 | R^{a1}-28 | R^{a1}-28 | R^{a1}-28 |
| 329 | Id-4 | — | — | H | H | R^{a1}-1 | H |
| 330 | Id-4 | — | — | H | H | R^{a1}-2 | H |
| 331 | Id-4 | — | — | H | H | R^{a1}-3 | H |
| 332 | Id-4 | — | — | H | H | R^{a1}-6 | H |
| 333 | Id-4 | — | — | H | H | R^{a1}-7 | H |
| 334 | Id-4 | — | — | H | H | R^{a1}-8 | H |
| 335 | Id-4 | — | — | H | H | R^{a1}-9 | H |
| 336 | Id-4 | — | — | H | H | R^{a1}-10 | H |
| 337 | Id-4 | — | — | H | H | R^{a1}-19 | H |
| 338 | Id-4 | — | — | H | H | R^{a1}-20 | H |
| 339 | Id-4 | — | — | H | H | R^{a1}-21 | H |
| 340 | Id-4 | — | — | H | H | R^{a1}-24 | H |
| 341 | Id-4 | — | — | H | H | R^{a1}-25 | H |
| 342 | Id-4 | — | — | H | H | R^{a1}-26 | H |
| 343 | Id-4 | — | — | H | H | R^{a1}-27 | H |
| 344 | Id-4 | — | — | H | H | R^{a1}-28 | H |
| 345 | Id-4 | — | — | H | H | R^{a1}-1 | R^{a1}-1 |
| 346 | Id-4 | — | — | H | H | R^{a1}-2 | R^{a1}-2 |
| 347 | Id-4 | — | — | H | H | R^{a1}-3 | R^{a1}-3 |
| 348 | Id-4 | — | — | H | H | R^{a1}-6 | R^{a1}-6 |
| 349 | Id-4 | — | — | H | H | R^{a1}-7 | R^{a1}-7 |
| 350 | Id-4 | — | — | H | H | R^{a1}-8 | R^{a1}-8 |
| 351 | Id-4 | — | — | H | H | R^{a1}-9 | R^{a1}-9 |
| 352 | Id-4 | — | — | H | H | R^{a1}-10 | R^{a1}-10 |
| 353 | Id-4 | — | — | H | H | R^{a1}-19 | R^{a1}-19 |
| 354 | Id-4 | — | — | H | H | R^{a1}-20 | R^{a1}-20 |
| 355 | Id-4 | — | — | H | H | R^{a1}-21 | R^{a1}-21 |
| 356 | Id-4 | — | — | H | H | R^{a1}-24 | R^{a1}-24 |
| 357 | Id-4 | — | — | H | H | R^{a1}-25 | R^{a1}-25 |
| 358 | Id-4 | — | — | H | H | R^{a1}-26 | R^{a1}-26 |
| 359 | Id-4 | — | — | H | H | R^{a1}-27 | R^{a1}-27 |
| 360 | Id-4 | — | — | H | H | R^{a1}-28 | R^{a1}-28 |
| 361 | Id-4 | — | — | R^{a1}-1 | R^{a1}-1 | R^{a1}-1 | R^{a1}-1 |
| 362 | Id-4 | — | — | R^{a1}-2 | R^{a1}-2 | R^{a1}-2 | R^{a1}-2 |
| 363 | Id-4 | — | — | R^{a1}-3 | R^{a1}-3 | R^{a1}-3 | R^{a1}-3 |
| 364 | Id-4 | — | — | R^{a1}-7 | R^{a1}-7 | R^{a1}-7 | R^{a1}-7 |
| 365 | Id-4 | — | — | R^{a1}-8 | R^{a1}-8 | R^{a1}-8 | R^{a1}-8 |
| 366 | Id-4 | — | — | R^{a1}-9 | R^{a1}-9 | R^{a1}-9 | R^{a1}-9 |
| 367 | Id-4 | — | — | R^{a1}-10 | R^{a1}-10 | R^{a1}-10 | R^{a1}-10 |
| 368 | Id-4 | — | — | R^{a1}-19 | R^{a1}-19 | R^{a1}-19 | R^{a1}-19 |
| 369 | Id-4 | — | — | R^{a1}-20 | R^{a1}-20 | R^{a1}-20 | R^{a1}-20 |
| 370 | Id-4 | — | — | R^{a1}-25 | R^{a1}-25 | R^{a1}-25 | R^{a1}-25 |
| 371 | Id-4 | — | — | R^{a1}-26 | R^{a1}-26 | R^{a1}-26 | R^{a1}-26 |
| 372 | Id-4 | — | — | R^{a1}-27 | R^{a1}-27 | R^{a1}-27 | R^{a1}-27 |
| 373 | Id-4 | — | — | R^{a1}-28 | R^{a1}-28 | R^{a1}-28 | R^{a1}-28 |

where the radicals R^{a1}-1, R^{a1}-2, R^{a1}-3, R^{a1}-6, R^{a1}-7, R^{a1}-8, R^{a1}-9, R^{a1}-10, R^{a1}-19, R^{a1}-20, R^{a1}-21, R^{a1}-24, R^{a1}-25, R^{a1}-26, R^{a1}-27 and R^{a1}-28 are as defined herein in the context of table A.

The compounds of the formula (Ia-1), where the radicals R^{a1}, R^{a2}, R^{a3} and R^{a4} differing from hydrogen have identical meanings, can be prepared from readily available 1,1′-binaphthol (compound VI) by the process according to the following reaction scheme 1a:

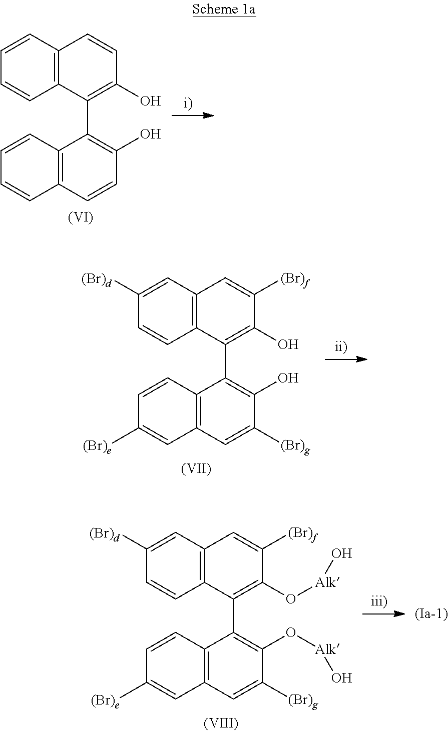

In step i) of the process according to scheme 1a, 1,1′-binaphthol is brominated to selectively yield the brominated 1,1′-binaphthol of formula (VII), where the variables d, e, f and g are either 1 or 0. Bromination in positions 6 and 6′ can be simply achieved by mixing 1,1′-binaphthol at low temperatures with a suitable brominating reagent in a polar aprotic solvent, which is inert against bromination. Suitable brominating agents are in particular elemental bromine. Suitable polar aprotic solvents for step i) include aliphatic halogenated hydrocarbon compounds, such as dichloromethane, trichloromethane, dichloroethane or dibromomethane, esters, such as isopropyl acetate or ethyl acetate, and mixtures thereof. Suitable reaction temperatures for bromination of 1,1′-binaphthol with bromine are below 0° C. and in particular in the range from −100 to −30° C. Further details can be taken from Bunzen et al. J. Am. Chem. Soc., 2009, 131(10), 3621-3630. As an alternative, N-bromosuccinimide can be used as a bromination agent. In this case, reaction temperatures will be higher than for the bromination with elemental bromine, e.g. from 0 to 50° C. Suitable solvents may then, in addition to aliphatic halogenated hydrocarbons, also include aliphatic ketones having from 3 to 6 carbon atoms, such as acetone, methyl ethyl ketone, methyl isobutyl ketone or diethyl ketone, cyclic ethers having from 4 to 6 carbon atoms, such as tetrahydrofuran, dioxan, diethyl ether, cyclopentyl methyl ether, and other solvents like acetonitrile, dimethylformamide, chloroform, methylene chloride, dichloroethane, as well as mixtures thereof with aliphatic halogenated hydrocarbons. Bromination in positions 3 and 3′ of 1,1-binaphthol or 6,6′-bibromo-1,1′-binaphthol is possible after introducing a suitable protection group for the hydroxyl functions followed by ortho-lithiation with butyl lithium and finally treating with bromine (see e.g. Y. Xu et al., J. Org. Chem. 2005, 70 (20), 8079-8087; and J. Yu et al., J. Am. Chem. Soc. 2008, 130 (25), 7845-47)

Therefore, by choosing the suitable bromination method and possibly combining them it is possible to effect bromination in positions 3 and 3′, positions 6 and 6′ or positions 3, 3′, 6 and 6′. Thus, compounds of formula (VII) can be obtained where the variables d, e, f and g have the following meanings:

(1) d=e=1 and f=g=0, (2) d=e=0 and f=g=1, or (3) d=e=f=g=1.

Alternatively, the brominated 1,1′-binaphthol compound of formula (VII), where d and e have the same meaning and f and g have the same meaning, can also be synthesized by copper(II)-catalyzed oxidative coupling of the corresponding mono- or dibromo-naphthols, e.g. in accordance with the procedure described in H. Egami et al., J. Am. Chem. Soc. 2009, 13 (17), 6082-83).

According to step ii) of scheme 1a the di- or tetrabrominated compound of formula (VII) is reacted with a cyclic carbonate of the formula (IX)

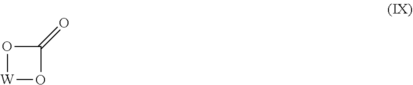

where W is an Alk′ moiety as defined above and in particular is 1,2-ethandiyl to yield the compound of formula (VIII). Hence, an example of a suitable compound of formula (IX) is ethylene carbonate. The compound of formula (IX) is usually applied in excess of the desired stoichiometry, i.e. the molar ratio of compound (IX) to the compound (VII) exceeds 2:1 and is in particular in the range from 2.2:1 to 5:1. The reaction according to step ii) of scheme 1a is usually performed in the presence of a base, in particular an oxo base, especially an alkaline carbonate such as sodium carbonate or potassium carbonate. The base is usually used in catalytic amounts, e.g. in amount from 0.1 to 0.5 mol per 1 mol of the compound (VII). Frequently, the reaction of the compound of formula (VII) with the compound of formula (IX) is performed in an aprotic organic solvent, in particular in an aromatic hydrocarbon solvent such as toluene, xylene or anisole and mixtures thereof. The reaction according to step ii) of scheme 1a is usually performed at temperatures in the range from 50 to 150° C.

In case the radicals R^{a} in the compound of formula (Ia-1) are moieties Ar—C≡C—R¹¹, as defined herein, the conversion in step iii) of scheme 1a can be accomplished e.g. by reacting the compound of formula (VIII) with a boronic compound of the formula (X)

R^{a}—B(OH)₂ (X)

where R^{a} is a radical Ar—C≡C—R¹¹, as defined herein, or with an ester or anhydride of (X), in particular a C₁-C₄-alkyl ester of (X), in the presence of a transition metal catalyst, in particular in the presence of a palladium catalyst. This conversion is frequently performed under the conditions of a so-called “Suzuki Reaction” or “Suzuki Coupling” (see e.g. A. Suzuki et al., Chem. Rev. 1995, 95, 2457-2483; N. Zhe et al., J. Med. Chem. 2005, 48 (5), 1569-1609; Young et al., J. Med. Chem. 2004, 47 (6), 1547-1552; C. Slee et al., Bioorg. Med. Chem. Lett. 2001, 9, 3243-3253; T. Zhang et al., Tetrahedron Lett., 52 (2011), 311-313, S. Bourrain et al., Synlett. 5 (2004), 795-798, B. Li et al., Europ. J. Org. Chem. 20113932-3937). Suitable transition metal catalysts are in particular palladium compounds, which bear at least one palladium atom and at least one tri-substituted phosphine ligand. Examples of palladium catalysts are tetrakis(triphenylphosphine) palladium, tetrakis(tritolylphosphine) palladium and [1,1-bis(diphenylphosphino)ferrocene]dichloropalladium(II) (PdCl₂(dppf)). Frequently, the palladium catalysts are prepared in situ from a suitable palladium precursor and a suitable phosphine ligand. Suitable palladium precursors are palladium compounds such as tris-(dibenzylideneacetone)dipalladium(0) (Pd₂(dba)₃) or palladium(II) acetate (Pd(OAc)₂). Suitable phosphine ligands are in particular tri(substituted)phosphines, e.g. a triarylphosphines such as triphenylphosphine, tritolylphosphine or 2,2′-bis(diphenyl-phosphino)-1,1′-binaphthalene (BINAP), tri(cyclo)alkylphosphine such as tris-n-butylphosphine, tris(tert-butyl)phosphine or tris(cyclohexylphosphine), or dicyclohexyl-(2′,4′,6′-tri-iso-propyl-biphenyl-2-yl)-phosphane (X-Phos). Usually, the reaction is performed in the presence of a base, in particular an oxo base, such as an alkaline alkoxide, earth alkaline alkoxide, alkaline hydroxides, earth alkaline hydroxides, alkaline carbonate or earth alkaline carbonate such as or sodium ethoxide, sodium tert-butoxide, potassium tert-butoxide, lithium hydroxide, barium hydroxide, sodium carbonate, potassium carbonate, or cesium carbonate. Frequently, the reaction according to step iii) of scheme 1a is performed in an organic solvent or in a mixture thereof with water. If the reaction is performed in a mixture of an organic solvent and water, the reaction mixture may be monophasic or biphasic. Suitable organic solvents include but are not limited to aromatic hydrocarbons such as toluene or xylene, acyclic and cyclic ethers, such as methyl tert.-butyl ether, ethyl tert.-butyl ether, diisopropylether, dioxane or tetrahydrofuran, and aliphatic alcohols having 1 to 4 carbon atoms, such as methanol, ethanol or isopropanol, as well as mixtures thereof.

The reaction according to step iii) of scheme 1a is usually performed at temperatures in the range from 50 to 150° C.

In case radicals R^{a} in the compound of formula (Ia-1) are moieties C≡C—R¹¹, as defined herein, the conversion in step iii) of scheme 1a can be accomplished e.g. by reacting the compound of formula (VIII) with an acetylene compound of the formula (XI)

H—C≡C—R¹¹ (XI)

where R¹¹ is as defined herein, in the presence of a transition metal catalyst, in particular a palladium catalyst, and a copper salt. This conversion is frequently performed under the conditions of a so-called “Sonogashira Coupling or Reaction” or “Sonogashira-Hagihara Coupling or Reaction” (see e.g. R. Chinchilla, C. Nájera, Chem. Soc. Rev. 2011, 40(10), 5084-5121; R. Chinchilla, C. Nájera, Chem. Rev. 2007, 107, 874-922; K. Sonogashira et al., Tetrahedron Lett. 1975, 50, 4467). Suitable transition metal catalysts are in particular palladium compounds, which bear at least one palladium atom and at least one tri-substituted phosphine ligand. Examples of palladium catalysts are tetrakis(triphenylphosphine) palladium, bis(triphenylphosphino)dichloropalladium (II) and [1,1-bis(diphenylphosphino)ferrocene]dichloropalladium(II) (PdCl₂(dppf)). Frequently, the palladium catalysts are prepared in situ from a suitable palladium precursor and a suitable phosphine ligand. Suitable palladium precursors are palladium compounds such as palladium(II) chloride or palladium(II) acetate (Pd(OAc)₂). Suitable phosphine ligands are in particular tri(substituted)phosphines, e.g. a triarylphosphines such as triphenylphosphine. The copper salt is typically selected from copper (I) iodide or copper(I) bromide. Usually, the reaction is performed in the presence of an amine base such as triethyl amine, piperidine or pyridine. Frequently, the reaction is performed in the amine base as solvent or in an organic solvent or in a mixture of the two. Suitable organic solvents are in particular those mentioned above in the context of the conversion with a compound of the formula (X) or an ester or anhydride thereof. The conversion with a compound of the formula (XI) is usually performed at temperatures in the range from 50 to 150° C.

The sequence of steps i), ii) and iii) can be changed as depicted in the following schemes 1b and 1c.

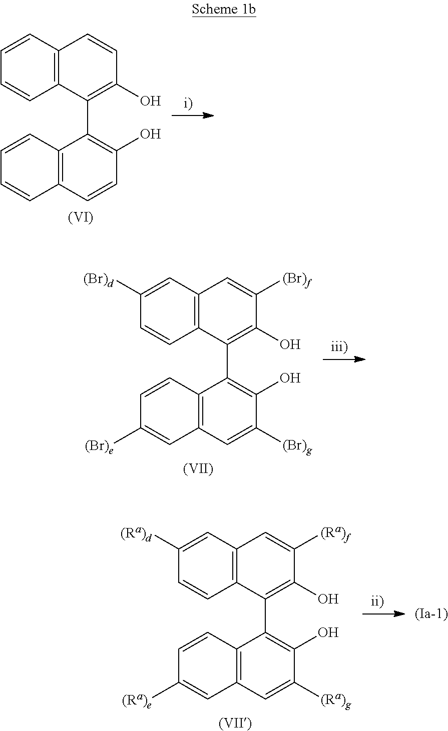

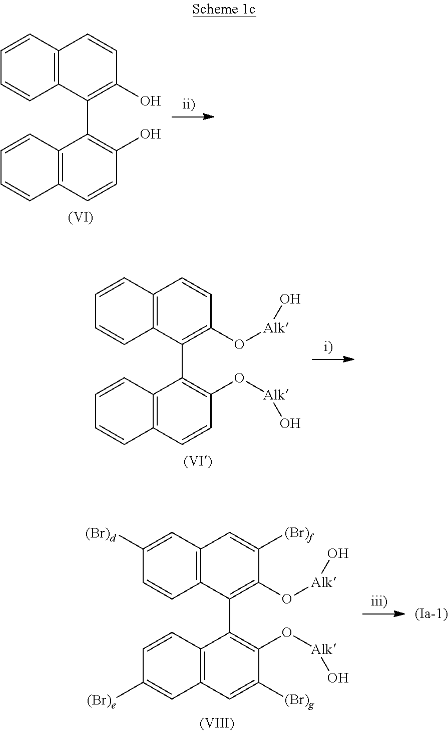

The reaction conditions in steps i), ii) and iii) of the processes according to schemes 1b and 1c are the same or almost the same as described for steps i), ii) and iii) of the process according to scheme 1a.

The compounds of the formula (Ib-1), where the radicals R^{a1}, R^{a2}, R^{a3} and R^{a4} differing from hydrogen are identical radicals R^{a} and where R¹ and R² both are hydrogen, phenyl, or have the same meaning as the radicals R^{a1}, R^{a2}, R^{a3} and R^{a4} differing from hydrogen, can be prepared by initially reacting the fluoren-9-one compound of formula (XII) with the phenol compound of formula (XIII) as shown in the following reaction scheme 2a.

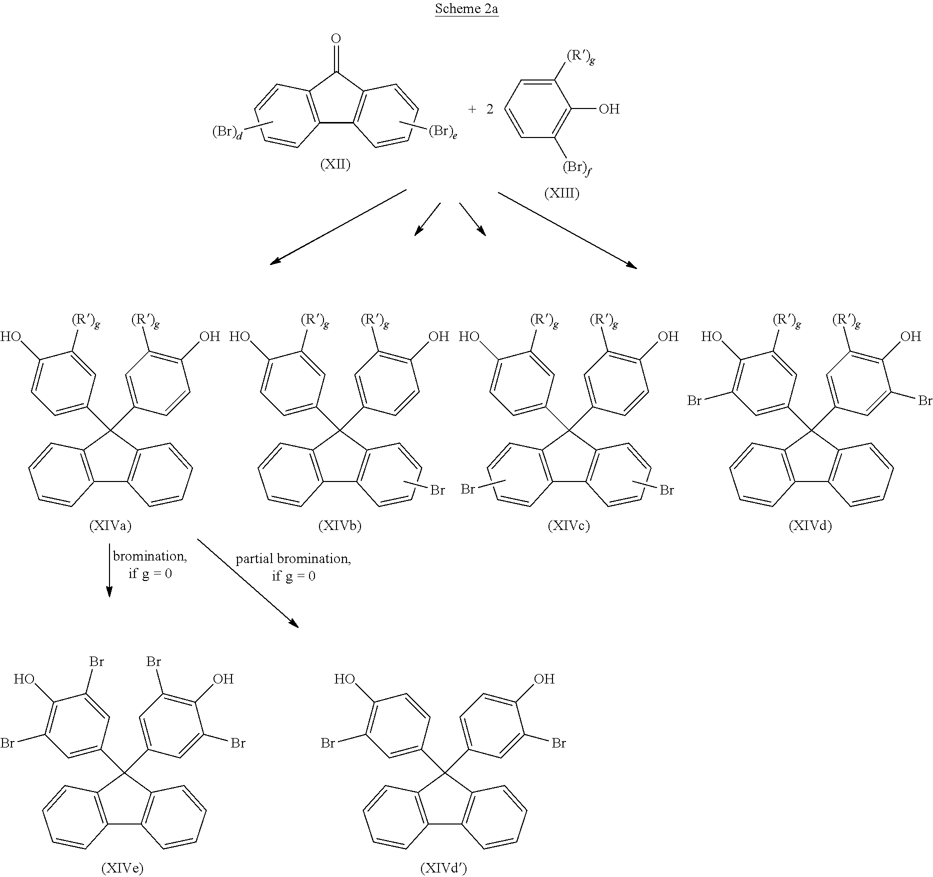

The fluoren-9-one compound of formula (XII) and about two equivalents the phenol compound of formula (XIII), where the radical is R′ is phenyl and the variables d, e, f and g are 0 or 1, are subjected to a condensation reaction using procedures well established in the art (see e.g. WO 1992/007812; U.S. Pat. No. 5,304,688, DE 4435475 and JPH09124530). If present, the one or two bromine substituents of the benzophenone compound (XII) are preferably located in positions 2 or 3 and positions 2 and 7 or 3 and 6, respectively, and in particular in position 2 or positions 2 and 7. The reaction affords one of the fluorene derivatives of formulae (XIVa) to (XIVd) depending on the bromine substitution of the educts of formulae (XII) and (XIII): if both educts are not brominated, i.e. the variables d, e and f are all 0, compound (XIVa) is obtained, if one of d and e is 0 and the other is 1 compound (XIVb) is obtained, if d and e are both 1 compound (XIVc) is obtained, and if d and e are both 0 and f is 1 compound (XIVd) is obtained. The one or two bromine substituents in the compounds (XIVb) and (XIVc) are preferably located in the positions 2 or 3 and positions 2 and 7 or 3 and 6, respectively, and in particular in position 2 or positions 2 and 7 of the fluorene moiety. In addition, compound (XIVe) is accessible by brominating the compound of formula (XIVa) using methods which correspond to those used in step i) described herein in the context of the reaction scheme 1a. Carrying out the bromination under conditions resulting in the introduction of only two bromine atoms enables an alternative access to the compound (XIVd′) which corresponds to compound (XIVd), where the variable g is 0.

Subsequently the two hydroxyl groups of the compounds of formulae (XIVb) to (XIVe) are converted into groups O-Alk′-OH and afterwards the one or more bromine substituents are replaced by radicals R^{a} by analogy with steps ii) and iii), respectively, described herein above in the context of reaction scheme 1a. It is often possible to change the sequence of these two steps in a similar manner as described above for steps ii) and iii) in the context of scheme 1b.

The compounds of the formula (Ib-2), where the radicals R^{a1}, R^{a2}, R^{a3} and R^{a4} differing from hydrogen are identical radicals R^{a} can be prepared by analogy with the procedures described above for the preparation of compounds of formula (Ib-1) by replacing the phenol compound (XIII) with a corresponding naphthol compound.

The compounds of the formula (Ic-1), where the radicals R^{a1} and R^{a2} differing from hydrogen are identical radicals R^{a} and where R¹ and R² both are hydrogen, phenyl, or have the same meaning as the radicals R^{a1}, R^{a2}, R^{a3} and R^{a4} differing from hydrogen, can be prepared by initially reacting the anthraquinone compound of formula (XV) with the phenol compound of formula (XIII) as shown in the following reaction scheme 3a.

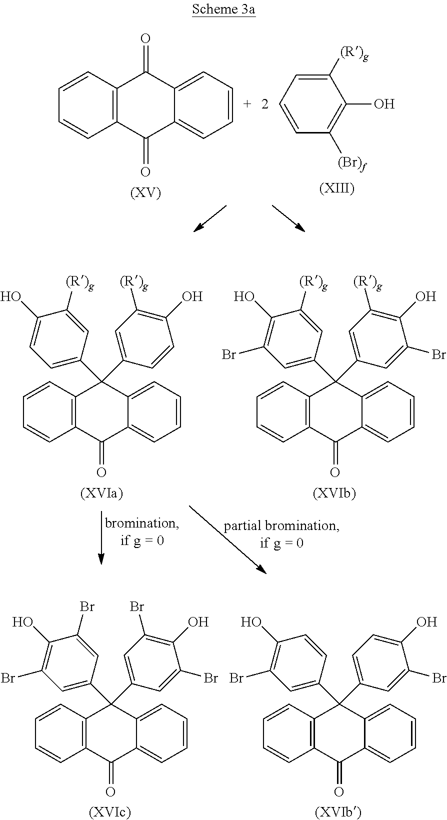

The anthraquinone compound of formula (XV) and about two equivalents the phenol compound of formula (XIII), where the radical R′ is phenyl and the variables d, e, f and g are 0 or 1, are subjected to a condensation reaction using procedures well established in the art (see e.g. JP2009249307; JP2014201551; CN107056725 and CN107068876). The reaction affords one of the anthraquinone derivatives of formulae (XVIa) or (XIVb) depending on whether the phenol compound of formula (XIII) is brominated. In addition, compound (XVIc) is accessible by brominating the compound of formula (XVIa) using methods which correspond to those used in step i) described herein in the context of the reaction scheme 1a. Carrying out the bromination under conditions resulting in the introduction of only two bromine atoms enables an alternative access to the compound (XVIb′) which corresponds to compound (XVIb), where the variable g is 0.

Subsequently the two hydroxyl groups of the compounds of formulae (XVIb) and (XVIc) are converted into groups O-Alk′-OH and afterwards the two or four bromine substituents are replaced by radicals R^{a} by analogy with steps ii) and iii), respectively, described herein above in the context of reaction scheme 1a. It is often possible to change the sequence of these two steps in a similar manner as described above for steps ii) and iii) in the context of scheme 1b.

The compounds of the formula (Ic-2), where the radicals R^{a1} and R^{a2} differing from hydrogen are identical radicals R^{a} can be prepared by analogy with the procedures described above for the preparation of compounds of formula (Ic-1) by replacing the phenol compound (XIII) with a corresponding naphthol compound.

The compounds of the formulae (Id-1) and (Id-2), where the radicals R^{a1}, R^{a2}, R^{a3} and R^{a4} differing from hydrogen are identical radicals R^{a} and where R¹ and R² both are hydrogen, phenyl, or have the same meaning as the radicals R^{a1}, R^{a2}, R^{a3} and R^{a4} differing from hydrogen, are accessible by initially preparing a diol compound of the formula (XX′), where the variables d, e and g are independently of one another 0 or 1, as shown in the following reaction scheme 4a.

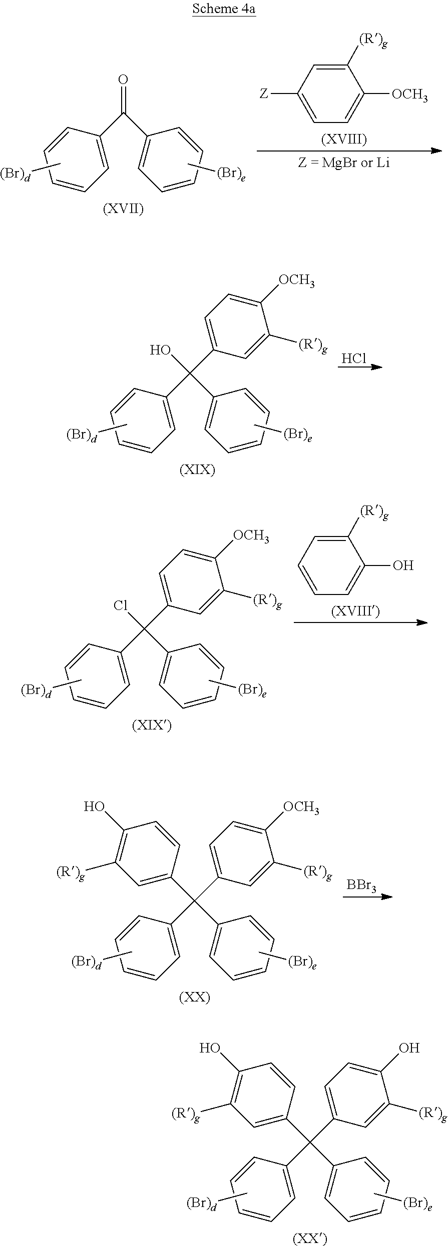

The benzophenone compound of formula (XVII) is reacted with the anisole compound of formula (XVIII), where the variables d, e and g are independently 0 or 1, R′ is phenyl and Z is MgBr or Li, i.e. the anisole compound (XVIII) is either a Grignard reagent or an organolithium reagent. The reaction results in the formation of the (4-methoxyphenyl)(diphenyl)methanol compound of formula (XIX) which is treated with hydrogen chloride to afford the corresponding chloride of formula (XIX′). Subsequent conversion with the phenol compound (XVIII′), which like the anisole compound (XVIII) may or may not bear a substituent R′ in ortho position to the hydroxyl or methoxy group, yields the tetraphenylmethane compound (XX). The intended diol compound (XX′) is then obtained by demethylation with boron tribromide. This reaction sequence shown in scheme 4a can be carried out e.g. by analogy with the methods described in M. P. L. Werts et al., Macromolecules 2003, 36(19), 7004-7013; P. Noesel et al., Adv. Synth. Catal. 2014, 356(18), 3755-3760; M. Singh et al., Bioorg. Med. Chem. Lett., 2012, 22(19), 6252-6255; and V. Theodorou et al., Tetrahedron 2007, 63(20), 4284-4289.

Two alternative routes toward the diol compound of formula (XX′) are depicted in the reaction scheme 4b below.

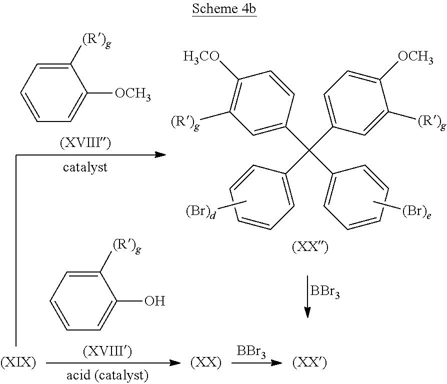

The (4-methoxyphenyl)(diphenyl)methanol compound of formula (XIX), which is obtained as described above in the context of scheme 4a, is directly converted into the tetraphenylmethane compound (XX) by reaction with the phenol compound of formula (XVIII′) in the presence of mineral acid or Lewis acid as a catalyst, such as sulfuric acid, methanesulfonic acid, p-toluenesulfonic acid or aluminum phenoxide (Al(OPh)₃), by analogy with the procedures disclosed e.g. in V. A. Koshchii et al., Zh. Org. Khim. 1988, 24(7), 1508-1512. Alternatively, the (4-methoxyphenyl)(diphenyl)methanol compound (XIX) is reacted with the anisole compound of formula (XVIII″) in the presence of an acid or a transition-metal-complex as catalyst to yield the tetraphenylmethane compound (XX″), by analogy with the methods described e.g. in J. E. Chateauneuf, K. Nie, ACS Symposium Series 2002, 819, 136-150; J. Choudhury et al., J. Am. Chem. Soc. 2005, 127(17), 6162-6163; and S. Roy et al., J. Chem. Sci. 2008, 120(5), 429-439. In the final steps of both routes the tetraphenylmethane compounds of formula (XX) or of formula (XX″) are converted by demethylation with boron tribromide to the intended diol compound (XX′).

If present, the one or two bromine substituents of the benzophenone compound (XVII) used for the preparation of the diol compound (XX′) are preferably located in position 2, 3 or 4 and in positions 2 and 2′, 3 and 3′ or 4 and 4′, respectively, of the phenyl rings. In case the benzophenone compound (XVII) is singly brominated, i.e. one of the variables d and e is 0 and the other is 1, the bromine atom is preferably located in position 2, 3 or 4 of one of the phenyl groups. Accordingly, the reaction sequences shown in schemes 4a or 4b afford the diol compound of formula (XX′), where one of d and e is 0 and the other is 1, with the single bromine atom preferably in position 2, 3 or 4 of one of the otherwise unsubstituted phenyl rings of (XX′). In case the benzophenone compound (XVII) is doubly brominated, i.e. the variables d and e are both 1, the bromine atoms are preferably located in positions 2 and 2′, 3 and 3′ or 4 and 4′ of the two phenyl groups. Accordingly, the reaction sequences shown in schemes 4a or 4b afford the diol compound of formula (XX′), where d and e are both 1, with the two bromine atoms preferably in position 2 and 2′, 3 and 3′ or 4 and 4′ of the two otherwise unsubstituted phenyl rings of (XX′).

In addition, the compounds of formulae (XXI), (XXI) and (XXI″) are accessible starting from the diol compound (XX′) by introducing one or two bromine substituents at each of its two phenyl rings that carry a hydroxyl group, as shown in the reaction scheme 4c below.

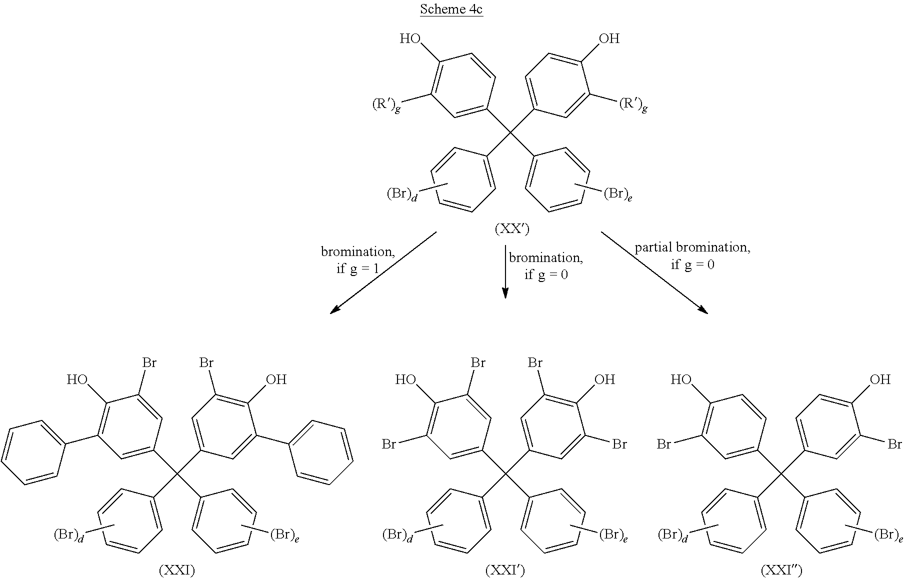

The diol compound (XX′), where R′ is phenyl and the variables d, e and g independently of one another are 0 or 1, is brominated using methods which correspond to those applied in step i) described herein in the context of the reaction scheme 1a. If the bromination of compound (XX′) with g=0 is carried out under suitable conditions, such as in particular a reduced amount of bromination agent, the introduction of only two bromine atoms can be achieved to yield the partially brominated compound (XXI″).

In a subsequent reaction step the two hydroxyl groups of the compounds of formulae (XX′), (XXI), (XXI′) and (XXI″) are converted into groups O-Alk′-OH and afterwards the one or more bromine substituents are replaced by radicals R^{a} by analogy with steps ii) and iii), respectively, described herein above in the context of reaction scheme 1a. It is often possible to change the sequence of these two steps in a similar manner as described above for steps ii) and iii) in the context of scheme 1b.

The compounds of the formulae (Id-3) and (Id-4), where the radicals R^{a1}, R^{a2}, R^{a3} and R^{a4} differing from hydrogen are identical radicals R^{a} can be prepared by analogy with the procedures described above for the preparation of compounds of formulae (Id-1) and (Id-2) by replacing the anisole and phenol compounds of formulae (XVIII), (XVIII′) and (XVIII″) with the corresponding naphthol derivatives.

Instead of converting the hydroxyl groups of the diol compounds of formulae (VI), (VII), VII′), (XIVb) to (XIVe), (XVIb), (XVIc), (XX′), (XXI), (XXI′) and (XXI″) into groups O-Alk′-OH as described above, they can alternatively be converted into groups O-Alk-C(O)O—C₁-C₄-alkyl by reaction with a compound Hal-Alk-C(O)O—C₁-C₄-alkyl, where Hal is bromine or chlorine and Alk is in particular methylene, as described e.g. in T. Ema J. Org. Chem. 2010, 75(13), 4492-4500. If desired, the thus introduced groups O-Alk-C(O)O—C₁-C₄-alkyl can afterwards be converted into groups O-Alk-C(O)OH using well known procedures of ester hydrolysis. Accordingly, compounds of the formula (I), where R³ is O-Alk-C(O)—, are generally accessible in this way via conversion of the corresponding aromatic diols.

The reaction mixtures obtained in the individual steps of the syntheses for preparing compounds of formulae (Ia-1), (Ib-1), (Ib-2), (Ic-1), (Ic-2), (Id-1), (Id-2), (Id-3) and (Id-4) described above are worked up in a conventional way, e.g. by mixing with water, separating the phases and, where appropriate, purifying the crude products by washing, chromatography or crystallization. The intermediates in some cases result in the form of colourless or pale brownish, viscous oils, which are freed of volatiles or purified under reduced pressure and at moderately elevated temperature. If the intermediates are obtained as solids, the purification can be achieved by recrystallization or washing procedures, such as slurry washing.

The compounds of the formulae (VI), (IX), (X), (XI), (XII), (XV), (XVII) as well as the phenol and anisole compounds of formulae (XIII), (XVIII), (XVIII′) and (XVIII″) and the corresponding naphthol derivatives are commercially available or can be prepared by methods known from the art.

It is apparent to a skilled person that compounds, where the two radicals R^{a} are different, can be obtained by analogy with the methods for preparing compounds (Ia-1), (Ib-1), (Ib-2), (Ic-1), (Ic-2), (Id-1), (Id-2), (Id-3) and (Id-4), e.g. by using mixtures of boronic compounds (X) or mixtures of acetylene compounds (XI) having different radicals R^{a} or by applying a step-wise reaction of the di- tri or tetrabromo compounds of formulae (VII), (VIII), (XIVb), (XIVc), (XIVd), (XIVe), (XVIb), (XVIc), (XVIIIb), (XVIIIc), (XVIIId) or (XVIIIe) with different reagents selected from boronic compounds (X) and acetylene compounds (XI). By these methods, usually mixtures of differently substituted compounds of the formula (I) will be obtained. These mixtures can be separated, e.g. by chromatography, to obtain the individual compounds of formula (I).

For the purpose of the invention, i.e. the use of the compounds of formula (I) as monomers in the preparation of optical resins, it may not be necessary to resolve these mixtures. Rather, the mixtures may also be used as monomers.

Compounds, where the two radicals R^{a} are different can also be obtained by processes similar to the processes for preparing compounds (Ia-1), (Ib-1), (Ib-2), (Ic-1), (Ic-2), (Id-1), (Id-2), (Id-3) and (Id-4), where instead of introducing two or more bromine atoms only one bromine atom is introduced, followed by reaction with a boronic compound (X) or an acetylene compound (XI). Then, a second bromination step is performed followed by a further reaction with a different boronic compound R¹¹—C≡C—Ar—B(OH)₂ or acetylene compound R¹¹—C≡C—H. One or two further iterations of bromination and subsequent introduction of a different radical R^{a} may be performed to yield a compound of formula (I) bearing different radicals R^{a}.

As stated above, the compounds of the present invention can be obtained in high purity, which means that a product is obtained, which does not contain significant amounts of organic impurities different from the compound of formula (I), except for volatiles. Usually, the purity of compounds of formula (I) is at least 95%, in particular at least 98% and especially at least 99%, based on the non-volatile organic matter, i.e. the product contains at most 5%, in particular at most 2% and especially at most 1% of non-volatile impurities different from the compound of formula (I).

The term “volatiles” refers to organic compounds, which have a boiling point of less than 200° C. at standard pressure (10⁵ Pa). Consequently, non-volatile organic matter is understood to mean compounds having a boiling point, which exceeds 200° C. at standard pressure.

It is a particular benefit of the invention that the compounds of formulae (I), (Ia), (Ia-1), (Ib), (Ib-1), (Ib-2), (Ic), (Ic-1), (Ic-2), (Id), (Id-1), (Id-2), (Id-3) and (Id-4), and likewise their solvates, can often be obtained in crystalline form. In the crystalline form the compound of formula (I) may be present in pure form or in the form of a solvate with water or an organic solvent. Therefore, a particular aspect of the invention relates to the compounds of formula (I), which are essentially present in crystalline form. In particular, the invention relates to crystalline forms, where the compound of formula (I) is present without solvent and to the crystalline solvates of the compounds of formula (I), where the crystals contain a solvent incorporated.

It is a particular benefit of the invention that the compounds of the formulae (I), (Ia), (Ia′), (Ia-1), (Ib), (Ib-1), (Ib-2), (Ic), (Ic-1), (Ic-2), (Id), (Id-1), (Id-2), (Id-3) and (Id-4), and likewise their solvates, can often be easily crystallized from conventional organic solvents. This allows for an efficient purification of the compounds of formula (I). Suitable organic solvents for crystallizing the compounds of the formula (I) or their solvates, include but are not limited to aromatic hydrocarbons such as toluene or xylene, aliphatic ketones in particular ketones having from 3 to 6 carbon atoms, such as acetone, methyl ethyl ketone, methyl isopropyl ketone or diethyl ketone, aliphatic and alicyclic ethers, such as diethyl ester, dipropyl ether, methyl isobutyl ether, methyl tert-butyl ether, ethyl tert-butyl ether, dioxane or tetrahydrofuran, and aliphatic alcohols having 1 to 4 carbon atoms, such as methanol, ethanol or isopropanol, as well as mixtures thereof.

Alternatively, the compounds of the formulae (I), (Ia), (Ia′), (Ia-1), (Ib), (Ib-1), (Ib-2), (Ic), (Ic-1), (Ic-2), (Id), (Id-1), (Id-2), (Id-3) and (Id-4), and likewise their solvates, can be obtained in purified form by employing other simple and efficient methods for purifying the raw products of the compounds of the formula (I), such as in particular slurry washing the raw solids obtained directly after the conversion to prepare the compounds of formula (I). Slurry washing is typically conducted at ambient temperature or elevated temperatures of usually about 30 to 90° C., in particular 40 to 80° C. Suitable organic solvents here are in principle the same as those listed above as being suitable for crystallizing the compounds of formula (I), such as in particular the mentioned aromatic hydrocarbons, aliphatic ketones and aliphatic ethers, e.g. toluene, methyl ethyl ketone and methyl tert-butyl ether.

Accordingly, the compounds of formulae (I), (Ia), (Ia′), (Ia-1), (Ib), (Ib-1), (Ib-2), (Ic), (Ic-1), (Ic-2), (Id), (Id-1), (Id-2), (Id-3) and (Id-4), respectively, used for the preparation of the thermoplastic polymers, in particular the polycarbonates, as defined herein, can be easily prepared and obtained in high yield and high purity. In particular, compounds of formulae (I), (Ia), (Ia′), (Ia-1), (Ib), (Ib-1), (Ib-2), (Ic), (Ic-1), (Ic-2), (Id), (Id-1), (Id-2), (Id-3) and (Id-4), respectively, can be obtained in crystalline form, which allows for an efficient purification to the degree required in the preparation of optical resins. In particular, these compounds can be obtained in a purity which provides for high refractive indices and also low haze, which is particularly important for the use in the preparation of optical resins of which the optical devise is made of. In conclusion, the compounds of formulae (I), (Ia), (Ia′), (Ia-1), (Ib), (Ib-1), (Ib-2), (Ic), (Ic-1), (Ic-2), (Id), (Id-1), (Id-2), (Id-3) and (Id-4), respectively, are particularly useful as monomers in the preparation of the optical resins.

A skilled person will readily appreciate that the formula (I) of the monomer used corresponds to the formula (II) of the structural unit comprised in the thermoplastic resin. Likewise, the formulae (I), (Ia), (Ia′), (Ia-1), (Ib), (Ib-1), (Ib-2), (Ic), (Ic-1), (Ic-2), (Id), (Id-1), (Id-2), (Id-3) and (Id-4), respectively, of the monomer used corresponds to the formulae (II), (IIa), (IIa-1), (IIb), (IIb-1), (IIb-2), (IIc), (IIc-1), (IIc-2), (IId), (IId-1), (IId-2), (IId-3) and (IId-4), respectively, of the structural unit comprised in the thermoplastic resin.

A skilled person will also appreciate that the structural units of the formulae (II), (IIa), (IIa′), (IIa-1), (IIb), (IIb-1), (IIb-2), (IIc), (IIc-1), (IIc-2), (IId), (IId-1), (IId-2), (IId-3) and (IId-4), are repeating units within the polymer chains of the thermoplastic resin.

In addition to the structural units of the formulae (II), (IIa), (IIa′), (IIa-1), (Mb), (IIb-1), (IIb-2), (IIc), (IIc-1), (IIc-2), (IId), (IId-1), (IId-2), (IId-3) and (IId-4), respectively, the thermoplastic resin may have structural units different therefrom. In a preferred embodiment, these further structural units are derived from aromatic monomers of the formula (IV) resulting in structural units of the formula (V):

HO—R^{z}-A³-R^{z}—OH (IV)

#—O—R^{z}-A³-R^{z}—O—# (V)

where
A³ is a polycyclic radical bearing at least 2 benzene rings, wherein the benzene rings may be connected by A′ and/or directly fused to each other and/or fused by a non-benzene carbocycle, where A³ is unsubstituted or substituted by 1, 2, 3, 4, 5 or 6 radicals R^{aa}, which are selected from the group consisting of halogen, C₁-C₆-alkyl, C₅-C₆-cycloalkyl and phenyl, in particular phenyl and methyl;
A′ is selected from the group consisting of a single bond, O, C═O, S, SO₂, CH₂, CH—Ar″, CHAr″₂, CH(CH₃), C(CH₃)₂ and a radical A″

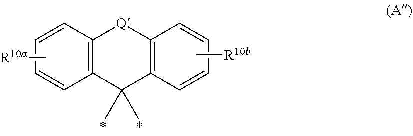

where
Q′ represents a single bond, O, NH, C═O, CH₂ or CH═CH, in particular a single bond or C═O; and
R^{10a}, R^{10b}, independently of each other are selected from the group consisting of hydrogen, fluorine, CN, R, OR, CHₖR₃₋ₖ, NR₂, C(O)R and C(O)NH₂ and where R^{10a}, R^{10b} are in particular hydrogen;
Ar″ is selected from the group consisting of mono- or polycyclic aryl having from 6 to 26 carbon atoms and mono- or polycyclic hetaryl having a total of 5 to 26 atoms, which are ring members, where 1, 2, 3 or 4 of these atoms are selected from nitrogen, sulphur and oxygen, while the remainder of these atoms are carbon atoms, in particular phenyl or naphthyl, where Ar″ is unsubstituted or substituted by 1, 2 or 3 radicals R^{ab}, which are selected from the group consisting of halogen, C₁-C₆-alkyl, C5-C6-cycloalkyl and phenyl, in particular phenyl and methyl;
R^{z} is a single bond, Alk¹, O-Alk²-, O-Alk²-[O-Alk²-]ₚ- or O-Alk³-C(O)—, where O is bound to A³, and where
p is an integer from 1 to 10;
Alk¹ is C₁-C₄-alkandiyl, in particular CH₂;
Alk² is C₂-C₄-alkandiyl, in particular linear alkandiyl having 2 to 4 carbon atoms and especially CH₂CH₂;
Alk³ is C₁-C₄-alkandiyl, in particular CH₂, and
#represents a connection point to a neighboring structural unit.

If R^{z} in formula (IV) is O-Alk³-C(O), the esters, in particular the C₁-C₄-alkyl esters of the monomers of formula (IV) may be used instead.

In the context of formulae (IV) and (V), A³ is in particular a polycyclic radical bearing 2 benzene or naphthaline rings, wherein the benzene rings are connected by A′. In this context A′ is in particular selected from the group consisting of a single bond, CH—Ar″, CHAr″₂, and a radical A″.

In the context of formulae (IV) and (V), R^{z} is in particular O-Alk²-, where Alk² is in particular linear alkandiyl having 2 to 4 carbon atoms and especially CH₂CH₂

Amongst the monomers of formula (IV) preference is given to monomers of the general formulae (IV-1) to (IV-6)

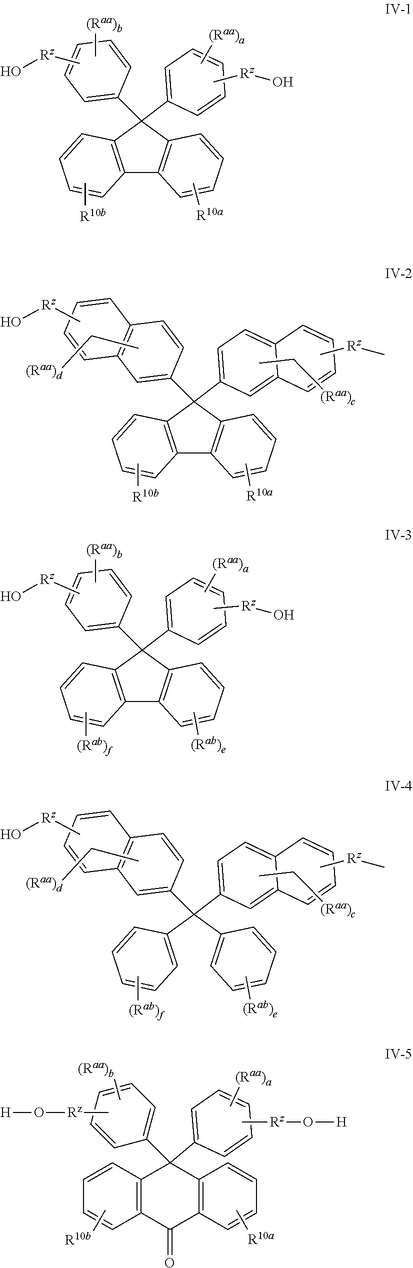

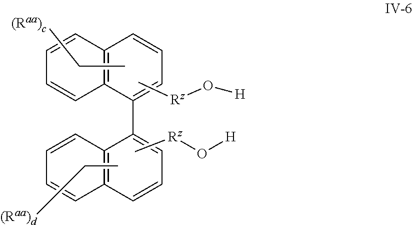

where

a and b are 0, 1, 2 or 3, in particular 0 or 1;

c and d are 0, 1, 2, 3, 4 or 5, in particular 0 or 1;

e and f are 0, 1, 2, 3, 4 or 5, in particular 0 or 1;

and where R^{z}, R^{aa}, R^{ab}, R^{10a} and R^{10b} are as defined for formula (IV) and where R^{z} is in particular selected from a single bond, CH₂ and OCH₂CH₂.

Amongst the monomers of formula (IV) particular preference is given to monomers of the general formulae (IV-11) to (IV-18), where R^{z} and R^{aa} are as defined herein and R^{z} is in particular selected from a single bond, CH₂ and OCH₂CH₂:

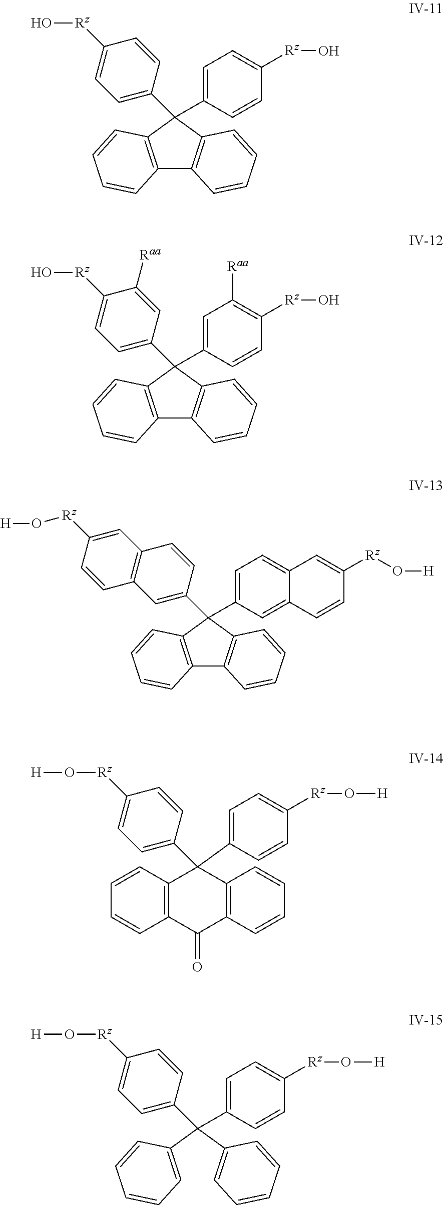

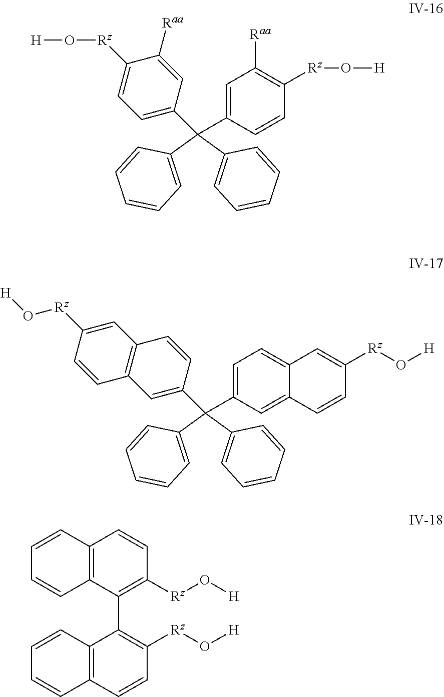

Examples of compounds of the formulae (IV-11) to (IV-18) are 9,9-bis(4-hydroxyphenyl)fluorene, 9,9-bis(4-hydroxy-3-methylphenyl)fluorene, 9,9-bis(4-hydroxy-3-isopropylphenyl)fluorene, 9,9-bis(4-hydroxy-3-tert.-butylphenyl)fluorene, 9,9-bis(4-hydroxy-3-cyclohexylphenyl)fluorene, 9,9-bis(4-hydroxy-3-phenylphenyl)fluorene, 9,9-bis(4-(2-hydroxyethoxy)phenyl)fluorene, 9,9-bis(4-(2-hydroxyethoxy)-3-methylphenyl)fluorene, 9,9-bis(4-(2-hydroxyethoxy)-3-isopropylphenyl)fluorene, 9,9-bis(4-(2-hydroxyethoxy)-3-tert.-butylphenyl)fluorene, 9,9-bis(4-(2-hydroxyethoxy)-3-cyclohexylphenyl)fluorene, 9,9-bis(4-(2-hydroxyethoxy)-3-phenylphenyl)fluorene also termed 9,9-bis(4-(2-hydroxyethoxy)-3-phenylphenyl-)fluorene (BPPEF), 9,9-bis(6-hydroxy-2-naphthyl)fluorene, 9,9-bis(6-(2-hydroxyethoxy)-2-naphthyl)fluorene, also termed 9,9-bis(6-(2-hydroxy-ethoxy)naphthalene-2-yl)fluorene (BNEF), 10,10-bis(4-hydroxyphenyl)anthracen-9-on, 10,10-bis(4-(2-hydroxyethoxy)phenyl)anthracen-9-on, 4,4′-dihydroxy-tetraphenylmethane, 4,4′-di-(2-hydroxyethoxy)-tetraphenylmethane, 3,3′-diphenyl-4,4′-dihydroxy-tetraphenylmethane, di-(6-hydroxy-2-naphthyl)-diphenylmethane, 2,2′-[1,1′-binaphthalene-2,2′-diylbis(oxy)]diethanol also termed 2,2′-bis(2-hydroxyethoxy)-1,1′-binaphtyl or 2,2′-bis(2-hydroxyethoxy)-1,1′-binaphthalene (BNE), 2,2′-bis(1-hydroxymethoxy)-1,1-binaphtyl, 2,2′-bis(3-hydroxypropyloxy)-1,1′-binaphtyl, 2,2′-bis(4-hydroxybutoxy)-1,1′-binaphtyl, 2,2′-bis(2-hydroxyethoxy)-6,6′-diphenyl-1,1′-binaphthalene, 2,2′-bis(2-hydroxyethoxy)-6,6′-di(naphthalene-1-yl)-1,1′-binaphthalene, 2,2′-bis(2-hydroxymethoxy)-6,6′-diphenyl-1,1′-binaphthalene, 2,2′-bis(2-hydroxy-methoxy)-6,6′-di(naphthalene-1-yl)-1,1′-binaphthalene, 2,2′-bis(2-hydroxypropoxy)-6,6′-diphenyl-1,1′-binaphthalene, 2,2′-bis(2-hydroxypropoxy)-6,6′-di(naphthalene-1-yl)-1,1′-binaphthalene, 2,2′-bis(2-hydroxyethoxy)-6,6′-di(naphthalene-2-yl)-1,1′-binaphthalene, 2,2′-bis(2-hydroxyethoxy)-6,6′-di(9-phenanthryl)-1,1′-binaphthalene and the like. Among the monomers of the general formula (IV) or of formulae (IV-1) to (IV-8), particular preference is given to the monomers of formulae (IV-1), (IV-2), (IV-3) and (IV-8) with more preference given to monomers of formulae (IV-2), (IV-3) and (IV-8) and special preference given to 2,2′-bis(2-hydroxyethoxy)-1,1′-binaphtyl (BNE or BHBNA), 9,9-bis(6-(2-hydroxyethoxy)-2-naphthyl)fluorene (BNEF) and 9,9-bis(4-(2-hydroxyethoxy)-3-phenylphenyl)fluorene (BPPEF).

Accordingly, amongst the structural units of formula (V) that may be comprised in the thermoplastic resin preference is given to structural units of the general formulae (V-1) to (V-6),

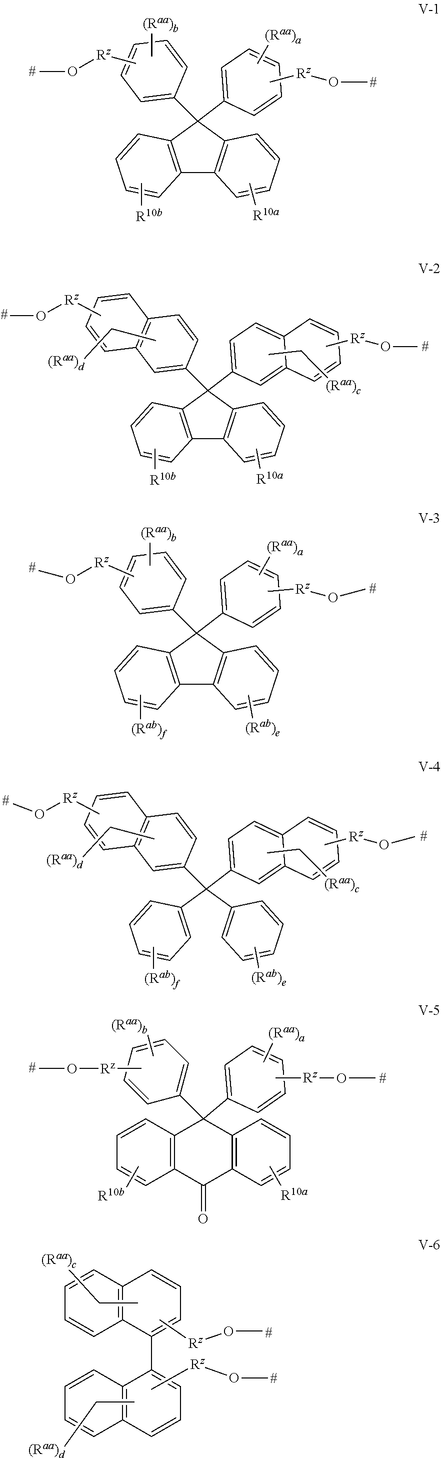

where

a and b are 0, 1, 2 or 3, in particular 0 or 1;

c and d are 0, 1, 2, 3, 4 or 5, in particular 0 or 1;

e and f are 0, 1, 2, 3, 4 or 5, in particular 0 or 1;

and where R^{z}, R^{aa}, R^{ab}, R^{10a} and R^{10b} are as defined for formula (IV) and where R^{z} is in particular selected from a single bond, CH₂ and OCH₂CH₂.

Particular preference is given to structural units of the general formulae (V-11) to (V-18), where R^{z} and R^{aa} are as defined herein and where R^{z} is in particular selected from a single bond, CH₂ and OCH₂CH₂:

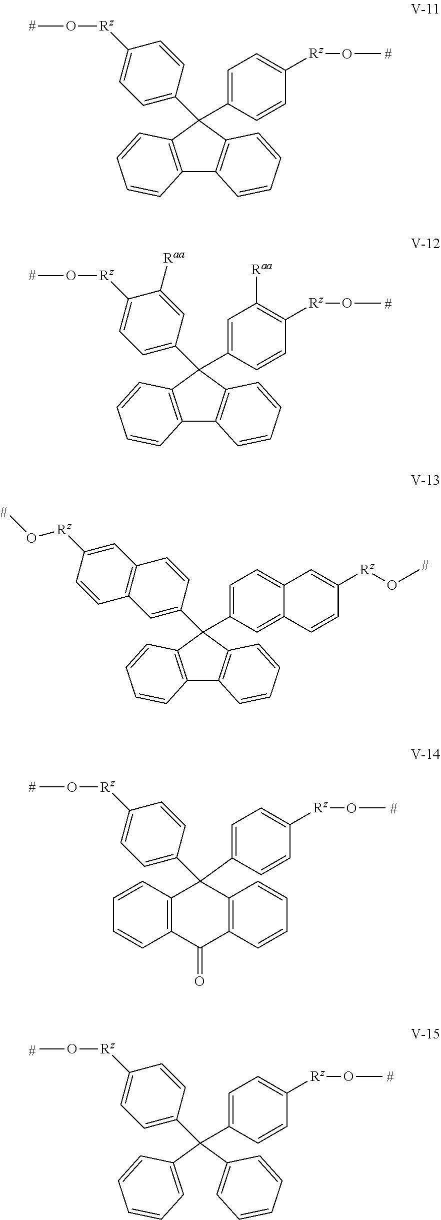

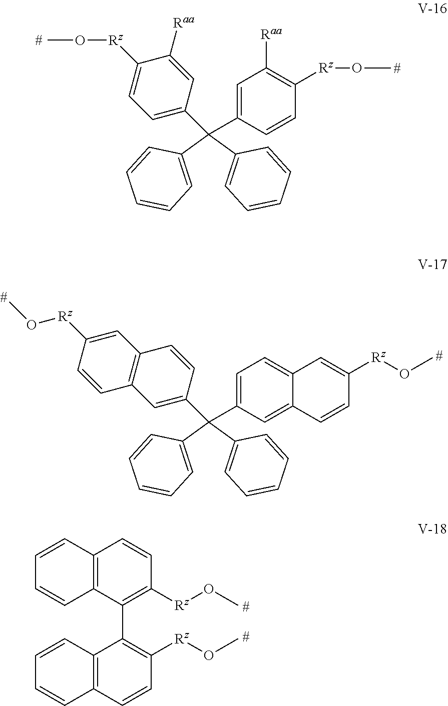

Among the structural units of the formulae (V-1) to (V-6), particular preference is given to the structural units of formulae (V-1), (V-2) and (V-6). Among the structural units of the formulae (V-11) to (V-18), particular preference is given to the structural units of formulae (V-11), (V-12), (V-13) and (V-18) with more preference given to structural units of formulae (V-12), (V-13) and (V-18) and special preference given to structural units derived from 2,2′-bis(2-hydroxyethoxy)-1,1′-binaphtyl (BNE or BHBNA), 9,9-bis(6-(2-hydroxyethoxy)-2-naphthyl)fluorene (BNEF) and 9,9-bis(4-(2-hydroxyethoxy)-3-phenylphenyl)fluorene (BPPEF).

In a particular preferred group of embodiments, the thermoplastic resin of the present invention comprises at least one structural unit of the formula (IIa-1) and at least one structural unit selected from the group consisting of structural units of the formula (V-13), structural units of the formula (V-16) and structural units of the formula (V-18). In this particular group of embodiments, those thermoplastic resins are preferred, where in the structural unit of the formula (IIa-1) R^{a1} and R^{a2} are identical and selected from the group consisting of phenylethynyl, naphthalene-1-ylethynyl and 2-naphthalene-2-ylethynyl and where R^{a3} and R^{a4} in formula (IIa-1) are hydrogen, i.e. where these structural units (IIa-1) are derived from monomers (I) selected from the group consisting of D1NACBHBNA, D2NACBHBNA and DPACBHBNA and combinations thereof. In this particular group of embodiments, those thermoplastic resins are preferred, where in the structural unit of the formulae (V-13), (V-16) and (V-18) the radicals R^{z} are O—CH₂CH₂, i.e. where these structural units (V-13), (V-16) and (V-18) are derived from monomers (IV) selected from the group consisting of BPPEF, BNEF and BNE and combinations thereof.

In the thermoplastic resins of this particular preferred group of embodiments, it is preferred that the total molar ratio of the structural units of the formula (IIa-1), in particular those derived from D1NACBHBNA, D2NACBHBNA and/or DPACBHBNA, is in the range from 1 to 70 mol-%, preferably in the range from 5 to 60 mol-%, further preferably in the range from 8 to 45 mol-%, and even further preferably in the range from 10 to 30 mol-% of the total amount of structural units of the formulae (II) and (V).

In the thermoplastic resins of this particular preferred group of embodiments, it is preferred that the total molar ratio of the structural units of the formula (IIa-1), in particular those derived from BPPEF, BNEF and/or BNE, is in the range from 30 to 99 mol-%, in particular in the range from 40 to 95 mol-%, further preferably in the range from 55 to 92 mol-%, and even further preferably in the range from 70 to 90 mol-%. It is also preferable that each of the molar ratio of the structural unit derived from BPPEF, BNEF or BNE in the total structural units of the thermoplastic resin is 10 to 70%, more preferably 15 to 65%, further preferably 20 to 60%, and even further preferably 25 to 55%.

The compounds of the formulae (IV), (IV-1), (IV-2), (IV-3), (IV-4), (IV-5), (IV-6), (IV-11), (IV-12), (IV-13), (IV-14), (IV-15), (IV-16), (IV-17) and (IV-18) are known or can be prepared by analogy to known methods.

For example, the compounds of the formula (IV-6) can be prepared by various synthesis methods, as disclosed e.g. in JP Publication No. 2014-227387, JP Publication No. 2014-227388, JP Publication No. 2015-168658, and JP Publication No. 2015-187098. For example, 1,1′-binaphthols may be reacted with ethylene glycol monotosylates; alternatively, 1,1′-binaphthols may be reacted with alkylene oxides, halogenoalkanols, or alkylene carbonates; and alternatively, 1,1′-binaphthols may be reacted with ethylene carbonates. Thereby, the compounds of the formula (IV-6) is obtained, where R^{z}—OH is O-Alk²- or O-Alk²-[O-Alk²-]ₚ-.

For example, the compounds of the formula (V-2) can be prepared by various synthesis methods, as disclosed e.g. in JP Patent Publication No. 5442800, and JP Publication No. 2014-028806. Examples include:

(a) reacting fluorenes with hydroxy naphthalenes in the presence of hydrochloride gas and mercapto-carboxylic acid;

(b) reacting 9-fluorene with hydroxy naphthalenes in the presence of acid catalyst (and alkyl mercaptan);

(c) reacting fluorenes with hydroxy naphthalenes in the presence of hydrochloride and thiols (such as, mercapto-carboxylic acid);

(d) reacting fluorenes with hydroxy naphthalenes in the presence of sulfuric acid and thiols (such as, mercapto-carboxylic acid) and thereafter to crystallize the product from a crystallization solvent which consists of hydrocarbons and a polar solvent(s) to form bisnaphthol fluorene; and the like

Thereby compounds of the formula (IV-2) can be obtained, where R^{z} is a single bond.

The compounds of formulae (IV), where R^{z} is O-Alk²- or O-Alk²-[O-Alk²-]ₚ-can be prepared form compounds of formulae (IV), where R^{z} is a single bond, by reaction with alkylene oxides or haloalkanols. For example, reacting 9,9-bis(hydroxynaphthyl)-fluorenes of the formula (IV-2) where R^{z} is a single bond with alkylene oxides or haloalkanols results in the compounds of the formula (IV-2) where R^{z} is O-Alk²- or O-Alk²-[O-Alk²-]ₚ-. For example, 9,9-bis[6-(2-hydroxyethoxy)naphthyl] fluorene can be prepared by reacting 9,9-bis[6-(2-hydroxynaphthyl] fluorene with 2-chloroethanol under alkaline conditions.

The monomers of formula (I) and likewise the co-monomers of formula (IV) used for producing the thermoplastic resin may contain certain impurities resulting from their preparation, e.g. hydroxy compounds, which bear an OH group instead of a group HO—R³ or it may contain a group O-Alk′-[O-Alk′]₀ instead of a group O-Alk′-, or it may contain a halogen atom instead of a radical R^{a}. The total amount of such impurity compounds is preferably 1000 ppm or lower, more preferably 500 ppm or lower, still more preferably 200 ppm or lower, and especially preferably 100 ppm or lower. The total content of the impurities in the monomers used for preparing the thermoplastic resin is preferably 100 ppm or lower in particular 50 ppm or lower, and more preferably 20 ppm or lower. In particular, the total amount of dihydroxy compounds in which a carbon number of at least one of the radicals R³ differs from the formula (I), is preferably 1000 ppm or lower, more preferably 500 ppm or lower, still more preferably 200 ppm or lower, and especially preferably 100 ppm or lower; in the monomer(s) of which main component is the dihydroxy compound(s) represented by the formula (I). The total content of the dihydroxy compounds in which a carbon number of at least one of the radicals R³ differs from the formula (I) is further preferably 50 ppm or lower, and more preferably 20 ppm or lower. Likewise, the amount of impurities in the co-monomers of formula (IV) will be in the range given for the monomers of formula (I).

Suitable thermoplastic resins for the preparation of optical devices, such as lenses, are in particular polycarbonates, polyestercarbonates and polyesters. Preferred thermoplastic resins for the preparation of optical devices, such as lenses, are in particular polycarbonates.

Said polycarbonates are structurally characterized by having structural units of at least one of the formulae (II), (IIa), (IIa′), (IIa-1), (Mb), (IIb-1), (IIb-2), (IIc), (IIc-1), (IIc-2), (IId), (IId-1), (IId-2), (IId-3) and (IId-4), respectively, optionally structural units derived from diol monomers, which are different from the monomer compound of the formula (I), e.g. structural units of the formula (V),

#—O—R^{z}-A³-R^{z}—O—# (V)
where
#, R^{z} and A³ are as defined herein above;

and a structural unit of formula (III-1) stemming from the carbonate forming component:

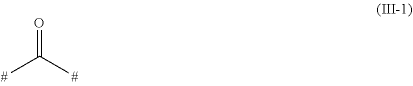

where each #represents a connection point to a neighboring structural unit, i.e. to O at the connection point of the structural unit of the formula (II) and, if present, to O at the connection point of the structural unit of the formula (V).

Said polyesters are structurally characterized by having structural units of at least one of the formulae (II), (IIa), (IIa′), (IIa-1), (IIb), (IIb-1), (IIb-2), (IIc), (IIc-1), (IIc-2), (IId), (IId-1), (IId-2), (IId-3) and (IId-4), respectively, optionally structural units derived from diol monomers which are different from the monomer compound of the formula (I), e.g. structural units of the formula V, and structural units derived from dicarboxylic acid, e.g. of formula (III-2) in case of a benzene dicarboxylic acid, of formula (III-3) in case of a naphthalene carboxylic acid, of formula (III-4) in case of oxalic acid and of formula (III-5) in case of malonic acid:

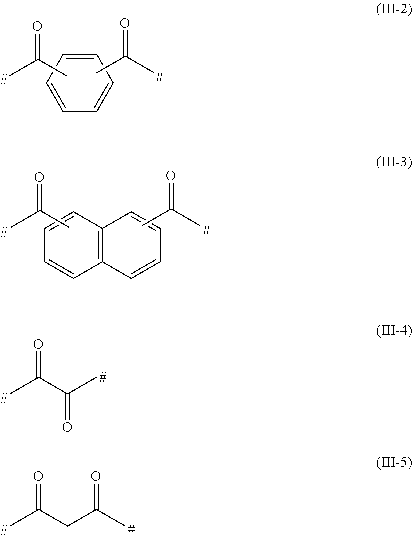

In formula (III-2) to (III-5) each variable #represents a connection point to a neighboring structural unit, i.e. to O of the connection point of the structural unit of the formula (II) and, if present, to O of the connection point of the structural unit of the formula (V).

Said polyestercarbonates are structurally characterized by having structural units of at least one of the formulae (II), (IIa), (IIa′), (IIa-1), (IIb), (IIb-1), (IIb-2), (IIc), (IIc-1), (IIc-2), (IId), (IId-1), (IId-2), (IId-3) and (IId-4), respectively, optionally structural units derived from diol monomers which are different from the monomer compound of the formula (I), e.g. structural units of the formula V, a structural unit of formula (III-1) stemming from the carbonate forming component and structural units derived from dicarboxylic acid, e.g. of formula (III-2) in case of a benzene dicarboxylic acid, of formula (III-3) in case of a naphthalene carboxylic acid, of formula (III-4) in case of oxalic acid and of formula (III-5) in case of malonic acid.

A particular group of embodiments relates to thermoplastic copolymer resins, in particular polycarbonates, polyestercarbonates and polyesters, which have both structural units of formula (II) and one or more structural units of formula (IV), i.e. resins, in particular polycarbonates, polyestercarbonates and polyesters, which are obtainable by reacting at least one monomer of formula (I) with one or more monomers of formula (IV). In this case the molar ratio of monomers of formula (I) to monomers of formula (IV) and likewise the molar ratio of the structural units of formula (II) to structural units of formula (V) are in the range from 5:95 to 80:20, in particular in the range from 10:90 to 70:30 and especially in the range from 15:85 to 60:40 or in the range from 1:99 to 70:30, in particular in the range from 5:95 to 60:40, more preferably in the range from 8:92 to 45:55 or in the range from 10:90 to 40:60 and especially in the range from 12:88 to 30:70 or in the range from 12:88 to 20:80.

Accordingly, the molar ratio of the structural units of the formula (II) is usually from 1 to 70 mol-% in particular from 5 to 60 mol-%, more preferably in the range from 8 to 45 mol-% or in the range from 10 to 40 mol-% and especially in the range from 12 to 30 mol-% or in the range from 12 to 20 mol-%, based on the total molar amount of structural units of the formulae (II) and (V). Accordingly, the molar ratio of the structural units of the formula (V) is usually from 30 to 99 mol-% in particular from 40 to 95 mol-%, more preferably in the range from 55 to 92 mol-% or in the range from 60 to 90 mol-% and especially in the range from 70 to 88 mol-% or in the range from 80 to 88 mol-%, based on the total molar amount of structural units of the formulae (II) and (V).

The thermoplastic copolymer resins of the present invention, such as a polycarbonate resin may include either one of a random copolymer structure, a block copolymer structure, and an alternating copolymer structure. The thermoplastic resin according to the present invention does not need to include all of structural units (II) and one or more different structural units (V) in one, same polymer molecule. Namely, the thermoplastic copolymer resin according to the present invention may be a blend resin as long as the above-described structures are each included in any of a plurality of polymer molecules. For example, the thermoplastic resin including all of structural units (II) and structural units (V) described above may be a copolymer including all of structural units (II) and structural units (V), it may be a mixture of a homopolymer or a copolymer including at least one structural unit (II) and a homopolymer or a copolymer including at least one structural unit (V) or it may be a blend resin of a copolymer including at least one structural unit (II) and a first structural unit (V) and a copolymer including at least one structural unit (II) and at least one other structural unit (V) different from the first structural units (V); etc.

Thermoplastic polycarbonates are obtainable by polycondensation of a diol component and a carbonate forming component. Similarly, thermoplastic polyesters and polyestercarbonates are obtainable by polycondensation of a diol component and a dicarboxylic acid, or an ester forming derivative thereof, and optionally a carbonate forming component.

Specifically, thermoplastic resins (polycarbonate resins) can be prepared by the following methods.

A method for preparing the thermoplastic resin of the present invention, such as a polycarbonate resin, includes a process of melt polycondensation of a dihydroxy component corresponding to the above-mentioned structural units and a diester carbonate. According to the present invention the dihydroxy compound comprises at least one dihydroxy compound represented by the formula (I), in particular by the formulae (Ia), (Ia′), (Ia-1), (Ib), (Ib-1), (Ib-2), (Ic), (Ic-1), (Ic-2), (Id), (Id-1), (Id-2), (Id-3) and (Id-4), especially by the formulae (Ia′) or (Ia-1), respectively, as defined herein. In addition to the compound of formula (I), the dihydroxy compound may also comprise one or more dihydroxy compounds represented by the formula (IV), preferably by the formulae (IV-1) to (IV-6), in particular by the formulae (IV-11) to (IV-18), more particularly by the formulae (IV-1 and especially by the formulae (IV-12), (IV-13) or (IV-18).

As is clear from the above, the polycarbonate resin can be formed by reacting a dihydroxy component with a carbonate precursor, such as a diester carbonate, where the dihydroxy component comprises at least one compound represented by the formulae (I), (Ia), (Ia′), (Ia-1), (Ib), (Ib-1), (Ib-2), (Ic), (Ic-1), (Ic-2), (Id), (Id-1), (Id-2), (Id-3) and (Id-4), respectively, or a combination of at least one compound represented by the formulae (I), (Ia), (Ia′), (Ia-1), (Ib), (Ib-1), (Ib-2), (Ic), (Ic-1), (Ic-2), (Id), (Id-1), (Id-2), (Id-3) and (Id-4), respectively, and at least one compound represented by the formulae (IV), (IV-1), (IV-2), (IV-3), (IV-4-), (IV-5), (IV-6), (IV-11), (IV-12), (IV-13), (IV-14), (IV-15), (IV-16) (IV-17) or (IV-18). Specifically, a polycarbonate resin can be formed by a melt polycondensation process in which the compound represented by the formulae (I), (Ia), (Ia′), (Ia-1), (Ib), (Ib-1), (Ib-2), (Ic), (Ic-1), (Ic-2), (Id), (Id-1), (Id-2), (Id-3) and (Id-4), respectively, or a combination thereof with at least one compound of the formulae (IV), (IV-1), (IV-2), (IV-3), (IV-4-), (IV-5), (IV-6), (IV-11), (IV-12), (IV-13), (IV-14), (IV-15), (IV-16) (IV-17) or (IV-18) and a carbonate precursor, such as a diester carbonate, are reacted in the presence of a basic compound catalyst, a transesterification catalyst, or a mixed catalyst thereof, or in the absence of a catalyst.

A thermoplastic resin (or a polymer) other than a polycarbonate resin, such as polyestercarbonates and polyesters is obtained by using the dihydroxy compound represented by the formulae (I), (Ia), (Ia′), (Ia-1), (Ib), (Ib-1), (Ib-2), (Ic), (Ic-1), (Ic-2), (Id), (Id-1), (Id-2), (Id-3) and (Id-4), respectively, or a combination thereof with at least one compound represented by the formulae (IV), (IV-1), (IV-2), (IV-3), (IV-4-), (IV-5), (IV-6); (IV-11), (IV-12), (IV-13), (IV-14), (IV-15), (IV-16) (IV-17) or (IV-18) as a material (or a monomer).

The dihydroxy component which is used for preparing the thermoplastic resin of the present invention comprises at least one compound, which is selected from the group consisting of the compounds of formula (Ia-1), with more preference given to those compounds and structural units of formulae (Ia-1), where R^{a1} and R^{a2} are identical and selected from the group consisting of phenylethynyl, naphthalene-1-ylethynyl and 2-naphthalene-2-ylethynyl and where R^{a3} and R^{a4} are hydrogen. In other words, the dihydroxy component which is used for preparing the thermoplastic resin of the present invention comprises at least one compound of the formula (Ia-1), which is selected from the group consisting of:
2,2′-bis(2-hydroxyethoxy)-6,6′-di(naphthalene-2-yl-ethynyl)-1,1′-binaphthalene (R^{a1} and R^{a2} are naphthalene-1-ylethynyl, R^{a3} and R^{a4} are hydrogen: D2NACBHBNA),
2,2′-bis(2-hydroxyethoxy)-6,6′-di(naphthalene-1-yl-ethynyl)-1,1′-binaphthalene (R^{a1} and R^{a2} are naphthalene-1-ylethynyl, R^{a3} and R^{a4} are hydrogen: D1NACBHBNA), and
2,2′-bis(2-hydroxyethoxy)-6,6′-di(phenylethynyl)-1,1′-binaphthalene (R^{a1} and R^{a2} are phenylethynyl, R^{a3} and R^{a4} are hydrogen: DPACBHBNA).

In particular, the dihydroxy component which is used for preparing the thermoplastic resin of the present invention comprises a combination of
i) at least one compound, which is selected from the group consisting of the compounds of formula (Ia-1), with more preference given to those compounds and structural units of formulae (Ia-1), where R^{a1} and R^{a2} are identical and selected from the group consisting of phenylethynyl, naphthalene-1-ylethynyl and 2-naphthalene-2-ylethynyl and where R^{a3} and R^{a4} are hydrogen, with most preference given to D2NACBHBNA, D1NACBHBNA and DPACBHBNA and combinations thereof;

and
ii) at least one compound of the formula (IV), in particular, at least one compound which is selected from the compounds of the formulae (IV-1), (IV-2), (IV-3), (IV-4), (IV-5), (IV-6), (IV-11), (IV-12), (IV-13), (IV-14), (IV-15), (IV-16) (IV-17) or (IV-18), with particular preference given to the compounds of the formulae (IV-12), (IV-13) or (IV-18), with more preference given to the compounds selected from the group consisting of
2,2′-bis(2-hydroxyethoxy)-1,1′-binaphtyl,
9,9-bis[6-(2-hydroxyethoxy)naphthalene-2-yl]fluorene
9,9-bis[6-(2-hydroxymethoxy)naphthalene-2-yl]fluorene
9,9-bis[6-(2-hydroxypropyloxy)naphthalene-2-yl]fluorene
9,9-bis[6-(2-hydroxybutoxy)naphthalene-2-yl]fluorene
9,9-bis[4-(2-hydroxyethoxy)-phenyl]fluorene,
9,9-bis[4-(2-hydroxyethoxy)-3-methylphenyl]fluorene,
9,9-bis[4-(2-hydroxyethoxy)-3-tert-butylphenyl]fluorene,
9,9-bis[4-(2-hydroxyethoxy)-3-isopropylphenyl]fluorene,
9,9-bis[4-(2-hydroxyethoxy)-3-cyclohexylphenyl]fluorene,
9,9-bis[4-(2-hydroxy ethoxy)-3-phenylphenyl]fluorene
   and combinations thereof,
   and with special preference given to the compounds selected from the group consisting of 2,2′-bis(2-hydroxyethoxy)-1,1′-binaphtyl (BNE or BHBNA), 9,9-bis(6-(2-hydroxyethoxy)-2-naphthyl)fluorene (BNEF) and 9,9-bis(4-(2-hydroxyethoxy)-3-phenylphenyl)fluorene (BPPEF) and combinations thereof.

As mentioned before, the monomers of formula (I) and likewise the co-monomers of formula (IV) used for producing the thermoplastic resin may contain certain impurities resulting from their preparation.

For example, the compound of the formula (Ia-1), where R^{a} is phenylethynyl, i.e. the compound 2,2′-bis(2-hydroxyethoxy)-6,6′-di(phenylethynyl)-1,1′-binaphthalene (DPACBHBNA) represented by the formula (Ia-1.1)

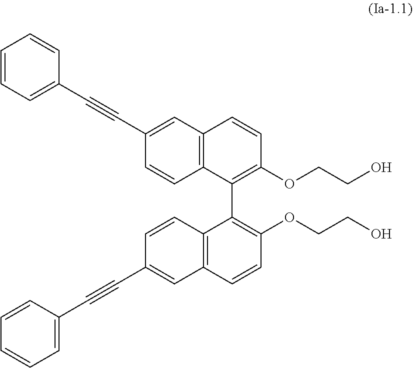 [SMILES: OCCOc1ccc2cc(C#Cc3ccccc3)ccc2c1-c1c(OCCO)ccc2cc(C#Cc3ccccc3)ccc12]

may include the compounds TPACBHBNA, BrPACBHBNA, DPACTHBNA, PACBHBNA, DPACMHBNA, BisPAC (homo-coupling product of Ph-C═CH), bis-DPACBHBNA-carbonate, and Ph-C═CH as impurities, as presented in the following scheme:

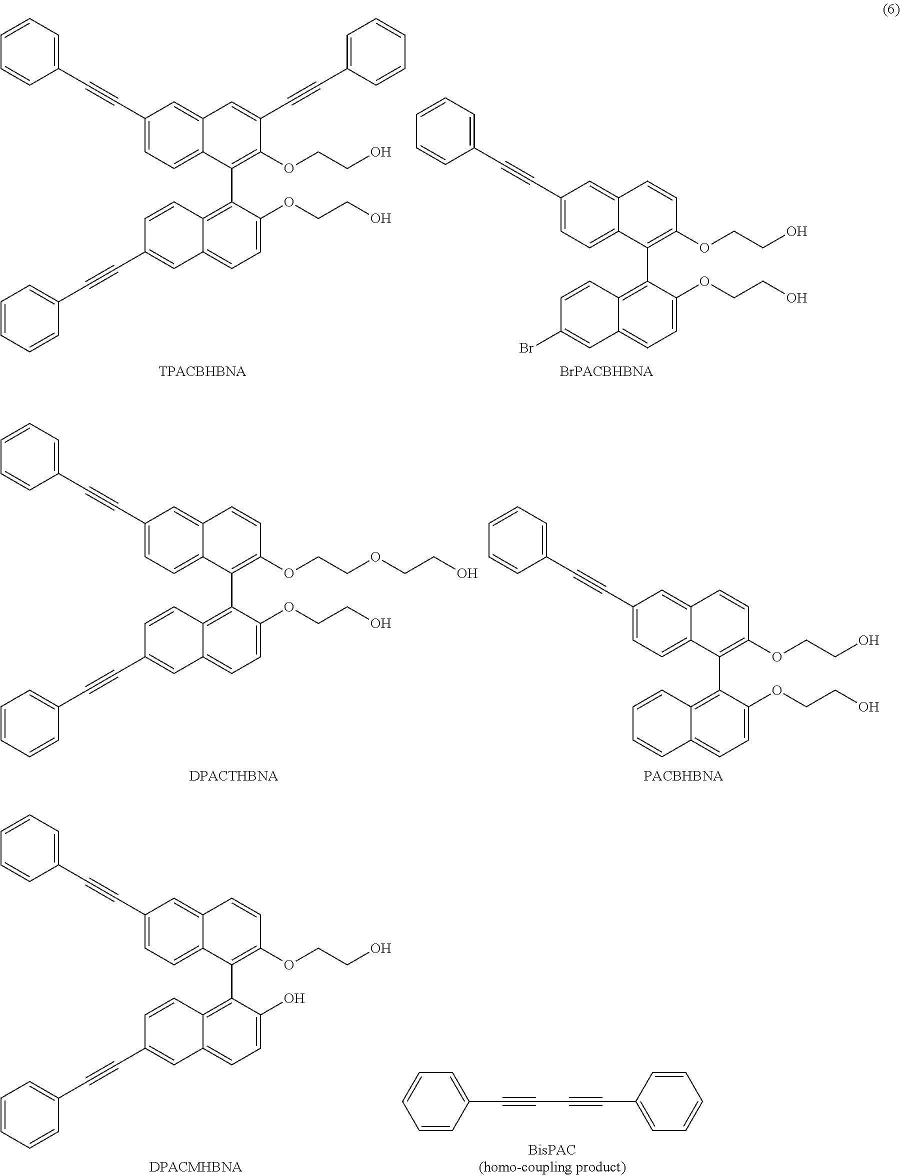

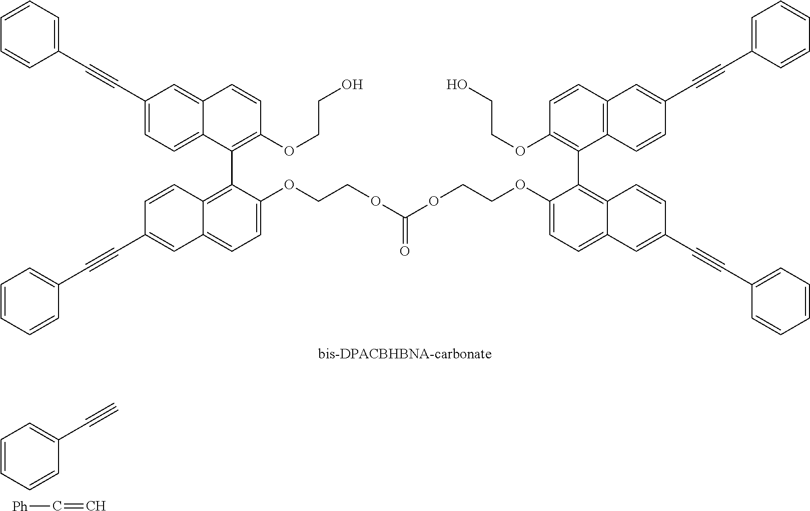

For example, the compound of the formula (Ia-1), where R^{a} is naphthalene-2-ylethynyl, i.e. the compound 2,2′-bis(2-hydroxyethoxy)-6,6′-di(naphthalene-2-yl-ethynyl)-1,1′-binaphthalene (D2NACBHBNA) represented by the formula (Ia-1.7)

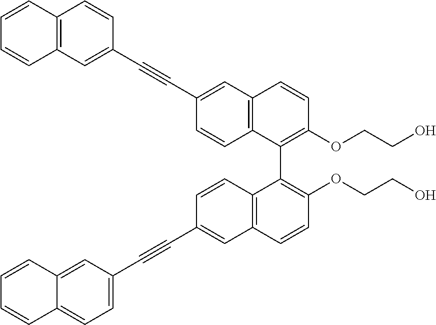 [SMILES: OCCOc1ccc2cc(C#Cc3ccc4ccccc4c3)ccc2c1-c1c(OCCO)ccc2cc(C#Cc3ccc4ccccc4c3)ccc12]

may include the compounds T2NACBHBNA, Br2NACBHBNA, D2NACTHBNA, 2NACBHBNA, D2NACMHBNA, BisD2NACBHBNA-carbonate, bis-2NAC (homo-coupling product of 2Npht-C═CH), and 2Npht-C═CH as impurities, as presented in the following scheme:

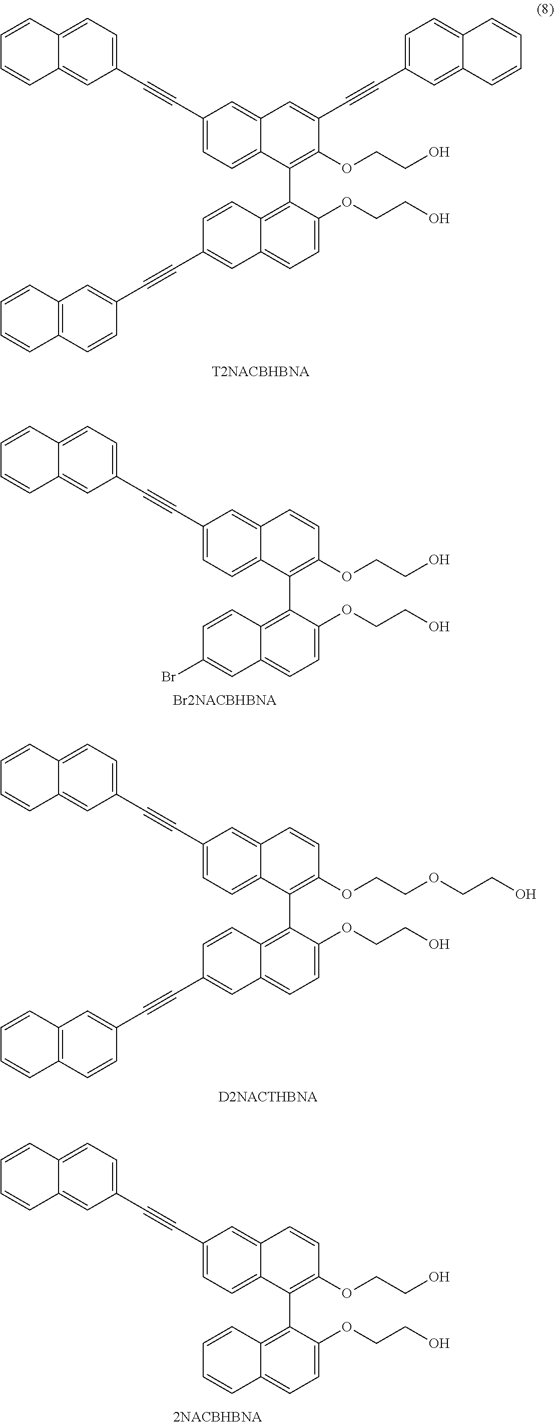 [SMILES: OCCOc1ccc2cc(C#Cc3ccc4ccccc4c3)ccc2c1-c1c(OCCO)c(C#Cc2ccc3ccccc3c2)cc2cc(C#Cc3ccc4ccccc4c3)ccc12 ; OCCOc1ccc2cc(Br)ccc2c1-c1c(OCCO)ccc2cc(C#Cc3ccc4ccccc4c3)ccc12 ; OCCOCCOc1ccc2cc(C#Cc3ccc4ccccc4c3)ccc2c1-c1c(OCCO)ccc2cc(C#Cc3ccc4ccccc4c3)ccc12 ; OCCOc1ccc2ccccc2c1-c1c(OCCO)ccc2cc(C#Cc3ccc4ccccc4c3)ccc12]

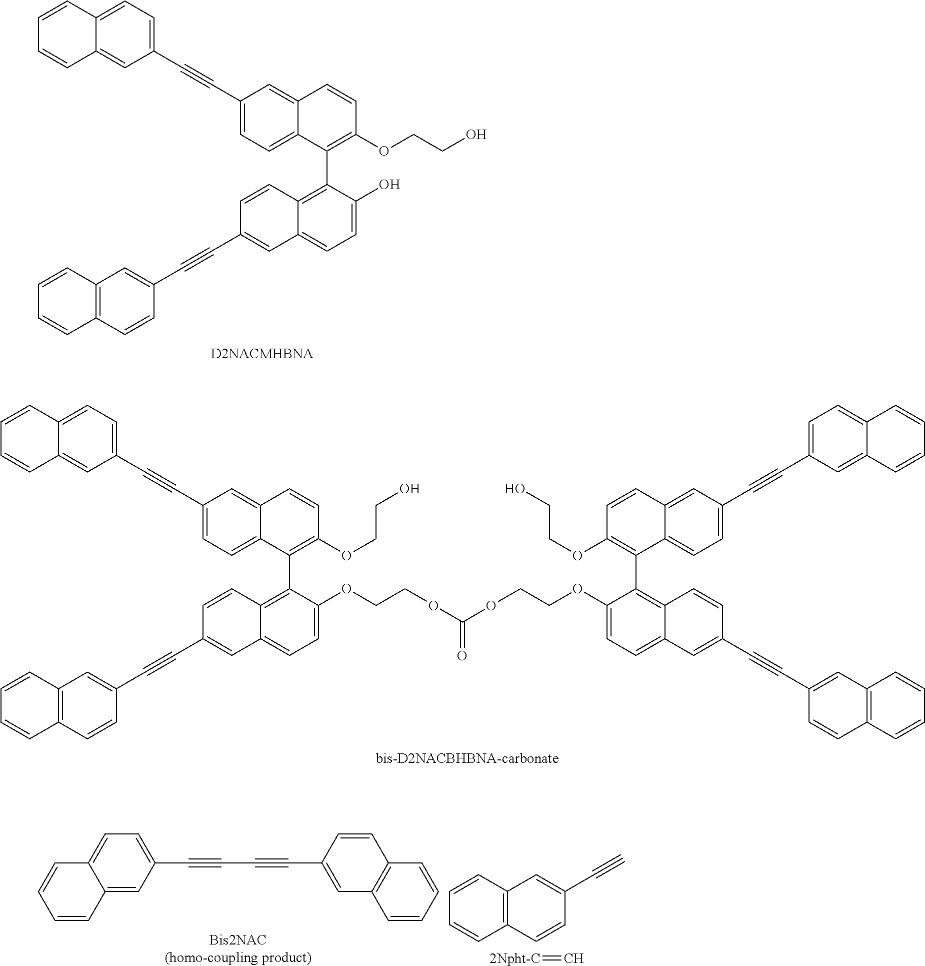

In particular, the total amount of impurities in the compounds of formulae (IVa-1.1) and (IVa-1.7) is preferably 1000 ppm or lower, more preferably 500 ppm or lower, still more preferably 200 ppm or lower, and especially preferably 100 ppm or lower. The total content of dihydroxy compounds in which a carbon number of at least one of the radical R³ differs from the formulae (IVa-1.1) and (IVa-1.7) is further preferably 50 ppm or lower, and more preferably 20 ppm or lower.

For example, the monomers of the formulae (IV-2) and (IV-3), where R^{z} is O-Alk²- or O-Alk²-[O-Alk²-]ₚ-, may include a dihydroxy compound in which both R^{z} are a single bond, or a dihydroxy compound in which one of R^{z} is a single bond, instead of O-Alk²- or O-Alk²-[O-Alk²-]ₚ-.

The total amount of such dihydroxy compounds of the formulae (IV-2) or (IV-3) in which at least one of R^{z} differs from O-Alk²- or O-Alk²-[O-Alk²-]ₚ-, is preferably 1000 ppm or lower, more preferably 500 ppm or lower, still more preferably 200 ppm or lower, and especially preferably 100 ppm or lower; in the monomer(s) of which main component is the dihydroxy compound(s) represented by the formulae (IV-2) or (IV-3). The total content of the dihydroxy compounds in which at least one of the values of c and d differs from the formula (IV-2) or (IV-3) is still preferably 50 ppm or lower, and more preferably 20 ppm or lower.

The polycarbonate resins can be obtained by reacting the monomer compounds of the formula (I) or by reacting combination of at least one monomer compound of the formula (I), in particular of the formulae (Ia) or (Ia-1) and especially of the formulae (Ia-1.1) or (Ia-1.7), and one or more monomer compounds of the formula (IV), in particular of the formulae (IV-1) or (IV) and especially of the formulae (IV-12), (IV-13) or (IV-18), and the like, as dihydroxy components; with carbonate precursors, such as diester carbonates.

However, in a polymerization process for manufacturing the polycarbonate resins, some compounds which are basically represented by the formulae (I) and (IV), but one of or both of the terminal —R³OH or —R^{z}OH radicals is replaced with a different radical, such as a vinyl terminal radical represented by —OCH═CH₂ can be formed as impurities. Because the amount of such impurities is generally small, the products of the formed polymers can be used as polycarbonate resins without a purification process.

The thermoplastic resin of the present invention may also contain minor amount of impurities, for example, as extra contents of thermoplastic resin composition or a part of the polymer skeleton of the thermoplastic resin. The examples of such impurities include phenols formed by a process for forming the thermoplastic resin, unreacted diester carbonates and monomers. The total amount of impurities in the thermoplastic resin may be 5000 ppm or lower, or 2000 ppm or lower. The total amount of impurities in the thermoplastic resin is preferably 1000 ppm or lower, more preferably 500 ppm or lower, still more preferably 200 ppm or lower, and especially preferably 100 ppm or lower.

The total amount of phenols as impurities in the thermoplastic resin may be 3000 ppm or lower, or 2000 ppm or lower. The total amount of phenols as impurities is preferably 1000 ppm or lower, more preferably 800 ppm or lower, still more preferably 500 ppm or lower, and especially preferably 300 ppm or lower.

The total amount of diester carbonates as impurities in the thermoplastic resin is preferably 1000 ppm or lower, more preferably 500 ppm or lower, still more preferably 100 ppm or lower, and especially preferably 50 ppm or lower.

The total amount of unreacted monomers as impurities in the thermoplastic resin is preferably 3000 ppm or lower, more preferably 2000 ppm or lower, still more preferably 1000 ppm or lower, and especially preferably 500 ppm or lower.

The lower limit of the total amount of these impurities is not important, but may be 0.1 ppm, or 1.0 ppm.

Resins having targeted characteristics can be formed by adjusting the amounts of phenols and diester carbonates. The amounts of phenols, diester carbonates, and monomers can be suitably adjusted by arranging the conditions for polycondensation, the working conditions of devices used for polymerization, or the conditions for extrusion molding after the polycondensation process.

The weight-average molecular weight (Mw), as determined by GPC described below, of the thermoplastic resin according to the present invention is preferably in the range from 5000 to 100000 Dalton, more preferably 10000 to 80000 Dalton, and still more preferably 15000 to 50000 Dalton. The number-average molecular weight (Mn) of the thermoplastic resin according to the present invention is preferably 3000 to 20000, more preferably 5000 to 15000, and still more preferably 7000 to 14000.

The value of the molecular weight distribution (Mw/Mn) of the thermoplastic resin according to the present invention is preferably 1.5 to 9.0, more preferably 1.8 to 7.0, and still more preferably 2.0 to 4.0.

When a thermoplastic resin has the value of the weight-average molecular weight (Mw) within the above-mentioned suitable range, a molded article made from the thermoplastic resin has high strength. In addition, such a thermoplastic resin with the suitable Mw value is advantageous for molding because of its excellent fluidity.

The above-mentioned polycarbonate resin has a high refractive index (nD or nd) and thus is suitable to an optical lens. The values of the refractive index as referred herein are values of a film having a thickness of 0.1 mm may be measured by use of an Abbe refractive index meter by a method of JIS-K-7142. The refractive index of the polycarbonate resin according to the present invention at 23° C. at a wavelength of 589 nm is, in case the resin includes the structural unit (2), preferably 1.660 or higher, more preferably 1.680 or higher, still more preferably 1.690 or higher. For example, the refractive index of the copolycarbonate resin including the structural unit (2) and a structural unit (V) according to the present invention is preferably 1.660 to 1.720, preferably 1.680 to 1.720, still more preferably 1.690 to 1.720.

The Abbe number (v) of the polycarbonate resin is preferably 20 or lower, more preferably 18 or lower, and still more preferably 17 or lower. The Abbe number may be calculated by use of the following equation based on the refractive index at wavelengths of 487 nm, 589 nm and 656 nm at 23° C.

v=(nD−1)/(nF−nC)
nD: refractive index at a wavelength of 589 nm
nC: refractive index at a wavelength of 656 nm
nF: refractive index at a wavelength of 486 nm

The glass transition temperature (Tg) of the polycarbonate resin as an example of the thermoplastic resin according to the present invention is, in consideration of that the polycarbonate is usable for injection molding, preferably 90 to 185° C., more preferably 125 to 175° C., and still more preferably 140 to 165° C. With regard to the molding fluidity and the molding heat resistance, the lower limit of Tg is preferably 130° C. and more preferably 135° C., and the upper limit of Tg is preferably 185° C. and more preferably 175° C. A glass transition temperature (Tg) in the above given ranges provides a significant range of usable temperature and avoids the risk that the melting temperature of the resin may be too high, and thus the resin may be undesirably decomposed or colored. What is more, it allows for preparing molds having have a high surface accuracy.

An optical molded body such as an optical element produced by using a polycarbonate resin of the present invention has a total light transmittance of preferably 85% or higher, more preferably 87% or higher, and especially preferably 88% or higher. A total light transmittance of preferably 85% or higher is as good as that provided by bisphenol A type polycarbonate resin or the like.

The thermoplastic resin according to the present invention has high moisture and heat resistance. The moisture and heat resistance may be evaluated by performing a “PCT test” (pressure cooker test) on a molded body such as an optical element produced by use of the thermoplastic resin and then measuring the total light transmittance of the molded body after the PCT test. In the PCT test, first, an injection molded body having a diameter of 50 mm and a thickness of 3 mm is kept for 20 hours with PC305S III made by HIRAYAMA Corporation under the conditions of 120° C., 0.2 MPa, 100% RH for 20 hours Then, the sample of the injection molded body is removed from the device and the total light transmittance is measured using the SE2000 type spectroscopic parallax measuring instrument made by Nippon Denshoku Industries Co., Ltd in accordance with the method of JIS-K-7361-1.

The thermoplastic resin according to the present invention has a post-PCT test total light transmittance of 60% or higher, preferably 70% or higher, more preferably 75% or higher, still more preferably 80% or higher, and especially preferably 85% or higher. As long as the total light transmittance is 60% or higher, the thermoplastic resin is considered to have a higher moisture and heat resistance than that of the conventional thermoplastic resin.

The thermoplastic resin according to the present invention has a b value, which represents the hue, of preferably 5 or lower. As the b value is smaller, the color is less yellowish, which is good as a hue.

According to the invention, the diol component, which is used in the preparation of the polycarbonates or polyesters, may additionally comprise one or more diol monomers, which are different from the monomer compound of the formula (I), such as one or more monomers of the formula (IV).

Suitable diol monomers, which are different from the monomer compound of the formula (I), are those, which are conventionally used in the preparation of polycarbonates, e.g.
aliphatic diols such as ethylene glycol, propanediol, butanediol, pentanediol and hexanediol;
alicyclic diols such as tricyclo[5.2.1.02,6]decane dimethanol, cyclohexane-1,4-dimethanol, decalin-2,6-dimethanol, norbornane dimethanol, pentacyclopentadecane dimethanol, cyclopentane-1,3-dimethanol, spiroglycol,
1,4:3,6-dianhydro-D-sorbitol, 1,4:3,6-dianhydro-D-mannitol and 1,4:3,6-dianhydro-L-iditol are also included in examples of the diol; and
aromatic diols, in particular aromatic diols of the formula (IV) such as bis(4-hydrophenyl)methane, 1,1-bis(4-hydroxyphenyl)ethane, bis(4-hydroxyhenyl)ether, bis(4-hydroxyphenyl)sulfoxide, bis(4-hydroxyphenyl)sulfide, bis(4-hydroxyphenyl)sulfone, bis(4-hydroxyphenyl)ketone, 2,2-bis(4-hydroxyphenyl)propane, 2,2-bis(4-hydroxy-3-t-butylphenyl)propane, 2,2-bis(4-hydroxy-3-methylphenyl)propane, 1,1-bis(4-hydroxyphenyl)cyclopentane, 1,1-bis(4-hydroxyphenyl)cyclohexane, 2,2-bis(4-hydroxyphenyl)hexafluoro-propane, bis(4-hydroxyphenyl)diphenylmethane, 1,1-bis(4-hydroxyphenyl)-1-phenylethane, α,ω-bis[2-(p-hydroxyphenyl)ethyl]polydimethylsiloxane, α,ω-bis[3-(o-hydroxyphenyl)propyl]polydimethylsiloxane, 4,4′-[1,3-phenylenebis(1-methylethylidene)hydroxyphenyl]-1-phenylethane, 9,9-bis(4-hydroxyphenyl)fluorene, 9,9-bis[4-(2-hydroxyethoxy)-3-methylphenyl]fluorene, 9,9-bis[4-(2-hydroxyethoxy)-3-tert-butylphenyl]fluorene, 9,9-bis[4-(2-hydroxyethoxy)-3-isopropylphenyl]fluorene, 9,9-bis[4-(2-hydroxyethoxy)-3-cyclohexylphenyl]fluorene, 9,9-bis(4-hydroxy-3-phenylphenyl)fluorene, 9,9-bis(4-(2-hydroxyethyl)phenyl)fluorene, 9,9-bis(4-(2-hydroxyethyl)-3-phenylphenyl)fluorene, 9,9-bis(6-hydroxy-2-naphthyl)fluorene, 9,9-bis(6-(2-hydroxyethyl)-2-naphthyl)fluorene, 10,10-bis(4-hydroxyphenyl)anthracen-9-on, 10,10-bis(4-(2-hydroxyethyl)phenyl)anthracen-9-on and 2,2′-[1,1′-binaphthalene-2,2′-diylbis(oxy)]diethanol, also termed 2,2′-bis(2-hydroxyethoxy)-1,1′-binaphthyl or 2,2′-bis(2-hydroxyethoxy)-1,1′-binaphthalene (BNE).

Preferably, the diol component comprises at least one monomer of the formula (IV) in addition to the monomer of formula (I). In particular, the total amount of monomers of formulae (I) and (IV) contribute to the diol component by at least 90% by weight, based on the total weight of the diol component or by at least 90 mol-%, based on the total molar amount of the diol monomers of the diol component. In particular, the diol component comprises at least one monomer selected from the monomers of formulae (IV-1) to (IV-8) in addition to the monomer of formula (I). More particularly, the diol component comprises at least one monomer selected from the monomers of formulae (IV-1), (IV-2), (IV-3) and (IV-8) in addition to the monomer of formula (I). Especially, the diol component comprises at least one monomer selected from 2,2′-bis(2-hydroxyethoxy)-1,1′-2,2′-bis(2-hydroxyethoxy)-1,1′-binaphtyl, 9,9-bis(6-(2-hydroxyethoxy)-2-naphthyl)fluorene 9,9-bis(4-(2-hydroxyethoxy)-3-phenylphenyl)-fluorene and 9,9-bis(4-(2-hydroxyethoxy)-3-phenylphenyl)fluorene and combinations thereof in addition to the monomer of formula (I).

Frequently, the relative amount of monomer compound of formula (I), based on the total weight of the diol component, is at least 1% by weight, preferably at least 2% or at least 5% by weight, in particular at least 8% by weight or at least 10% by weight and especially at least 12% by weight or at least 15% by weight, preferably in the range of 1 to 90% by weight or in the range of 5 to 90% by weight, in particular in the range of 2 to 80% by weight or in the range of 5 to 80% by weight or in the range of 8 to 80% by weight or in the range 10 to 80% by weight, especially in the range of 5 to 70% by weight or in the range of 8 to 70% by weight or in the range 10 to 70% by weight or in the range of 15 to 70% by weight, but may also be as high as 100% by weight.

Frequently, the relative molar amount of monomer compound of formula (I), based on the total molar of the diol component, is at least 1 mol-%, preferably at least 2 mol-% or at least 5 mol-%, in particular at least 8 mol-% or at least 10 mol-% and especially at least 12 mol-% or at least 15 mol-%, preferably in the range of 1 to 80 mol-% or in the range of 2 to 80 mol-% or in the range of 5 to 80 mol-% or in the range of 8 to 80 mol-%, in particular in the range of 2 to 70 mol-% or in the range of 5 to 70 mol-% or in the range of 8 to 70 mol-% or in the range of 10 to 70 mol-%, especially in the range of 5 to 60 mol-% or in the range of 8 to 60 mol-% or in the range of 10 to 60 mol-% or in the range of 12 to 60 mol-% or in the range of 15 to 60 mol-%, but may also be as high as 100 mol-%.

Consequently, the relative molar amount of monomer compound of formula (IV), based on the total molar of the diol component, will not exceed 99 mol-% or 98 mol-% or 95 mol-%, in particular not exceed 92 mol-% or 90 mol-% and especially not exceed 88 mol-% or 85 mol-%, and is preferably in the range of 20 to 99 mol-% or in the range of 20 to 98 mol-% or in the range of 20 to 95 mol-% or in the range of 20 to 92 mol-%, in particular in the range of 30 to 98 mol-% or in the range of 30 to 95 mol-% or in the range of 30 to 92 mol-% or in the range of 30 to 90 mol-%, especially in the range of 40 to 95 mol-% or in the range of 40 to 92 mol-% or in the range of 40 to 90 mol-% or in the range of 40 to 88 mol-% or in the range of 40 to 85 mol-%, but may also be as high as 99.9 mol-%.

Frequently, the total molar amount of monomers of formula (I) and monomers of formula (IV) is at least 80 mol-%, in particular at least 90 mol-%, especially at least 95 mol-% or up to 100 mol-%, based on the total molar amount of the diol monomers in the diol component.

Examples of further preferred aromatic dihydroxy compound, which can be used in addition to the monomers of formula (I) and optionally monomers of formula (IV) include, but are not limited to bisphenol A, bisphenol AP, bisphenol AF, bisphenol B, bisphenol BP, bisphenol C, bisphenol E, bisphenol F, bisphenol G, bisphenol M, bisphenol S, bisphenol P, bisphenol PH, bisphenol TMC, bisphenol Z and the like.

In order to adjust the molecular weight and the melt viscosity, the monomers forming the thermoplastic polymer may also include a monofunctional compound, in case of polycarbonates a monofunctional alcohol and in case of polyesters a monofunctional alcohol or a monofunctional carboxylic acid. Suitable monoalcohols are butanol, hexanol and octanol. Suitable monocarboxylic acids include e.g. benzoic acid, propionic acid and butyric acid. In order to increase the molecular weight and the melt viscosity, the monomers forming the thermoplastic polymer may also include a polyfunctional compound, in case of polycarbonates a polyfunctional alcohol having three or more hydroxyl groups and in case of polyesters a polyfunctional alcohol having three or more hydroxyl groups or a polyfunctional carboxylic acid having three or more carboxyl groups. Suitable polyfunctional alcohols are e.g. glycerine, trimethylol propane, pentaerythrite and 1,3,5-trihydroxy pentane. Suitable polyfunctional carboxylic acids having three or more carboxyl groups are e.g. trimellitic acid and pyromellitic acid. The total amount of these compounds, will frequently not exceed 10 mol-%, based on the molar amount of the diol component.

Suitable carbonate forming monomers, are those, which are conventionally used as carbonate forming monomers in the preparation of polycarbonates, include, but are not limited to phosgene, diphosgene and diester carbonates such as diethyl carbonate, diphenyl carbonate, di-p-tolyl carbonate, phenyl-p-tolyl carbonate, di-p-chlorophenyl carbonate and dinaphthyl carbonate. Out of these, diphenyl carbonate is particularly preferred. The carbonate forming monomer is frequently used at a ratio of 0.97 to 1.20 mol, and more preferably 0.98 to 1.10 mol, with respect to 1 mol of the dihydroxy compound(s) in total.

Suitable dicarboxylic acids include, but are not limited to
aliphatic dicarboxylic acids such as oxalic acid, malonic acid, succinic acid, glutaric acid, adipic acid, pimelic acid, suberic acid, azelaic acid;
alicyclic dicarboxylic acids such as tricyclo[5.2.1,02,6]decane dicarboxylic acid, cyclohexane-1,4-dicarboxylic acid, decalin-2,6-dicarboxylic acid, and norbornanedicarboxylic acid; and
aromatic dicarboxylic acids, such as benzene dicarboxylic acids, specifically phthalic acid, isophthalic acid, 2-methylterephthalic acid or terephthalic acid, and naphthalene dicarboxylic acids, specifically naphthalene-1,3-dicarboxylic acid, naphthalene-1,4-dicarboxylic acid, naphthalene-1,5-dicarboxylic acid, naphthalene-1,6-dicarboxylic acid, naphthalene-1,7-dicarboxylic acid, naphthalene-2,5-dicarboxylic acid, naphthalene-2,6-dicarboxylic acid and naphthalene-2,7-dicarboxylic acid.

Suitable ester forming derivatives of dicarboxylic acids include, but are not limited to the dialkyl esters, the diphenyl esters and the ditolyl esters.

In case of polyesters, the ester forming monomer is frequently used at a ratio of 0.97 to 1.20 mol, and more preferably 0.98 to 1.10 mol, with respect to 1 mol of the dihydroxy compound(s) in total.

The polycarbonates of the present invention can be prepared by reacting a diol component comprising a monomer of formula (I) and optionally a further diol monomer such as a monomer of the formula (IV) and a carbonate forming monomer by analogy to the well known preparation of polycarbonates as described e.g. in U.S. Pat. No. 9,360,593, US 2016/0319069 and US 2017/0276837, to which full reference is made.

The polyesters of the present invention can be prepared by reacting a diol component comprising a monomer of formula (I) and optionally a further diol monomer such as a monomer of the formula (IV) and a dicarboxylic acid or its ester forming derivative by analogy to the well known preparation of polyesters as described e.g. in US 2017/044311 and the references cited therein, to which full reference is made.

The polyestercarbonates of the present invention can be prepared by reacting a diol component comprising a monomer of formula (I) and optionally a further diol monomer such as a monomer of the formula (IV), a carbonate forming monomer and a dicarboxylic acid or its ester forming derivative by analogy to the well known preparation of polyestercarbonates as described in the art.

The polycarbonates, polyesters and polyestercarbonates are usually prepared by reacting the monomers of the diol component with the carbonate forming monomers and/or the ester forming monomers, i.e. the dicarboxylic acids or the ester forming derivatives thereof, in the presence of an esterification catalyst, in particular a transesterification catalyst, in case a carbonate forming monomer or an ester forming derivative of a polycarboxylic acid is used.

Suitable transesterification catalysts are basic compounds, which specifically include but are not limited to alkaline metal compounds, alkaline earth metal compound, nitrogen-containing compounds, and the like. Likewise, suitable transesterification catalysts are acidic compounds, which specifically include but are not limited to Lewis acid compounds of polyvalent metals, including compounds of as zinc, tin, titanium, zirconium, lead, and the like.

Examples of suitable alkaline metal compound include alkaline metal salts of an organic acid such as acetic acid, stearic acid, benzoic acid, or phenylphosphoric acid, alkaline metal phenolates, alkaline metal oxides, alkaline metal carbonates, alkaline metal borohydrides, alkaline metal hydrogen carbonates, alkaline metal phosphate, alkaline metal hydrogenphosphate, alkaline metal hydroxides, alkaline metal hydrides, alkaline metal alkoxides, and the like. Specific examples thereof include sodium hydroxide, potassium hydroxide, cesium hydroxide, lithium hydroxide, sodium hydrogen carbonate, sodium carbonate, potassium carbonate, cesium carbonate, lithium carbonate, sodium acetate, potassium acetate, cesium acetate, lithium acetate, sodium stearate, potassium stearate, cesium stearate, lithium stearate, sodium borohydride, sodium borophenoxide, sodium benzoate, potassium benzoate, cesium benzoate, lithium benzoate, disodium hydrogen phosphate, dipotassium hydrogen phosphate, dilithium hydrogen phosphate, and disodium phenylphosphate; and also include disodium salt, dipotassium salt, dicesium salt, dilithium salt of bisphenol A, sodium salt, potassium salt, cesium salt and lithium salt of phenol; and the like.

Examples of the alkaline earth metal compound include alkaline earth metal salts of an organic acid such as acetic acid, stearic acid, benzoic acid, or phenylphosphoric acid, alkaline earth metal phenolates, alkaline earth metal earth oxides, alkaline earth metal carbonates, alkaline metal borohydrides, alkaline earth metal hydrogen carbonates, alkaline earth metal hydroxides, alkaline earth metal hydrides, alkaline earth metal alkoxides, and the like. Specific examples thereof include magnesium hydroxide, calcium hydroxide, strontium hydroxide, barium hydroxide, magnesium hydrogen carbonate, calcium hydrogen carbonate, strontium hydrogen carbonate, barium hydrogen carbonate, magnesium carbonate, calcium carbonate, strontium carbonate, barium carbonate, magnesium acetate, calcium acetate, strontium acetate, barium acetate, magnesium stearate, calcium stearate, calcium benzoate, magnesium phenylphosphate, and the like.

Examples of the nitrogen-containing compound include quaternary ammoniumhydroxide, salt thereof, amines, and the like. Specific examples thereof include quaternary ammoniumhydroxides including an alkyl group, an aryl group or the like, such as tetramethylammoniumhydroxide, tetraethylammoniumhydroxide, tetrapropylammoniumhydroxide, tetrabutylammoniumhydroxide, trimethylbenzylammoniumhydroxide, and the like; tertiary amines such as triphenylamine, dimethylbenzylamine, triphenylamine, and the like; secondary amines such as diethylamine, dibutylamine, and the like; primary amines such as propylamine, butylamine, and the like; imidazoles such as 2-methylimidazole, 2-phenylimidazole, benzoimidazole, and the like; bases or basic salts such as ammonia, tetramethylammoniumborohydride, tetrabutylammoniumborohydride, tetrabutylammoniumtetraphenylborate, tetraphenylammoniumtetraphenylborate, and the like.

Preferred examples of the transesterification catalyst include salts of polyvalent metals such as zinc, tin, titanium, zirconium, lead, and the like, in particular the chlorides, alkoxides, alkanoates, benzoates, acetylacetonates and the like. They may be used independently or in a combination of two or more. Specific examples of such transesterification catalyst include zinc acetate, zinc benzoate, zinc 2-ethylhexanoate, tin chloride (II), tin chloride (IV), tin acetate (II), tin acetate (IV), dibutyltinlaurate, dibutyltinoxide, dibutyltinmethoxide, zirconiumacetylacetonate, zirconium oxyacetate, zirconiumtetrabutoxide, lead acetate (II), lead acetate (IV), and the like.

The transesterification catalyst are frequently used at a ratio of 10⁻⁹ to 10⁻³ mol, preferably 10⁻⁷ to 10⁻⁴ mol, with respect to 1 mol of the dihydroxy compound(s) in total.

Frequently, the polycarbonates, polyesters and polyestercarbonates are prepared by a melt polycondensation method. In the melt polycondensation the monomers are reacted in the absence of an additional inert solvent. While the reaction is performed any byproduct formed in the transesterification reaction is removed by heating the reaction mixture at ambient pressure or reduced pressure.

The melt polycondensation reaction preferably comprises charging the monomers and catalyst into a reactor and subjecting the reaction mixture to conditions, where the reaction between the monomers and the formation of the byproduct takes place. It has been found advantages, if the byproduct resides for at least a while in the polycondensation reaction. However, in order to drive the polycondensation reaction to the product side, it is beneficial to remove at least a portion of the formed byproduct during or preferably at the end of the polycondensation reaction. In order to allow the byproduct in the reaction mixture, the pressure may be controlled by closing the reactor, or by increasing or decreasing the pressure. The reaction time for this step is 20 minutes or longer and 240 minutes or shorter, preferably 40 minutes or longer and 180 minutes or shorter, and especially preferably 60 minutes or longer and 150 minutes or shorter. In this step, in the case where the byproduct is removed by distillation soon after being generated, the finally obtained thermoplastic resin has a low content of high molecular-weight resin molecules. By contrast, in the case where the byproduct is allowed to reside in the reactor for a certain time, the finally obtained thermoplastic resin has a high content of high molecular-weight resin molecules.

The melt polycondensation reaction may be performed in a continuous system or in a batch system. The reactor usable for the reaction may be of a vertical type including an anchor-type stirring blade, a Marbled® stirring blade, a helical ribbon-type stirring blade or the like; of a horizontal type including a paddle blade, a lattice blade, an eye glass-type blade or the like; or an extruder type including a screw. A reactor including a combination of such reactors is preferably usable in consideration of the viscosity of the polymerization product.

According to the method for producing the thermoplastic resin, such as a polycarbonate resin, after the polymerization reaction is finished, the catalyst may be removed or deactivated in order to maintain the thermal stability and the hydrolysis stability. A preferred method for deactivating the catalyst is the addition of an acidic substance. Specific examples of the acidic substance include esters such as butyl benzoate and the like; aromatic sulfonates such as p-toluenesulfonic acid and the like; aromatic sulfonic acid esters such as butyl p-toluenesulfonate, hexyl p-toluenesulfonate, and the like; phosphoric acids such as phosphorous acid, phosphoric acid, phosphonic acid, and the like; phosphorous acid esters such as triphenyl phosphite, monophenyl phosphite, diphenyl phosphite, diethyl phosphite, di-n-propyl phosphite, di-n-butyl phosphite, di-n-hexyl phosphite, dioctyl phosphite, monooctyl phosphite, and the like; phosphoric acid esters such as triphenyl phosphate, diphenyl phosphate, monophenyl phosphate, dibutyl phosphate, dioctyl phosphate, monooctyl phosphate, and the like; phosphonic acids such as diphenyl phosphonic acid, dioctyl phosphonic acid, dibutyl phosphonic acid, and the like; phosphonic acid esters such as diethyl phenylphosphonate, and the like; phosphines such as triphenylphosphine, bis(diphenylphosphino)ethane, and the like; boric acids such as boric acid, phenylboric acid, and the like; aromatic sulfonic acid salts such as tetrabutylphosphonium dodecylbenzenesulfonate salt, and the like; organic halides such as chloride stearate, benzoyl chloride, chloride p-toluenesulfonate, and the like; alkylsulfonic acids such as dimethylsulfonic acid, and the like; organic halides such as benzyl chloride, and the like. These deactivators are frequently used at 0.01 to 50 mol, preferably 0.3 to 20 mol, with respect to the catalyst. After the catalyst has been deactivated, there may be a step of removing low boiling point compounds from the polymer by distillation. The distillation is preferably performed at reduced pressure, e.g. at a pressure of 0.1 to 1 mmHg at a temperature of 200 to 350° C. For this step, a horizontal device including a stirring blade having a high surface renewal capability such as a paddle blade, a lattice blade, an eye glass-type blade or the like, or a thin film evaporator is preferably used.

It is desirable that the thermoplastic resin such as a polycarbonate resin has a very small amount of foreign objects. Therefore, the molten product is preferably filtered to remove and solids from the melt. The mesh of the filter is preferably 5 μm or less, and more preferably 1 μm or less. It is preferred that the generated polymer is filtrated by a polymer filter. The mesh of the polymer filter is preferably 100 μm or less, and more preferably 30 μm or less. A step of sampling a resin pellet needs to be performed in a low dust environment, needless to say. The dust environment is preferably of class 6 or lower, and more preferably of class 5 or lower.

The thermoplastic resin may be molded by any conventional molding procedure for producing optical elements. Suitable molding procedures include but are not limited to injection molding, compression molding, casting, roll processing, extrusion molding, extension and the like.

While it is possible to mold the thermoplastic resin of the invention as such, it is also possible to mold a resin composition, which contains at least one thermoplastic resin of the invention and which further contains at least one additive and/or further resin.

Suitable additives include antioxidants, processing stabilizers, photostabilizers, polymerization metal deactivators, flame retardants, lubricants, antistatic agents, surfactants, antibacterial agents, releasing agents, ultraviolet absorbers, plasticizers, compatibilizers, and the like. Suitable further resins are e.g. another polycarbonate resin, polyester carbonate resin, polyester resin, polyamide, polyacetal and the like, which does not contain repeating units of the formula (I).

Examples of the antioxidant include but are not limited to triethyleneglycol-bis[3-(3-tert-butyl-5-methyl-4-hydroxyphenyl)propionate], 1,6-hexanediol-bis[3-(3,5-di-tert-butyl-4-hydroxyphenyl)propionate], pentaerythritol-tetrakis[3-(3,5-di-tert-butyl-4-hydroxyphenyl)propionate], octadecyl-3-(3,5-di-tert-butyl-4-hydroxyphenyl)propionate, 3,9-bis(2,6-di-tert-butyl-4-methylphenoxy)-2,4,8,10-tetraoxa-3,9-diphosphaspiro[5.5]undecane, 5,7-Di-tert-butyl-3-(3,4-dimethylphenyl)benzofuran-2(3H)-one, 5,7-Di-tert-butyl-3-(1,2dimethylphenyl)benzofuran-2(3H)-one, 1,3,5-trimethyl-2,4,6-tris(3,5-di-tert-butyl-4-hydroxybenzyl)benzene, N,N-hexamethylenebis(3,5-di-tert-butyl-4-hydroxy-hydrocinnamide, 3,5-di-tert-butyl-4-hydroxy-benzylphosphonate-diethylester, tris(3,5-di-tert-butyl-4-hydroxybenzyl)isocyanurate, and 3,9-bis{1,1-dimethyl-2-[(3-(3-tert-butyl-4-hydroxy-5-methylphenyl)propionyloxy]ethyl}-2,4,8,10-tetraoxaspiro(5,5)undecane, and the like. Among these examples, 3,9-bis(2,6-di-tert-butyl-4-methylphenoxy)-2,4,8,10-tetraoxa-3,9-diphosphaspiro[5.5]undecane, 5,7-Di-tert-butyl-3-(3,4-dimethylphenyl)benzofuran-2(3H)-one, and 5,7-Di-tert-butyl-3-(1,2dimethylphenyl)benzofuran-2(3H)-one are more preferred. The content of the antioxidant in the thermoplastic resin is preferably 0.001 to 0.3 parts by weight with respect to 100 parts by weight of the thermoplastic resin.

Examples of the processing stabilizer include but are not limited to phosphorus-based processing stabilizers, sulfur-based processing stabilizers, and the like. Examples of the phosphorus-based processing stabilizer include phosphorous acid, phosphoric acid, phosphonous acid, phosphonic acid, esters thereof, and the like. Specific examples thereof include triphenylphosphite, tris(nonylphenyl)phosphite, tris(2,4-di-tert-butylphenyl)phosphite, tris(2,6-di-tert-butylphenyl)phosphite, tridecylphosphite, trioctylphosphite, trioctadecylphosphite, didecylmonophenylphosphite, dioctylmonophenylphosphite, diisopropylmonophenylphosphite, monobutyl-diphenylphosphite, monodecyldiphenylphosphite, monooctyldiphenylphosphite, bis(2,6-di-tert-butyl-4-methylphenyl)pentaerythritoldiphosphite, 2,2-methylenebis(4,6-di-tert-butylphenyl)octylphosphite, bis(nonylphenyl)pentaerythritoldiphosphite, bis(2,4-dicumylphenyl)pentaerythritoldiphosphite, bis(2,4-di-tert-butylphenyl)pentaerythritol-diphosphite, distearylpentaerythritoldiphosphite, tributylphosphate, triethylphosphate, trimethylphosphate, triphenylphosphate, diphenylmonoorthoxenylphosphate, dibutylphosphate, dioctylphosphate, diisopropylphosphate, dimethyl benzenephosphonate, diethyl benzenephosphonate, dipropyl benzenephosphonate, tetrakis(2,4-di-t-butylphenyl)-4,4′-biphenylenediphosphonite, tetrakis(2,4-di-t-butylphenyl)-4,3′-biphenylenediphosphonite, tetrakis(2,4-di-t-butylphenyl)-3,3′-biphenylenediphosphonite, bis(2,4-di-tert-butylphenyl)-4-phenyl-phenylphosphonite, bis(2,4-di-tert-butylphenyl)-3-phenyl-phenylphosphonite, and the like. The content of the phosphorus-based processing stabilizer in the thermoplastic resin composition is preferably 0.001 to 0.2 parts by weight with respect to 100 parts by weight of the thermoplastic resin.

Examples of the sulfur-based processing stabilizer include but are not limited to pentaerythritol-tetrakis(3-laurylthiopropionate), pentaerythritol-tetrakis(3-myristylthiopropionate), pentaerythritol-tetrakis(3-stearylthiopropionate), dilauryl-3,3′-thiodipropionate, dimyristyl-3,3′-thiodipropionate, distearyl-3,3′-thiodipropionate, and the like. The content of the sulfur-based processing stabilizer in the thermoplastic resin composition is preferably 0.001 to 0.2 parts by weight with respect to 100 parts by weight of the thermoplastic resin.

Preferred releasing agents contain at least 90% by weight of an ester of an alcohol and a fatty acid. Specific examples of the ester of an alcohol and a fatty acid include an ester of a monovalent alcohol and a fatty acid, and a partial ester or a total ester of a polyvalent alcohol and a fatty acid. Preferred examples of the above-described ester of an alcohol and a fatty acid include the esters of a monovalent alcohol having a carbon number of 1 to 20 and a saturated fatty acid having a carbon number of 10 to 30. Preferred examples of partial or total esters of a polyvalent alcohol and a fatty acid include the partial or total ester of a polyvalent alcohol having a carbon number of 2 to and a saturated fatty acid having a carbon number of 10 to 30. Specific examples of the ester of a monovalent alcohol and a fatty acid include stearyl stearate, palmityl palmitate, butyl stearate, methyl laurate, isopropyl palmitate, and the like. Specific examples of the partial or total ester of a polyvalent alcohol and a fatty acid include monoglyceride stearate, monoglyceride stearate, diglyceride stearate, triglyceride stearate, monosorbitate stearate, monoglyceride behenate, monoglyceride caprylate, monoglyceride laurate, pentaerythritol monostearate, pentaerythritol tetrastearate, pentaerythritol tetrapelargonate, propyleneglycol monostearate, biphenyl biphenate, sorbitan monostearate, 2-ethylhexylstearate, total or partial esters of dipentaerythritol such as dipentaerythritol hexastearate and the like, etc. The content of the releasing agent in the resin composition is preferably 0.005 to 2.0 parts by weight, more preferably 0.01 to 0.6 parts by weight, and still more preferably 0.02 to 0.5 parts by weight, with respect to 100 parts by weight of the thermoplastic resin.

Preferred ultraviolet absorbers are selected from the group consisting of benzotriazole-based ultraviolet absorbers, benzophenone-based ultraviolet absorbers, triazine-based ultraviolet absorbers, cyclic iminoester-based ultraviolet absorbers, and cyanoacrylate-based ultraviolet absorbers. Namely, the following ultraviolet absorbers may be used independently or in a combination of two or more.

Examples of benzotriazole-based ultraviolet absorbers include 2-(2-hydroxy-5-methylphenyl)benzotriazole, 2-(2-hydroxy-5-tert-octylphenyl)benzotriazole, 2-(2-hydroxy-3,5-dicumylphenyl)phenylbenzotriazole, 2-(2-hydroxy-3-tert-butyl-5-methylphenyl)-5-chlorobenzotriazole, 2,2′-methylenebis[4-(1,1,3,3-tetramethylbutyl)-6-(2N-benzotriazole-2-yl)phenol)], 2-(2-hydroxy-3,5-di-tert-butylphenyl)benzotriazole, 2-(2-hydroxy-3,5-di-tert-butylphenyl)-5-chlorobenzotriazole, 2-(2-hydroxy-3,5-di-tert-amylphenyl)benzotriazole, 2-(2-hydroxy-5-tert-octylphenyl)benzotriazole, 2-(2-hydroxy-5-tert-butylphenyl)benzotriazole, 2-(2-hydroxy-4-octoxyphenyl)benzotriazole, 2,2′-methylenebis(4-cumyl-6-benzotriazolephenyl), 2,2′-p-phenylenebis(1,3-benzoxazine-4-one), 2-[2-hydroxy-3-(3,4,5,6-tetrahydrophthalimidemethyl)-5-methylphenyl]benzotriazole, and the like.

Examples of benzophenone-based ultraviolet absorbers include 2,4-dihydroxybenzophenone, 2-hydroxy-4-methoxybenzophenone, 2-hydroxy-4-octoxybenzophenone, 2-hydroxy-4-benzyloxybenzophenone, 2-hydroxy-4-methoxy-5-sulfoxybenzophenone, 2-hydroxy-4-methoxybenzophenone-5-sulfonic acid hydrate, 2,2′-dihydroxy-4-methoxybenzophenone, 2,2′,4,4′-tetrahydroxybenzophenone, 2,2′-dihydroxy-4,4′-dimethoxybenzophenone, 2,2′-dihydroxy-4,4′-dimethoxy-5-sodiumsulfoxybenzophenone, bis(5-benzoyl-4-hydroxy-2-methoxyphenyl)methane, 2-hydroxy-4-n-dodecyloxybenzophenone, 2-hydroxy-4-methoxy-2′-carboxybenzophenone, and the like.

Examples of triazine-based ultraviolet absorbers include 2-(4,6-diphenyl-1,3,5-triazine-2-yl)-5-([(hexyl)oxy]-phenol, 2-(4,6-bis(2,4-dimethylphenyl)-1,3,5-triazine-2-yl)-5-(([(octyl)oxy]-phenol, and the like.

Examples of cyclic iminoester-based ultraviolet absorbers include 2,2′-bis(3,1-benzoxazine-4-one), 2,2′-p-phenylenebis(3,1-benzoxazine-4-one), 2,2′-m-phenylenebis(3,1-benzoxazine-4-one), 2,2′-(4,4′diphenylene)bis(3,1-benzoxazine-4-one), 2,2′-(2,6-naphthalene)bis(3,1-benzoxazine-4-one), 2,2′-(1,5-naphthalene)bis(3,1-benzoxazine-4-one), 2,2′-(2-methyl-p-phenylene)bis(3,1-benzoxazine-4-one), 2,2′-(2-nitro-p-phenylene)bis(3,1-benzoxazine-4-one), 2,2′-(2-chloro-p-phenylene)bis(3,1-benzoxazine-4-one), and the like.

Examples of cyanoacrylate-based ultraviolet absorbers include 1,3-bis-[(2′-cyano-3′,3′-diphenylacryloyl)oxy]-2,2-bis[(2-cyano-3,3-diphenylacryloyl)oxy]methylpropane, 1,3-bis-[(2-cyano-3,3-diphenylacryloyl)oxy]benzene, and the like.

The content of the ultraviolet absorber in the resin composition is preferably 0.01 to 3.0 parts by weight, more preferably 0.02 to 1.0 parts by weight, and still more preferably 0.05 to 0.8 parts by weight, with respect to 100 parts by weight of the thermoplastic resin. The ultraviolet absorber contained in such a range of content in accordance with the use may provide a sufficient climate resistance to the thermoplastic resin.

As mentioned above, the thermoplastic polymer resins, in particular the polycarbonate resins, comprising repeating units of formulae (II), (IIa), (IIa-1), (IIb), (IIb-1), (IIb-2), (IIc), (IIc-1), (IIc-2), (IId), (IId-1), (IId-2), (IId-3) and (IId-4), respectively, as described herein, provide high transparency and high refractive index to thermoplastic resins, which therefore are suitable for preparing optical devices, where high transparency and high refractive index is required. More precisely, the thermoplastic polycarbonates having structural units of formulae (II), (IIa), (IIa-1), (IIb), (IIb-1), (IIb-2), (IIc), (IIc-1), (IIc-2), (IId), (IId-1), (IId-2), (IId-3) and (IId-4), respectively, are characterized by having a high refractive index, which is preferably at least 1.660, more preferably at least 1.680, in particular at least 1.690.

The contribution of the monomer of the formulae (I), (Ia), (Ia-1), (Ib), (Ib-1), (Ib-2), (Ic), (Ic-1), (Ic-2), (Id), (Id-1), (Id-2), (Id-3) and (Id-4), respectively, to the refractive index of the thermoplastic resin, in particular a polycarbonate resin, will depend from the refractive index of said monomer and the relative amount of said monomer in the thermoplastic resin. In general, a higher refractive index of the monomer contained in the thermoplastic resin will result in a higher refractive index of the resulting thermoplastic resin. Apart from that, the refractive index of a thermoplastic resin comprising structural units of the formula (II) can be calculated from the refractive indices of the monomers used for preparing the thermoplastic resin, either from the refractive index of the monomers or ab initio, e.g. by using the computer software ACD/ChemSketch 2012 (Advanced Chemistry Development, Inc.).

In case of thermoplastic copolymer resins, the refractive index of the thermoplastic resin, in particular a polycarbonate resin, can be calculated from the refractive indices of the homopolymers of the respective monomers, which form the copolymer resin, by the following so called “Fox equation”:

1/n_{D}=x₁/n_{D1}+x₂/n_{D2}+ . . . xₙ/n_{Dn},

where n_{D} is the refractive index of the copolymer, x₁, x₂, . . . xₙ are the mass fractions of the monomers 1, 2, . . . n in the copolymer and n_{D1}, n_{D2}, . . . n_{Dn} are the refractive indices of the homopolymers synthesized from only one of the monomers 1, 2 n at a time. In case of polycarbonates, x₁, x₂, . . . xₙ are the mass fractions of the OH monomers 1, 2, . . . n, based on the total amount of OH monomer. It is apparent that a higher refractive index of a homopolymer will result in a higher refractive index of the copolymer.

The refractive indices of the thermoplastic resins can be determined directly or indirectly. For direct determination, the refractive indices n_{D} of the thermoplastic resins are measured at wavelength of 589 nm in accordance with the protocol JIS-K-7142 using an Abbe refractometer and applying a 0.1 mm film of the thermoplastic resin. In case of the refractive indices of the homopolycarbonates of the compounds of formula (I), the refractive indices can also be determined indirectly. For this, a co-polycarbonate of the respective monomer of formula (I) with 9,9-bis(4-(2-hydroxyethoxy)phenyl)-fluorene and diphenyl carbonate is prepared according to the protocol of example 1 in column 48 of U.S. Pat. No. 9,360,593 and the refractive indices n_{D} of the co-polycarbonate is measured at wavelength of 589 nm in accordance with the protocol JIS-K-7142 using an Abbe refractometer and applying a 0.1 mm film of the co-polycarbonate. From the thus measured refractive indices n_{D}, the refractive index of the homopolycarbonate of the respective monomer can be calculated by applying the Fox equation and the known refractive index of 9,9-bis(4-(2-hydroxyethoxy)phenyl)fluorene (n_{D}(589 nm)=1.639).

As mentioned before, compounds of formula (I), which do not bear color-imparting radicals, such as some of the radicals R¹¹, Ar′ and R, can also be obtained in a purity, which provides for a low yellowness index Y.I., as determined in accordance with ASTM E313, which may also be important for the use in the preparation of optical resins.

More precisely, the yellowness index Y.I., as determined in accordance with ASTM E313, of the compounds of formula (I) preferably does not exceed 200, more preferably 100, even more preferably 50, in particular 20 or 10.

The thermoplastic resin according to the present invention has a high refractive index and a low Abbe number. The thermoplastic resin of the present invention can be used for producing a transparent conductive substrate usable for a liquid crystal display, an organic EL display, a solar cell and the like. Also, the thermoplastic resin of the present invention can be used as a structural material for optical parts, such as, optical disks, liquid crystal panels, optical cards, optical sheets, optical fibers, connectors, evaporated plastic reflecting mirrors, displays, and the like; or used as optical devices suitable for functional material purpose.

Accordingly, molded articles, such as optical devices can be formed using the thermoplastic resins of the present invention. The optical devices include optical lenses, and optical films. The specific examples of the optical devices include lenses, films, mirrors, filters, prisms, and so on. These optical devices can be formed by arbitrary production process, for example, by injection molding, compression molding, injection compression molding, extrusion molding, or solution casting.

Because of an excellent moldability and a high heat resistance, the thermoplastic resins of the present invention are very suitable for production of optical lenses which requires injection molding. For molding, the thermoplastic resins of the present invention, such as the polycarbonate resin, can be used with other thermoplastic resins, for example, different polycarbonate resin, polyestercarbonate resin, polyester resin, and other resins, as a mixture.

In addition, the thermoplastic resins of the present invention can be mixed with additives for forming the optical devices. As the additives for forming the optical devices, above-mentioned ones can be used. The additives may include antioxidants, processing stabilizers, photostabilizers, polymerization metal deactivators, flame retardants, lubricants, antistatic agents, surfactants, antibacterial agents, releasing agents, ultraviolet absorbers, plasticizers, compatibilizers, and the like.

As is clear from the above, another aspect of the present invention relates to an optical device made of a thermoplastic resin as defined above, where the thermoplastic resin comprising a structural unit represented by the formula (II) and optionally of formula (V). As regards to the preferred meanings and preferred embodiments of the structural units of the formulae (II) and (VI), reference is made to the statements given above.

An optical device made of an optical resin comprising the repeating units of the formula (II) and optionally repeating units of the formula (V) as defined herein are usually optical molded articles such as optical lenses, for example car head lamp lenses, Fresnel lenses, fθ lenses for laser printers, camera lenses, lenses for glasses and projection lenses for rear projection TV's, CD-ROM pick-up lenses, but also optical disks, optical elements for image display media, optical films, film substrates, optical filters or prisms, liquid crystal panels, optical cards, optical sheets, optical fibers, optical connectors, eposition plastic reflective mirrors, and the like. It is also useful for producing a transparent conductive substrate usable for an optical device suitable as a structural member or a functional member of a transparent conductive substrate for a liquid crystal display, an organic EL display, a solar cell and the like.

The optical lens produced from the thermoplastic resin according to the present invention has a high refractive index and a low Abbe number, and is highly moisture and heat resistant. Therefore, the optical lens can be used in the field in which a costly glass lens having a high refractive index is conventionally used, such as for a telescope, binoculars, a TV projector and the like. It is preferred that the optical lens is used in the form of an aspherical lens. Merely one aspherical lens may make the spherical aberration substantially zero. Therefore, it is not necessary to use a plurality of spherical lenses to remove the spherical aberration. Thereby the weight and the production cost of a device including the spherical aberration is decreased. An aspherical lens is useful especially as a camera lens among various types of optical lenses. The present invention easily provides an aspherical lens having a high refractive index and a low level of birefringence, which is technologically difficult to produce by processing glass.

An optical lens of the present invention may be formed, for example, by injection molding, compression molding, injection compression molding or casting the resin the repeating units of the formula (II) and optionally repeating units of the formula (V) as defined herein.

The optical lens of the present invention is characterized by a small optical distortion. An optical lens comprising a conventional optical resin has a large optical distortion. Although it is not impossible to reduce the value of an optical distortion by molding conditions, the condition widths are very small, thereby making molding extremely difficult. Since the resin having repeating units of the formula (II) and optionally repeating units of the formula (V) as defined herein has an extremely small optical distortion caused by the orientation of the resin and a small molding distortion, an excellent optical element can be obtained without setting molding conditions strictly.

To manufacture the optical lens of the present invention by injection molding, it is preferred that the lens should be molded at a cylinder temperature of 260° C. to 320° C. and a mold temperature of 100° C. to 140° C.

The optical lens of the present invention is advantageously used as an aspherical lens as required. Since spherical aberration can be substantially nullified with a single aspherical lens, spherical aberration does not need to be removed with a combination of spherical lenses, thereby making it possible to reduce the weight and the production cost. Therefore, out of optical lenses, the aspherical lens is particularly useful as a camera lens.

Since resins having repeating units of the formula (II) and optionally repeating units of the formula (V) as defined herein have a high moldability, they are particularly useful as the material of an optical lens which is thin and small in size and has a complex shape. As a lens size, the thickness of the center part of the lens is 0.05 to 3.0 mm, preferably 0.05 to 2.0 mm, more preferably 0.1 to 2.0 mm. The diameter of the lens is 1.0 to 20.0 mm, preferably 1.0 to 10.0 mm, more preferably 3.0 to 10.0 mm. It is preferably a meniscus lens which is convex on one side and concave on the other side.

The surface of the optical lens of the present invention may have a coating layer such as an antireflection layer or a hard coat layer as required. The anti reflection layer may be a single layer or a multi-layer and composed of an organic material or inorganic material but preferably an inorganic material. Examples of the inorganic material include oxides and fluorides such as silicon oxide, aluminum oxide, zirconium oxide, titanium oxide, cerium oxide, magnesium oxide and magnesium fluoride.

The optical lens of the present invention may be formed by an arbitrary method such as metal molding, cutting, polishing, laser machining, discharge machining or edging. Metal molding is preferred.

An optical film produced by the use of the thermoplastic resin according to the present invention is high in transparency and heat resistance, and therefore is preferably usable for a liquid crystal substrate film, an optical memory card or the like. In order to avoid foreign objects from being incorporated into the optical film as much as possible, the molding needs to be performed in a low dust environment, needless to say. The dust environment is preferably of class 6 or lower, and more preferably of class 5 or lower.

The following examples serve as further illustration of the invention.

### 1. Abbreviations

DCM: dichloromethane
EtOH: ethanol
EtOAc: ethyl acetate
MEK: 2-butanone
MeOH: methanol
MTBE: methyl tert-butyl ether
RT: room temperature
THF: tetrahydrofuran
TLC: thin layer chromatography
TMEDA: N,N,N′,N′-tetramethylethylendiamine
BNEF: 9,9-bis(6-(2-hydroxyethoxy)naphthalene-2-yl)fluorene
BNE: 2,2′-bis(2-hydroxyethoxy)-1,1′-binaphthalene
D2NACBHB or D2NACBHBNA: 2,2′-bis(2-hydroxyethoxy)-6,6′-di(naphthalene-2-yl-ethynyl)-1,1′-binaphthalene (=compound of example 4)
DPACBHBNA: 2,2′-bis(2-hydroxyethoxy)-6,6′-di(phenylethynyl)-1,1′-binaphthalene (=compound of example 3)
DPC: Diphenyl carbonate

### 2. Preparation of the Compounds of Formula (I)

### 2.1 Analytics Relating to Compounds of the Formula (I)

¹H-NMR spectra were determined at 23° C. using a 400 MHz NMR-spectrometer Avance III 400 HD from Bruker BioSpin GmbH. If not stated otherwise the solvent was CDCl₃

IR spectra were recorded by ATR FT-IR, using a Shimadzu FTIR-8400S spectrometer (45 no. of scans, resolution 4 cm¹; apodization: Happ-Genzel).

Melting points of the compounds were determined by Büchi Melting Point B-545.

UPLC (Ultra Performance Liquid Chromatography) analyses were carried out using the following system and conditions:

Waters Acquity UPLC H-Class Systems; column: Acquity UPLC BEH C18, 1.7 μm, 2×100 mm; column temperature: 40° C., gradient: acetonitrile/water: with acetonitrile at 0 min 50%, at 4 min 100%; at 5.8 min 100%; at 6.0 min 50%; at 8.0 min 50%); injection volume: 0.4 μl; run time: 8 min; detection at 210 nm.

The yellowness index YI of the compounds of formula (I) can be determined by analogy with ASTM E313 using the following protocol: 1 g of the compound of formula (I) is dissolved in 19 g of a mixture of MEK/water 95:5 (v/v). The solution is transferred into a 50 mm cuvette and transmission is determined in the range 300-800 nm by a Shimadzu UV-Visible spectrophotometer UV-1650PC. A mixture of MEK/water 95:5 (v/v) is used as a reference. From the spectra the yellowness index can be calculated by using the Software “RCA-software UV2DAT” in accordance with ASTM E308 (Standard practice for computing the colors of objects by using the CIE System) und ASTM E 313 (Standard practice for calculating yellowness and whiteness indices from instrumentally measured color coordinates).

The haze can be determined by measuring the transmission at 860 nm of a 5% solution of the respective compound of formula (I) in a mixture of MEK/water 95:5 (v/v) by a standard nephelometer.

### 2.2 Preparation Examples

### Example 1: Preparation of 6,6′-dibromo-2,2′-bis(2-hydroxyethoxy)-1,1′-binaphthyl (Compound of Formula (VIII), with Alk′=1,2-ethandiyl, d=e=1 and f=g=0)— Procedure 1 (Reference Example)

### 1.1: 6,6′-dibromo-1,1′-bi-2-naphthol (Compound (VII), with d=e=1 and f=g=0)

155 g (541.34 mmol) of 1,1′-bi-2-naphthol (compound (VI)) was suspended in 2.6 L DCM under argon atmosphere and the suspension was cooled to a temperature of −78° C. 2.3 to 2.5 equivalents of bromine, either neat or as a solution in DCM, was then added dropwise over a period of about 2 hours to the suspension. After continued stirring for about 1 hour at 22° C., TLC analysis (mobile phase: MTBE/n-heptane 2:1 (v/v)) revealed approximately complete consumption of the starting material and the reaction was then quenched by the addition of 1.16 kg of a saturated aqueous solution of sodium metabisulfite. Following phase separation the organic phase was washed with brine, dried over sodium sulfate and concentrated with a rotary evaporator until the product started to precipitate. After the precipitation was completed, the obtained solids were filtered off, washed with ice-cold toluene and dried. By concentrating the mother liqueur further product was obtained, which was also filtered off, washed with ice-cold toluene and dried. Combining the product fractions resulted in 205-210 g (ca. 85.3%-87.3%) of the raw title compound.

### 1.2: Alternative Preparation of 6,6′-dibromo-1,1′-bi-2-naphthol (Compound (VII), with d=e=1 and f=g=0) Via Oxidative Coupling of 6-bromo-2-naphthol

To a solution of 750 g (3.36 mol) 6-bromo-2-naphthol in 750 g methanol was added 5.5 g copper(II) chloride and 7.5 g TMEDA. The mixture was heated to 35° C. and a stream of air was passed through the mixture for 36 h under stirring. The mixture was cooled to 20° C. and the solid product was filtered off, washed with methanol and dried to yield the 6,6′-dibromo-1,1′-bi-2-naphthol (529 g; 1.19 mmol; 71%) with a chemical purity of about 97% (UPLC). Approximately 20% of additional product was isolated by concentrating the mother liquor, which was also filtered off, washed with methanol and dried to afford additional 164 g of the title compound with a chemical purity of about 90%. Further purification could be achieved by recrystallization from toluene.

### 1.3: Alternative Synthesis for the Preparation of 6,6′-dibromo-1,1′-bi-2-naphthol (Compound (VII), with d=e=1 and f=g=0)

44.87 g of 1,1′-bi-2-naphthol (compound (VI)) was suspended in 350 mL (305 g) of isopropyl acetate (IPAC) under an atmosphere of argon and the mixture was cooled to 0° C. Bromine (76.71 g) is slowly added (over approximately 1 h) in such a manner that the temperature did not rise above 5° C. After addition of the total amount of bromine, the reaction mixture is allowed to warm to RT. Following complete conversion (after approximately 2 h), the now homogeneous mixture was cooled down to 0° C. and a solution of Na₂S₂O₅ (25 g) in water (100 mL) was added to quench unreacted bromine. The aqueous and organic phases were separated and the organic phase was washed consecutively with water (60 mL), with a saturated aqueous solution of Na₂CO₃ (120 mL) until the pH value of the aqueous phase remained above 7 and with brine (50 mL). The organic phase was then dried over Na₂SO₄ and the solvent was removed in vacuo to yield 78.4 g of 6,6′-dibromo-1,1′-bi-2-naphthol as a brownish solid having a chemical purity of 91% (UPLC). This raw product was crystallized from a 2.5- to 3.5-fold volume of toluene and thoroughly washed with pentane to afford 58.3 g of the title compound (yellowish to white crystals) with a chemical purity of 98.8% (UPLC). Recrystallization from a 4.2- to 4.6-fold volume of toluene followed by thoroughly washing with pentane resulted in 54.4 g of the title compound (white crystals) having a chemical purity of 99.5% (UPLC).

### 1.4: 6,6′-dibromo-2,2′-bis-(2-hydroxyethoxy)-1,1′-binaphthyl (Compound (VIII), with Alk′=1,2-ethandiyl, d=e=1 and f=g=0)

71.1 g (160 mmol) of 6,6′-dibromo-1,1′-bi-2-naphthol obtained according to protocol 1.1, 42.27 g (480 mmol) of ethylene carbonate (3 equiv.) and 6.634 g (48 mmol) of potassium carbonate (30 mol-%) in 360 g (415 mL) toluene were heated under reflux for at least 5 hours (Caution: CO₂ gas evolution!), while monitoring the reaction progress by TLC (mobile phase: acetyl acetate or MTBE). Afterwards the reaction mixture was cooled to 80° C., additional 300 mL MEK was added to dissolve precipitated solids and obtain a clear solution. Then to reaction mixture 150 mL water was slowly added. Caution: gas evolution! After completion of a gas evolution and phase separation, the organic phase was washed successively twice with 5% or 10% aqueous solution of sodium hydroxide and twice or more with water until aqueous wash solution is neutral (pH=7). The organic phase was then concentrated with a rotary evaporator until the product started to precipitate. Following complete precipitation the obtained solids were filtered off, washed with toluene and dried to afford 17.1 g of the raw title compound (ca. 80.3%).

### Example 2: Preparation of 6,6′-dibromo-2,2′-bis-(2-hydroxyethoxy)-1,1′-binaphthyl (Compound of Formula (VIII), with Alk′=1,2-ethandiyl, d=e=1 and f=g=0)—Procedure 2 (Reference Example)

### 2.1: 2,2′-bis-(2-hydroxyethoxy)-1,1′-binaphthyl (Compound VI′, with Alk′=1,2-ethandiyl)

150.0 g (523.88 mmol) of 1,1′-bi-2-naphthol (compound VI), 138.37 g (1571.3 mmol) of ethylene carbonate (3 equiv.) and 21.75 g (157.13 mmol) of potassium carbonate (30 mol-%) in 1 L toluene were heated under reflux for at least 5 to 6 hours, by maintaining argon atmosphere. During the reaction gas evolves. The reaction is monitored by TLC using MTBE as solvent. When TLC indicates complete reaction the slightly yellow reaction mixture is cooled to 70° C. and mixed with 100 g of water (Caution: CO₂ gas evolution!) The reaction mixture is then stirred for further 10-15 min at 70° C. to dissolve potassium carbonate. The stirrer is stopped and phases are separated at about 70° C. The organic phase is washed with 100 g of 5% w/w aqueous solution of NaOH at 80-90° C. for at least 1 h (Caution: CO₂ gas evolution!), followed by washing with water (each 100 mL) at 70° C., until the pH of the washing water is neutral (pH 7). 15 g of charcoal is optionally added to the organic phase and the mixture is stirred at 70° C. for 30 min. Then the warm solution is filtered through Celite®. The clear and slightly yellowish filtrate is cooled to RT and product crystallizes in the form of thin platelets. The solid is filtered off, washed with toluene and dried. 142-170 g (72.4-86.7%) of the title compound are obtained as a white, dry solid.

### 2.2: 6,6′-dibromo-2,2′-bis-(2-hydroxyethoxy)-1,1′-binaphthyl (Compound (VIII), with Alk′=1,2-ethandiyl, d=e=1 and f=g=0)

A suspension of 37.44 g (100 mmol) of 2,2′-bis-(2-hydroxyethoxy)-1,1′-binaphthyl in 485 mL DCM was cooled to a temperature of −10° C. 40 g Bromine (2.3 to 2.5 equivalents) as a solution in DCM (120 mL) were then added dropwise over a period of between 1 and 2 hours to the suspension. After continued stirring for about 1 to 2 hours at RT, TLC analysis (mobile phase: MTBE/n-heptane 2:1 (v/v) or MeOH/water 7:3 (v/v)) revealed approximately complete consumption of the starting material and the reaction was then quenched by the addition of aqueous solution of sodium metabisulfite (12 g of Na₂S₂O₅ dissolved in 50 g water). Since product slowly precipitates, additionally 2.35 L MEK and 750 mL water were added in order to homogenize both organic and aqueous layers and to obtain two clear phases. Following phase separation the organic phase was successively washed with water (500 g), then saturated Na₂CO₃-solution (80 mL) [gas evolution] and brine (500 mL), dried over magnesium sulfate. The dried organic phase was filtered through Celite® and concentrated with a rotary evaporator until the product started to precipitate. After the precipitation was completed the obtained solids were filtered off, washed with ice-cold toluene and dried. By concentrating the mother liqueur further product was obtained, which was also filtered off, washed with ice-cold toluene and dried. Combined the product fractions were suspended in MTBE and purified twice by slurry wash at 45-50° C. for 2 hours, finally resulting in 44.5 g of the purified title compound (83%), which was used without additional recrystallization for the next step.

### 2.3: Alternative Preparation of 6,6′-dibromo-2,2′-bis-(2-hydroxyethoxy)-1,1′-binaphthyl (Compound (VIII), with Alk′=1,2-ethandiyl, d=e=1 and f=g=0)

In a reaction vessel, which had previously been dried and flushed with nitrogen or argon, 44.9 g of 2,2′-bis-(2-hydroxyethoxy)-1,1′-binaphthyl were suspended under argon or nitrogen in 337 mL of dry THF (peroxides-free and stabilized) at a temperature of 20-22° C. To the suspension were added 43.5 g of N-bromosuccinimide (2.1-2.2 equiv.) as a solid in four portions over 1.5 h. The reaction mixture turned into a yellow solution and was stirred overnight after which TLC analysis showed approximately complete consumption of the starting material. The reaction was then quenched by the addition of 25 mL of a saturated aqueous solution of sodium metabisulfite. Following phase separation the organic phase was washed successively with water and brine, dried over sodium sulfate and concentrated with a rotary evaporator until the product started to precipitate. Then 300 mL of water were added and the residual THF was removed in the rotary evaporator at a temperature of 60° C. The obtained solids were slurried in the remaining water at a temperature of 60° C., filtered off, washed with water and dried in an oven at a temperature of 60° C. and filtered off. The solids were slurried again in 300 mL of water at 60° C., filtered off and washed with water and dried in an oven at a temperature of 60° C. overnight. Further washing was achieved by slurrying the solids in 337 mL of MTBE at a temperature of 45° C. After cooling the slurry to RT the solids were filtered off, washed with MTBE and dried to afford 57.2 g of the title compound (90%) with a chemical purity of 91.34%, based on the non-volatile matter.

### Example 3: Preparation of 6,6′-di-(2-phenylacetylenyl)-2,2′-bis(2-hydroxyethoxy)-1,1′-binaphthyl (Compound of Formula (Ia-1), with R^{a1}=R^{a2}=2-phenylacetylenyl and R^{a3}=R^{a4}=Hydrogen)

A mixture of 6,6′-dibromo-2,2′-bis(2-hydroxyethoxy)-1,1′-binaphthyl obtained according to protocol 1.4, 2.2 or 2.3 (50.4 g, 94.7 mmol, 1.0 eq), ethynylbenzene (29.0 g, 284 mmol, 3.0 eq), PdCl₂ (840 mg, 4.74 mmol, 5.0 mol %), PPh₃ (2.49 g, 9.48 mmol, 10 mol %) and CuI (541 mg, 2.84 mmol, 3.0 mol %) in freshly degassed triethylamine (950 g) was heated to reflux (100-120° C.) for approx. 2 to 4 h. The reaction was followed by TLC (mobile phase: MeOH/H₂O 7:3 (v/v). After complete conversion, the solvent was removed under reduced pressure and THF (400 g) was added. The organic layer was washed with an aqueous solution of HCl (1 M, 200 g) and brine (100 g). The aqueous phases were extracted with THF (2×100 g) and the combined organic layers were concentrated to approximately one quarter of their original volume. The crystallization was completed at 0° C. and the precipitate filtered off to give the crude product as a grey solid (56.8 g, 98.8 mmol, 104%). Recrystallisation from THF with activated carbon followed by stirring with acetone at RT gave the desired product as a white solid (30.0 g, 52.3 mmol, 55%, chemical purity >99.8%).

Melting point: 156° C.

¹H NMR (400 MHz, CDCl₃): δ=8.11 (d, J=1.7 Hz, 2H), 7.97 (d, J=8.9 Hz, 2H), 7.60-7.50 (m, 4H), 7.47 (d, J=9.0 Hz, 2H), 7.40-7.29 (m, 8H), 7.09 (dt, J=8.8, 0.8 Hz, 2H), 4.24 (ddd, J=10.3, 6.6, 2.8 Hz, 2H), 4.05 (ddd, J=10.3, 5.4, 2.7 Hz, 2H), 3.70-3.50 (m, 4H), 2.38 (t, J=5.7 Hz, 2H).

IR [cm⁻¹]: 823.63, 846.78, 889.21, 956.72, 985.66, 1026.16, 1047.38, 1087.89, 1145.75, 1201.69, 1220.98, 1242.20, 1253.77, 1336.71, 1442.80, 1456.30, 1477.52, 151595.18, 1620.26, 2874.03, 2920.32, 3059.20 and 3321.53.

### Example 4: Preparation of 6,6′-di-(2-(naphth-2-yl)acetylenyl)-2,2′-bis(2-hydroxyethoxy)-1,1′-binaphthyl (Compound of Formula (Ia-1), with R^{a1}=R^{a2}=2-(naphth-2-yl)acetylenyl and R^{a3}=R^{a4}=Hydrogen)

A mixture of 6,6′-dibromo-2,2′-bis(2-hydroxyethoxy)-1,1′-binaphthyl obtained according to protocol 1.4, 2.2 or 2.3 (40.3 g, 75.7 mmol, 1.0 eq), 2-ethynylnaphthalene (34.6 g, 227 mmol, 3.0 eq), PdCl₂ (671 mg, 3.79 mmol, 5.0 mol %), PPh₃ (1.99 g, 7.58 mmol, 10 mol %) and CuI (424 mg, 2.22 mmol, 3.0 mol %) in freshly degassed triethylamine (480 g) was heated to reflux (100-120° C.) for approx. 4-6 h. The reaction was followed by TLC (mobile phase: MeOH/H₂O/EtOAc 7:3:1 (v/v). After complete conversion, the solvent was removed under reduced pressure and THF (500 g) was added. The organic layer was washed with an aqueous solution of HCl (1 M, 200 g) and brine (100 g). The aqueous phases were extracted with THF (2×100 g). The solvent of the combined organic layers was removed under vacuum and EtOAc (500 g) was added. After stirring at RT for 1 h, the formed precipitate was filtered off to give the crude product as a dark yellow solid (51.1 g, 75.7 mmol, 100%). Recrystallization from THF with activated carbon followed by stirring with acetone at RT gave the desired product as a white solid (25.6 g, 37.9 mmol, 50%, chemical purity >99.0% (UPLC)).

Melting point: 174° C.

¹H NMR (400 MHz, CDCl₃): δ=8.17 (d, J=1.6 Hz, 2H), 8.08 (d, J=1.6 Hz, 2H), 8.00 (d, J=9.0 Hz, 2H), 7.87-7.79 (m, 6H), 7.61 (dd, J=8.5, 1.6 Hz, 2H), 7.55-7.45 (m, 6H), 7.42 (dd, J=8.8, 1.7 Hz, 2H), 7.13 (d, J=8.7 Hz, 2H), 4.26 (ddd, J=10.4, 6.6, 2.7 Hz, 2H), 4.07 (ddd, J=10.3, 5.4, 2.7 Hz, 2H), 3.62 (me, 3H), 2.34 (t, J=6.4 Hz, 2H).

IR [cm⁻¹]: 813.99, 856.42, 885.36, 954.80, 964.44, 1047.38, 1084.03, 1215.19, 1244.13, 1253.77, 1332.86, 1454.38, 1481.38, 1591.33, 1618.33, 2872.10, 2939.61, 53053.42 and 3321.53.

### Example 5: Preparation of 6,6′-di-(2-(naphth-1-yl)acetylenyl)-2,2′-bis(2-hydroxyethoxy)-1,1′-binaphthyl (Compound of Formula (Ia-1), with R^{a1}=R^{a2}=2-(naphth-1-yl)acetylenyl and R^{a3}=R^{a4}=Hydrogen)

A mixture of 6,6′-dibromo-2,2′-bis(2-hydroxyethoxy)-1,1′-binaphthyl obtained according to protocol 1.4, 2.2 or 2.3 (16.0 g, 30.0 mmol, 1.0 eq), 1-ethynylnaphthalene (13.7 g, 90.0 mmol, 3.0 eq), PdCl₂ (266 mg, 1.5 mmol, 5.0 mol %), PPh₃ (814 mg, 3.0 mmol, 10 mol %) and CuI (173 mg, 0.9 mmol, 3.0 mol %) in freshly degassed triethylamine (480 g) was heated to reflux (100-120° C.) for approx. 4-6 h. The reaction was followed by TLC (mobile phase: MeOH). After complete conversion, the solvent was removed under reduced pressure and THF (150 g) was added. The organic layer was washed with an aqueous solution of HCl (1 M, 100 g) and brine (100 g). The aqueous phases were extracted with THF (2×50 g). The solvent of the combined organic layers was removed under vacuum and EtOAc (250 g) was added. After stirring at RT for 1 h, the formed precipitate was filtered off to give the crude product as a light brown solid (19.0 g, 28.2 mmol, 94%). Recrystallisation from methyl ethyl ketone gave the desired product as a slightly off-white solid (10.0 g, 14.8 mmol, 49%, chemical purity >98.0% (UPLC)).

Melting point: 227° C. ¹H NMR (400 MHz, CDCl₃): δ=8.48 (dd, J=8.3, 1.2 Hz, 2H), 8.24 (d, J=1.6 Hz, 2H), 8.03 (d, J=9.0 Hz, 2H), 7.86 (ddt, J=9.6, 8.5, 1.0 Hz, 4H), 7.79 (dd, J=7.2, 1.2 Hz, 2H), 7.65-7.43 (m, 10H), 7.16 (dd, J=8.8, 0.9 Hz, 2H), 4.27 (ddd, J=10.4, 6.6, 2.8 Hz, 2H), 4.08 (ddd, J=10.4, 5.4, 2.7 Hz, 2H), 3.72-3.56 (m, 4H), 2.37 (brs, 2H).

### 2.3 Refractive Indices Np and Yellowness Indices Y.I

In the following table C the calculated and in one case the measured refractive indices of some monomers of formula (I) are given. Also provided in table C are the measured refractive indices for some of the corresponding homopolycarbonates consisting of structural units of the formulae (II) and (111-1). The monomers are referenced by their entries in tables A and B presented herein above. In addition, for some of the monomers of formula (I) also the yellowness indices Y.I. are listed in table C.

TABLE C

| | | | n_{D} | n_{D} | |
|---|---|---|---|---|---|
| | | | (calc./measured) | (measured) | Y.I. |
| # | table | entry | monomer | polymer | monomer |
| 1 | A | 1 | 1.740/1.75 | 1.73 | 5 |
| 2 | A | 2 | 1.740 | | |
| 3 | A | 3 | 1.763 | | |
| 4 | A | 4 | 1.784 | 1.77 | |
| 5 | A | 5 | 1.784 | | |
| 6 | A | 6 | 1.824 | | |
| 7 | A | 7 | 1.784 | 1.78 | 16 |
| 8 | A | 8 | 1.784 | | |
| 9 | A | 9 | 1.824 | | |
| 10 | A | 10 | 1.754 | | |
| 11 | A | 11 | 1.754 | | |
| 12 | A | 12 | 1.777 | | |
| 13 | A | 13 | 1.754 | | |
| 14 | A | 14 | 1.754 | | |
| 15 | A | 15 | 1.777 | | |
| 16 | A | 16 | 1.870 | | |
| 17 | A | 17 | 1.820 | | |
| 18 | A | 18 | 1.870 | | |
| 19 | A | 19 | 1.821 | | |
| 20 | A | 20 | 1.821 | | |
| 21 | A | 21 | 1.875 | | |
| 22 | A | 22 | 1.821 | | |
| 23 | A | 23 | 1.821 | | |
| 24 | A | 24 | 1.875 | | |
| 25 | A | 25 | 1.842 | | |
| 26 | A | 26 | 1.842 | | |
| 27 | A | 27 | 1.903 | | |
| 28 | A | 28 | 1.842 | | |
| 29 | A | 29 | 1.842 | | |
| 30 | A | 30 | 1.903 | | |
| 31 | A | 31 | 1.850 | | |
| 32 | A | 32 | 1.850 | | |
| 33 | A | 33 | 1.905 | | |
| 34 | A | 34 | 1.748 | | |
| 35 | A | 35 | 1.776 | | |
| 36 | A | 36 | 1.748 | | |
| 37 | A | 37 | 1.776 | | |
| 38 | A | 38 | 1.748 | | |
| 39 | A | 39 | 1.776 | | |
| 40 | A | 40 | 1.793 | | |
| 41 | A | 41 | 1.793 | | |
| 42 | A | 42 | 1.838 | | |
| 43 | A | 43 | 1.793 | | |
| 44 | A | 44 | 1.838 | | |
| 45 | A | 45 | 1.793 | | |
| 46 | A | 46 | 1.838 | | |
| 47 | A | 47 | 1.793 | | |
| 48 | A | 48 | 1.838 | | |
| 49 | A | 49 | 1.754 | | |
| 50 | A | 50 | 1.754 | | |
| 51 | A | 51 | 1.777 | | |
| 52 | A | 52 | 1.789 | | |
| 53 | A | 53 | 1.789 | | |
| 54 | A | 54 | 1.823 | | |
| 55 | A | 55 | 1.789 | | |
| 56 | A | 56 | 1.789 | | |
| 57 | A | 57 | 1.823 | | |
| 58 | A | 58 | 1.819 | | |
| 59 | A | 59 | 1.819 | | |
| 60 | A | 60 | 1.860 | | |
| 61 | A | 61 | 1.819 | | |
| 62 | A | 62 | 1.819 | | |
| 63 | A | 63 | 1.862 | | |
| 64 | A | 64 | 1.819 | | |
| 65 | A | 65 | 1.819 | | |
| 66 | A | 66 | 1.862 | | |
| 67 | A | 67 | 1.837 | | |
| 68 | A | 68 | 1.837 | | |
| 69 | A | 69 | 1.885 | | |
| 70 | A | 70 | 1.837 | | |
| 71 | A | 71 | 1.837 | | |
| 72 | A | 72 | 1.885 | | |
| 73 | A | 73 | 1.845 | | |
| 74 | A | 74 | 1.845 | | |
| 75 | A | 75 | 1.890 | | |
| 76 | A | 76 | 1.760 | | |
| 77 | A | 77 | 1.760 | | |
| 78 | A | 78 | 1.787 | | |
| 79 | A | 79 | 1.760 | | |
| 80 | A | 80 | 1.760 | | |
| 81 | A | 81 | 1.787 | | |
| 82 | A | 82 | 1.760 | | |
| 83 | A | 83 | 1.760 | | |
| 84 | A | 84 | 1.787 | | |
| 85 | A | 85 | 1.796 | | |
| 86 | A | 86 | 1.796 | | |
| 87 | A | 87 | 1.834 | | |
| 88 | A | 88 | 1.796 | | |
| 89 | A | 89 | 1.796 | | |
| 90 | A | 90 | 1.834 | | |
| 91 | A | 91 | 1.796 | | |
| 92 | A | 92 | 1.796 | | |
| 93 | A | 93 | 1.834 | | |
| 94 | A | 94 | 1.796 | | |
| 95 | A | 95 | 1.796 | | |
| 96 | A | 96 | 1.834 | | |
| 97 | B | 1 | 1.711 | | |
| 98 | B | 2 | 1.737 | | |
| 99 | B | 3 | 1.759 | | |
| 100 | B | 4 | 1.759 | | |
| 101 | B | 5 | 1.772 | | |
| 102 | B | 6 | 1.772 | | |
| 103 | B | 7 | 1.722 | | |
| 104 | B | 8 | 1.745 | | |
| 105 | B | 9 | 1.766 | | |
| 106 | B | 10 | 1.764 | | |
| 107 | B | 11 | 1.764 | | |
| 108 | B | 12 | 1.775 | | |
| 109 | B | 13 | 1.775 | | |
| 110 | B | 14 | 1.728 | | |
| 111 | B | 15 | 1.728 | | |
| 112 | B | 16 | 1.770 | | |
| 113 | B | 17 | 1.770 | | |
| 114 | B | 18 | 1.804 | | |
| 115 | B | 19 | 1.804 | | |
| 116 | B | 20 | 1.804 | | |
| 117 | B | 21 | 1.824 | | |
| 118 | B | 22 | 1.824 | | |
| 119 | B | 23 | 1.743 | | |
| 120 | B | 24 | 1.777 | | |
| 121 | B | 25 | 1.777 | | |
| 122 | B | 26 | 1.806 | | |
| 123 | B | 27 | 1.806 | | |
| 124 | B | 28 | 1.822 | | |
| 125 | B | 29 | 1.822 | | |
| 126 | B | 30 | 1.752 | | |
| 127 | B | 31 | 1.811 | | |
| 128 | B | 32 | 1.811 | | |
| 129 | B | 33 | 1.856 | | |
| 130 | B | 34 | 1.859 | | |
| 131 | B | 35 | 1.859 | | |
| 132 | B | 36 | 1.887 | | |
| 133 | B | 37 | 1.887 | | |
| 134 | B | 38 | 1.769 | | |
| 135 | B | 39 | 1.814 | | |
| 136 | B | 40 | 1.814 | | |
| 137 | B | 41 | 1.849 | | |
| 138 | B | 42 | 1.851 | | |
| 139 | B | 43 | 1.851 | | |
| 140 | B | 44 | 1.873 | | |
| 141 | B | 45 | 1.873 | | |
| 142 | B | 46 | 1.731 | | |
| 143 | B | 47 | 1.751 | | |
| 144 | B | 48 | 1.768 | | |
| 145 | B | 49 | 1.768 | | |
| 146 | B | 50 | 1.779 | | |
| 147 | B | 51 | 1.779 | | |
| 148 | B | 52 | 1.739 | | |
| 149 | B | 53 | 1.757 | | |
| 150 | B | 54 | 1.774 | | |
| 151 | B | 55 | 1.772 | | |
| 152 | B | 56 | 1.772 | | |
| 153 | B | 57 | 1.782 | | |
| 154 | B | 58 | 1.782 | | |
| 155 | B | 59 | 1.743 | | |
| 156 | B | 60 | 1.743 | | |
| 157 | B | 61 | 1.777 | | |
| 158 | B | 62 | 1.777 | | |
| 159 | B | 63 | 1.806 | | |
| 160 | B | 64 | 1.806 | | |
| 161 | B | 65 | 1.806 | | |
| 162 | B | 66 | 1.822 | | |
| 163 | B | 67 | 1.822 | | |
| 164 | B | 68 | 1.754 | | |
| 165 | B | 69 | 1.783 | | |
| 166 | B | 70 | 1.783 | | |
| 167 | B | 71 | 1.808 | | |
| 168 | B | 72 | 1.807 | | |
| 169 | B | 73 | 1.807 | | |
| 170 | B | 74 | 1.822 | | |
| 171 | B | 75 | 1.822 | | |
| 172 | B | 76 | 1.760 | | |
| 173 | B | 77 | 1.781 | | |
| 174 | B | 78 | 1.800 | | |
| 175 | B | 79 | 1.800 | | |
| 176 | B | 80 | 1.811 | | |
| 177 | B | 81 | 1.811 | | |
| 178 | B | 82 | 1.766 | | |
| 179 | B | 83 | 1.801 | | |
| 180 | B | 84 | 1.801 | | |
| 181 | B | 85 | 1.811 | | |
| 182 | B | 86 | 1.811 | | |
| 183 | B | 87 | 1.770 | | |
| 184 | B | 88 | 1.770 | | |
| 185 | B | 89 | 1.834 | | |
| 186 | B | 90 | 1.852 | | |
| 187 | B | 91 | 1.777 | | |
| 188 | B | 92 | 1.806 | | |
| 189 | B | 93 | 1.806 | | |
| 190 | B | 94 | 1.832 | | |
| 191 | B | 95 | 1.832 | | |
| 192 | B | 96 | 1.847 | | |
| 193 | B | 97 | 1.847 | | |
| 194 | B | 98 | 1.778 | | |
| 195 | B | 99 | 1.784 | | |
| 196 | B | 100 | 1.726 | | |
| 197 | B | 101 | 1.768 | | |
| 198 | B | 102 | 1.768 | | |
| 199 | B | 103 | 1.802 | | |
| 200 | B | 104 | 1.801 | | |
| 201 | B | 105 | 1.801 | | |
| 202 | B | 106 | 1.820 | | |
| 203 | B | 107 | 1.820 | | |
| 204 | B | 108 | 1.742 | | |
| 205 | B | 109 | 1.775 | | |
| 206 | B | 110 | 1.775 | | |
| 207 | B | 111 | 1.804 | | |
| 208 | B | 112 | 1.803 | | |
| 209 | B | 113 | 1.803 | | |
| 210 | B | 114 | 1.820 | | |
| 211 | B | 115 | 1.820 | | |
| 212 | B | 116 | 1.750 | | |
| 213 | B | 117 | 1.809 | | |
| 214 | B | 118 | 1.809 | | |
| 215 | B | 119 | 1.856 | | |
| 216 | B | 120 | 1.856 | | |
| 217 | B | 121 | 1.883 | | |
| 218 | B | 122 | 1.883 | | |
| 219 | B | 123 | 1.768 | | |
| 220 | B | 124 | 1.812 | | |
| 221 | B | 125 | 1.812 | | |
| 222 | B | 126 | 1.849 | | |
| 223 | B | 127 | 1.849 | | |
| 224 | B | 128 | 1.871 | | |
| 225 | B | 129 | 1.871 | | |
| 226 | B | 130 | 1.742 | | |
| 227 | B | 131 | 1.775 | | |
| 228 | B | 132 | 1.775 | | |
| 229 | B | 133 | 1.804 | | |
| 230 | B | 134 | 1.803 | | |
| 231 | B | 135 | 1.803 | | |
| 232 | B | 136 | 1.820 | | |
| 233 | B | 137 | 1.820 | | |
| 234 | B | 138 | 1.753 | | |
| 235 | B | 139 | 1.781 | | |
| 236 | | 140 | 1.781 | | |
| 237 | | 141 | 1.806 | | |
| 238 | | 142 | 1.805 | | |
| 239 | | 143 | 1.805 | | |
| 240 | | 144 | 1.820 | | |
| 241 | | 145 | 1.820 | | |
| 242 | | 146 | 1.768 | | |
| 243 | | 147 | 1.802 | | |
| 244 | | 148 | 1.802 | | |
| 245 | | 149 | 1.831 | | |
| 246 | | 150 | 1.831 | | |
| 247 | | 151 | 1.849 | | |
| 248 | | 152 | 1.849 | | |
| 249 | | 153 | 1.775 | | |
| 250 | | 154 | 1.804 | | |
| 251 | | 155 | 1.829 | | |
| 252 | | 156 | 1.829 | | |
| 253 | | 157 | 1.844 | | |
| 254 | | 158 | 1.844 | | |
| 255 | | 159 | 1.680 | | |
| 256 | | 160 | 1.707 | | |
| 257 | | 161 | 1.707 | | |
| 258 | | 162 | 1.731 | | |
| 259 | | 163 | 1.728 | | |
| 260 | | 164 | 1.728 | | |
| 261 | | 165 | 1.740 | | |
| 262 | | 166 | 1.740 | | |
| 263 | | 167 | 1.694 | | |
| 264 | | 168 | 1.717 | | |
| 265 | | 169 | 1.717 | | |
| 266 | | 170 | 1.736 | | |
| 267 | | 171 | 1.736 | | |
| 268 | | 172 | 1.747 | | |
| 269 | | 173 | 1.747 | | |
| 270 | | 174 | 1.701 | | |
| 271 | | 175 | 1.743 | | |
| 272 | | 176 | 1.743 | | |
| 273 | | 177 | 1.779 | | |
| 274 | | 178 | 1.777 | | |
| 275 | | 179 | 1.777 | | |
| 276 | | 180 | 1.796 | | |
| 277 | | 181 | 1.796 | | |
| 278 | | 182 | 1.720 | | |
| 279 | | 183 | 1.755 | | |
| 280 | | 184 | 1.755 | | |
| 281 | | 185 | 1.784 | | |
| 282 | | 186 | 1.783 | | |
| 283 | | 187 | 1.783 | | |
| 284 | | 188 | 1.799 | | |
| 285 | | 189 | 1.799 | | |
| 286 | | 190 | 1.701 | | |
| 287 | | 191 | 1.743 | | |
| 288 | | 192 | 1.743 | | |
| 289 | | 193 | 1.779 | | |
| 290 | | 194 | 1.777 | | |
| 291 | | 195 | 1.777 | | |
| 292 | | 196 | 1.796 | | |
| 293 | | 197 | 1.796 | | |
| 294 | | 198 | 1.720 | | |
| 295 | | 199 | 1.755 | | |
| 296 | | 200 | 1.755 | | |
| 297 | | 201 | 1.783 | | |
| 298 | | 202 | 1.783 | | |
| 299 | | 203 | 1.799 | | |
| 300 | | 204 | 1.799 | | |
| 301 | | 205 | 1.729 | | |
| 302 | | 206 | 1.838 | | |
| 303 | | 207 | 1.838 | | |
| 304 | | 208 | 1.865 | | |
| 305 | | 209 | 1.865 | | |
| 306 | | 210 | 1.752 | | |
| 307 | | 211 | 1.834 | | |
| 308 | | 212 | 1.834 | | |
| 309 | | 213 | 1.956 | | |
| 310 | | 214 | 1.856 | | |
| 311 | | 215 | 1.720 | | |
| 312 | | 216 | 1.755 | | |
| 313 | | 217 | 1.755 | | |
| 314 | | 218 | 1.784 | | |
| 315 | | 219 | 1.783 | | |
| 316 | | 220 | 1.783 | | |
| 317 | | 221 | 1.799 | | |
| 318 | | 222 | 1.799 | | |
| 319 | | 223 | 1.734 | | |
| 320 | | 224 | 1.763 | | |
| 321 | | 225 | 1.788 | | |
| 322 | | 226 | 1.788 | | |
| 323 | | 227 | 1.802 | | |
| 324 | | 228 | 1.802 | | |
| 325 | | 229 | 1.720 | | |
| 326 | | 230 | 1.755 | | |
| 327 | | 231 | 1.755 | | |
| 328 | | 232 | 1.784 | | |
| 329 | | 233 | 1.783 | | |
| 330 | | 234 | 1.783 | | |
| 331 | | 235 | 1.799 | | |
| 332 | | 236 | 1.799 | | |
| 333 | | 237 | 1.734 | | |
| 334 | | 238 | 1.763 | | |
| 335 | | 239 | 1.788 | | |
| 336 | | 240 | 1.788 | | |
| 337 | | 241 | 1.802 | | |
| 338 | | 242 | 1.802 | | |
| 339 | | 243 | 1.680 | | |
| 340 | | 244 | 1.707 | | |
| 341 | | 245 | 1.707 | | |
| 342 | | 246 | 1.731 | | |
| 343 | | 247 | 1.728 | | |
| 344 | | 248 | 1.728 | | |
| 345 | | 249 | 1.740 | | |
| 346 | | 250 | 1.740 | | |
| 347 | | 251 | 1.694 | | |
| 348 | | 252 | 1.717 | | |
| 349 | | 253 | 1.717 | | |
| 350 | | 254 | 1.738 | | |
| 351 | | 255 | 1.736 | | |
| 352 | | 256 | 1.736 | | |
| 353 | | 257 | 1.747 | | |
| 354 | | 258 | 1.747 | | |
| 355 | | 259 | 1.701 | | |
| 356 | | 260 | 1.743 | | |
| 357 | | 261 | 1.743 | | |
| 358 | | 262 | 1.777 | | |
| 359 | | 263 | 1.777 | | |
| 360 | | 264 | 1.796 | | |
| 361 | | 265 | 1.796 | | |
| 362 | | 266 | 1.720 | | |
| 363 | | 267 | 1.755 | | |
| 364 | | 268 | 1.783 | | |
| 365 | | 269 | 1.783 | | |
| 366 | | 270 | 1.799 | | |
| 367 | | 271 | 1.799 | | |
| 368 | | 272 | 1.731 | | |
| 369 | | 273 | 1.752 | | |
| 370 | | 274 | 1.752 | | |
| 371 | | 275 | 1.771 | | |
| 372 | | 276 | 1.770 | | |
| 373 | | 277 | 1.770 | | |
| 374 | | 278 | 1.781 | | |
| 375 | | 279 | 1.781 | | |
| 376 | | 280 | 1.738 | | |
| 377 | | 281 | 1.758 | | |
| 378 | | 282 | 1.774 | | |
| 379 | | 283 | 1.774 | | |
| 380 | | 284 | 1.784 | | |
| 381 | | 285 | 1.784 | | |
| 382 | | 286 | 1.743 | | |
| 383 | | 287 | 1.779 | | |
| 384 | | 288 | 1.779 | | |
| 385 | | 289 | 1.809 | | |
| 386 | | 290 | 1.808 | | |
| 387 | | 291 | 1.808 | | |
| 388 | | 292 | 1.826 | | |
| 389 | | 293 | 1.826 | | |
| 390 | | 294 | 1.755 | | |
| 391 | | 295 | 1.784 | | |
| 392 | | 296 | 1.784 | | |
| 393 | | 297 | 1.810 | | |
| 394 | | 298 | 1.810 | | |
| 395 | | 299 | 1.810 | | |
| 396 | | 300 | 1.825 | | |
| 397 | | 301 | 1.825 | | |
| 398 | | 302 | 1.743 | | |
| 399 | | 303 | 1.779 | | |
| 400 | | 304 | 1.779 | | |
| 401 | | 305 | 1.809 | | |
| 402 | | 306 | 1.808 | | |
| 403 | | 307 | 1.808 | | |
| 404 | | 308 | 1.826 | | |
| 405 | | 309 | 1.826 | | |
| 406 | | 310 | 1.755 | | |
| 407 | | 311 | 1.784 | | |
| 408 | | 312 | 1.784 | | |
| 409 | | 313 | 1.810 | | |
| 410 | | 314 | 1.810 | | |
| 411 | | 315 | 1.810 | | |
| 412 | | 316 | 1.825 | | |
| 413 | | 317 | 1.825 | | |
| 414 | | 318 | 1.762 | | |
| 415 | | 319 | 1.815 | | |
| 416 | | 320 | 1.858 | | |
| 417 | | 321 | 1.858 | | |
| 418 | | 322 | 1.883 | | |
| 419 | | 323 | 1.883 | | |
| 420 | | 324 | 1.776 | | |
| 421 | | 325 | 1.851 | | |
| 422 | | 326 | 1.851 | | |
| 423 | | 327 | 1.871 | | |
| 424 | | 328 | 1.871 | | |
| 425 | | 329 | 1.731 | | |
| 426 | | 330 | 1.752 | | |
| 427 | | 331 | 1.752 | | |
| 428 | | 332 | 1.771 | | |
| 429 | | 333 | 1.770 | | |
| 430 | | 334 | 1.770 | | |
| 431 | | 335 | 1.781 | | |
| 432 | | 336 | 1.781 | | |
| 433 | | 337 | 1.738 | | |
| 434 | | 338 | 1.758 | | |
| 435 | | 339 | 1.758 | | |
| 436 | | 340 | 1.775 | | |
| 437 | | 341 | 1.774 | | |
| 438 | | 342 | 1.774 | | |
| 439 | | 343 | 1.784 | | |
| 440 | | 344 | 1.784 | | |
| 441 | | 345 | 1.743 | | |
| 442 | | 346 | 1.779 | | |
| 443 | | 347 | 1.779 | | |
| 444 | | 348 | 1.809 | | |
| 445 | | 349 | 1.808 | | |
| 446 | | 350 | 1.808 | | |
| 447 | | 351 | 1.826 | | |
| 448 | | 352 | 1.826 | | |
| 449 | | 353 | 1.755 | | |
| 450 | | 354 | 1.784 | | |
| 451 | | 355 | 1.784 | | |
| 452 | | 356 | 1.810 | | |
| 453 | | 357 | 1.810 | | |
| 454 | | 358 | 1.810 | | |
| 455 | | 359 | 1.825 | | |
| 456 | | 360 | 1.825 | | |
| 457 | | 361 | 1.762 | | |
| 458 | | 362 | 1.815 | | |
| 459 | | 363 | 1.815 | | |
| 460 | | 364 | 1.858 | | |
| 461 | | 365 | 1.858 | | |
| 462 | | 366 | 1.883 | | |
| 463 | | 367 | 1.883 | | |
| 464 | | 368 | 1.776 | | |
| 465 | | 369 | 1.816 | | |
| 466 | | 370 | 1.851 | | |
| 467 | | 371 | 1.851 | | |
| 468 | | 372 | 1.871 | | |
| 469 | | 373 | 1.871 | | |

### 3. Preparation of the Resins

### 3.1 ANALYTICS RELATING TO RESINS

### 3.1.1 Measurement Method of Weight Average Molecular Weight (Mw)

The weight average molecular weight (Mw) was measured using the HLC-8320GPC device from Tosoh Corporation as a GPC device, the TSKguardcolumn SuperMPHZ-Mone as a guard column, and three TSKgel SuperMultiporeHZ-M(s) connected in series as analysis columns. The measurement conditions were as follows.
Solvent: HPLC grade tetrahydrofuran
Injection Volume: 10 μL
Concentration of Sample: 0.2 w/v % HPLC grade chloroform solution
Solvent flow velocity”: 0.35 ml/min
Measurement Temperature: 40° C.
Detecting Device: RI

The weight average molecular weights (Mw) of the resins are calculated using a previously prepared standard curve of polystyrene. Specifically, the standard curve was prepared using standard polystyrene of defined molecular weight (“PStQuick MP-M” from Tosoh Corporation which has molecular weight distribution value of 1). Further, a calibration curve was obtained by plotting the elution time and molecular weight value of each of the peaks based on the measured data of the standard polystyrene, and conducting three-dimensional approximation. The values for Mw are calculated based on the following formula.

Mw=Σ(Wi×Mi)÷Σ(Wi)

In the formula, “i” represents the “i”th dividing point, “Wi” represents the molecular weight (g) of the polymer at the “i”th dividing point, and “Mi” represents the molecular mass at the “i”th dividing point. The molecular mass (M) represents the value of the molecular mass of polystyrene at the corresponding elution time in the calibration curve.

### 3.1.2 Refractive Index (nD)

The refractive index of a film having a thickness of 0.1 mm formed of a polycarbonate resin produced in an example was measured by use of an Abbe refractive index meter by a method of JIS-K-7142 at a wavelength of 589 hm.

### 3.1.3 Abbe Number (v)

The refractive index of a film having a thickness of 0.1 mm formed of a polycarbonate resin produced in an example was measured by use of an Abbe refractive index meter at 23° C. at wavelengths of 486 nm, 589 nm and 656 nm. Then, the Abbe number was calculated by use of the following equation (formula (a)):

v=(nD−1)/(nF−nC) formula (a)
nD: refractive index at a wavelength of 589 nm
nC: refractive index at a wavelength of 656 nm
nF: refractive index at a wavelength of 486 nm

### 3.1.3-1 Measurement and Calculation of Relative Partial Dispersion (θgf)

In addition to the refractive index values regarding D-line, C-line, and F-line (nC, nD, and nF), refractive index value regarding g-line was measured in a similar manner. The value of relative partial dispersion (θgf) was calculated based on the formula (b) below.

θgf=(ng−nF)/(nF−nC) formula (b)

In the formula (b), nC represents the measured refractive index value regarding C-line, nF represents the measured refractive index value regarding F-line, and ng represents the measured refractive index value regarding g-line.

### 3.1.3-2 Measurement and Calculation of Degree of Anomalous Dispersion (Δθgf)

The values of degree of anomalous relative partial dispersion (Δθgf) were calculated based on the Abbe numbers (v) and the values of relative partial dispersion (θgf) which were calculated from formulae (a) and (b) above, respectively. First, a graph in which the Abbe numbers (v) are plotted on the X-axis and the values of relative partial dispersion (θgf) are plotted on the Y-axis was prepared. Then, a straight line connecting two points having coordinates (v, θgf) regarding optical glasses was added to the graph; one point for NSL7 (made by Ohara, Inc.) as a standard dispersion glass chosen from among normal glasses which represent no anomalous dispersion (v=60.5, and θgf=0.5436); and the other point for PBM2 (made by Ohara Inc.) as another standard dispersion glass also chosen from normal glasses which represent no anomalous dispersion (v=36.3, and θgf=0.5828). Finally, a point of a polycarbonate resin also having coordinates (v, θgf) was plotted on the graph and the difference on the Y-coordinate direction between the θgf values of the point of the polycarbonate resin and the above-mentioned straight line was calculated as a degree of anomalous relative partial dispersion (or a value of Δθgf).

Specifically, the value of Δθgf was calculated as below. The straight line which connected the points of the two standard dispersion glasses is represented by the formula (c) below, in which “v0” represents the Abbe number of a point on the straight line and “θgf0” represents the value of relative partial dispersion of the point on the straight line.

θgf0=0.001618×v0+0.6415 formula (c)

Then, the value of Δθgf of a polycarbonate resin was calculated based on the formula (d) below in which “v” represents the Abbe number of the polycarbonate resin calculated from the formula (a) above and “θgf” represents the value of relative partial dispersion of the polycarbonate resin calculated from the formula (b) above.

$$\begin{matrix}Δ θ gf = θ gf - θ gf 0 \\ = θ gf - ( - 0.001618 \times v + 0.6415 )\end{matrix} \qquad formula ( d )$$

The value of Δθgf of a resin is an indicator of anomalous dispersion which corresponds to the distance between the straight line connecting the points of NSL7 and PBM2 and a plotted point of the resin as mentioned above and indicates how much the resin refracts blue light (or light in short wavelengths). The higher the value of Δθgf, the more highly the resin refracts blue light, and when a resin has a high value of Δθgf an optical device including the resin efficiently corrects chromatic aberrations and enables a clear image.

### 3.1.4 Glass Transition Temperature (Tg)

The glass transition temperature was measured by differential scanning calorimetry (DSC) according to JIS K 7121-1987. The measuring device was a X-DSC7000 from Hitachi High-Technologies.

### 3.1.5 Measurement of b Value

The respective resin was dried at 120° C. for 4 hours in vacuum, and then injection-molded by an injection molding device (FANUC ROBOSHOT α-S30iA) at a cylinder temperature of 270° C. and a mold temperature of Tg −10° C. to obtain a disc-shaped test plate piece having a diameter of 50 mm and a thickness of 3 mm. This test plate piece was used to measure the b value by a method according to JIS-K7105. When the b value is smaller, the plate is less yellowish and thus the hue is better. For the measurement, a spectral color difference meter type SE2000 of Nippon Denshoku Industries Co., Ltd. was used.

### 3.1.6 Total Light Transmittance (TLT)

A plate having a thickness of 3 mm was produced from the respective polycarbonate resin by the protocol described in section 3.1.5 for the measurement of the b value. The total light transmittance of measured by use of SE2000 spectral color difference meter produced by Nippon Denshoku Industries Co., Ltd. by a method of JIS-K-7361-1.

The total light transmittance of these plates were measured before a PCT treatment (i.e. leaving the plates under the saturation water vapor pressure of 100° C. for one week) and thereafter. The value is given in table D in column TLT-PCT.

### 3.1.7 Amount of Vinyl Terminal Group

The amount of vinyl terminal groups was determined by ¹H-NMR measurement under the following conditions.
Device: AVANZE III HD 500 MHz produced by Bruker
Flip angle: 30 degrees
Wait time: 1 second
Accumulate number of times: 500 times
Measurement temperature: room temperature (298K)
Concentration: 5 wt %
Solvent: Deuterated chloroform
Inner standard substance: tetramethylsilane (TMS) 0.05 wt %

### 3.1.8 Determination of Impurities in the Resin

Concentrations of phenol, diphenylcarbonate (DPC) and monomer in the polycarbonate resin was measured according to the following protocol.

0.5 g of the resin sample was dissolved in 50 ml of tetrahydrofuran to obtain a resin solution. A calibration curve was created from a pure form of each of compounds as a preparation. 2 μL of sample solution was quantitatively analyzed by LC-MS under the following measurement conditions. The detection limit under the measurement conditions is 0.01 ppm.
Measurement device (LC part): Agilent Infinity 1260 LC System
Column: ZORBAX Eclipse XDB-18 and guard cartridge
Mobile phase:
   Eluent A: 0.01 mol/L—aqueous solution of ammonium acetate
   Eluent B: 0.01 mol/L—methanol solution of ammonium acetate
   Eluent C: THF
   Gradient program of the mobile phase:

As shown in Table 1, different mixtures of eluents A through C were used as mobile phases. The mobile phases were caused to flow in the column for 30 minutes while the compositions of the mobile phases were switched when the time (minutes) shown in Table 1 lapsed.

TABLE 1

| | Mobile Phase Composition | | |
|---|---|---|---|
| Time | (% by Volume) | | |
| (min.) | A | B | C |
| 0 | 10 | 75 | 15 |
| 10.0 | 9 | 67.5 | 23.5 |
| 10.1 | 0 | 25 | 75 |
| 30.0 | 0 | 25 | 75 |

Flow rate: 0.3 ml/min.
Column temperature: 45° C.
Detector: UV (225 nm)
Measurement device (MS part): Agilent 6120 single quad LCMS System
Ionization source: ESI
Polarity: Positive (DPC) and negative (PhOH)
Fragmentor: 70 V
Dry gas: 10 L/min., 350° C.
Nebulizer: 50 psi
Capillary voltage: 3000 V (positive), 2500 V (negative)
Ion measured

TABLE 2

| Monomer | Ion Type | m/z |
|---|---|---|
| PhOH | [M − H]⁻ | 93.1 |
| DPC | [M + NH₄]⁺ | 232.1 |

Amount of injected sample: 2 μL

### 3.1.9 Moldability of Resins

The moldability of the polycarbonate resins was evaluated preparing plates as described in protocol 3.1.5 and visually assessing the quality of the plates according to the following grades A to D⁺ and D:
A: Metal mold used for injection molding had no stain; and molded piece had no void space and no wave was found on the surface of the molded piece.
B: Metal mold used for injection molding had no stain; and molded piece had void space while no wave was found on the surface of the molded piece.
C: Metal mold used for injection molding had almost no stain; and molded piece had no void space while waves were found on the surface of the molded piece.
D⁺: Metal mold used for injection molding had some stain; and molded piece had a little void space while waves were found on the surface of the molded piece.
D: Metal mold used for injection molding had much stain and therefore cleaning was required; and molded piece had void space while waves were found on the surface of the molded piece.

### 3.2 Preparation Examples

### Example 6-1

9.7 kg (18.0 mol) of BNEF, 6.7 kg (18.0 mol) of BNE, 16.2 kg (24.0 mol) of D2NACBHB, 13.5 kg (63.0 mol) of DPC and 32 μl (8.0*10⁻⁷ mol) of a 2.5×10⁻² mol/L aqueous solution of sodium hydrogen carbonate were put into a 300 ml four-neck flask reactor in a nitrogen atmosphere. The mixture was heated to 190° C. to start the reaction. The reaction mixture was stirred at 190° C. for 60 minutes and then heated to 200° C. The reaction conditions were maintained for further 20 minutes. Then, the pressure was adjusted to 200 mmHg, and the reaction conditions were maintained for further 20 minutes. At this point, phenol generated as a byproduct started to distill off. Then, the reaction mixture was heated to 230° C. and the reaction conditions were maintained for further 10 minutes. Then, the pressure was adjusted to 150 mmHg, and the reaction conditions were maintained for further 10 minutes. The reaction mixture was heated to 240° C. while the pressure was adjusted to lower than or equal to 1 mmHg. The reaction mixture was stirred for 30 minutes with maintaining the temperature and pressure. After the reaction was completed, pressure equalization was achieved by introducing nitrogen into the reactor and the generated polycarbonate was removed from the reactor and analyzed. The results are summarized in table

### Example 6-2

Substantially the same operation was performed as in example 6-1 except that 11.3 kg (21.0 mol) of BNEF, 7.9 kg (21.1 mol) of BNE, 12.1 kg (18.0 mol) of D2NACBHB, and 13.5 kg (63.0 mol) of DPC were used as materials to obtain a polycarbonate resin.

### Example 6-3

Substantially the same operation was performed as in example 6-1 except that 12.9 kg (23.9 mol) of BNEF, 9.0 kg (24.1 mol) of, 8.1 kg (12.0 mol) of D2NACBHB, and 13.5 kg (63.0 mol) of DPC were used as materials to obtain a polycarbonate resin.

### Example 6-4

Substantially the same operation was performed as in example 6-1 except that 12.9 kg (23.9 mol) of BNEF, 10.1 kg (27.0 mol) of BNE, 6.1 kg (9.0 mol) of D2NACBHB, and 13.5 kg (63.0 mol) of DPC were used as materials to obtain a polycarbonate resin.

### Example 6-5

Substantially the same operation was performed as in example 6-1 except that 12.9 kg (23.9 mol) of BNEF, 11.2 kg (29.9 mol) of BNE), 4.0 kg (5.9 mol) of D2NACBHB, and 13.5 kg (63.0 mol) of DPC were used as materials to obtain a polycarbonate resin.

### Example 6-6

Substantially the same operation was performed as in example 6-1 except that 9.7 kg (18.0 mol) of BNEF, 6.7 kg (18.0 mol) of BNE, 13.8 kg (24.0 mol) of DPACBHBNA and 13.5 kg (63.0 mol) of DPC were used as materials to obtain a polycarbonate resin.

### Example 6-7

Substantially the same operation was performed as in example 6-1 except that 11.3 kg (21.0 mol) of BNEF, 7.9 kg (21.1 mol) of BNE, 10.4 kg (18.01 mol) of DPACBHBNA and 13.5 kg (63.0 mol) of DPC were used as materials to obtain a polycarbonate resin.

### Example 6-8

Substantially the same operation was performed as in example 6-1 except that 12.9 kg (23.9 mol) of BNEF, 9.0 kg (24.1 mol) of BNE, 6.9 kg (12.0 mol) of DPACBHBNA and 13.5 kg (63.0 mol) of DPC were used as materials to obtain a polycarbonate resin.

### Example 6-9

Substantially the same operation was performed as in example 6-1 except that 12.9 kg (24.0 mol) of BNEF, 10.1 kg (27.0 mol) of BNE, 5.2 kg (9.0 mol) of DPACBHBNA and 13.5 kg (63.0 mol) of DPC were used as materials to obtain a polycarbonate resin.

### Example 6-10

Substantially the same operation was performed as in example 6-1 except that 12.9 kg (23.9 mol) of BNEF, 11.2 kg (29.9 mol) of BNE, 3.5 kg (6.1 mol) of DPACBHBNA, and 13.5 kg (63.0 mol) of DPC were used as materials to obtain a polycarbonate resin.

### Reference Example 7-1

Substantially the same operation was performed as in example 6-1 except that 22.5 kg (60.1 mol) of BNE, and 13.5 kg (63.0 mol) of DPC were used as materials to obtain a polycarbonate resin.

### Reference Example 7-2

Substantially the same operation was performed as in example 6-1 except that 12.9 kg (23.9 mol) of BNEF, 10.1 kg (27.0 mol) of BNE, 4.7 kg (8.9 mol) of BINL-2EO and 13.5 kg (63.0 mol) of DPC were used as materials to obtain a polycarbonate resin.

### Reference Example 7-3

Substantially the same operation was performed as in example 6-1 except that 12.9 kg (23.9 mol) of BNEF, 10.1 kg (27.0 mol) of BNE, 5.6 kg (8.9 mol) of 2DNBINOL-2EO and 13.5 kg (63.0 mol) of DPC were used as materials to obtain a polycarbonate resin.

Properties of the resins obtained in Examples 6-1 to 6-10 and Reference Examples 7-1 to 7-3 are shown in Table D.

TABLE D

| | Molar Ratio of Dihydroxy Compounds | | | | | | | | |
|---|---|---|---|---|---|---|---|---|---|
| | D2NACB | DPACBH | | | BINL- | 2DNBIN | Molecular Weight | | |
| Example | HBNA | BNA | BNEF | BNE | 2EO | OL-2EO | M_{w} | Mn | Mw/Mn |
| 6-1 | 40 | 0 | 30 | 30 | 0 | 0 | 25800 | 11000 | 2.35 |
| 6-2 | 30 | 0 | 35 | 35 | 0 | 0 | 30700 | 11500 | 2.67 |
| 6-3 | 20 | 0 | 40 | 40 | 0 | 0 | 33100 | 13500 | 2.45 |
| 6-4 | 15 | 0 | 40 | 45 | 0 | 0 | 25800 | 10500 | 2.46 |
| 6-5 | 10 | 0 | 40 | 50 | 0 | 0 | 28300 | 12200 | 2.32 |
| 6-6 | 0 | 40 | 30 | 30 | 0 | 0 | 25900 | 11100 | 2.33 |
| 6-7 | 0 | 30 | 35 | 35 | 0 | 0 | 32500 | 12600 | 2.58 |
| 6-8 | 0 | 20 | 40 | 40 | 0 | 0 | 30500 | 12300 | 2.48 |
| 6-9 | 0 | 15 | 40 | 45 | 0 | 0 | 26000 | 11000 | 2.36 |
| 6-10 | 0 | 10 | 40 | 50 | 0 | 0 | 25000 | 12300 | 2.03 |
| 7-1* | 0 | 0 | 0 | 100 | 0 | 0 | 23000 | 7800 | 2.95 |
| 7-2 | 0 | 0 | 40 | 45 | 15 | 0 | 26000 | 10600 | 2.45 |
| 7-3 | 0 | 0 | 40 | 45 | 0 | 15 | 26100 | 10500 | 2.49 |

Table D (sequel)

| | | | | | | | | | TLT- |
|---|---|---|---|---|---|---|---|---|---|
| | Tg | | Abbe | | | TLT¹⁾ | | | PCT²⁾ |
| Example | [C.] | nD | value (v) | θgF | ΔθgF | [%] | b value | Moldability | [%] |
| 6-1 | 157 | 1.728 | 13 | 0.79 | 0.170 | 86 | 4.90 | C | 84 |
| 6-2 | 155 | 1.717 | 14 | 0.77 | 0.151 | 87 | 4.20 | B | 87 |
| 6-3 | 154 | 1.705 | 15 | 0.74 | 0.123 | 88 | 4.10 | A | 88 |
| 6-4 | 151 | 1.698 | 16 | 0.73 | 0.114 | 90 | 3.80 | A | 90 |
| 6-5 | 148 | 1.691 | 17 | 0.71 | 0.096 | 87 | 4.20 | B | 87 |
| 6-6 | 153 | 1.702 | 15 | 0.74 | 0.123 | 85 | 4.80 | C | 84 |
| 6-7 | 152 | 1.697 | 15 | 0.73 | 0.113 | 87 | 4.20 | B | 87 |
| 6-8 | 151 | 1.691 | 16 | 0.71 | 0.094 | 88 | 4.10 | A | 88 |
| 6-9 | 149 | 1.687 | 17 | 0.70 | 0.086 | 91 | 3.90 | A | 90 |
| 6-10 | 147 | 1.684 | 17 | 0.69 | 0.076 | 87 | 4.20 | B | 87 |
| 7-1* | 115 | 1.669 | 19 | 0.68 | 0.069 | 86 | 4.40 | D | 86 |
| 7-2 | 150 | 1.681 | 18 | 0.68 | 0.068 | 87 | 5.50 | D | 79 |
| 7-3 | 156 | 1.690 | 17 | 0.70 | 0.086 | 87 | 5.40 | D | 79 |

The molecular structures of the material compounds used in the above Examples are represented by formulae (XXII) to (XXVII) below.

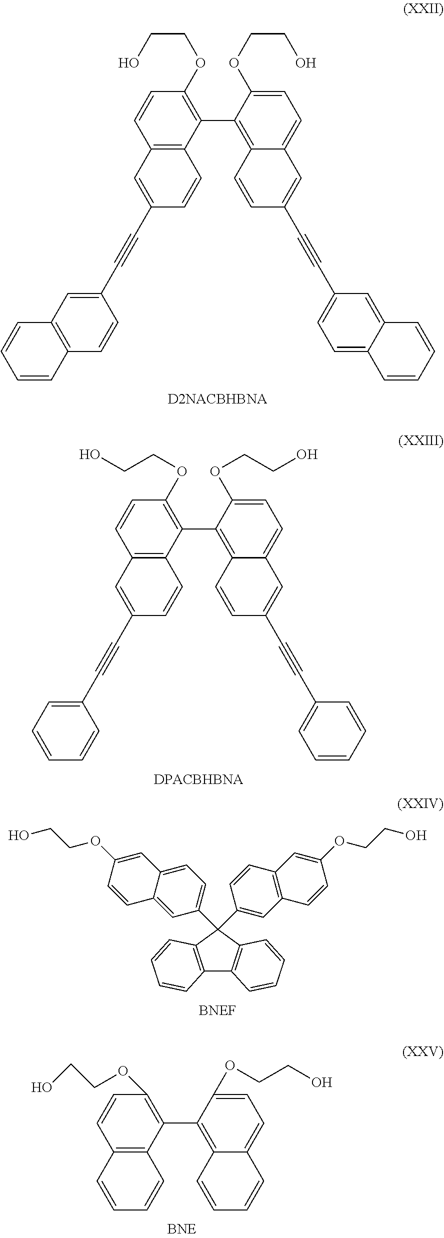 [SMILES: OCCOc1ccc2cc(C#Cc3ccc4ccccc4c3)ccc2c1-c1c(OCCO)ccc2cc(C#Cc3ccc4ccccc4c3)ccc12 ; OCCOc1ccc2cc(C#Cc3ccccc3)ccc2c1-c1c(OCCO)ccc2cc(C#Cc3ccccc3)ccc12 ; OCCOc1ccc2cc(C3(c4ccc5cc(OCCO)ccc5c4)c4ccccc4-c4ccccc43)ccc2c1 ; OCCOc1ccc2ccccc2c1-c1c(OCCO)ccc2ccccc12]

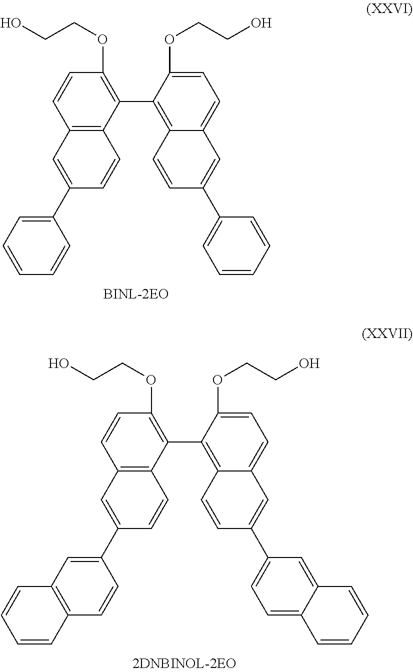 [SMILES: OCCOc1ccc2cc(-c3ccccc3)ccc2c1-c1c(OCCO)ccc2cc(-c3ccccc3)ccc12 ; OCCOc1ccc2cc(-c3ccc4ccccc4c3)ccc2c1-c1c(OCCO)ccc2cc(-c3ccc4ccccc4c3)ccc12]

1. A compound of the formula (I) 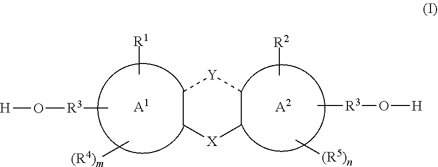
where
A¹, A² are selected from mono- or bicyclic aromatic radicals and mono- or bicyclic heteroaromatic radicals,
X represents a single bond, O, NH, CR⁶R⁷ or a moiety of the formula A,
Y is absent or represents a single bond, O, NH, CR⁸R⁹ or a moiety of the formula A, 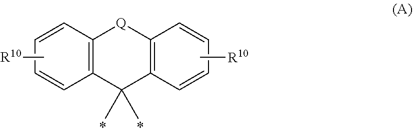
where
* indicate the points of attachment to the ring carbon atoms of A¹ and A², respectively and
Q is absent or represents a single bond, O, NH, C═O, CH₂ or CH═CH;
R¹, R² are hydrogen, a radical Ar′ or a radical R^{a};
R³ is Alk, O-Alk′-, O-Alk′-[O-Alk′]ₒ, O—CH₂—Ar—C(O)—, O—C(O)—Ar—C(O)— or O-Alk-C(O)—, where in the last five moieties the left O is bound to A¹ and A², respectively,
m, n are 0, 1 or 2;
o is an integer from 1 to 10;
R⁴, R⁵ are selected from the group consisting of fluorine, CN, R, OR, CH_{w}R_{3-w}, NR₂, C(O)R, C(O)NH₂, a radical Ar′ and a radical R^{a};
R⁶ is selected from the group consisting of hydrogen, a radical Ar′ and a radical R^{a};
R⁷ is selected from the group consisting of hydrogen, C₁-C₄-alkyl and a radical Ar′;
R⁸ is selected from the group consisting of hydrogen, a radical Ar′ and a radical R^{a},
R⁹ is selected from the group consisting of hydrogen, C₁-C₄-alkyl and a radical Ar′;
R¹⁰ is selected from the group consisting of hydrogen, fluorine, CN, R, OR, CHₖR₃₋ₖ, NR₂, C(O)R, C(O)NH₂ and a radical R^{a};
R^{a} is selected from the group consisting of C≡C—R¹¹ and Ar—C≡C—R¹¹;
R¹¹ is selected from hydrogen, methyl, mono- or polycyclic aryl having from 6 to 26 carbon atoms and mono- or polycyclic hetaryl having a total of 5 to 26 atoms, which are ring members, where 1, 2, 3 or 4 of the ring atoms of hetaryl are selected from nitrogen, sulphur and oxygen, while the remainder of these atoms are carbon atoms, where mono- or polycyclic aryl are unsubstituted or substituted by 1, 2, 3 or 4 identical or different radicals R¹²;
R¹² is selected from fluorine, phenyl, CN, OCH₃, CH₃, N(CH₃)₂, C(O)CH₃, C≡CH, C≡C—CH₃, CH₂—C≡CH and CH₂—C≡C—CH₃;
Alk is C₁-C₄-alkandiyl, where 1 or 2 of the hydrogen atoms of C₁-C₄-alkandiyl may be replaced by Ar′;
Alk′ is C₂-C₄-alkandiyl, where 1 or 2 of the hydrogen atoms of C₁-C₄-alkandiyl may be replaced by Ar′, or CH₂—Ar—CH₂;
Ar is a bivalent radical selected from phenylene and naphthylene, which are unsubstituted or carry 1, 2, 3 or 4 radicals R^{Ar};
Ar′ is selected from the group consisting of mono- or polycyclic aryl having from 6 to 26 carbon atoms and mono- or polycyclic hetaryl having a total of 5 to 26 atoms, which are ring members, where 1, 2, 3 or 4 of these atoms are selected from nitrogen, sulphur and oxygen, while the remainder of these atoms are carbon atoms,
where mono- or polycyclic aryl and mono- or polycyclic hetaryl are unsubstituted or carry 1, 2, 3 or 4 radicals R^{Ar};
R^{Ar} is selected from the group consisting of fluorine, bromine, chlorine, CN, R, OR, CHₖR₃₋ₖ, NR₂, C(O)R, C(O)NH₂ and a radical R^{a}, it being possible that R^{Ar} is identical or different, if more than 1 is present on each ring;
R is selected from methyl, mono- or polycyclic aryl having from 6 to 26 carbon atoms and mono- or polycyclic hetaryl having a total of 5 to 26 atoms, which are ring members, where 1, 2, 3 or 4 of the ring atoms of hetaryl are selected from nitrogen, sulphur and oxygen, while the remainder of these atoms are carbon atoms, where mono- or polycyclic aryl are unsubstituted or substituted by 1, 2, 3 or 4 identical or different radicals R¹²;
w on each occurrence is 0, 1, 2 or 3;
k on each occurrence is 0, 1, 2 or 3;
and, if R³ is O—CH₂—Ar—C(O)—, O—C(O)—Ar—C(O)— or O-Alk-C(O)—, the esters thereof, in particular the C₁-C₄-alkyl esters thereof;
provided that the compound of formula (I) bears at least 1 radical R^{a} and in particular 2 to 4 radicals R^{a}.

2. The compound of claim 1, where the variable R³—OH is O-Alk′-OH, where Alk′ is a linear alkylene group having 2, 3 or 4 carbon atoms, and wherein Alk′ is in particular CH₂CH₂, or where R³—OH is O-Alk-C(O)—OH, where Alk′ is a linear alkylene group having 1, 2 or 3 carbon atoms, and wherein Alk is in particular CH₂.

3. The compound of any one of the preceding claims, where the A¹ and A² are identical and selected from the group consisting of a phenylene and naphthylene.

4. The compound of any one of the preceding claims, where R^{a} is selected from the group consisting of ethynyl, 2-methylethynyl, 2-phenylethynyl, 2-(1-naphthyl)ethynyl, 2-(2-naphthyl)ethynyl, 2-(2-phenylphenyl)ethynyl, 2-(4-phenylphenyl)ethynyl, 2-(phenanthren-9-yl)ethynyl, 2-(dibenzofuran-2-yl)ethynyl, 2-(dibenzofuran-4-yl)ethynyl, 2-(dibenzothiophen-2-yl)ethynyl, 2-(dibenzothiophen-4-yl)ethynyl, 2-(triphenylen-2-yl)ethynyl, 2-(pyridin-2-yl)ethynyl, 2-(pyridin-3-yl)ethynyl, 2-(pyridin-4-yl)ethynyl, 2-(quinoline-2-yl)ethynyl, 2-(quinoline-3-yl)ethynyl, 2-(quinoline-4-yl)ethynyl, 2-(quinoline-8-yl)ethynyl, 2-(2-phenylethynyl)phenyl, 3-(2-phenylethynyl)phenyl, 4-(2-phenylethynyl)phenyl, 2-(2-(2-naphthyl)ethynyl)phenyl, 3-(2-(2-naphthyl)ethynyl)phenyl, 4-(2-(2-naphthyl)ethynyl)phenyl, 2-(2-(1-naphthyl)ethynyl)phenyl, 3-(2-(1-naphthyl)ethynyl)phenyl, 4-(2-(1-naphthyl)ethynyl)phenyl, 4-(2-(2-phenylphenyl)ethynyl)phenyl, 4-(2-(4-phenylphenyl)ethynyl)phenyl, 4-(2-(phenanthren-9-yl)ethynyl)phenyl, 4-(2-(dibenzofuran-2-yl)ethynyl)phenyl, 4(2-(dibenzofuran-4-yl)ethynyl)phenyl, 4-(2-(dibenzothiophen-2-yl)ethynyl)phenyl, 4-(2-(dibenzothiophen-4-yl)ethynyl)phenyl, 4-(2-(triphenylen-2-yl)ethynyl)phenyl, 4-(2-(pyridin-2-yl)ethynyl)phenyl, 4-(2-(pyridin-3-yl)ethynyl)phenyl, 4-(2-(pyridin-4-yl)ethynyl)phenyl, 4-(2-(quinoline-2-yl)ethynyl)phenyl, 4-(2-(quinoline-3-yl)ethylenyl)phenyl, 4-(2-(quinoline-4-yl)ethynyl)phenyl, 4-(2-(quinoline-8-yl)ethynyl)phenyl, 2-(2-phenylethynyl)-1-naphthyl, 3-(2-phenylethynyl)-1-naphthyl, 4-(2-phenylethynyl)-1-naphthyl, 5-(2-phenylethynyl)-1-naphthyl, 6-(2-phenylethynyl)-1-naphthyl, 7-(2-phenylethynyl)-1-naphthyl, 8-(2-phenylethynyl)-1-naphthyl, 1-(2-phenylethynyl)-2-naphthyl, 3-(2-phenylethynyl)-2-naphthyl, 4-(2-phenylethynyl)-2-naphthyl, 5-(2-phenylethynyl)-2-naphthyl, 6-(2-phenylethynyl)-2-naphthyl, 7-(2-phenylethynyl)-2-naphthyl, 8-(2-phenylethynyl)-2-naphthyl 2-(2-(1-naphthyl)ethynyl)-1-naphthyl, 3-(2-(1-naphthyl)ethynyl)-1-naphthyl, 4-(2-(1-naphthyl)ethynyl)-1-naphthyl, 5-(2-(1-naphthyl)ethynyl)-1-naphthyl, 6-(2-(1-naphthyl)ethynyl)-1-naphthyl, 7-(2-(1-naphthyl)ethynyl)-1-naphthyl, 8-(2-(1-naphthyl)ethynyl)-1-naphthyl, 1-(2-(1-naphthyl)ethynyl)-2-naphthyl, 3-(2-(1-naphthyl)ethynyl)-2-naphthyl, 4-(2-(1-naphthyl)ethynyl)-2-naphthyl, 5-(2-(1-naphthyl)ethynyl)-2-naphthyl, 6-(2-(1-naphthyl)ethynyl)-2-naphthyl, 7-(2-(1-naphthyl)ethynyl)-2-naphthyl 8-(2-(1-naphthyl)ethynyl)-2-naphthyl 2-(2-(2-naphthyl)ethynyl)-1-naphthyl, 3-(2-(2-naphthyl)ethynyl)-1-naphthyl, 4-(2-(2-naphthyl)ethynyl)-1-naphthyl, 5-(2-(2-naphthyl)ethynyl)-1-naphthyl, 6-(2-(2-naphthyl)ethynyl)-1-naphthyl, 7-(2-(2-naphthyl)ethynyl)-1-naphthyl, 8-(2-(2-naphthyl)ethynyl)-1-naphthyl, 1-(2-(2-naphthyl)ethynyl)-2-naphthyl, 3-(2-(2-naphthyl)ethynyl)-2-naphthyl, 4-(2-(2-naphthyl)ethynyl)-2-naphthyl, 5-(2-(2-naphthyl)ethynyl)-2-naphthyl, 6-(2-(2-naphthyl)ethynyl)-2-naphthyl, 7-(2-(2-naphthyl)ethynyl)-2-naphthyl 8-(2-(2-naphthyl)ethynyl)-2-naphthyl.

05. The compound of any one of the preceding claims, where X represents a single bond and Y is absent.

6. The compound of claim 5, where formula (I) is represented by formula (Ia): 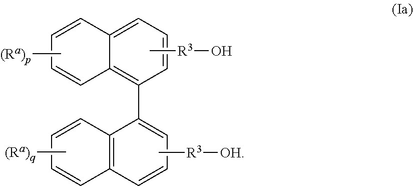
where q is 1 or 2 and p is 1 or 2.

7. The compound of claim 6, where formula (I) is represented by formula (Ia-1): 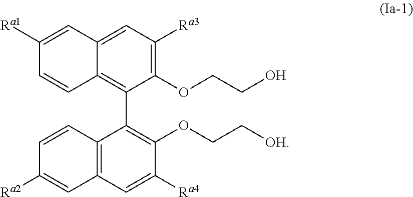
where
R^{a1} is hydrogen or R^{a},
R^{a2} is hydrogen or R^{a},
R^{a3} is hydrogen or R^{a} and
R^{a4} is hydrogen or R^{a},
provided that at least two of R^{a1}, R^{a2}, R^{a3} and R^{a4} are R^{a}.

8. The compound of claim 7, where R^{a1}, R^{a2}, R^{a3} and R^{a4} in formula (Ia-1) are as defined in the following table A:
TABLE A

| | R^{a1} | R^{a2} | R^{a3} | R^{a4} |
|---|---|---|---|---|
| 1 | R^{a1}-1 | R^{a1}-1 | H | H |
| 2 | H | H | R^{a1}-1 | R^{a1}-1 |
| 3 | R^{a1}-1 | R^{a1}-1 | R^{a1}-1 | R^{a1}-1 |
| 4 | R^{a1}-2 | R^{a1}-2 | H | H |
| 5 | H | H | R^{a1}-2 | R^{a1}-2 |
| 6 | R^{a1}-2 | R^{a1}-2 | R^{a1}-2 | R^{a1}-2 |
| 7 | R^{a1}-3 | R^{a1}-3 | H | H |
| 8 | H | H | R^{a1}-3 | R^{a1}-3 |
| 9 | R^{a1}-3 | R^{a1}-3 | R^{a1}-3 | R^{a1}-3 |
| 10 | R^{a1}-4 | R^{a1}-4 | H | H |
| 11 | H | H | R^{a1}-4 | R^{a1}-4 |
| 12 | R^{a1}-4 | R^{a1}-4 | R^{a1}-4 | R^{a1}-4 |
| 13 | R^{a1}-5 | Rai-5 | H | H |
| 14 | H | H | R^{a1}-5 | R^{a1}-5 |
| 15 | R^{a1}-5 | R^{a1}-5 | R^{a1}-5 | R^{a1}-5 |
| 16 | R^{a1}-6 | R^{a1}-6 | H | H |
| 17 | H | H | R^{a1}-6 | R^{a1}-6 |
| 18 | R^{a1}-6 | R^{a1}-6 | R^{a1}-6 | R^{a1}-6 |
| 19 | R^{a1}-7 | R^{a1}-7 | H | H |
| 20 | H | H | R^{a1}-7 | R^{a1}-7 |
| 21 | R^{a1}-7 | R^{a1}-7 | R^{a1}-7 | R^{a1}-7 |
| 22 | R^{a1}-8 | R^{a1}-8 | H | H |
| 23 | H | H | R^{a1}-8 | R^{a1}-8 |
| 24 | R^{a1}-8 | R^{a1}-8 | R^{a1}-8 | R^{a1}-8 |
| 25 | R^{a1}-9 | R^{a1}-9 | H | H |
| 26 | H | H | R^{a1}-9 | R^{a1}-9 |
| 27 | R^{a1}-9 | R^{a1}-9 | R^{a1}-9 | R^{a1}-9 |
| 28 | R^{a1}-10 | R^{a1}-10 | H | H |
| 29 | H | H | R^{a1}-10 | R^{a1}-10 |
| 30 | R^{a1}-10 | R^{a1}-10 | R^{a1}-10 | R^{a1}-10 |
| 31 | R^{a1}-11 | R^{a1}-11 | H | H |
| 32 | H | H | R^{a1}-11 | R^{a1}-11 |
| 33 | R^{a1}-11 | R^{a1}-11 | R^{a1}-11 | R^{a1}-11 |
| 34 | R^{a1}-12 | R^{a1}-12 | H | H |
| 35 | R^{a1}-12 | R^{a1}-12 | R^{a1}-12 | R^{a1}-12 |
| 36 | R^{a1}-13 | R^{a1}-13 | H | H |
| 37 | R^{a1}-13 | R^{a1}-13 | R^{a1}-13 | R^{a1}-13 |
| 38 | R^{a1}-14 | R^{a1}-14 | H | H |
| 39 | R^{a1}-14 | R^{a1}-14 | R^{a1}-14 | R^{a1}-14 |
| 40 | R^{a1}-15 | R^{a1}-15 | H | H |
| 41 | H | H | R^{a1}-15 | R^{a1}-15 |
| 42 | R^{a1}-15 | R^{a1}-15 | R^{a1}-15 | R^{a1}-15 |
| 43 | R^{a1}-16 | R^{a1}-16 | H | H |
| 44 | R^{a1}-16 | R^{a1}-16 | R^{a1}-16 | R^{a1}-16 |
| 45 | R^{a1}-17 | R^{a1}-17 | H | H |
| 46 | R^{a1}-17 | R^{a1}-17 | R^{a1}-17 | R^{a1}-17 |
| 47 | R^{a1}-18 | R^{a1}-18 | H | H |
| 48 | R^{a1}-18 | R^{a1}-18 | R^{a1}-18 | R^{a1}-18 |
| 49 | R^{a1}-19 | R^{a1}-19 | H | H |
| 50 | H | H | R^{a1}-19 | R^{a1}-19 |
| 51 | R^{a1}-19 | R^{a1}-19 | R^{a1}-19 | R^{a1}-19 |
| 52 | R^{a1}-20 | R^{a1}-20 | H | H |
| 53 | H | H | R^{a1}-20 | R^{a1}-20 |
| 54 | R^{a1}-20 | R^{a1}-20 | R^{a1}-20 | R^{a1}-20 |
| 55 | R^{a1}-21 | R^{a1}-21 | H | H |
| 56 | H | H | R^{a1}-21 | R^{a1}-21 |
| 57 | R^{a1}-21 | R^{a1}-21 | R^{a1}-21 | R^{a1}-21 |
| 58 | R^{a1}-24 | R^{a1}-24 | H | H |
| 59 | H | H | R^{a1}-24 | R^{a1}-24 |
| 60 | R^{a1}-24 | R^{a1}-24 | R^{a1}-24 | R^{a1}-24 |
| 61 | R^{a1}-25 | Rai-25 | H | H |
| 62 | H | H | R^{a1}-25 | R^{a1}-25 |
| 63 | R^{a1}-25 | R^{a1}-25 | R^{a1}-25 | R^{a1}-25 |
| 64 | R^{a1}-26 | R^{a1}-26 | H | H |
| 65 | H | H | R^{a1}-26 | R^{a1}-26 |
| 66 | R^{a1}-26 | R^{a1}-26 | R^{a1}-26 | R^{a1}-26 |
| 67 | R^{a1}-27 | R^{a1}-27 | H | H |
| 68 | H | H | R^{a1}-27 | R^{a1}-27 |
| 69 | R^{a1}-27 | R^{a1}-27 | R^{a1}-27 | R^{a1}-27 |
| 70 | R^{a1}-28 | R^{a1}-28 | H | H |
| 71 | H | H | R^{a1}-28 | R^{a1}-28 |
| 72 | R^{a1}-28 | R^{a1}-28 | R^{a1}-28 | R^{a1}-28 |
| 73 | R^{a1}-29 | R^{a1}-29 | H | H |
| 74 | H | H | R^{a1}-29 | R^{a1}-29 |
| 75 | R^{a1}-29 | R^{a1}-29 | R^{a1}-29 | R^{a1}-29 |
| 76 | R^{a1}-30 | R^{a1}-30 | H | H |
| 77 | H | H | R^{a1}-30 | R^{a1}-30 |
| 78 | R^{a1}-30 | R^{a1}-30 | R^{a1}-30 | R^{a1}-30 |
| 79 | R^{a1}-31 | R^{a1}-31 | H | H |
| 80 | H | H | R^{a1}-31 | R^{a1}-31 |
| 81 | R^{a1}-31 | R^{a1}-31 | R^{a1}-31 | R^{a1}-31 |
| 82 | R^{a1}-32 | R^{a1}-32 | H | H |
| 83 | H | H | R^{a1}-32 | R^{a1}-32 |
| 84 | R^{a1}-32 | R^{a1}-32 | R^{a1}-32 | R^{a1}-32 |
| 85 | R^{a1}-33 | R^{a1}-33 | H | H |
| 86 | H | H | R^{a1}-33 | R^{a1}-33 |
| 87 | R^{a1}-33 | R^{a1}-33 | R^{a1}-33 | R^{a1}-33 |
| 88 | R^{a1}-34 | R^{a1}-34 | H | H |
| 89 | H | H | R^{a1}-34 | R^{a1}-34 |
| 90 | R^{a1}-34 | R^{a1}-34 | R^{a1}-34 | R^{a1}-34 |
| 91 | R^{a1}-35 | R^{a1}-35 | H | H |
| 92 | H | H | R^{a1}-35 | R^{a1}-35 |
| 93 | R^{a1}-35 | R^{a1}-35 | R^{a1}-35 | R^{a1}-35 |
| 94 | R^{a1}-36 | R^{a1}-36 | H | H |
| 95 | H | H | R^{a1}-36 | R^{a1}-36 |
| 96 | R^{a1}-36 | R^{a1}-36 | R^{a1}-36 | R^{a1}-36 |

where:
R^{a1}-1=2-phenylethynyl,
R^{a1}-2=2-(1-naphthyl)ethynyl,
R^{a1}-3=2-(2-naphthyl)ethynyl,
R^{a1}-4=2-(2-phenylphenyl)ethynyl,
R^{a1}-5=2-(4-phenylphenyl)ethynyl,
R^{a1}-6=2-(phenanthren-9-yl)ethynyl,
R^{a1}-7=2-(dibenzofuran-2-yl)ethynyl,
R^{a1}-8=2-(dibenzofuran-4-yl)ethynyl,
R^{a1}-9=2-(dibenzothiophen-2-yl)ethynyl,
R^{a1}-10=2-(dibenzothiophen-4-yl)ethynyl,
R^{a1}-11=2-(triphenylen-2-yl)ethynyl,
R^{a1}-12=2-(pyridin-2-yl)ethynyl,
R^{a1}-13=2-(pyridin-3-yl)ethynyl,
R^{a1}-14=2-(pyridin-4-yl)ethynyl,
R^{a1}-15=2-(quinoline-2-yl)ethynyl,
R^{a1}-16=2-(quinoline-3-yl)ethynyl,
R^{a1}-17=2-(quinoline-4-yl)ethynyl,
R^{a1}-18=2-(quinoline-8-yl)ethynyl,
R^{a1}-19=4-(2-phenylethynyl)phenyl,
R^{a1}-20=4-(2-(2-naphthyl)ethynyl)phenyl,
R^{a1}-21=4-(2-(1-naphthyl)ethynyl)phenyl,
R^{a1}-22=4-(2-(2-phenylphenyl)ethynyl)phenyl,
R^{a1}-23=4-(2-(4-phenylphenyl)ethynyl)phenyl,
R^{a1}-24=4-(2-(phenanthren-9-yl)ethynyl)phenyl,
R^{a1}-25=4-(2-(dibenzofuran-2-yl)ethynyl)phenyl,
R^{a1}-26=4(2-(dibenzofuran-4-yl)ethynyl)phenyl,
R^{a1}-27=4-(2-(dibenzothiophen-2-yl)ethynyl)phenyl,
R^{a1}-28=4-(2-(dibenzothiophen-4-yl)ethynyl)phenyl,
R^{a1}-29=4-(2-(triphenylen-2-yl)ethynyl)phenyl,
R^{a1}-30=4-(2-(pyridin-2-yl)ethynyl)phenyl,
R^{a1}-31=4-(2-(pyridin-3-yl)ethynyl)phenyl,
R^{a1}-32=4-(2-(pyridin-4-yl)ethynyl)phenyl,
R^{a1}-33=4-(2-(quinoline-2-yl)ethynyl)phenyl,
R^{a1}-34=4-(2-(quinoline-3-yl)ethylenyl)phenyl,
R^{a1}-35=4-(2-(quinoline-4-yl)ethynyl)phenyl, and
R^{a1}-36=4-(2-(quinoline-8-yl)ethynyl)phenyl.

9. The compound of any one of claims 1 to 4, where X represents a radical of the formula A or a radical CR⁶R⁷, where both R⁶ and R⁷ are Ar′ and where Y is absent.

10. The compound of claim 9, where formula (I) is represented by formula (Ib): 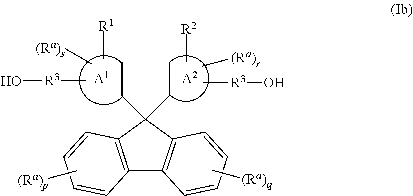
where p, q, r and s are identical or different and are 0 or 1, provided that at least one of R¹ and R² are a radical R^{a}, if p=0, q=0, r=0 and s=0.

11. The compound of claim 10, where formula (Ib) is represented by one of the formulae (Ib-1) or (Ib-2): 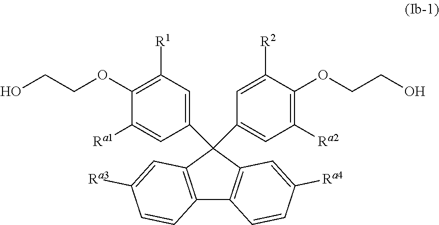
where in formula (Ib-1) the variables R¹, R² are hydrogen, phenyl or a radical R^{a} and R^{a1}, R^{a2}, R^{a3} and R^{a4}, each independently are hydrogen or a radical R^{a}, provided that at least one of R¹, R², R^{a1}, R^{a2}, R^{a3} and R^{a4} in formula (Ib-1) is a radical R^{a}; 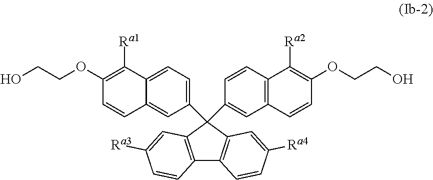
where in formula (Ib-2) the variables R^{a1}, R^{a2}, R^{a3} and R^{a4}, each independently are hydrogen or a radical R^{a}, provided that at least one of R^{a1}, R^{a2}, R^{a3} and R^{a4} in formula (Ib-2) is a radical R^{a}.

12. The compound of claim 9, where formula (I) is represented by formula (Ic): 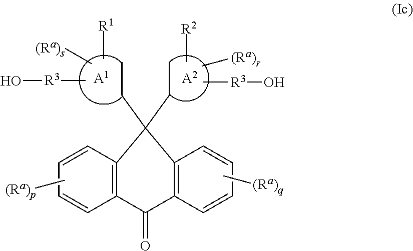
where p, q, r and s are identical or different and are 0 or 1, provided that at least one of R¹ and R² are a radical R^{a}, if p=0, q=0, r=0 and s=0.

13. The compound of claim 12, where formula (Ic) is represented by one of the formulae (Ic-1) or (Ic-2): 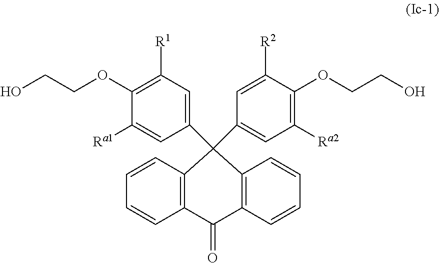
where in formula (Ic-1) the radicals R¹, R² are hydrogen, phenyl or a radical R^{a} and R^{a1} and R^{a2}, each independently are hydrogen or a radical R^{a}, provided that at least one of R¹, R², R^{a1} and R^{a2} in formula (Ic-1) is a radical R^{a}; 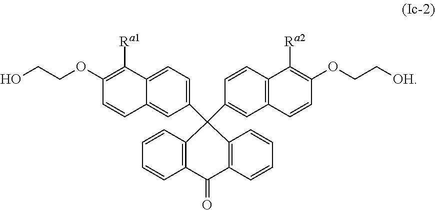
where in formula (Ic-2) the radicals R^{a1} and R^{a2}, each independently are hydrogen or a radical R^{a}, provided that at least one of R^{a1} and R^{a2} in formula (Ic-2) is a radical R^{a}.

14. The compound of claim 9, where formula (I) is represented by formula (Id): 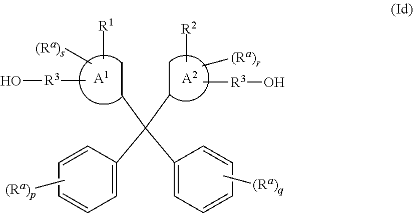
where p, q, r and s are identical or different and are 0 or 1, provided that at least one of R¹ and R² are a radical R^{a}, if p=0 q=0, r=0 and s=0.

15. The compound of claim 14, where formula (Id) is represented by one of the formulae (Id-1), (Id-2), (Id-3) or (Id-4): 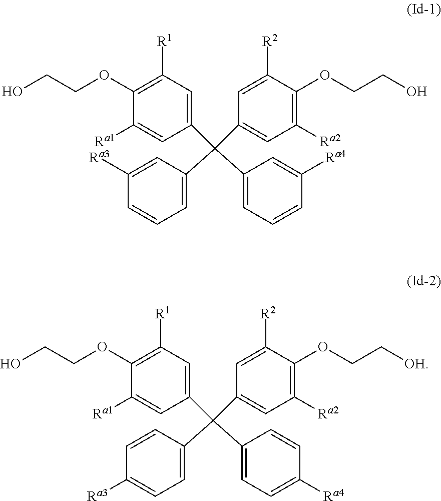
where in formulae (Id-1) and (Id-2) the radicals R¹ and R² are hydrogen, phenyl or a radical R^{a} and R^{a1}, R^{a2}, R^{a3} and R^{a4}, each independently are hydrogen or a radical R^{a}, provided that at least one of R¹, R², R^{a1}, R^{a2}, R^{a3} and R^{a4} in formula (Id-1) and (Id-2) is a radical R^{a}; 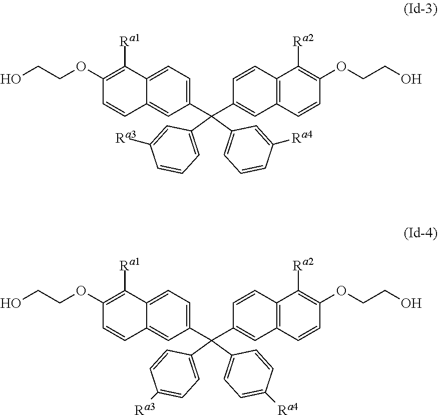
where in formulae (Id-3) and (Id-4), R^{a1}, R^{a2}, R^{a3} and R^{a4}, each independently are hydrogen or a radical R^{a}, provided that at least one of R^{a1}, R^{a2}, R^{a3} and R^{a4} in formulae (Id-3) and (Id-4) is a radical R^{a}.

16. The compound of any one of claims 11, 13 or 15 where the variables in formulae (Ib-1), (Ib-2), (Ic-1), (Ic-2), (Id-1), (Id-2), (Id-3) and (Id-4) are as defined in the rows of the following table B:
TABLE B

| | Formula | R¹ | R² | R^{a1} | R^{a2} | R^{a3} | R^{a4} |
|---|---|---|---|---|---|---|---|
| 1 | Ib-1 | H | H | H | H | R^{a1}-1 | H |
| 2 | Ib-1 | H | H | H | H | R^{a1}-3 | H |
| 3 | Ib-1 | H | H | H | H | R^{a1}-7 | H |
| 4 | Ib-1 | H | H | H | H | R^{a1}-8 | H |
| 5 | Ib-1 | H | H | H | H | R^{a1}-9 | H |
| 6 | Ib-1 | H | H | H | H | R^{a1}-10 | H |
| 7 | Ib-1 | H | H | H | H | R^{a1}-19 | H |
| 8 | Ib-1 | H | H | H | H | R^{a1}-20 | H |
| 9 | Ib-1 | H | H | H | H | R^{a1}-24 | H |
| 10 | Ib-1 | H | H | H | H | R^{a1}-25 | H |
| 11 | Ib-1 | H | H | H | H | R^{a1}-26 | H |
| 12 | Ib-1 | H | H | H | H | R^{a1}-27 | H |
| 13 | Ib-1 | H | H | H | H | R^{a1}-28 | H |
| 14 | Ib-1 | H | H | H | H | R^{a1}-1 | R^{a1}-1 |
| 15 | Ib-1 | H | H | R^{a1}-1 | R^{a1}-1 | H | H |
| 16 | Ib-1 | H | H | R^{a1}-2 | R^{a1}-2 | H | H |
| 17 | Ib-1 | H | H | R^{a1}-3 | R^{a1}-3 | H | H |
| 18 | Ib-1 | H | H | R^{a1}-6 | R^{a1}-6 | H | H |
| 19 | Ib-1 | H | H | R^{a1}-7 | R^{a1}-7 | H | H |
| 20 | Ib-1 | H | H | R^{a1}-8 | R^{a1}-8 | H | H |
| 21 | Ib-1 | H | H | R^{a1}-9 | R^{a1}-9 | H | H |
| 22 | Ib-1 | H | H | R^{a1}-10 | R^{a1}-10 | H | H |
| 23 | Ib-1 | H | H | R^{a1}-19 | R^{a1}-19 | H | H |
| 24 | Ib-1 | H | H | R^{a1}-20 | R^{a1}-20 | H | H |
| 25 | Ib-1 | H | H | R^{a1}-21 | R^{a1}-21 | H | H |
| 26 | Ib-1 | H | H | R^{a1}-25 | R^{a1}-25 | H | H |
| 27 | Ib-1 | H | H | R^{a1}-26 | R^{a1}-26 | H | H |
| 28 | Ib-1 | H | H | R^{a1}-27 | R^{a1}-27 | H | H |
| 29 | Ib-1 | H | H | R^{a1}-28 | R^{a1}-28 | H | H |
| 30 | Ib-1 | R^{a1}-1 | R^{a1}-1 | R^{a1}-1 | R^{a1}-1 | H | H |
| 31 | Ib-1 | R^{a1}-2 | R^{a1}-2 | R^{a1}-2 | R^{a1}-2 | H | H |
| 32 | Ib-1 | R^{a1}-3 | R^{a1}-3 | R^{a1}-3 | R^{a1}-3 | H | H |
| 33 | Ib-1 | R^{a1}-6 | R^{a1}-6 | R^{a1}-6 | R^{a1}-6 | H | H |
| 34 | Ib-1 | R^{a1}-7 | R^{a}1-7 | R^{a1}-7 | R^{a1}-7 | H | H |
| 35 | Ib-1 | R^{a1}-8 | R^{a1}-8 | R^{a1}-8 | R^{a1}-8 | H | H |
| 36 | Ib-1 | R^{a1}-9 | R^{a1}-9 | R^{a1}-9 | R^{a1}-9 | H | H |
| 37 | Ib-1 | R^{a1}-10 | R^{a1}-10 | R^{a1}-10 | R^{a1}-10 | H | H |
| 38 | Ib-1 | R^{a1}-19 | R^{a1}-19 | R^{a1}-19 | R^{a1}-19 | H | H |
| 39 | Ib-1 | R^{a1}-20 | R^{a1}-20 | R^{a1}-20 | R^{a1}-20 | H | H |
| 40 | Ib-1 | R^{a1}-21 | R^{a1}-21 | R^{a1}-21 | R^{a1}-21 | H | H |
| 41 | Ib-1 | R^{a1}-24 | R^{a1}-24 | R^{a1}-24 | R^{a1}-24 | H | H |
| 42 | Ib-1 | R^{a1}-25 | R^{a1}-25 | R^{a1}-25 | R^{a1}-25 | H | H |
| 43 | Ib-1 | R^{a1}-26 | R^{a1}-26 | R^{a1}-26 | R^{a1}-26 | H | H |
| 44 | Ib-1 | R^{a1}-27 | R^{a1}-27 | R^{a1}-27 | R^{a1}-27 | H | H |
| 45 | Ib-1 | R^{a1}-28 | R^{a1}-28 | R^{a1}-28 | R^{a1}-28 | H | H |
| 46 | Ib-1 | phenyl | phenyl | H | H | R^{a1}-1 | H |
| 47 | Ib-1 | phenyl | phenyl | H | H | R^{a1}-3 | H |
| 48 | Ib-1 | phenyl | phenyl | H | H | R^{a1}-7 | H |
| 49 | Ib-1 | phenyl | phenyl | H | H | R^{a1}-8 | H |
| 50 | Ib-1 | phenyl | phenyl | H | H | R^{a1}-9 | H |
| 51 | Ib-1 | phenyl | phenyl | H | H | R-10 | H |
| 52 | Ib-1 | phenyl | phenyl | H | H | R^{a1}-19 | H |
| 53 | Ib-1 | phenyl | phenyl | H | H | R^{a1}-20 | H |
| 54 | Ib-1 | phenyl | phenyl | H | H | R^{a1}-24 | H |
| 55 | Ib-1 | phenyl | phenyl | H | H | R^{a1}-25 | H |
| 56 | Ib-1 | phenyl | phenyl | H | H | R^{a1}-26 | H |
| 57 | Ib-1 | phenyl | phenyl | H | H | R^{a1}-27 | H |
| 58 | Ib-1 | phenyl | phenyl | H | H | R^{a1}-28 | H |
| 59 | Ib-1 | phenyl | phenyl | H | H | R^{a1}-1 | R^{a1}-1 |
| 60 | Ib-1 | phenyl | phenyl | R^{a1}-1 | R^{a1}-1 | H | H |
| 61 | Ib-1 | phenyl | phenyl | R^{a1}-2 | R^{a1}-2 | H | H |
| 62 | Ib-1 | phenyl | phenyl | R^{a1}-3 | R^{a1}-3 | H | H |
| 63 | Ib-1 | phenyl | phenyl | R^{a1}-6 | R^{a1}-6 | H | H |
| 64 | Ib-1 | phenyl | phenyl | R^{a1}-7 | R^{a1}-7 | H | H |
| 65 | Ib-1 | phenyl | phenyl | R^{a1}-8 | R^{a1}-8 | H | H |
| 66 | Ib-1 | phenyl | phenyl | R^{a1}-9 | R^{a1}-9 | H | H |
| 67 | Ib-1 | phenyl | phenyl | R^{a1}-10 | R^{a1}-10 | H | H |
| 68 | Ib-1 | phenyl | phenyl | R^{a1}-19 | R^{a1}-19 | H | H |
| 69 | Ib-1 | phenyl | phenyl | R^{a1}-20 | R^{a1}-20 | H | H |
| 70 | Ib-1 | phenyl | phenyl | R^{a1}-21 | R^{a1}-21 | H | H |
| 71 | Ib-1 | phenyl | phenyl | R^{a1}-24 | R^{a1}-24 | H | H |
| 72 | Ib-1 | phenyl | phenyl | R^{a1}-25 | R^{a1}-25 | H | H |
| 73 | Ib-1 | phenyl | phenyl | R^{a1}-26 | R^{a1}-26 | H | H |
| 74 | Ib-1 | phenyl | phenyl | R^{a1}-27 | R^{a1}-27 | H | H |
| 75 | Ib-1 | phenyl | phenyl | R^{a1}-28 | R^{a1}-28 | H | H |
| 76 | Ib-2 | — | — | H | H | R^{a1}l | H |
| 77 | Ib-2 | — | — | H | H | R^{a1}-3 | H |
| 78 | Ib-2 | — | — | H | H | R^{a1}-7 | H |
| 79 | Ib-2 | — | — | H | H | R^{a1}-8 | H |
| 80 | Ib-2 | — | — | H | H | R^{a1}-9 | H |
| 81 | Ib-2 | — | — | H | H | R^{a1}-10 | H |
| 82 | Ib-2 | — | — | H | H | R^{a1}-19 | H |
| 83 | Ib-2 | — | — | H | H | R^{a1}-25 | H |
| 84 | Ib-2 | — | — | H | H | R^{a1}-26 | H |
| 85 | Ib-2 | — | — | H | H | R^{a1}-27 | H |
| 86 | Ib-2 | — | — | H | H | R^{a1}-28 | H |
| 87 | Ib-2 | — | — | H | H | Ra1l | R^{a1}-1 |
| 88 | Ib-2 | — | — | R^{a1}-1 | R^{a1}-1 | H | H |
| 89 | Ib-2 | — | — | R^{a1}-8 | R^{a1}-8 | H | H |
| 90 | Ib-2 | — | — | R^{a1}-9 | R^{a1}-9 | H | H |
| 91 | Ib-2 | — | — | R^{a1}-19 | R^{a1}-19 | H | H |
| 92 | Ib-2 | — | — | R^{a1}-20 | R^{a1}-20 | H | H |
| 93 | Ib-2 | — | — | R^{a1}-21 | R^{a1}-21 | H | H |
| 94 | Ib-2 | — | — | R^{a1}-25 | R^{a1}-25 | H | H |
| 95 | Ib-2 | — | — | R^{a1}-26 | R^{a1}-26 | H | H |
| 96 | Ib-2 | — | — | R^{a1}-27 | R^{a1}-27 | H | H |
| 97 | Ib-2 | — | — | R^{a1}-28 | R^{a1}-28 | H | H |
| 98 | Ib-2 | — | — | R^{a1}-1 | R^{a1}-1 | R^{a1}-1 | H |
| 99 | Ib-2 | — | — | R^{a1}-1 | R^{a1}-1 | R^{a1}-1 | R^{a1}-1 |
| 100 | Ic-1 | H | H | R^{a1}-1 | R^{a1}-1 | — | — |
| 101 | Ic-1 | H | H | R^{a1}-2 | R^{a1}-2 | — | — |
| 102 | Ic-1 | H | H | R^{a1}-3 | R^{a1}-3 | — | — |
| 103 | Ic-1 | H | H | R^{a1}-6 | R^{a1}-6 | — | — |
| 104 | Ic-1 | H | H | R^{a1}-7 | R^{a1}-7 | — | — |
| 105 | Ic-1 | H | H | R^{a1}-8 | R^{a1}-8 | — | — |
| 106 | Ic-1 | H | H | R^{a1}-9 | R^{a1}-9 | — | — |
| 107 | Ic-1 | H | H | R^{a1}-10 | R^{a1}-10 | — | — |
| 108 | Ic-1 | H | H | R^{a1}-19 | R^{a1}-19 | — | — |
| 109 | Ic-1 | H | H | R^{a1}-20 | R^{a1}-20 | — | — |
| 110 | Ic-1 | H | H | R^{a1}-21 | R^{a1}-21 | — | — |
| 111 | Ic-1 | H | H | R^{a1}-24 | R^{a1}-24 | — | — |
| 112 | Ic-1 | H | H | R^{a1}-25 | R^{a1}-25 | — | — |
| 113 | Ic-1 | H | H | R^{a1}-26 | R^{a1}-26 | — | — |
| 114 | Ic-1 | H | H | R^{a1}-27 | R^{a1}-27 | — | — |
| 115 | Ic-1 | H | H | R^{a1}-28 | R^{a1}-28 | — | — |
| 116 | Ic-1 | R^{a1}-1 | R^{a1}-1 | R^{a1}-1 | R^{a1}-1 | — | — |
| 117 | Ic-1 | R^{a1}-2 | R^{a1}-2 | R^{a1}-2 | R^{a1}-2 | — | — |
| 118 | Ic-1 | R^{a1}-3 | R^{a1}-3 | R^{a1}-3 | R^{a1}-3 | — | — |
| 119 | Ic-1 | R^{a1}-7 | R^{a1}-7 | R^{a1}-7 | R^{a1}-7 | — | — |
| 120 | Ic-1 | R^{a1}-8 | R^{a1}-8 | R^{a1}-8 | R^{a1}-8 | — | — |
| 121 | Ic-1 | Ra1-9 | Ra1-9 | Ra1-9 | Ra1-g | — | — |
| 122 | Ic-1 | R^{a1}-10 | R^{a1}-10 | R^{a1}-10 | R^{a1}-10 | — | — |
| 123 | Ic-1 | R^{a1}-19 | R^{a1}-19 | R^{a1}-19 | R^{a1}-19 | — | — |
| 124 | Ic-1 | R^{a1}-20 | R^{a1}-20 | R^{a1}-20 | R^{a1}-20 | — | — |
| 125 | Ic-1 | R^{a1}-21 | R^{a1}-21 | R^{a1}-21 | R^{a1}-21 | — | — |
| 126 | Ic-1 | R^{a1}-25 | R^{a1}-25 | R^{a1}-25 | R^{a1}-25 | — | — |
| 127 | Ic-1 | R^{a1}-26 | R^{a1}-26 | R^{a1}-26 | R^{a1}-26 | — | — |
| 128 | Ic-1 | R^{a1}-27 | R^{a1}-27 | R^{a1}-27 | R^{a1}-27 | — | — |
| 129 | Ic-1 | R^{a1}-28 | R^{a1}-28 | R^{a1}-28 | R^{a1}-28 | — | — |
| 130 | Ic-1 | phenyl | phenyl | R^{a1}-1 | R^{a1}-1 | — | — |
| 131 | Ic-1 | phenyl | phenyl | R^{a1}-2 | R^{a1}-2 | — | — |
| 132 | Ic-1 | phenyl | phenyl | R^{a1}-3 | R^{a1}-3 | — | — |
| 133 | Ic-1 | phenyl | phenyl | R^{a1}-6 | R^{a1}-6 | — | — |
| 134 | Ic-1 | phenyl | phenyl | R^{a1}-7 | R^{a1}-7 | — | — |
| 135 | Ic-1 | phenyl | phenyl | R^{a1}-8 | R^{a1}-8 | — | — |
| 136 | Ic-1 | phenyl | phenyl | R^{a1}-9 | R^{a1}-9 | — | — |
| 137 | Ic-1 | phenyl | phenyl | R^{a1}-10 | R^{a1}-10 | — | — |
| 138 | Ic-1 | phenyl | phenyl | R^{a1}-19 | R^{a1}-19 | — | — |
| 139 | Ic-1 | phenyl | phenyl | R^{a1}-20 | R^{a1}-20 | — | — |
| 140 | Ic-1 | phenyl | phenyl | R^{a1}-21 | R^{a1}-21 | — | — |
| 141 | Ic-1 | phenyl | phenyl | R^{a1}-24 | R^{a1}-24 | — | — |
| 142 | Ic-1 | phenyl | phenyl | R^{a1}-25 | R^{a1}-25 | — | — |
| 143 | Ic-1 | phenyl | phenyl | R^{a1}-26 | R^{a1}-26 | — | — |
| 144 | Ic-1 | phenyl | phenyl | R^{a1}-27 | R^{a1}-27 | — | — |
| 145 | Ic-1 | phenyl | phenyl | R^{a1}-28 | R^{a1}-28 | — | — |
| 146 | Ic-2 | — | — | R^{a1}-1 | R^{a1}-1 | — | — |
| 147 | Ic-2 | — | — | R^{a1}-2 | R^{a1}-2 | — | — |
| 148 | Ic-2 | — | — | R^{a1}-3 | R^{a1}-3 | — | — |
| 149 | Ic-2 | — | — | R^{a1}-7 | R^{a1}-7 | — | — |
| 150 | Ic-2 | — | — | R^{a1}-8 | R^{a1}-8 | — | — |
| 151 | Ic-2 | — | — | R^{a1}-9 | R^{a1}-9 | — | — |
| 152 | Ic-2 | — | — | R^{a1}-10 | R^{a1}-10 | — | — |
| 153 | Ic-2 | — | — | R^{a1}-19 | R^{a1}-19 | — | — |
| 154 | Ic-2 | — | — | R^{a1}-20 | R^{a1}-20 | — | — |
| 155 | Ic-2 | — | — | R^{a1}-25 | R^{a1}-25 | — | — |
| 156 | Ic-2 | — | — | R^{a1}-26 | R^{a1}-26 | — | — |
| 157 | Ic-2 | — | — | R^{a1}-27 | R^{a1}-27 | — | — |
| 158 | Ic-2 | — | — | R^{a1}-28 | R^{a1}-28 | — | — |
| 159 | Id-1 | H | H | H | H | R^{a1}-1 | H |
| 160 | Id-1 | H | H | H | H | R^{a1}-2 | H |
| 161 | Id-1 | H | H | H | H | R^{a1}-3 | H |
| 162 | Id-1 | H | H | H | H | R^{a1}-6 | H |
| 163 | Id-1 | H | H | H | H | R^{a1}-7 | H |
| 164 | Id-1 | H | H | H | H | R^{a1}-8 | H |
| 165 | Id-1 | H | H | H | H | R^{a1}-9 | H |
| 166 | Id-1 | H | H | H | H | R^{a1}-10 | H |
| 167 | Id-1 | H | H | H | H | R^{a1}-19 | H |
| 168 | Id-1 | H | H | H | H | R^{a1}-20 | H |
| 169 | Id-1 | H | H | H | H | R^{a1}-21 | H |
| 170 | Id-1 | H | H | H | H | R^{a1}-25 | H |
| 171 | Id-1 | H | H | H | H | R^{a1}-26 | H |
| 172 | Id-1 | H | H | H | H | R^{a1}-27 | H |
| 173 | Id-1 | H | H | H | H | R^{a1}-28 | H |
| 174 | Id-1 | H | H | H | H | R^{a1}-1 | R^{a1}-1 |
| 175 | Id-1 | H | H | H | H | R^{a1}-2 | R^{a1}-2 |
| 176 | Id-1 | H | H | H | H | R^{a1}-3 | R^{a1}-3 |
| 177 | Id-1 | H | H | H | H | R^{a1}-6 | R^{a1}-6 |
| 178 | Id-1 | H | H | H | H | R^{a1}-7 | R^{a1}-7 |
| 179 | Id-1 | H | H | H | H | R^{a1}-8 | R^{a1}-8 |
| 180 | Id-1 | H | H | H | H | R^{a1}-9 | R^{a1}-9 |
| 181 | Id-1 | H | H | H | H | R^{a1}-10 | R^{a1}-10 |
| 182 | Id-1 | H | H | H | H | R^{a1}-19 | R^{a1}-19 |
| 183 | Id-1 | H | H | H | H | R^{a1}-20 | R^{a1}-20 |
| 184 | Id-1 | H | H | H | H | R^{a1}-21 | R^{a1}-21 |
| 185 | Id-1 | H | H | H | H | R^{a1}-24 | R^{a1}-24 |
| 186 | Id-1 | H | H | H | H | R^{a1}-25 | R^{a1}-25 |
| 187 | Id-1 | H | H | H | H | R^{a1}-26 | R^{a1}-26 |
| 188 | Id-1 | H | H | H | H | R^{a1}-27 | R^{a1}-27 |
| 189 | Id-1 | H | H | H | H | R^{a1}-28 | R^{a1}-28 |
| 190 | Id-1 | H | H | R^{a1}-1 | R^{a1}-1 | H | H |
| 191 | Id-1 | H | H | R^{a1}-2 | R^{a1}-2 | H | H |
| 192 | Id-1 | H | H | R^{a1}-3 | R^{a1}-3 | H | H |
| 193 | Id-1 | H | H | R^{a1}-6 | R^{a1}-6 | H | H |
| 194 | Id-1 | H | H | R^{a1}-7 | R^{a1}-7 | H | H |
| 195 | Id-1 | H | H | R^{a1}-8 | R^{a1}-8 | H | H |
| 196 | Id-1 | H | H | R^{a1}-9 | R^{a1}-9 | H | H |
| 197 | Id-1 | H | H | R^{a1}-10 | R^{a1}-10 | H | H |
| 198 | Id-1 | H | H | R^{a1}-19 | R^{a1}-19 | H | H |
| 199 | Id-1 | H | H | R^{a1}-20 | R^{a1}-20 | H | H |
| 200 | Id-1 | H | H | R^{a1}-21 | R^{a1}-21 | H | H |
| 201 | Id-1 | H | H | R^{a1}-25 | R^{a1}-25 | H | H |
| 202 | Id-1 | H | H | R^{a1}-26 | R^{a1}-26 | H | H |
| 203 | Id-1 | H | H | R^{a1}-27 | R^{a1}-27 | H | H |
| 204 | Id-1 | H | H | R^{a1}-28 | R^{a1}-28 | H | H |
| 205 | Id-1 | R^{a1}-1 | R^{a1}-1 | R^{a1}-1 | R^{a1}-1 | H | H |
| 206 | Id-1 | R^{a1}-7 | R^{a1}-7 | R^{a1}-7 | R^{a1}-7 | H | H |
| 207 | Id-1 | R^{a1}-8 | R^{a1}-8 | R^{a1}-8 | R^{a1}-8 | H | H |
| 208 | Id-1 | R^{a1}-g | R^{a1}-g | R^{a1}-g | R^{a1}-9 | H | H |
| 209 | Id-1 | R^{a1}-10 | R^{a1}-10 | R^{a1}-10 | R^{a1}-10 | H | H |
| 210 | Id-1 | R^{a1}-19 | R^{a1}-19 | R^{a1}-19 | R^{a1}-19 | H | H |
| 211 | Id-1 | R^{a1}-25 | R^{a1}-25 | R^{a1}-25 | R^{a1}-25 | H | H |
| 212 | Id-1 | R^{a1}-26 | R^{a1}-26 | R^{a1}-26 | R^{a1}-26 | H | H |
| 213 | Id-1 | R^{a1}-27 | R^{a1}-27 | R^{a1}-27 | R^{a1}-27 | H | H |
| 214 | Id-1 | R^{a1}-28 | R^{a1}-28 | R^{a1}-28 | R^{a1}-28 | H | H |
| 215 | Id-1 | phenyl | phenyl | R^{a1}-1 | R^{a1}-1 | H | H |
| 216 | Id-1 | phenyl | phenyl | R^{a1}-2 | R^{a1}-2 | H | H |
| 217 | Id-1 | phenyl | phenyl | R^{a1}-3 | R^{a1}-3 | H | H |
| 218 | Id-1 | phenyl | phenyl | R^{a1}-6 | R^{a1}-6 | H | H |
| 219 | Id-1 | phenyl | phenyl | R^{a1}-7 | R^{a1}-7 | H | H |
| 220 | Id-1 | phenyl | phenyl | R^{a1}-8 | R^{a1}-8 | H | H |
| 221 | Id-1 | phenyl | phenyl | R^{a1}-9 | R^{a1}-9 | H | H |
| 222 | Id-1 | phenyl | phenyl | R^{a1}-10 | R^{a1}-10 | H | H |
| 223 | Id-1 | phenyl | phenyl | R^{a1}-19 | R^{a1}-19 | H | H |
| 224 | Id-1 | phenyl | phenyl | R^{a1}-20 | R^{a1}-20 | H | H |
| 225 | Id-1 | phenyl | phenyl | R^{a1}-25 | R^{a1}-25 | H | H |
| 226 | Id-1 | phenyl | phenyl | R^{a1}-26 | R^{a1}-26 | H | H |
| 227 | Id-1 | phenyl | phenyl | R^{a1}-27 | R^{a1}-27 | H | H |
| 228 | Id-1 | phenyl | phenyl | R^{a1}-28 | R^{a1}-28 | H | H |
| 229 | Id-1 | phenyl | phenyl | H | H | R^{a1}-1 | Ra1-1 |
| 230 | Id-1 | phenyl | phenyl | H | H | R^{a1}-2 | R^{a1}-2 |
| 231 | Id-1 | phenyl | phenyl | H | H | R^{a1}-3 | R^{a1}-3 |
| 232 | Id-1 | phenyl | phenyl | H | H | R^{a1}-6 | R^{a1}-6 |
| 233 | Id-1 | phenyl | phenyl | H | H | R^{a1}-7 | R^{a1}-7 |
| 234 | Id-1 | phenyl | phenyl | H | H | R^{a1}-8 | R^{a1}-8 |
| 235 | Id-1 | phenyl | phenyl | H | H | R^{a1}-9 | R^{a1}-9 |
| 236 | Id-1 | phenyl | phenyl | H | H | R^{a1}-1 0 | R^{a1}-10 |
| 237 | Id-1 | phenyl | phenyl | H | H | R^{a1}-19 | R^{a1}-19 |
| 238 | Id-1 | phenyl | phenyl | H | H | R^{a1}-20 | R^{a1}-20 |
| 239 | Id-1 | phenyl | phenyl | H | H | R^{a1}-25 | R^{a1}-25 |
| 240 | Id-1 | phenyl | phenyl | H | H | R^{a1}-26 | R^{a1}-26 |
| 241 | Id-1 | phenyl | phenyl | H | H | R^{a1}-27 | R^{a1}-27 |
| 242 | Id-1 | phenyl | phenyl | H | H | R^{a1}-28 | R^{a1}-28 |
| 243 | Id-2 | H | H | H | H | R^{a1}-1 | H |
| 244 | Id-2 | H | H | H | H | R^{a1}-2 | H |
| 245 | Id-2 | H | H | H | H | R^{a1}-3 | H |
| 246 | Id-2 | H | H | H | H | R^{a1}-6 | H |
| 247 | Id-2 | H | H | H | H | R^{a1}-7 | H |
| 248 | Id-2 | H | H | H | H | R^{a1}-8 | H |
| 249 | Id-2 | H | H | H | H | R^{a1}-9 | H |
| 250 | Id-2 | H | H | H | H | R^{a1}-10 | H |
| 251 | Id-2 | H | H | H | H | R^{a1}-19 | H |
| 252 | Id-2 | H | H | H | H | R^{a1}-20 | H |
| 253 | Id-2 | H | H | H | H | R^{a1}-21 | H |
| 254 | Id-2 | H | H | H | H | R^{a1}-24 | H |
| 255 | Id-2 | H | H | H | H | R^{a1}-25 | H |
| 256 | Id-2 | H | H | H | H | R^{a1}-26 | H |
| 257 | Id-2 | H | H | H | H | R^{a1}-27 | H |
| 258 | Id-2 | H | H | H | H | R^{a1}-28 | H |
| 259 | Id-2 | H | H | H | H | R^{a1}-1 | R^{a1}-1 |
| 260 | Id-2 | H | H | H | H | R^{a1}-2 | R^{a1}-2 |
| 261 | Id-2 | H | H | H | H | R^{a1}-3 | R^{a1}-3 |
| 262 | Id-2 | H | H | H | H | R^{a1}-7 | R^{a1}-7 |
| 263 | Id-2 | H | H | H | H | R^{a1}-8 | R^{a1}-8 |
| 264 | Id-2 | H | H | H | H | R^{a1}-9 | R^{a1}-9 |
| 265 | Id-2 | H | H | H | H | R^{a1}-10 | R^{a1}-10 |
| 266 | Id-2 | H | H | H | H | R^{a1}-19 | R^{a1}-19 |
| 267 | Id-2 | H | H | H | H | R^{a1}-20 | R^{a1}-20 |
| 268 | Id-2 | H | H | H | H | R^{a1}-25 | R^{a1}-25 |
| 269 | Id-2 | H | H | H | H | R^{a1}-26 | R^{a1}-26 |
| 270 | Id-2 | H | H | H | H | R^{a1}-27 | R^{a1}-27 |
| 271 | Id-2 | H | H | H | H | R^{a1}-28 | R^{a1}-28 |
| 272 | Id-3 | — | — | H | H | R^{a1}-1 | H |
| 273 | Id-3 | — | — | H | H | R^{a1}-2 | H |
| 274 | Id-3 | — | — | H | H | R^{a1}-3 | H |
| 275 | Id-3 | — | — | H | H | R^{a1}-6 | H |
| 276 | Id-3 | — | — | H | H | R^{a1}-7 | H |
| 277 | Id-3 | — | — | H | H | R^{a1}-8 | H |
| 278 | Id-3 | — | — | H | H | R^{a1}-9 | H |
| 279 | Id-3 | — | — | H | H | R^{a1}-10 | H |
| 280 | Id-3 | — | — | H | H | R^{a1}-19 | H |
| 281 | Id-3 | — | — | H | H | R^{a1}-20 | H |
| 282 | Id-3 | — | — | H | H | R^{a1}-25 | H |
| 283 | Id-3 | — | — | H | H | R^{a1}-26 | H |
| 284 | Id-3 | — | — | H | H | R^{a1}-27 | H |
| 285 | Id-3 | — | — | H | H | R^{a1}-28 | H |
| 286 | Id-3 | — | — | H | H | R^{a1}-1 | R^{a1}-1 |
| 287 | Id-3 | — | — | H | H | R^{a1}-2 | R^{a1}-2 |
| 288 | Id-3 | — | — | H | H | R^{a1}-3 | R^{a1}-3 |
| 289 | Id-3 | — | — | H | H | R^{a1}-6 | R^{a1}-6 |
| 290 | Id-3 | — | — | H | H | R^{a1}-7 | R^{a1}-7 |
| 291 | Id-3 | — | — | H | H | R^{a1}-8 | R^{a1}-8 |
| 292 | Id-3 | — | — | H | H | R^{a1}-9 | R^{a1}-9 |
| 293 | Id-3 | — | — | H | H | R^{a1}-10 | R^{a1}-10 |
| 294 | Id-3 | — | — | H | H | R^{a1}-19 | R^{a1}-19 |
| 295 | Id-3 | — | — | H | H | R^{a1}-20 | R^{a1}-20 |
| 296 | Id-3 | — | — | H | H | R^{a1}-21 | R^{a1}-21 |
| 297 | Id-3 | — | — | H | H | R^{a1}-24 | R^{a1}-24 |
| 298 | Id-3 | — | — | H | H | R^{a1}-25 | R^{a1}-25 |
| 299 | Id-3 | — | — | H | H | R^{a1}-26 | R^{a1}-26 |
| 300 | Id-3 | — | — | H | H | R^{a1}-27 | R^{a1}-27 |
| 301 | Id-3 | — | — | H | H | R^{a1}-28 | R^{a1}-28 |
| 302 | Id-3 | — | — | R^{a1}-1 | R^{a1}-1 | H | H |
| 303 | Id-3 | — | — | R^{a1}-2 | R^{a1}-2 | H | H |
| 304 | Id-3 | — | — | R^{a1}-3 | R^{a1}-3 | H | H |
| 305 | Id-3 | — | — | R^{a1}-6 | R^{a1}-6 | H | H |
| 306 | Id-3 | — | — | R^{a1}-7 | R^{a1}-7 | H | H |
| 307 | Id-3 | — | — | R^{a1}-8 | R^{a1}-8 | H | H |
| 308 | Id-3 | — | — | R^{a1}-9 | R^{a1}-9 | H | H |
| 309 | Id-3 | — | — | R^{a1}-10 | R^{a1}-10 | H | H |
| 310 | Id-3 | — | — | R^{a1}-19 | R^{a1}-19 | H | H |
| 311 | Id-3 | — | — | R^{a1}-20 | R^{a1}-20 | H | H |
| 312 | Id-3 | — | — | R^{a1}-21 | R^{a1}-21 | H | H |
| 313 | Id-3 | — | — | R^{a1}-24 | R^{a1}-24 | H | H |
| 314 | Id-3 | — | — | R^{a1}-25 | R^{a1}-25 | H | H |
| 315 | Id-3 | — | — | R^{a1}-26 | R^{a1}-26 | H | H |
| 316 | Id-3 | — | — | R^{a1}-27 | R^{a1}-27 | H | H |
| 317 | Id-3 | — | — | R^{a1}-28 | R^{a1}-28 | H | H |
| 318 | Id-3 | — | — | R^{a1}-1 | R^{a1}-1 | R^{a1}-1 | R^{a1}-1 |
| 319 | Id-3 | — | — | R^{a1}-3 | R^{a1}-3 | R^{a1}-3 | R^{a1}-3 |
| 320 | Id-3 | — | — | R^{a1}-7 | R^{a1}-7 | R^{a1}-7 | R^{a1}-7 |
| 321 | Id-3 | — | — | R^{a1}-8 | R^{a1}-8 | R^{a1}-8 | R^{a1}-8 |
| 322 | Id-3 | — | — | R^{a1}-9 | R^{a1}-9 | R^{a1}-9 | R^{a1}-9 |
| 323 | Id-3 | — | — | R^{a1}-10 | R^{a1}-10 | R^{a1}-10 | R^{a1}-10 |
| 324 | Id-3 | — | — | R^{a1}-19 | R^{a1}-19 | R^{a1}-19 | R^{a1}-19 |
| 325 | Id-3 | — | — | R^{a1}-25 | R^{a1}-25 | R^{a1}-25 | R^{a1}-25 |
| 326 | Id-3 | — | — | R^{a1}-26 | R^{a1}-26 | R^{a1}-26 | R^{a1}-26 |
| 327 | Id-3 | — | — | R^{a1}-27 | R^{a1}-27 | R^{a1}-27 | R^{a1}-27 |
| 328 | Id-3 | — | — | R^{a1}-28 | R^{a1}-28 | R^{a1}-28 | R^{a1}-28 |
| 329 | Id-4 | — | — | H | H | R^{a1}-1 | H |
| 330 | Id-4 | — | — | H | H | R^{a1}-2 | H |
| 331 | Id-4 | — | — | H | H | R^{a1}-3 | H |
| 332 | Id-4 | — | — | H | H | R^{a1}-6 | H |
| 333 | Id-4 | — | — | H | H | R^{a1}-7 | H |
| 334 | Id-4 | — | — | H | H | R^{a1}-8 | H |
| 335 | Id-4 | — | — | H | H | R^{a1}-9 | H |
| 336 | Id-4 | — | — | H | H | R^{a1}-10 | H |
| 337 | Id-4 | — | — | H | H | R^{a1}-19 | H |
| 338 | Id-4 | — | — | H | H | R^{a1}-20 | H |
| 339 | Id-4 | — | — | H | H | R^{a1}-21 | H |
| 340 | Id-4 | — | — | H | H | R^{a1}-24 | H |
| 341 | Id-4 | — | — | H | H | R^{a1}-25 | H |
| 342 | Id-4 | — | — | H | H | R^{a1}-26 | H |
| 343 | Id-4 | — | — | H | H | R^{a1}-27 | H |
| 344 | Id-4 | — | — | H | H | R^{a1}-28 | H |
| 345 | Id-4 | — | — | H | H | R^{a1}-1 | R^{a1}-1 |
| 346 | Id-4 | — | — | H | H | R^{a1}-2 | R^{a1}-2 |
| 347 | Id-4 | — | — | H | H | R^{a1}-3 | Ra1-3 |
| 348 | Id-4 | — | — | H | H | R^{a1}-6 | R^{a1}-6 |
| 349 | Id-4 | — | — | H | H | R^{a1}-7 | Ra1-7 |
| 350 | Id-4 | — | — | H | H | R^{a1}-8 | R^{a1}-8 |
| 351 | Id-4 | — | — | H | H | R^{a1}-9 | R^{a1}-9 |
| 352 | Id-4 | — | — | H | H | R^{a1}-10 | R^{a1}-10 |
| 353 | Id-4 | — | — | H | H | R^{a1}-19 | R^{a1}-19 |
| 354 | Id-4 | — | — | H | H | R^{a1}-20 | R^{a1}-20 |
| 355 | Id-4 | — | — | H | H | R^{a1}-21 | R^{a1}-21 |
| 356 | Id-4 | — | — | H | H | R^{a1}-24 | R^{a1}-24 |
| 357 | Id-4 | — | — | H | H | R^{a1}-25 | R^{a1}-25 |
| 358 | Id-4 | — | — | H | H | R^{a1}-26 | R^{a1}-26 |
| 359 | Id-4 | — | — | H | H | R^{a1}-27 | R^{a1}-27 |
| 360 | Id-4 | — | — | H | H | R^{a1}-28 | R^{a1}-28 |
| 361 | Id-4 | — | — | R^{a1}-1 | R^{a1}-1 | R^{a1}-1 | R^{a1}-1 |
| 362 | Id-4 | — | — | R^{a1}-2 | R^{a1}-2 | R^{a1}-2 | R^{a1}-2 |
| 363 | Id-4 | — | — | R^{a1}-3 | R^{a1}-3 | R^{a1}-3 | R^{a1}-3 |
| 364 | Id-4 | — | — | R^{a1}-7 | R^{a1}-7 | R^{a1}-7 | R^{a1}-7 |
| 365 | Id-4 | — | — | R^{a1}-8 | R^{a1}-8 | R^{a1}-8 | R^{a1}-8 |
| 366 | Id-4 | — | — | R^{a1}-9 | R^{a1}-9 | R^{a1}-9 | R^{a1}-9 |
| 367 | Id-4 | — | — | R^{a1}-10 | R^{a1}-10 | R^{a1}-10 | R^{a1}-10 |
| 368 | Id-4 | — | — | R^{a1}-19 | R^{a1}-19 | R^{a1}-19 | R^{a1}-19 |
| 369 | Id-4 | — | — | R^{a1}-20 | R^{a1}-20 | R^{a1}-20 | R^{a1}-20 |
| 370 | Id-4 | — | — | R^{a1}-25 | R^{a1}-25 | R^{a1}-25 | R^{a1}-25 |
| 371 | Id-4 | — | — | R^{a1}-26 | R^{a1}-26 | R^{a1}-26 | R^{a1}-26 |
| 372 | Id-4 | — | — | R^{a1}-27 | R^{a1}-27 | R^{a1}-27 | R^{a1}-27 |
| 373 | Id-4 | — | — | R^{a1}-28 | R^{a1}-28 | R^{a1}-28 | R^{a1}-28 |

where R^{a1}-1, R^{a1}-2, R^{a1}-3, R^{a1}-6, R^{a1}-7, R^{a1}-8, R^{a1}-9, R^{a1}-10, R^{a1}-19, R^{a1}-20, R^{a1}-21, R^{a1}-24, R^{a1}-25, R^{a1}-26, R^{a1}-27 and R^{a1}-28 are as defined in claim 6.

17. A thermoplastic resin comprising a structural unit represented by formulae (II) below 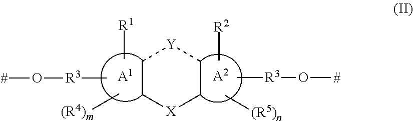
where
#represents a connection point to a neighboring structural unit;
and where A¹, A², n, m, R¹, R², R³, R⁴, R⁵, X and Y are as defined in any one of claims 1 to 4.

18. The thermoplastic resin of claim 17, where X represents a single bond and Y is absent.

19. The thermoplastic resin of claim 17, where formula (II) is represented by formula (IIa): 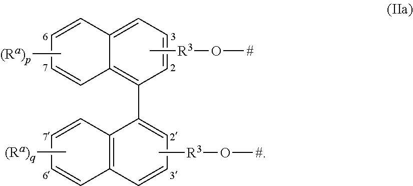
where q is 1 or 2 and p is 1 or 2.

20. The thermoplastic resin of claim 17, where formula (II) is represented by formula (IIa-1): 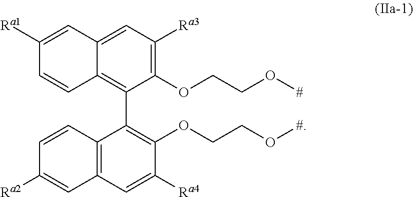
where
R^{a1} is hydrogen or R^{a},
R^{a2} is hydrogen or R^{a},
R^{a3} is hydrogen or R^{a} and
R^{a4} is hydrogen or R^{a},
provided that at least two of R^{a1}, R^{a2}, R^{a3} and R^{a4} are R^{a}.

21. The thermoplastic resin of claim 20, where R^{a1}, R^{a2}, R^{a3} and R^{a4} in formula (IIa-1) are as defined in the table A recited in claim 8.

22. The thermoplastic resin of claim 20, where R^{a1} and R^{a2} are identical and selected from the group consisting of phenylethynyl, naphthalene-1-ylethynyl and 2-naphthalene-2-ylethynyl and where R^{a3} and R^{a4} in formula (IIa-1) are hydrogen.

23. The thermoplastic resin of any one of claims 17 to 22, where the structural unit of the formula (II) is connected to one of the structures represented by formulae (III-1) to (III-5) below, 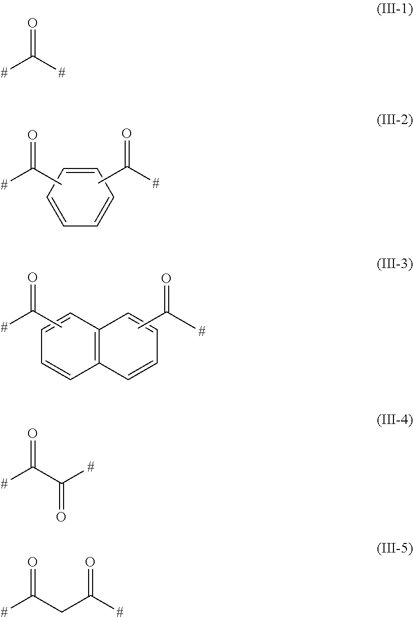
where
#represents a connection point to a neighboring structural unit.

24. The thermoplastic resin of any one of claims 17 to 23, which selected from copolycarbonate resins, copolyestercarbonate resins and copolyester resins, where the thermoplastic resin in addition to structural units represented by formula (II) comprises a structural unit of the formula (V),
#—O—R^{z}-A³-R^{z}—O—#- (V)
where
#represents a connection point to a neighboring structural unit;
A³ is a polycyclic radical bearing at least 2 benzene rings, wherein the benzene rings may be connected by A′ and/or directly fused to each other and/or fused by a non-benzene carbocycle, where A³ is unsubstituted or substituted by 1, 2 or 3 radicals R^{aa}, which are selected from the group consisting of halogen, C₁-C₆-alkyl, C₅-C₆-cycloalkyl and phenyl;
A′ is selected from the group consisting of a single bond, O, C═O, S, SO₂, CH₂, CH—Ar″, CAr″₂, CH(CH₃), C(CH₃)₂ and a radical of the formula (A″) 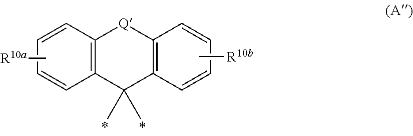
where
Q′ represents a single bond, O, NH, C═O, CH₂ or CH═CH; and
R^{10a}, R^{10b}, independently of each other are selected from the group consisting of hydrogen, fluorine, CN, R, OR, CHₖR₃₋ₖ, NR₂, C(O)R and C(O)NH₂;
Ar″ is selected from the group consisting of mono- or polycyclic aryl having from 6 to 26 carbon atoms and mono- or polycyclic hetaryl having a total of 5 to 26 atoms, which are ring members, where 1, 2, 3 or 4 of these atoms are selected from nitrogen, sulphur and oxygen, while the remainder of these atoms are carbon atoms, where Ar″ is unsubstituted or substituted by 1, 2 or 3 radicals R^{ab}, which are selected from the group consisting of halogen, phenyl and C₁-C₄-alkyl;
R^{z} is a single bond, Alk¹, O-Alk²-, O-Alk²-[O-Alk²-]ₚ- or O-Alk³-C(O)— where O is bound to A³, and where
p is an integer from 1 to 10;
Alk¹ is C₁-C₄-alkandiyl;
Alk² is C₂-C₄-alkandiyl; and
Alk³ is C₁-C₄-alkandiyl.

25. The thermoplastic resin of claim 24, where the structural unit of the formula V is represented by one of the following formulae V-1 to V-6: 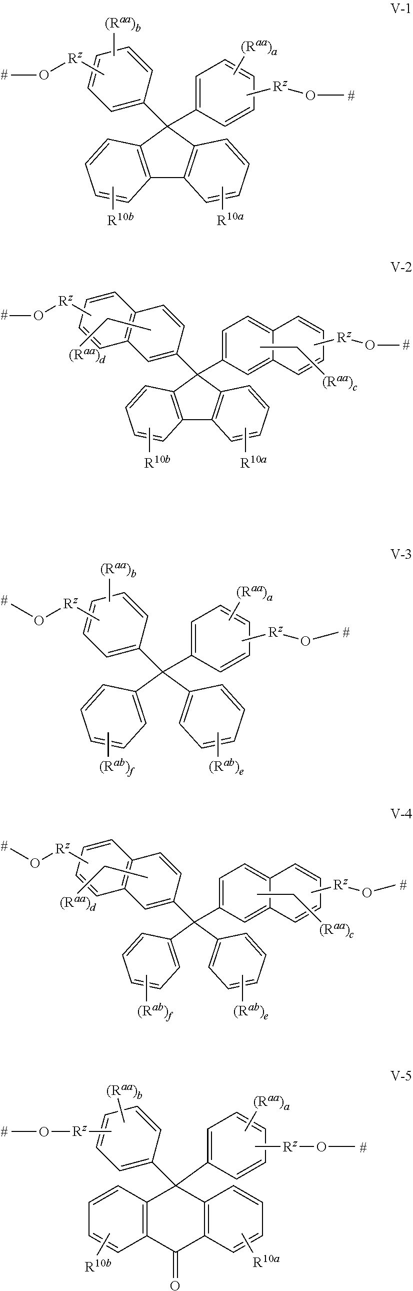 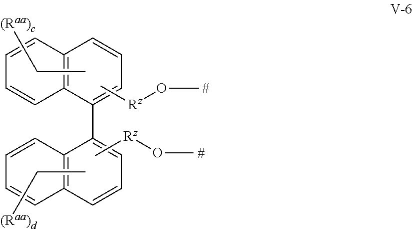
where
a and b are 0, 1, 2 or 3, in particular 0 or 1;
c and d are 0, 1, 2, 3, 4 or 5, in particular 0 or 1;
e and f are 0, 1, 2, 3, 4 or 5, in particular 0 or 1;
and where R^{z}, R^{aa}, R^{ab}, R^{10a} and R^{10b} are as defined for formula (IV).

26. The thermoplastic resin of claim 24, where the structural unit of the formula V is represented by one of the following formulae V-11 to V-18:

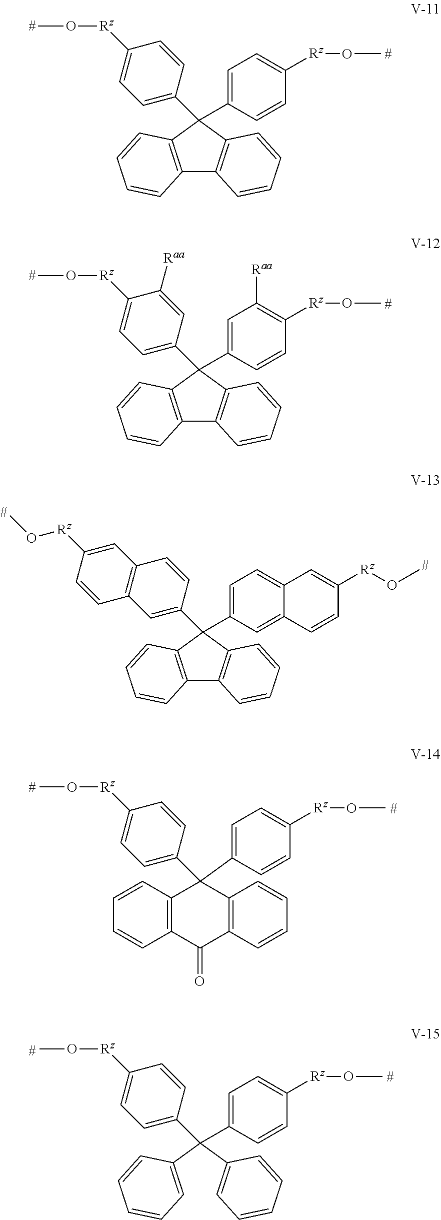

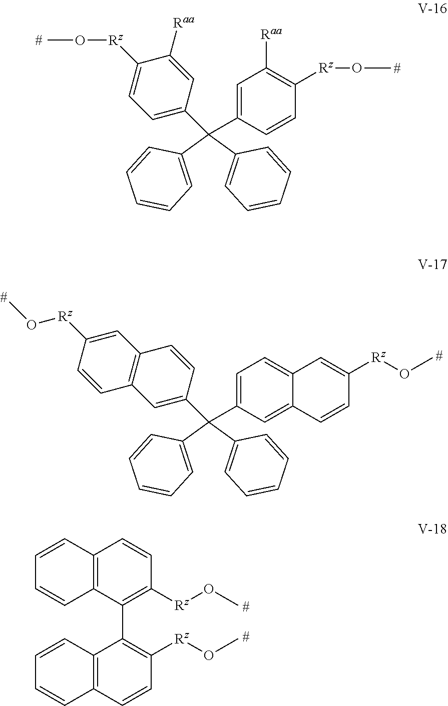

27. The thermoplastic resin of claim 26, comprising at least one structural unit of the formula (IIa-1) as recited in claim 20 and at least one structural unit selected from the group consisting of structural units of the formula (V-12), structural units of the formula (V-13) and structural units of the formula (V-18).

28. The thermoplastic resin of claim 27, where in the structural unit of the formula (IIa-1) R^{a1} and R^{a2} are identical and selected from the group consisting of phenylethynyl, naphthalene-1-ylethynyl and 2-naphthalene-2-ylethynyl and where R^{a3} and R^{a4} in formula (IIa-1) are hydrogen and where in the structural units of the formulae (V-12), (V-13) and (V-18) the radicals R^{z} are O—CH₂CH₂.

29. The thermoplastic resin of any one of claims 24 to 28, where the molar ratio of the structural units of the formula (II) is from 1 to 70 mol-% based on the total molar amount of structural units of the formulae (II) and (V).

30. The thermoplastic resin of any one of claims 24 to 29, where the molar ratio of the structural units of the formula (V) is from 30 to 99 mol-% based on the total molar amount of structural units of the formulae (II) and (V).

31. An optical device made of a thermoplastic resin as defined in any one of claims 17 to 30.

32. Use of the compound as defined in any one of claims 1 to 16, as a monomer of the thermoplastic resin as defined in any one of claims 17 to 30.
